SEQUENCE LISTING (279 sequences)

SEQ ID NO: 1 - DNA, 4659 bp, Diabrotica virgifera
agaattcgcaagagcatagttttgtttgagtgttcgtaaatacagtcgtagaataaggaatatcttgaattggggcctacttagcaaggtcatgcttacttgattgttacctaaggaaatctcaaagcaatcatgagaaggagggaattatatgatgacgaactcgaaccccttgtggagaatactcaaacacaaccgaaatatcaatgggatctatttaataatgtacctagagagacaatatctggatccacaattgaatctaagtacattgatttatctgtaaaatatctaaaaattttcacagttatatttacaatgttaatagtcttagggtgcgccgtagtgtcaaaagcaagtttactatttatgacatctcaaataaagccaaatattactaggttatattgtaatgaaaacttggatacacgacaacaatatattgtgatccacccacaagaagaaagaacagtatggatttggataataatattttcgtatatggtaccagaactaggaacatttatcagatcggtcagaataataagtttcaaaagttggaattatcccactaagctggactttttgattttggcaacaacagaaattttgccagtcatcgggagcgctcttcttgtatttatagtttttcctgaaattgatgtaattaaagcagccatgttaaccaatggcgtttgcttggtaccaggaattgtcgcaatgatatcaagatcaacatcgaaagttcgccaaggactatgttatggactggatatagcagccatagttattcaagcaagtgctttagttgtgtggcctctggtagaaaacaggaaaatattgtatatcattcctatagctctcagtctaatatctgttggatggtgggaaaattttctctcggagaaaactccaattccctgggtaaaaaaatttgcaaaggcaaaaaaacaatttaaaacagatgtttattttacatatgccttaatcacccccatcaaatgtttgatcttccttttatctgcagtaacaattatatggataagagaaggagatgttggcttcttgttcgataaatttggagatgcatttagtagtcactcgtttaatgttacacagatcgaaccagtggtaggaacatccaacattaattacacagatgcagtagcagatatacatcctactacaatggaaacgtcatattggacaccaatagttacttggctaataaatattctcagtacatatatttgctatgcttttggaaaatttgcatgtaaaattatgattcagtccatgagcttcgcactacctgtaaatctagctgttcccgttcttttaacagctttggtgtccatgtgcggtatgtataacaagaatgaatgcgcttacaccaacgtttttcctgcttacttattcttcaatactccaaccatgtcgcgacttgaagactttattggccatcaatattcctggatatggtttttatggttgctttctcaaacatggataactattcatatttggtctaacgatcacgaaaaactgatgtcgacagaacaactgttaaagaaaccgatgtacgatgcctttttaattgaccaatcgcttgctatgaatagacgaagagaacatgacagacttattaaaattggaagagaacagactgaaaacgaaaatgtacctcatgataaaatcacaaggatatatgcatgcgcgacgatgtggcatgagactaaagaagaaatggttgaatttttaaaatctgtctttagattggatgaagatcaatgtgctcatagaattgtccgacagtatttacagttccaactgcctgattactacgagttagaaactcatatattcttcgatgacgcctttattcgacagtcacaaaatgataatgatccgcatgttaatgaatatgttctgtctttaattgatactgtcagtgatgcagctagtaaagtacatgctgtgaattgtaaaattaaaccgcccactgtgtactcaactccatatgggggcaggttagaatggacattaccgggtaaaactaagatgatagctcatttaaaagataaggccaaaataagggctaagaaaagatggtcacaggtcatgtatatgtattatttattaggatttaggattatggacaatgataagctccacccaacagaaattaaaaacatggcccacaatacatatatacttgctttagacggagatatcgattttcaacccgctgccgtacatctcttggtggattacatgaagaaaaactccagtttgggagctgcttgtggtagaattcatccagtcggttcaggtgcaatggcttggtatcaggtgtttgaatatgcggttggtcattggcttcaaaaggcaaccgaacacgtcattggttgtgtactttgtagtcctggttgtttttctttattcagagctggagcccttatggatgacaatgtaatggcaaaatataccacagaatcttcagaagcacgacactatgtacaatatgatcaaggtgaagatcggtggctgtgtactttattattgcagagaggttaccgtgtagaatattcagcagcttctgatgcctacacccattgtccagaaggttttagtgaattttataatcagcgaagaagatggatgccttccactaccgcaaatattatggatctactgacagattacaaacatattgtttcaatcaacgacaacatatccaaattgtacattttatatcaagtagtcattatgattggtacagtgattggtcccggaactatttttcttatgttagttggggcttttgtggctgcttttaaaatttcacaatacgagagcttgatttggaatgctataccagttctcatctttatagtagtatgtgccacgtgtaaatcagatacgcaattattctttgctgctcttatcactggagtatatggtcttgtcatgatgacagtattcgtcggtatgatgatccagatcgaacaagatgggtggctggctccttcatcccttttcttagtggccacgatgggagaatttttgatagccgccttgatgcatccccaagaattctactgcctcaaatatttaattatttactacgtaactattcccagcatgtatatgttgttggtaatatattcaatatttaacatgaataacgtatcatggggtacccgcgaggtcactgtagcacccaaaccgcaagaggcaactcaggaagctacaccaaatgcagcacctgctcctgcgcccagagcaaaccaaccattatcattttttggtaactccaaagataatgcgggatccttcgagttttctttcggaggactttttaaattattgtgttgtacctacgatagtaaaggcgaggaaagggaagtactcaggaacatacaaacttccattcaaaatatgcaaaaaaaattggaccagatggagaagaataaactaggagctagagattcgatcctggaaccaagaaaaaccgctagacaaactcagctttttgtgggtttacaagctgcaagagtttcacaagcagcaattcctctacaaacgcaggagttcgctgatgaagatggcgatgctgatgacgacgatgatgaggatgattcttcaaatatcactccttcggaagacgttgagcccaatagctggtactacgacggtagtttgattaaaagcggggtcgaattcattgataagaaagaaaaaaaattttgggagaaacttattgaaaaatatctgtctccattagatgaaactcataaaaaggacgctgtcgctaaagatttaaaggacttaagagacagaatggtgatgacattttttatgttgaatgcgctatttgtgttgataatatatcttctgaacttacaacaggatataattcacgttaactggcccattaaccctaaagtaaatttcacttatatcaccgacgaaaatatgataataattgagaagacttatttgcaactacaacctattggatttgtattccttgttatgttctctgcgctattgctagttcaatttattggtatgggtattcatcgtttcggtacctactgtcaaattatggccaatactcatatagatttcagcctttttggatcagaagttaaaaatattacagaaaaatctctgctggaaagagatcccataaagatctttaagcaactcatcagattgcaaggcataaatgacgaggatgataaagaggatacctcagtaataaggagggaaacagctcattggttagctttaaaaagagaaaagaaggctcagccagtaatagaagacttagaacaggccttctataaaaggctgagtctatttaacagcggaaaatataaagagcaaaataatccgtttagagacacaatgcctcaaatagcccacatacgaaataccatattgatgaggcactcccgtcttccagaattcacaggtggagtatcaagaaattacgaaagaccagatagtgcaatatttcttgacaaccccgctgaaaatgaagctgaaggttaaatatttagtaaaacttttaaaatgtaattattattgtctttgtattatattattattttatatttaataaaaatttttaaatcttaaaaaaaaaaa

SEQ ID NO: 2 - protein, 150 aa, Diabrotica virgifera
MADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGFISAAELRHVMTNLGEKLTDEEVDEMIREADIDGDGQVNYEEFIRSHLLAN

SEQ ID NO: 3 - DNA, 406 bp, Diabrotica virgifera
cgtagtgtcaaaagcaagtttactatttatgacatctcaaataaagccaaatattactaggttatattgtaatgaaaacttggatacacgacaacaatatattgtgatccacccacaagaagaaagaacagtatggatttggataataatattttcgtatatggtaccagaactaggaacatttatcagatcggtcagaataataagtttcaaaagttggaattatcccactaagctggactttttgattttggcaacaacagaaattttgccagtcatcgggagcgctcttcttgtatttatagtttttcctgaaattgatgtaattaaagcagccatgttaaccaatggcgtttgcttggtaccaggaattgtcgcaatgatatcaagatcaacataagttcgc

SEQ ID NO: 4 - DNA, 1048 bp, Diabrotica virgifera
gttgtgtggcctctggtagaaaacaggaaaatattgtatatcattcctatagctctcagtctaatatctgttggatggtgggaaaattttctctcggagaaaactccaattccctgggtaaaaaaatttgcaaaggcaaaaaaacaatttaaaacagatgtttattttacatatgccttaatcacccccatcaaatgtttgatcttccttttatctgcagtaacaattatatggataagagaaggagatgttggcttcttgttcgataaatttggagatgcatttagtagtcactcgtttaatgttacacagatcgaaccagtggtaggaacatccaacattaattacacagatgcagtagcagatatacatcctactacaatggaaacgtcatattggacaccaatagttacttggctaataaatattctcagtacatatatttgctatgcttttggaaaatttgcatgtaaaattatgattcagtccatgagcttcgcactacctgtaaatctagctgttcccgttcttttaacagctttggtgtccatgtgcggtatgtataacaagaatgaatgcgcttacaccaacgtttttcctgcttacttattcttcaatactccaaccatgtcgcgacttgaagactttattggccatcaatattcctggatatggtttttatggttgctttctcaaacatggataactattcatatttggtctaacgatcacgaaaaactgatgtcgacagaacaactgttaaagaaaccgatgtacgatgcctttttaattgaccaatcgcttgctatgaatagacgaagagaacatgacagacttattaaaattggaagagaacagactgaaaacgaaaatgtacctcatgatgataaaatcacaaggatatatgcatgcgcgacgatgtggcatgagactaaagaagaaatggttgaatttttaaaatctgtctttagattggatgaagatcaatgtgccatagaattgtccgacagtatttacagttccaactgcctgattactacgagttaga

SEQ ID NO: 5 - DNA, 506 bp, Diabrotica virgifera
tcttgtcatgatgacagtattcgtcggtatgatgatccagatcgaacaagatgggtggatggctccttcatcccttttcttagtggccacgatgggagaatttttgatagccgccttgatgcatccccaagaattctactgcctcaaatatttaattatttactacgtaactattcccagcatgtatatgttgttggtaatatattcaatatttaacatgaataacgtatcatggggtacccgcgaggtcactgtagcacccaaaccgcaagaggcaactcaggaagctacaccaaatgcagcacctgctcctgcgcccagagcaaaccaaccattatcattttttggtaactccaaagataatgcgggatccttcgagttttctttcggaggactttttaaattattgtgttgtacctacgatagtaaaggcgaggaaagggaagtactcagctacatacaaacttccattcaaaaatatgcaaaaaaaaattaggaccagatgg

SEQ ID NO: 6 - DNA, 585 bp, Diabrotica virgifera
cgctgtcgctaaagatttaaaggacttaagagacagaatggtgatgacattttttatgttgaatgcgctatttgtgttgataatatatcttctgaacttacaacaggatataattcacgttaactggcccattaaccctaaagtaaatttcacttatatcaccgacgaaaatatgataataattgagaagacttatttgcaactacaacctattggatttgtattccttgttatgttctctgcgctattgctagttcaatttattggtatgggtattcatcgtttcggtacctactgtcaaattatggccaatactcatatagatttcagcctttttggatcagaagttaaaaatattacagaaaaatctctgctggaaagagatcccataaagatctttaagcaactcatcagattgcaaggcataaatgacgaggatgataaagaggatacctcagtaataaggagggaaacagctcattggttagctttaaaaagagaaaagaaggctcagccagtaatagaagacttagaacaggccttctataaaaggctgagtctatttaacagcggaaaataataaag

SEQ ID NO: 7 - DNA, 935 bp, Artificial Sequence
  hairpin forming sequence
ctaagttgtgtggcctctggtagaaaacaggaaaatattgtatatcattcctatagctctcagtctaatatctgttggatggtgggaaaattttctctcggagaaaactccaattccctgggtaaaaaaatttgcaaaggcaaaaaaacaatttaaaacagatgtttattttacatatgccttaatcacccccatcaaatgtttgatcttccttttatctgcagtaacaattatatggataagagaaggagatgttggcttcttgttcgataaatttggagatgcatttagtagtcactcgtttaatgttacacagatcgaaccagtggtaggaacatccaacattaattacacgactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttagtgtaattaatgttggatgttcctaccactggttcgatctgtgtaacattaaacgagtgactactaaatgcatctccaaatttatcgaacaagaagccaacatctccttctcttatccatataattgttactgcagataaaaggaagatcaaacatttgatgggggtgattaaggcatatgtaaaataaacatctgttttaaattgtttttttgcctttgcaaatttttttacccagggaattggagttttctccgagagaaaattttcccaccatccaacagatattagactgagagctataggaatgatatacaatattttcctgttttctaccagaggccacacaacagagct

SEQ ID NO: 8 - DNA, 4239 bp, Diabrotica virgifera
gatttttaacgaacagttgtgttgttaggttatttaatgtctaaacttccaattctcgtgattttcaacaagagaagaaacagaaacagaaaataatacaaatgaacagatgagtcagtcagaaatacttttattttaaataggtaacctatttttaagatctttatttataggatctctagaacgaacgagaaaactctgccggaaaagttatgatataaaagcatgtgagtttaagtctcggttaagaaagtagtgaattaaattgatataaaatttcaatagaaatggaaaatgaaattcaagctgaagtttttaaagaacaaggaaatactgcatttaagaatggtgattgggaccaagcaataaacctttactcaaaagctattaacctctctaaagaagatactagaattatatctgtttattataaaaatcgggctgctgcatatttgaagcaagaaaactatgaagcagcactacaagactgtgataagtgtttacaaattgttccaactgatcctaaagcactctttagaagatgtcaagctctggaagccctaaaaagatacgaagaagcatataaggatgcaacacaaatattcaaagatgatcccacaaataaacccatccaacctgttttggagagactgtacaaaatagtgcaagagagagcaaaacagaatgaaaaaactagtagtaaattagaaagtatgacaaaaattgcctttgatttgaaagagactacagagaaacgggagacttcaatgaacaatcttttagtattagcaaggcagcatgctggagctgaattgatggttaattctccaattcttcaggagattaagaaattgttaaaggttgaaaaaaacaaagttgttgttacttctgctattagaataataggagaactatgtaaacacagtgagactcgtactaaaaaagttatagaaactatgggagttccttggtttcttgaaatacttgattctgaagatgtaaaacaagtgtctgccgcagagcactgtatgcaatctattataaacgcattctctggcatggaaaatcgaccagacactaaacctaaacaggaattgatagaaagtaacaagaaacaaatagatacattattgacttgcttagtatattctgttaataacagagtgatttcaggactagctagggatgcattgatagaactcattatgaggaatattcactatgatcagctctgttgggccaaagagctagtagatataggcggtgtagaaagattaatggaatgtgctagtgaattggaggagtacaagtatgagagtgctatgaacatcacaccttctacaagaacaatagctgcagtttgtttagctagggtctatgaaaatatgtactatgatactctaagggagaaatttatgggaaaaattgagaacttcattaaagacaaattgttaacaccagatgttgaatccaaagtgagagtgactgttgcattaacatctctattaaggggtcctctagaagtagggaatgctcttataggcaaagaaggcatcatggaaatgattctggtaatggccaacacagaagatgaactccaacaaaaagttgcctgtgaatgtattattgcagcagctagtaaagcagataaggccaaaactattatcaatcaaggcattaatattttgaaacacctttataaatcaaagaatgaccacatcagaattagagctttggtgggactgtgtaagttgggcagttctggcggtaccgacgcatccttgaagccttttgcagaaggatcttctttaaaactagcagaagcttgtagacggttcctgctccatccaggcaaagacacggatatccgaaagtgggcagctgaaggtctctcttatttaacattagatgctgaagtaaaagaaaagttgatagaggattcagcggcacttcaagctctagtagaattagcgaaaacagaagatcagtcggttctttttggcgtcataacgactttggtgaacctttgtaacgcctatgaaaaacaagaagttatcccagaaatgttggagttagcaaaatttgcaaagcagcatattcccgaagaacacgaactagatgactccgattttgtgacaaaacggattttggtattgggaaggagtaatgtgacaacggctttggttgctttatcgaagacagagagtaacaattctaaagaattgatagcaagagtgtttaatgccttgtgcagcgaacacgagttaagaggtgtcgttgttgctcaaggtggcagtaaggcattgctacctttggctttaaatggtacagataaaggaaagagacaagcagctcaagcgttagcaaggatcggtatcaccatgaatccagaaatagcttttccaggtcagagatccttagaagtaatccgacctctcctcaattcgttgcatcaagactgttcaggtttagaaaacttcgaagcacttatggccttatgcaacatcgcccaaatgaacgaaacggccaggatgaggattataaaggaagggggtttctccaagattgagcattatatgtttgaggatcatgaatacatatgtagagctgctgtgcagtgcatgtgcaatatgactcagtccgaagaggtagttaagatgttcgaaggagaaaatgataggatgaagttaactgtatctctctgtctggatgaggacaaagacacatctattgcagctgcaggaatgcttgctatcctaacaggagccagcaaaaattgctgcgagaaaattttcgactcaaaaaaattggttggaatctttacactgtcttttagctaacccagaccttagtatgcagtacaggggagtctgcgtagctttcaatatactttcagcgtccaaaacagtagccgagaaattggtcgaaacagatgttttagaaattttaatggctttatcgaagttgccaccgtatgatggaagggagaagattgtggagattgcaggacaagcgttgaaggaagccgagaagtggggggttatcaagaaacctggagaggaagatgatgatgaggaatagatggctgacaagaaaataagtgccaaattaaaaaaaaacagtgccgaatctgtatgatgtgtgtgaaatcagaataacttttcccttcattttgtacttggtcacgctgattctaaatcgtccatataatatttacattttactattagccggtttcatcaacaacaaataaatcactgaatagttattggtcaaataaaatttattagtcgattatatttttattgtttttcaatgcaatttttgataaatttaaaattaaactgctgaattttcttggttatagatttaaagggataacttatcaatttattcgaagaataaaatatttatttgataaataatgtcttatttgacgttggtgaaacgtagatgtgccagttctattggtcgaaataactttattcatcgaataaagtactagtcgtcgcgtcgtaggtgaaaccgaccatatgcatagtagattattattatgaaaaaacttaaacataaattttgtttgcgacaaacttagtttttaattattatcctattttatacttggcgcttttccctatggaatgttcacttacatttttacaattcctttagatatttgaattttggatctttcttcaacaatatgaagtgtcgaatttcgtttgataacatgttgcttcatatacttattccttactcgatttgtcttttgtatcatttttttaagaactttcattactaaagcattcaacatcatcaccatttctttcactattttaacaatttggattgttctctgcaacaagtattgtttctttattcgtatgcgcggtccaaattgtcattaaatgtttttcttacaatgaaccaatacactatcgttttttgttttttttttgtctcttgaaacgttgtttatgtattcgagtcacataatttagagcagatcatttttagcgtcactcaatagtgtctattatgtaagtgctgtgttactaactaatgtgaaaatagaaataaaaaacatacctaagagagtcacaaataaaatatatgattaagtctaattaccacaaaatgattttttgggaattattttggaatctgaaatgattttgaacctttaactattcagtatcagttatttttactttttgtaatatcagaggcagtatttat

SEQ ID NO: 9 - protein, 220 aa, Diabrotica virgifera
MGVPWFLEILDSEDVKQVSAAEHCMQSIINAFSGMENRPDTKPKQELIESNKKQIDTLLTCLVYSINNRVISGLARDALIELIMRNIHYDQLCWAKELVDIGGVERLMECASELEEYKYESAMNITPSTRTIAAVCLARVYENMYYDTLREKFMGKIENFIKDKLLTPDVESKVRVTVALTSLLRGPLEVGNALIGKEGIMEMILVMANTEDELQQKVSL

SEQ ID NO: 10 - DNA, 395 bp, Diabrotica virgifera
aggttgaaaaaacaaagttgttgttacttctgctattagaataataggagaactatgtaaacacagtgagactcgtactaaaaaagttatagaaactatgggagttccttggtttcttgaaatacttgattctgaagatgtaaaacaagtgtctgccgcagagcactgtatgcaatctattataaacgcattctctggcatggaaaatcgaccagacactaaacctaaacaggaattgatagaaagtaacaagaaacaaatagatacattattgacttgcttagtatattctattaataacagagtgatttcaggactagctagggatgcattgatagaactcattatgaggaatattcactatgatcagctctgttgggccaaagagctagtag

SEQ ID NO: 11 - DNA, 358 bp, Diabrotica virgifera
cggtgtagaaagattaatggaatgtgctagtgaattggaggagtacaagtatgagagtgctatgaacatcacaccttctacaagaacaatagctgcagtttgtttagctagggtctatgaaaatatgtactatgatactctaagggagaaatttatgggaaaaattgagaacttcattaaagacaaattgttaacaccagatgttgaatccaaagtgagagtgactgttgcattaacatctctattaaggggtcctctagaagtagggaatgctcttataggcaaagaaggcatcatggaaatgattctggtaatggccaacacagaagatgaactccaacaaaaagttagcctgtga

SEQ ID NO: 12 - DNA, 957 bp, Artificial Sequence
  hairpin forming sequence
ctaaggagttccttggtttcttgaaatacttgattctgaagatgtaaaacaagtgtctgccgcagagcactgtatgcaatctattataaacgcattctctggcatggaaaatcgaccagacactaaacctaaacaggaattgatagaaagtaacaagaaacaaatagatacattattgacttgcttagtatattctattaataacagagtgatttcaggactagctagggatgcattgatagaactcattatgaggaatattcactatgatcagctctgttgggccaaagagctagtagatataggcggtgtagaaagattaatggaatgtgctagtgaattggaggagtacaatagttagttgagactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttatcaactaactattgtactcctccaattcactagcacattccattaatctttctacaccgcctatatctactagctctttggcccaacagagctgatcatagtgaatattcctcataatgagttctatcaatgcatccctagctagtcctgaaatcactctgttattaatagaatatactaagcaagtcaataatgtatctatttgtttcttgttactttctatcaattcctgtttaggtttagtgtctggtcgattttccatgccagagaatgcgtttataatagattgcatacagtgctctgcggcagacacttgttttacatcttcagaatcaagtatttcaagaaaccaaggaactccagagct

SEQ ID NO: 13 - DNA, 510 bp, Diabrotica virgifera
tttaaaaggaataccaaaagccctgctggaaaaagtaaggcaaaagcaagctgccaaagccttactgtcaatgacgcgatcagaagataaagaaaaagaacttaaggtttactcgagactccccgaactggcacgtttaacaagaaatgtatttgtatccgaaaagaagaacgtactacaactagacatcctcgtcgataagttaggaaactgttatagatcacatttaaccaaactagaaatggaagaacacttaaggattctttcaaaagaatttcccacgtggttggtgttccatgttgtaaggaattgcgtgtatgtgaagatagccaaaagtgatgatttaagtttgataattaataagttggagagtatagttaaacaaaaaagtagaactttaacattttatattacaatatttctttgcagttttgactgatactttagtttattatttaactatctatacattagtattaagtaaaatcgtacaaattatattgaattgta

SEQ ID NO: 14 - protein, 122 aa, Diabrotica virgifera
MTRSEDKEKELKVYSRLPELARLTRNVFVSEKKNVLQLDILVDKLGNCYRSHLTKLEMEEHLRILSKEFPTWLVFHVVRNCVYVKIAKSDDLSLIINKLESIVKQKSRTLTFYITIFLCSFD

SEQ ID NO: 15 - DNA, 371 bp, Diabrotica virgifera
caatgacgcgatcagaagataaagaaaaagaacttaaggtttactcgagactccccgaactggcacgtttaacaagaaatgtatttgtatccgaaaagaagaacgtactacaactagacatcctcgtcgataagttaggaaactgttatagatcacatttaaccaaactagaaatggaagaacacttaaggattctttcaaaagaatttcccacgtggttggtgttccatgttgtaaggaattgcgtgtatgtgaagatagccaaaagtgatgatttaagtttgataattaataagttggagagtatagttaaacaaaaaagtagaactttaacattttatattacaatatttctttgcagttttgactga

SEQ ID NO: 16 - DNA, 995 bp, Artificial Sequence
  hairpin forming sequence
ctaatagttagttgagacgcgatcagaagataaagaaaaagaacttaaggtttactcgagactccccgaactggcacgtttaacaagaaatgtatttgtatccgaaaagaagaacgtactacaactagacatcctcgtcgataagttaggaaactgttatagatcacatttaaccaaactagaaatggaagaacacttaaggattctttcaaaagaatttcccacgtggttggtgttccatgttgtaaggaattgcgtgtatgtgaagatagccaaaagtgatgatttaagtttgataattaataagttggagagtatagttaaacaaaaaagtagaactttaacattttatattacaatatttctttgcagttttgactgagactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttatcagtcaaaactgcaaagaaatattgtaatataaaatgttaaagttctacttttttgtttaactatactctccaacttattaattatcaaacttaaatcatcacttttggctatcttcacatacacgcaattccttacaacatggaacaccaaccacgtgggaaattcttttgaaagaatccttaagtgttcttccatttctagtttggttaaatgtgatctataacagtttcctaacttatcgacgaggatgtctagttgtagtacgttcttcttttcggatacaaatacatttcttgttaaacgtgccagttcggggagtctcgagtaaaccttaagttctttttctttatcttctgatcgcgtcattgtagttagttgaagagct

SEQ ID NO: 17 - DNA, 1400 bp, Diabrotica virgifera
tcttctgctatatattgcaagatgtcgctgttgtagtcaaatgttctgacgtcataaagccattggctcaaaaagaaatatgtcggcgtgttgagaggaacaagtgatagtgttgttgtaaaaagtattggttttccgttattcaaaattaatcaaacaattttctattagacttctttaaattgttgagatggcgcttaaaatggctgtacatacaagtagtatcatcggtatcggaaagcctgattatgtggtcggaggaaaatatcgtctacttcgtaaaatcggtagtggatcttttggtgacatataccttggaataaatattacgaatggagaggaagtagcagtaaaacttgaaccaattagagcaaggcatccacagcttttatatgaaagtaaactatataaaattcttcatggaggcataggaataccacatatcagatattatggacaagaaaaagaatataatgtccttgtcatggacctgctcggtccgtcgttggaagacctcttcaatttttgtacccgcagattcaccattaaaaccgtactcatgttggcagatcaaatgatcactcgtatagaatttgtccattgcaaatcgttcatacatcgtgatataaagccggacaatttcctcatgggcatcggccgtcattgcaacaaattattcttgatcgattttggactggctaagaaatttagagacaccagaactagaatgcatataatataccgtgaagataaaaatttaactggtactgcccggtacgcgtccattaatgcacatcttggtattgagcagtcacgtagagatgacatggaatctttaggatatgtattgatgtatttcaatagaggctgtttgccatggcagggattgaaggcggctacaaagaaacaaaaatatgagaagatcagcgagaagaaaatgtccacgcccgtagaagttctgtgcaagggcttccccgctgaattttccatgtacctgaactattgccgtggacttcgcttcgaagaaaaccccgactacatgtatttgcggcaattgttccgtattctattccgtacgctgaaccaccagtacgactacacattcgattggactatgttgaaacaaaacgccgcctccggtactggcagttctgtggcaggacaagcttcgacacaagcccatcaagctcaagcgtccgtcacaggtcctcacttagcgaaccgagataaaaaagacgatgaaaagaacaagcagaattctgctaaaggtacagcacaaagtaaaacaatccatgcttcttccactgataatgttaaagtttttaaaggtcattgatttagttttttgtatattattttccccttattttgtaagagtttttaatttatataaaaatttt

SEQ ID NO: 18 - protein, 381 aa, Diabrotica virgifera
MALKMAVHTSSIIGIGKPDYVVGGKYRLLRKIGSGSFGDIYLGINITNGEEVAVKLEPIRARHPQLLYESKLYKILHGGIGIPHIRYYGQEKEYNVLVMDLLGPSLEDLFNFCTRRFTIKTVLMLADQMITRIEFVHCKSFIHRDIKPDNFLMGIGRHCNKLFLIDFGLAKKFRDTRTRMHIIYREDKNLTGTARYASINAHLGIEQSRRDDMESLGYVLMYFNRGCLPWQGLKAATKKQKYEKISEKKMSTPVEVLCKGFPAEFSMYLNYCRGLRFEENPDYMYLRQLFRILFRTLNHQYDYTFDWTMLKQNAASGTGSSVAGQASTQAHQAQASVTGPHLANRDKKDDEKNKQNSAKGTAQSKTIHASSTDNVKVFKGH

SEQ ID NO: 19 - DNA, 773 bp, Diabrotica virgifera
acaagtagtatcatcggtatcggaaagcctgattatgtggtcggaggaaaatatcgtctacttcgtaaaatcggtagtggatcttttggtgacatataccttggaataaatattacgaatggagaggaagtagcagtaaaacttgaaccaattagagcaaggcatccacagcttttatatgaaagtaaactatataaaattcttcatggaggcataggaataccacatatcagatattatggacaagaaaaagaatataatgtccttgtcatggacctgctcggtccgtcgttggaagacctcttcaatttttgtacccgcagattcaccattaaaaccgtactcatgttggcagatcaaatgatcactcgtatagaatttgtccattgcaaatcgttcatacatcgtgatataaagccggacaatttcctcatgggcatcggccgtcattgcaacaaattattcttgatcgattttggactggctaagaaatttagagacaccagaactagaatgcatataatataccgtgaagataaaaatttaactggtactgcccggtacgcgtccattaatgcacatcttggtattgagcagtcacgtagagatgacatggaatctttaggatatgtattgatgtatttcaatagaggctgtttgccatggcagggattgaaggcggctacaaagaaacaaaaatatgagaagatcagcgagaagaaaatgtccacgcccgtagaagttctgtgcaagggcttcccgctgaattttcc

SEQ ID NO: 20 - DNA, 946 bp, Artificial Sequence
  hairpin forming sequence
ctaaacaagtagtatcatcggtatcggaaagcctgattatgtggtcggaggaaaatatcgtctacttcgtaaaatcggtagtggatcttttggtgacatataccttggaataaatattacgaatggagaggaagtagcagtaaaacttgaaccaattagagcaaggcatccacagcttttatatgaaagtaaactatataaaattcttcatggaggcataggaataccacatatcagatattatggacaagaaaaagaatataatgtccttgtcatggacctgctcggtccgtcgttggaagacctcttcaatttttgtacccgcagattcaccattaaaaccgtactcatgttggcgactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttagccaacatgagtacggttttaatggtgaatctgcgggtacaaaaattgaagaggtcttccaacgacggaccgagcaggtccatgacaaggacattatattctttttcttgtccataatatctgatatgtggtattcctatgcctccatgaagaattttatatagtttactttcatataaaagctgtggatgccttgctctaattggttcaagttttactgctacttcctctccattcgtaatatttattccaaggtatatgtcaccaaaagatccactaccgattttacgaagtagacgatattttcctccgaccacataatcaggctttccgataccgatgatactacttgttcagtagagct

SEQ ID NO: 21 - DNA, 1270 bp, Diabrotica virgifera
gttttgctttgaaaattaaataaaaaatcaataaaattacttttcacaaccgtggatttcagtcgtagaagaagtcgtaataagaagatgattctgagagaactgtaactgttgtgacatacgtcattgatgacaaccgttaaccgtaaacttaaccacattatttactttgtgattgtgaatgccgatttattataagaaaaacttaatttaagttagtaaccccgtttaaaataatatataaatatgtattcggttttacacaaaaatgaaaatgctcatgaagaatgtatttggagttgtttttggggccgatatgcccccgaaaagaagaaaaaatccgaggacgacaatcaaggagacgatgagaaatccagagattcaattctatcagatgagcctccgactgactatattgttactggtggagtagatgatttagttaaagtttgggaattgcaggatgatcgtctagttcttaagcataacctagaaggccattctcttggagttgtgtcagtagctgtcagtcataatggcaaattatgtgcatcaagctccctagattcaagtatgaggatttgggatttggagaagggtgaaaagattgcaaatgttgatgtgggccctgtagatctttggactgttgcttttagtccagatgataaatacattatttctggatctggaaaaattacagcctatagtgttgaaacagccaaagctgaacaaacttttgaaactcgaggaaaatttactttaagcatagcttatagcccagatggaaaatacatagcaagtggagctattgaaggcattattaatatatttgatgtagcagccaacaaactgttgcacacattagaaggtcacgctatgcccatccgttctctttgcttttctcctgactcccaactactgctaacaggatctgatgatgggtatatgaaactttatgatgtgcgagatcaaaaaacccgtctgataggaacattatcaggccatgcttcttgggttcttagtgtagcattttctcctgatggcaagaactttgtttctggcagttccgataaaacggtaaaagtttgggatgtcgctaccagacagtgtcttcatacatttaatgaacatacagatcaggtatggggcgtgacatataacccagatagtaataaaattctttcagtatctgaagataaaagtatcaatgtatatagttgtcctgtttagatttagcaattatattttttaacgcgaaaaaaaaaaaaaa

SEQ ID NO: 22 - protein, 327 aa, Diabrotica virgifera
MYSVLHKNENAHEECIWSCFWGRYAPEKKKKSEDDNQGDDEKSRDSILSDEPPTDYIVTGGVDDLVKVWELQDDRLVLKHNLEGHSLGVVSVAVSHNGKLCASSSLDSSMRIWDLEKGEKIANVDVGPVDLWTVAFSPDDKYIISGSGKITAYSVETAKAEQTFETRGKFTLSIAYSPDGKYIASGAIEGIINIFDVAANKLLHTLEGHAMPIRSLCFSPDSQLLLTGSDDGYMKLYDVRDQKTRLIGTLSGHASWVLSVAFSPDGKNFVSGSSDKTVKVWDVATRQCLHTFNEHTDQVWGVTYNPDSNKILSVSEDKSINVYSCPV

SEQ ID NO: 23 - DNA, 460 bp, Diabrotica virgifera
gtattcggttttacacaaaaatgaaaatgctcatgaagaatgtatttggagttgtttttggggccgatatgcccccgaaaagaagaaaaaatccgaggacgacaatcaaggagacgatgagaaatccagagattcaattctatcagatgagcctccgattgactatattgttactggtggagtagatgatttagttaaagtttgggaattgcaggatgatcgtctagttcttaagcataacctagaaggccattctcttggagttgtgtcagtagctgtcagtcataatggcaaattatgtgcatcaagctccctagattcaagtatgaggatttgggatttggagaagggtgaaaagattgcaaatgttgatgtgggccctgtagatctttggactgttgcttttagtccagatgataaatacattatttctggatctggaaaaattacagcctatagt

SEQ ID NO: 24 - DNA, 288 bp, Diabrotica virgifera
atgatgccaataggaggagctcctccggtgcacggctctagtagtttatcacaaagtaatacctcacaaaatgctagtttaacacctacaggtggatccagcaaatccagtagtagcttgaagccaaattatgcccttaaatttacgctagctggacatacaaaagcagtgtcttctgttaaatttagccctaatggagaatggttagccagctcatctgcagataaacttgtaaaaatttggggagcatatgacgggaagtttgaaaaaccatttcaggacacaaac

SEQ ID NO: 25 - DNA, 1267 bp, Artificial Sequence
  hairpin forming sequence
gttgaaacagccaaagctgaacaaacttttgaaactcgaggaaaatttactttaagcatagcttatagcccagatggaaaatacatagcaagtggagctattgaaggcattattaatatatttgatgtagcagccaacaaactgttgcacacattagaaggtcacgctatgcccatccgttctctttgcttttctcctgactcccaactactgctaacaggatctgatgatgggtatatgaaactttatgatgtgcgagatcaaaaaacccgtctgataggaacattatcaggccatgcttcttgggttcttagtgtagcattttctcctgatggcaagaactttgtttctggcagttccgataaaacggtaaaagtttgggatgtcgctaccagacagtgtcttcatacatttaatgaacatacagatcaggtatggggcgtgacatataacccagatagtaataaaattctttcagtatctgaagataaaagtatcaatgtatatagttgtcctgagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagcaggacaactatatacattgatacttttatcttcagatactgaaagaattttattactatctgggttatatgtcacgccccatacctgatctgtatgttcattaaatgtatgaagacactgtctggtagcgacatcccaaacttttaccgttttatcggaactgccagaaacaaagttcttgccatcaggagaaaatgctacactaagaacccaagaagcatggcctgataatgttcctatcagacgggttttttgatctcgcacatcataaagtttcatatacccatcatcagatcctgttagcagtagttgggagtcaggagaaaagcaaagagaacggatgggcatagcgtgaccttctaatgtgtgcaacagtttgttggctgctacatcaaatatattaataatgccttcaatagctccacttgctatgtattttccatctgggctataagctatgcttaaagtaaattttcctcgagtttcaaaagtttgttcagctttggctgtttcaac

SEQ ID NO: 26 - DNA, 815 bp, Diabrotica virgifera
cttgttggggaaatctccccgtatcttcgccactgcatttgaattcgtgtgctttcggttctattggtattgaattcacgaacacaaaaattagcgctaaaggttgcctaaccctaaaggtggtgctgtttcaaaactgacagcttgtcaacaattctctctttttctgtccattttatgtcacaagtagggctaaaatgaagcaaattgtaacaaaccaaacggtaaaaattcctgagggaattacagtaaccactaagtcaagggtagtaactgtaaaaggaccccgaggtaatctgaagagatccttcaaacatcttaccctggacattcgaatgatcaaccccaggttactgaaagtagagaaatggttcggtaccaagaaggaattagctgctgtcagaacagtatgctctcatgttgaaaacatgttaaagggtgttacaaaaggataccaatacaagatgagagcagcatatgctcactttcccattaactgtgtcaccactgaaggaaacacagttatcgaaattagaaatttcttgggagaaaaatacatcagaagagtaaagatggccccaggtgttacagtagtaaactccaccaaacaaaaggatgaacttatcattgaaggaaactcattggaggatgtttcaaagtcagctgcccttattcaacaatctacaacagttaaaaataaggatatccgtaaattcttagatgggctttatgtatctgaaaaaacaactgttgtacaagaagaataacttgtatttttgaaataaatttaagtttatatttaaaaaaaaaaaaaa

SEQ ID NO: 27 - protein, 189 aa, Diabrotica virgifera
MKQIVTNQTVKIPEGITVTTKSRVVTVKGPRGNLKRSFKHLTLDIRMINPRLLKVEKWFGTKKELAAVRTVCSHVENMLKGVTKGYQYKMRAAYAHFPINCVTTEGNTVIEIRNFLGEKYIRRVKMAPGVTVVNSTKQKDELIIEGNSLEDVSKSAALIQQSTTVKNKDIRKFLDGLYVSEKTTVVQEE

SEQ ID NO: 28 - DNA, 563 bp, Diabrotica virgifera
gaagcaaattgtaacaaaccaaacggtaaaaattcctgagggaattacagtaaccactaagtcaagggtagtaactgtaaaaggaccccgaggtaatctgaagagatccttcaaacatcttaccctggacattcgaatgatcaaccccaggttactgaaagtagagaaatggttcggtaccaagaaggaattagctgctgtcagaacagtatgctctcatgttgaaaacatgttaaagggtgttacaaaaggataccaatacaagatgagagcagcatatgctcactttcccattaactgtgtcaccactgaaggaaacacagttatcgaaattagaaatttcttgggagaaaaatacatcagaagagtaaagatggccccaggtgttacagtagtaaactccaccaaacaaaaggatgaacttatcattgaaggaaactcattggaggatgtttcaaagtcagctgcccttattcaacaatctacaacagttaaaaataaggatatccgtaaattcttagatgggctttatgtatctgaaaaaacaactgttgtacaagaag

SEQ ID NO: 29 - DNA, 1359 bp, Artificial Sequence
  hairpin forming sequence
gaagcaaattgtaacaaaccaaacggtaaaaattcctgagggaattacagtaaccactaagtcaagggtagtaactgtaaaaggaccccgaggtaatctgaagagatccttcaaacatcttaccctggacattcgaatgatcaaccccaggttactgaaagtagagaaatggttcggtaccaagaaggaattagctgctgtcagaacagtatgctctcatgttgaaaacatgttaaagggtgttacaaaaggataccaatacaagatgagagcagcatatgctcactttcccattaactgtgtcaccactgaaggaaacacagttatcgaaattagaaatttcttgggagaaaaatacatcagaagagtaaagatggccccaggtgttacagtagtaaactccaccaaacaaaaggatgaacttatcattgaaggaaactcattggaggatgtttcaaagtcagctgcccttattcaacaatctacaacagttaaaaataaggatatccgtaaattcttagatgggctttatgtatctgaaaaaacaactgttgtacaagaagagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagcttcttgtacaacagttgttttttcagatacataaagcccatctaagaatttacggatatccttatttttaactgttgtagattgttgaataagggcagctgactttgaaacatcctccaatgagtttccttcaatgataagttcatccttttgtttggtggagtttactactgtaacacctggggccatctttactcttctgatgtatttttctcccaagaaatttctaatttcgataactgtgtttccttcagtggtgacacagttaatgggaaagtgagcatatgctgctctcatcttgtattggtatccttttgtaacaccctttaacatgttttcaacatgagagcatactgttctgacagcagctaattccttcttggtaccgaaccatttctctactttcagtaacctggggttgatcattcgaatgtccagggtaagatgtttgaaggatctcttcagattacctcggggtccttttacagttactacccttgacttagtggttactgtaattccctcaggaatttttaccgtttggtttgttacaatttgcttc

SEQ ID NO: 30 - DNA, 3209 bp, Diabrotica virgifera
ttttatttcataagcaaatcatttccagtttcgtcaaaaaaaaatcattacacaccgttgattactatgttattggaatttcctttttataaacacattttatcagctttggttttaaaaaccccaatgaattttacgaaaaagttgacaacgttgtgtatttcttctactactgcaaccgtgtgccgacgacgtgagacgcgagttcaaaataccagacttatttacatattattgtattttgttagtagagttgaggaagatgtgcaaagtgttgaaaagtttatgtacatgaattgtagaaactaattgtttattgtgtatcattcgaattcagtttcaaaattaaaagtgtgctagtggagaaaactaaacgataacttctgtttatactgttgtttttatcgtttaacaatggctctctttcttctaggtttgttgttctgcctatctagtcggatctctgccagtggtttctacgaaatcagtaacattgacttctataattctgtttttacagattctcttgattggagattgttacaggagctatcaaaccagtataaaaacgtagtgatatctcccatcagcctaaaaatcatattatccctattatatcaaggatctaccggacagacagaaagggaattccaaacacttttaaattatcaaaataaagaatatgtcaggaacaattacagccaaattgtcgctgccctttataatgcagacagaactgaatatatgctcaacattgggacatcgatgtttgtagatgaaggtctatacgttttatctaaattcgaagaccttgcaagaaaaagcttcaaaacagacatcataaagactaactttgacaaacaggaaaaggctagtaaagatattaattcgtgggtggaacagcttactcatggtaaagtgaagaaaatagtcaatgctgatgatctgtcgcaagcactaatgataattaccaatgccatttacttcaaaggaacatggactcgtaaatttgataaaaatagatcaacgcttggaaacttctatacatctcctgataatataagatctagcagaaacttaaaattagttcaatacatgactaccactgacgaatttctttattacgaggacagatctttggatgcgaaaattgtcaggttggaatacaagggaagtgactatgccatgtatatagtcctaccgaacactttgggaggattaagcagccttatcaaaaatgtggacataaacaaaataggaactataaagttccaaatgacacaacaagttgttgaggttactattccaaagtttaagttcaattttgacatcaagttgggaaagattcttcaaaagtttggactacgcgaagcattccaaaacacggccagttttacaggcatcgtgcaaaccaacaggaccttaagaaggagtctatacgtatctgacattgttcagaaatcgggaatagaagttgatgaagaaggaagtgaaatcttctcagcatcagctgtcaacgtgggtaataaattcgccgaaaatacgatgatattcaatgcatcacatccgttcatgttctttgttgatggtcccaacggtaccatattgtttgttggacaatatgaaaatccagatgcggtggacccgtcagaggtaccgaatcgttggagtcaagaagtagaagaaaaaaatcctactgaagctgtgcaattccaaaatactcgcccaagtagaccaaataatgaaattgatcaatccaataatgtacagttccaacagcctcgtccaaatagaccaagtgttgaaagtcaacgacccgacaatgtacaattccaacagaatcgcccaaatagaccaaatactgaaaatgaacagcccagcaatgtacaaggtcttaatttgaatccaaatctggaccccgactactatcaacctccagatgtagaaatggatgagattgcatatagattcaatctgtttgatattgacttattatattccttttctgatttatccacgaacgtattaatatctcccgctagcatcaaaacgacattagcaatgattttagaaggagcagaaggaacttgtgcagaagaaataagtgaagcactgaggatacctgatattaatcaaaaaggtgtcagaagtgttcttgtagaacttctaaataatcttaatgaaagatctacgccaaatagtgtcctagaaagccataatgccatatttgtctctgaaaaacatcgactggttgataattataagaatgtcgttataaagtattacaacgcttacctgaaaagcttagatttttcaaattcagattatgctgctagggtcattaatggatggatatctgactccactcatggcgaaataaccgatgtcatctcaccacaggggatagttcctgattgttacgtggtgctgtcaaacgccctgttctttaaagcacagtggaaatatgcttttgataaaagaagtgtacgaaaatgtttccacactcctaggggatgtgtttatgttgacatgatgcacatttcacataatttcaattacaattacatcacccgtttgcgagccaatgttgtggatattccttatcaggataaattctccatgctactgatcttaccatcttcagattcaaatgtaaggagtgtgattcgggatttacctcactttagactgtcgagcattttggagagtctaaatcaatccgagattgttctagaactaccaacatttagcttcgagtattcagcagatttggttgaatttttaaaacctcttaaaatccgagaaatatttggttcccgcgctaacttaaccaaaatgatagaaggccaccatggtatgataaacaacctgttacacaagaccaaaatcacagtagacgaaaaaggaacaactgccgcagcatcctccagtgccatgataattcccctgatgcagccagtgacagttgcggctgacagaccgtttgtttttatgatctatcacagagacactaaaaatataatctttgaaggaattgtacaaaatcctttagaaaaatagtatctaacattactgataattttttatttaatgtgccgaaaactcatctaggttagatctcttcaaactgtagttacgtgtaaaaattggtctgtatttgcttttttcataaaaacctttttaaataaattgtttaatataaaaaaaaa

SEQ ID NO: 31 - protein, 881 aa, Diabrotica virgifera
MALFLLGLLFCLSSRISASGFYEISNIDFYNSVFTDSLDWRLLQELSNQYKNVVISPISLKIILSLLYQGSTGQTEREFQTLLNYQNKEYVRNNYSQIVAALYNADRTEYMLNIGTSMFVDEGLYVLSKFEDLARKSFKTDIIKTNFDKQEKASKDINSWVEQLTHGKVKKIVNADDLSQALMIITNAIYFKGTWTRKFDKNRSTLGNFYTSPDNIRSSRNLKLVQYMTTTDEFLYYEDRSLDAKIVRLEYKGSDYAMYIVLPNTLGGLSSLIKNVDINKIGTIKFQMTQQVVEVTIPKFKFNFDIKLGKILQKFGLREAFQNTASFTGIVQTNRTLRRSLYVSDIVQKSGIEVDEEGSEIFSASAVNVGNKFAENTMIFNASHPFMFFVDGPNGTILFVGQYENPDAVDPSEVPNRWSQEVEEKNPTEAVQFQNTRPSRPNNEIDQSNNVQFQQPRPNRPSVESQRPDNVQFQQNRPNRPNTENEQPSNVQGLNLNPNLDPDYYQPPDVEMDEIAYRFNLFDIDLLYSFSDLSTNVLISPASIKTTLAMILEGAEGTCAEEISEALRIPDINQKGVRSVLVELLNNLNERSTPNSVLESHNAIFVSEKHRLVDNYKNVVIKYYNAYLKSLDFSNSDYAARVINGWISDSTHGEITDVISPQGIVPDCYVVLSNALFFKAQWKYAFDKRSVRKCFHTPRGCVYVDMMHISHNFNYNYITRLRANVVDIPYQDKFSMLLILPSSDSNVRSVIRDLPHFRLSSILESLNQSEIVLELPTFSFEYSADLVEFLKPLKIREIFGSRANLTKMIEGHHGMINNLLHKTKITVDEKGTTAAASSSAMIIPLMQPVTVAADRPFVFMIYHRDTKNIIFEGIVQNPLEK

SEQ ID NO: 32 - DNA, 600 bp, Diabrotica virgifera
cctactgaagctgtgcaattccaaaatactcgcccaagtagaccaaataatgaaattgatcaatccaataatgtacagttccaacagcctcgtccaaatagaccaagtgttgaaagtcaacgacccgacaatgtacaattccaacagaatcgcccaaatagaccaaatactgaaaatgaacagcccagcaatgtacaaggtcttaatttgaatccaaatctggaccccgactactatcaacctccagatgtagaaatggatgagattgcatatagattcaatctgtttgatattgacttattatattccttttctgatttatccacgaacgtattaatatctcccgctagcatcaaaacgacattagcaatgattttagaaggagcagaaggaacttgtgcagaagaaataagtgaagcactgaggatacctgatattaatcaaaaaggtgtcagaagtgttcttgtagaacttctaaataatcttaatgaaagatctacgccaaatagtgtcctagaaagccataatgccatatttgtctctgaaaaacatcgactggttgataattataagaatgtcgttataaagtattacaacgct

SEQ ID NO: 33 - DNA, 599 bp, Diabrotica virgifera
acctgaaaagcttagatttttcaaattcagattatgctgctagggtcattaatggatggatatctgactccactcatggcgaaataaccgatgtcatctcaccacaggggatagttcctgattgttacgtggtgctgtcaaacgccctgttctttaaagcacagtggaaatatgcttttgataaaagaagtgtacgaaaatgtttccacactcctaggggatgtgtttatgttgacatgatgcacatttcacataatttcaattacaattacatcacccgtttgcgagccaatgttgtggatattccttatcaggataaattctccatgctactgatcttaccatcttcagattcaaatgtaaggagtgtgattcgggatttacctcactttagactgtcgagcattttggagagtctaaatcaatccgagattgttctagaactaccaacatttagcttcgagtattcagcagatttggttgaatttttaaaacctcttaaaatccgagaaatatttggttcccgcgctaacttaaccaaaatgatagaaggccaccatggtatgataaacaacctgttacacaagaccaaaatcaca

SEQ ID NO: 34 - DNA, 1433 bp, Artificial Sequence
  hairpin forming sequence
cctactgaagctgtgcaattccaaaatactcgcccaagtagaccaaataatgaaattgatcaatccaataatgtacagttccaacagcctcgtccaaatagaccaagtgttgaaagtcaacgacccgacaatgtacaattccaacagaatcgcccaaatagaccaaatactgaaaatgaacagcccagcaatgtacaaggtcttaatttgaatccaaatctggaccccgactactatcaacctccagatgtagaaatggatgagattgcatatagattcaatctgtttgatattgacttattatattccttttctgatttatccacgaacgtattaatatctcccgctagcatcaaaacgacattagcaatgattttagaaggagcagaaggaacttgtgcagaagaaataagtgaagcactgaggatacctgatattaatcaaaaaggtgtcagaagtgttcttgtagaacttctaaataatcttaatgaaagatctacgccaaatagtgtcctagaaagccataatgccatatttgtctctgaaaaacatcgactggttgataattataagaatgtcgttataaagtattacaacgctagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagagcgttgtaatactttataacgacattcttataattatcaaccagtcgatgtttttcagagacaaatatggcattatggctttctaggacactatttggcgtagatctttcattaagattatttagaagttctacaagaacacttctgacacctttttgattaatatcaggtatcctcagtgcttcacttatttcttctgcacaagttccttctgctccttctaaaatcattgctaatgtcgttttgatgctagcgggagatattaatacgttcgtggataaatcagaaaaggaatataataagtcaatatcaaacagattgaatctatatgcaatctcatccatttctacatctggaggttgatagtagtcggggtccagatttggattcaaattaagaccttgtacattgctgggctgttcattttcagtatttggtctatttgggcgattctgttggaattgtacattgtcgggtcgttgactttcaacacttggtctatttggacgaggctgttggaactgtacattattggattgatcaatttcattatttggtctacttgggcgagtattttggaattgcacagcttcagtagg

SEQ ID NO: 35 - DNA, 722 bp, Diabrotica virgifera
gtttcgcagcagcagttcccccctctcttctcttacgtcgatcctaaacaaaacgagaaaagacagttggctggtgcactttgacgagaggcgttgttagttacttcgttgtgtcgacatttttctgtttagtgtaccgattaatcttcaaatattacaatggctgaccaactcaccgaagaacaaattgctgaattcaaagaagctttctcactattcgataaagatggtgatggtacaattacgactaaagaattaggaacagtaatgagatctctaggacaaaatccaacagaggctgaattacaggatatgatcaatgaagtagatgccgatggtaacggcacgatcgatttcccagaatttttaacgatgatggcacgtaaaatgaaagataccgatagtgaggaagaaattcgtgaagcattccgagtgttcgacaaagacggcaatggtttcatctcagcagcagaattgcgccacgtcatgaccaacttgggtgaaaaattgacagacgaagaagtcgatgaaatgattcgggaggccgatatcgatggtgatggtcaagtcaattacgaagagtttattagaagtcacctcttggccaactaagatattattgattacaaagaaaaatagaagaatatattataatttacagatactatattaatgataatagaataagaataaaatgtaaataaatagttttgctcattaaa

SEQ ID NO: 36 - protein, 150 aa, Diabrotica virgifera; identical to SEQ ID NO: 2

SEQ ID NO: 37 - DNA, 353 bp, Diabrotica virgifera
ccatgttttcgcaatctcaagtagctgaattcaaagaagctttccaacttatggaccacgacaaagatggcatcatctctaagagcgatttgagggccaccttcgatgctgttggtaaactcgccagtgagaaagagctcgacgagatgatcaacgaagcacccggaccaatcaacttcactcaattgttgggattgttcggtacccgtatggctgattccggcggtactgacgatgatgaagttgtcgtcaaggctttcagatcctttgacgaaaacggcaccattgacggagacagattccgccatgccctcatgacctggggagaaaaattcaccggcaaagaatgcgac

SEQ ID NO: 38 - DNA, 423 bp, Diabrotica virgifera
atggctgaccaactcaccgaagaacaaattgctgaattcaaagaagctttctcactattcgataaagatggtgatggtacaattacgactaaagaattaggaacagtaatgagatctctaggacaaaatccaacagaggctgaattacaggatatgatcaatgaagtagatgccgatggtaacggcacgatcgatttcccagaatttttaacgatgatggcacgtaaaatgaaagataccgatagtgaggaagaaattcgtgaagcattccgagtgttcgacaaagacggcaatggtttcatctcagcagcagaattgcgccacgtcatgaccaacttgggtgaaaaattgacagacgaagaagtcgatgaaatgattcgggaggccgatatcgatggtgatggtcaagtcaattacgaagag

SEQ ID NO: 39 - DNA, 939 bp, Artificial Sequence
  hairpin forming sequence
ccatgttttcgcaatctcaagtagctgaattcaaagaagctttccaacttatggaccacgacaaagatggcatcatctctaagagcgatttgagggccaccttcgatgctgttggtaaactcgccagtgagaaagagctcgacgagatgatcaacgaagcacccggaccaatcaacttcactcaattgttgggattgttcggtacccgtatggctgattccggcggtactgacgatgatgaagttgtcgtcaaggctttcagatcctttgacgaaaacggcaccattgacggagacagattccgccatgccctcatgacctggggagaaaaattcaccggcaaagaatgcgacagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagaggtcgcattctttgccggtgaatttttctccccaggtcatgagggcatggcggaatctgtctccgtcaatggtgccgttttcgtcaaaggatctgaaagccttgacgacaacttcatcatcgtcagtaccgccggaatcagccatacgggtaccgaacaatcccaacaattgagtgaagttgattggtccgggtgcttcgttgatcatctcgtcgagctctttctcactggcgagtttaccaacagcatcgaaggtggccctcaaatcgctcttagagatgatgccatctttgtcgtggtccataagttggaaagcttctttgaattcagctacttgagattgcgaaaacatgg

SEQ ID NO: 40 - DNA, 6961 bp, Diabrotica virgifera
tctaacttgccggcatgttggcgtcagaatgttttctcgttcattttgatctacgttaggccgttgcattttcaaagcgtaaattctaatcaatcccatagtttcagtgaccagtgaattatgttctaaaggttctttaataacgtttcaaacaaactctataatggaagactctttcgtttttggtagtgttcttagtgaagaagaatggaaagtagttccagtagatctaggaaaaaaaatcataaagtttgtgactgaaaatttcgatgacttaatcaaaggcaaagcgttattggaaacaaaatgttgtaatttggaaaaaaatgataaggatttacatgatgtcctagaaaaagcagaattagaaaataaaacacttaaatcaaaattagaagtagctgcaaacactataaatgaacatgaaacccaattgtctaatctagctgcagaactaaccaaattacatgcaaaatgcaatgcttttgaagccgaaacggccgaatttcgtcatcagaggaacaaagctgttgacgaaaaagatgaacatcttagaatgataaatagacgaaatggagaaatcgagagactccaagccgatttaaaagattccacaaagaagttggaatctgctatttcagcgaaatgtgaagcattagctcagattgaagaagttgcttcaatgaaaacaaatatagaatatcgtgaaaaaagaatggaacaagaaaaggcactatataacagccaaatagaaagtctacaagaagaactaaataagaaaactgaagaactacttaactcgaaacgtgacaactcatcaacttgtgttcaactggaaactaaacttattcagaagacacaggaactgactgtagcaacagaacagattaaatctctcaacgatattaacactaacttgaccgcgagaattgaagaactttctcaaaaaattgcgaatctaagggatgaagaagctaaggttaatgagtcatatgtatttgaaattgaagctaaaactaaaatggctaatgcctttaagtctagatatgaggaactagaacaccattctaaaacgctggaagatgctgttacagagttacaacaagtattgaaaaatgccactgagcaatatggagaattggaaacaaaacataaagagtctgagttggctaaagaggagattattgcaaagaagaatgaatgccttgagttattgaaaaaggaattagagacagccaatgaattattagataattcaagaccaatgctcgatagtgaaggtatttcaccaaaccaatcctcatcgaagcttgtgaagcctggtatgacctattcagagttatattcacgatatgtaaatgtttctaacagattaactgaaaaggaagaagagtgtgctagatcaaacaactacgtagatgcaatcgtcaaagaaatggatttcaatttaccaaaagtacgaaaaatgcaagaagactattcagaaagtttaacaactatagataccttaaaggatgccaatgaccaattatgcagtgaaatacaattacttcgtgatacaaatgctgagtgcaaaaaagctgagggaattcgaacacgagaaaatgaaagattgagaaatgaagtctctgatttatcacgacaagttgttgttttgttaaatgaggttgagaactccagagttgggtcgtcttcaacatcgactgataatgatttaagtgatagtgtcaattcagcggacattataagcaagaaattagttactttcaatgatatctctgaattgcagactaacaatttaaaacttctggctttggttagagaattatcggctcgccaagaagaagtggagagcctcgacccagctgcgattgccgcactcaaacttaaagtagaagaacttatgcaatcacaagatgaactactcgaagaacgtgaaaagcaaacaaaaatgatggtcaccctccgaaatcaaaaagacatgtataagaacttatacactcaagttgttaagggtgctggtgaagaaatccatatgtctgatgtttctgaaaatggggaacccaaaatccgcacggaaaccgaaacgcagctggaagaaaaggttcaagaatatgaagctaaagttgaaaaattaaagcgagatgttgaacatttgaaagaagaaaacgatatttatcgtaaagaaaagtcagccaatgaaaagattttgattgatcaattagacaatatgcgcggtgaggttaaggaactgacaagattaaattgtaaactgagcgctcgtgctgaacatgacgaagaaaagttcaaagtattcaacaacaacgttgacatttacaagaaacaaataacagcattagaaaagcagaataagatttatagtgagtcgataattaagcatgaacaagcagctacttatttgaaaaatgaaacaatacaaagccaaacgaagctttcacgagcagaagttatgctgggcaatttgcaaaaagaaaatgctttgctaaaggacgccgaacaacgtcttctcaaggaaagagattcgctgaaggtaaacttccatcaacaaaatcttatgaagacaaatatagagcttattaaagcttctctcgaacgcgcggacgcggaaggcaaacttaaattggaaaatagacttgatgaagctcatagagaatgtgctactctacgcagaagactgcaagaagaacaagatcattttagacaattgtccgatcatttatcagctcaaaataaaaccatcgagaagagagcagaagaagaacgggagatagcggaaaaattgcgcaaagaagttgcggaagttagggaagatctgatggctaaagtagcccaaaatgaagagttgggtaaaaaacttaagagtgaaatgtttatggtacctgacggtagcgctgagacaagaagagtaagagaattagaacagcagatcgctgatctacaagctgaaaatgaatcgttaaagaagaaaataaaaactgctaaagaagcttctgatgaatatttcaaggttgcagaggcgtcggaaaaacaagttaaagatgccatggagagaaatgaatcgttgatgcaagaagtagataagcataaagctcgcatacgagaactagaagaaaaatgcgctgaactggaaggcgaaatttctatacagttagatgatcaagatattacgaatgcaggaaagagcaagtccatgcagcttcaagaggaactgaatatcagaaatatggatttaaggacagcaaaacaacaattagaaactgctagatctgaaaatagacagttaattgaacaaatgaaagctgttgaaaataaatatgcacgagaggttaccttacactctgttgatttgcagtctcttactgctatgaaagaggatcttgataatgccttaggagaaattaataaattacaaagcgaaagacagaaggctactgaagctctcgaacaatttaaggagaattgggaaaaacaagaacatttgttccaaaacgaaaagcaggagcttaatgagagatttaaagatatggattctcagaatagtcttctgcatgatcaaatacaagccctcaatacacagcttgcactcctacaagcccaagtcagtgacagtcaaacccaaaatacatctatcggtgatacctccttctcgaaatcattcacagaagatgaaacgaaatcgtctgaacaacttctgaagattattaaatatctaagacaagagaaagatatcgctgtaagcaaagccgacatagttgaagctgaacattccagattgaagacacaatttgaaatgattaaaaagcaacttgatgaggctaaagagtctgttgaaacggagaggcagaaaacggagatctctgttgtatccgctgctaaacatgcagaagtacttcgtaaattagaaactcttaatgccatcaccgacagcaatagagctctaagacaggaacgagatactctagctgcccaattagcagacctccaaagcaaagcagaaacattcgaaactgaagtcgaacctttgcaggagaagaacagagagctcacaacaaaggctgaccaattacaaagtgaaaatatatcattacgtgccgaatgtaccagatggagacagagggctaatatgcttattgaaaagacgaacagaacaagtcccgaagattggaagaagttgcagacagaacgggagaccttatccaaacagttgataattgaaaaagccaatattaccaagttaacggatgatgttaacacattaaaacaggagaagagtaatcttgaggagaaactaaatactttgaagaaccagagtagtcagcagcaagaagaaataaccagattacgggacgaagtatctaacctacagtcacaagttacacaactcactaataccgtagatcaacaaagtgccgaaataattaggttcaaggaagaaactcgtggccttactgaggaaacggccgctaaagataccactattaacgagctaaagaataatatggcccaagttagaaagatagctaaaaagtacaagattcaatgcgaagaccaaactaaagaaataagcgcgctgaaacagcaaaatgaacagcacgaatctgatcaagtgagcagtgctgaaaagcaagaactgttggaaagccagcgttcagaattagaagaaaagataaatgaattagaaagaaaccataaagaaactgttgaccagttaaatcaagaagtttcttctacaacggagcaaatagagaattaccgaaaggaaattgatactctgaagcaaaccagtcaagagaaggaagaaagatttaaggcgctgtttaagaacgccaaggaaaggatcgtgagtttaacagaacagaacaacaacttgaaggaagaagttaatagaccgggaagatcagacggagctgaaaaggaaagcgaagactctgctaaagttgatgaattgctcgagaaaatagctttgctggaaagagagagggacgatattcttgaagaaaaacaacaggaacaagataagcatacaacagaagttgaaagtcttaaccaaaggatatcgcagttgcagaggcaattgggccttcaacaaggatcaaaacctagcactagttcaagtaccactgaaaagtcttccactgaacggccaactgctgatattaaaccaatggcaggccactctacaaatactcaaacacaatctgttcctatacaaccatggcgaagtggtggggaacctcctttagcaagtattagaccaatgtctgcacaactacgcactggtgttgtgttaccaactagtcagagtccaagcgcagttatggtaccaccacagcaacaggtgcacacaacaggatccagttcaatcgaagccctttctagcagccccactagttctcacacagaatacgttcctgccactagttctgctagtcctgcaatgttgggtcctcgccaagtcgccgtacctcccacccaatcgtctcaagatacagaagatgacgagagtagtatgcaggtccaggcagctccacaaacccaagccgttgctcttgtactaccaagagttgaaccacctagcagtggaccaactcaggaacagggtacgagcagtagcagctcgaacactgtgacgacgacacaggctgggcataaaagacagcgagaagctgatatggatagtagtcaatccgatgagcaaaataagacgcaacaacaaactaagaggacgaggatccagcaagctggaactgtaagtgatagtggtttagatgttgagtatcaagttcctacaagcagtcaaagggatcatgatgatgataatgtgatagtagttgaaagtgatgaagaaggtgctgctgatgaaggcgaaggtgctgatgatgagcaagatgatcctgatactgagggatatgatatggagggaatggaacaggataattatgaagatgctgattgccaagatgtggaagatgaagaggaggccggcaatgaagttgaggtgatagatgattccagtgaagtgccaaatcaaagtgagagagaagaagataacgtggaagaagaaacttcagaacaaccacagtctgaagcaataagtagtggcactgatggtaccggaaggagcgtacccacgacaccgctacagtcttctccacaagaaggaattcccggtagtgaagagcaaggtactagccaacagtcagatgctgaaaccccatcgattgtaataagtggcgaagttgacgataatgaaaccataggtgaaagtagttcgggcgatatgggtcccccagctgtagctggcacctcatcagaaggcgcccagcagcacgaacaagccgaagaaatgctcgaaggtgacgatggtgtaagttctgaaggagagaagccacccatagccgaagaaggcgaagaagaaggacgtgaagcggaggcttctcccagttctaattcacggggcgtagctcaaagaggctcgaattctgctcgaagatctacaaggtcgtcactaccgagaggtgctaatcaacgatctggtccaactccgattgtttggagcgacagatcgccccaacgacatcaacatccccaagacagaaatagatcaccccaaggttaccaatcgaggacggtagcaaggagagcccgcaatcgtggccagaggccatttgatagacgtttctaaacctcatttcgtttttaattcattccatgagcattgttaaggttttagtttcgtcttatattttgttttataaatgtacggaaaaaaatttattaataataggatttattatttttttttgtgaaagagttttgatgttcgactgagaaggattatttaatttattagcgatgtgtaaggccatccctgctgtgtaggcgtttgtaataaatatataatccagaactataatttgaagtttcaataacctgtctt

SEQ ID NO: 41 - protein, 2199 aa, Diabrotica virgifera
VTSELCSKGSLITFQTNSIMEDSFVFGSVLSEEEWKVVPVDLGKKIIKFVTENFDDLIKGKALLETKCCNLEKNDKDLHDVLEKAELENKTLKSKLEVAANTINEHETQLSNLAAELTKLHAKCNAFEAETAEFRHQRNKAVDEKDEHLRMINRRNGEIERLQADLKDSTKKLESAISAKCEALAQIEEVASMKTNIEYREKRMEQEKALYNSQIESLQEELNKKTEELLNSKRDNSSTCVQLETKLIQKTQELTVATEQIKSLNDINTNLTARIEELSQKIANLRDEEAKVNESYVFEIEAKTKMANAFKSRYEELEHHSKTLEDAVTELQQVLKNATEQYGELETKHKESELAKEEIIAKKNECLELLKKELETANELLDNSRPMLDSEGISPNQSSSKLVKPGMTYSELYSRYVNVSNRLTEKEEECARSNNYVDAIVKEMDFNLPKVRKMQEDYSESLTTIDTLKDANDQLCSEIQLLRDTNAECKKAEGIRTRENERLRNEVSDLSRQVVVLLNEVENSRVGSSSTSTDNDLSDSVNSADIISKKLVTFNDISELQTNNLKLLALVRELSARQEEVESLDPAAIAALKLKVEELMQSQDELLEEREKQTKMMVTLRNQKDMYKNLYTQVVKGAGEEIHMSDVSENGEPKIRTETETQLEEKVQEYEAKVEKLKRDVEHLKEENDIYRKEKSANEKILIDQLDNMRGEVKELTRLNCKLSARAEHDEEKFKVFNNNVDIYKKQITALEKQNKIYSESIIKHEQAATYLKNETIQSQTKLSRAEVMLGNLQKENALLKDAEQRLLKERDSLKVNFHQQNLMKTNIELIKASLERADAEGKLKLENRLDEAHRECATLRRRLQEEQDHFRQLSDHLSAQNKTIEKRAEEEREIAEKLRKEVAEVREDLMAKVAQNEELGKKLKSEMFMVPDGSAETRRVRELEQQIADLQAENESLKKKIKTAKEASDEYFKVAEASEKQVKDAMERNESLMQEVDKHKARIRELEEKCAELEGEISIQLDDQDITNAGKSKSMQLQEELNIRNMDLRTAKQQLETARSENRQLIEQMKAVENKYAREVTLHSVDLQSLTAMKEDLDNALGEINKLQSERQKATEALEQFKENWEKQEHLFQNEKQELNERFKDMDSQNSLLHDQIQALNTQLALLQAQVSDSQTQNTSIGDTSFSKSFTEDETKSSEQLLKIIKYLRQEKDIAVSKADIVEAEHSRLKTQFEMIKKQLDEAKESVETERQKTEISVVSAAKHAEVLRKLETLNAITDSNRALRQERDTLAAQLADLQSKAETFETEVEPLQEKNRELTTKADQLQSENISLRAECTRWRQRANMLIEKTNRTSPEDWKKLQTERETLSKQLIIEKANITKLTDDVNTLKQEKSNLEEKLNTLKNQSSQQQEEITRLRDEVSNLQSQVTQLTNTVDQQSAEIIRFKEETRGLTEETAAKDTTINELKNNMAQVRKIAKKYKIQCEDQTKEISALKQQNEQHESDQVSSAEKQELLESQRSELEEKINELERNHKETVDQLNQEVSSTTEQIENYRKEIDTLKQTSQEKEERFKALFKNAKERIVSLTEQNNNLKEEVNRPGRSDGAEKESEDSAKVDELLEKIALLERERDDILEEKQQEQDKHTTEVESLNQRISQLQRQLGLQQGSKPSTSSSTTEKSSTERPTADIKPMAGHSTNTQTQSVPIQPWRSGGEPPLASIRPMSAQLRTGVVLPTSQSPSAVMVPPQQQVHTTGSSSIEALSSSPTSSHTEYVPATSSASPAMLGPRQVAVPPTQSSQDTEDDESSMQVQAAPQTQAVALVLPRVEPPSSGPTQEQGTSSSSSNTVTTTQAGHKRQREADMDSSQSDEQNKTQQQTKRTRIQQAGTVSDSGLDVEYQVPTSSQRDHDDDNVIVVESDEEGAADEGEGADDEQDDPDTEGYDMEGMEQDNYEDADCQDVEDEEEAGNEVEVIDDSSEVPNQSEREEDNVEEETSEQPQSEAISSGTDGTGRSVPTTPLQSSPQEGIPGSEEQGTSQQSDAETPSIVISGEVDDNETIGESSSGDMGPPAVAGTSSEGAQQHEQAEEMLEGDDGVSSEGEKPPIAEEGEEEGREAEASPSSNSRGVAQRGSNSARRSTRSSLPRGANQRSGPTPIVWSDRSPQRHQHPQDRNRSPQGYQSRTVARRARNRGQRPFDRRF

SEQ ID NO: 42 - DNA, 420 bp, Diabrotica virgifera
tgattaaaaaagcaacttgatgaggctaaagagtctgttgaaacggagaggcagaaaacggagatctctgttgtatcckctgctaaacatgcagaagtacttcgtaaattagaaactcttaatgccatcaccgacagcaatagagctytaagacaggaacgagatactctagctgcccaattagcagacctccaaagcaaagcagaaacattcgaaactgaagtcgaacctttgcaggagaagaacagagagctcacaacaaaggctgaccaattacaaagtgaaaatatatcattacgtgccgaatgtaccagatggagacagagggctaatatgcttattgaaaagacgaacargaacaagtacccgaagattggaagaagttgcagacagaacgggagaccttatccaaacagttga

SEQ ID NO: 43 - DNA, 429 bp, Diabrotica virgifera
agtgccgaaataattaggttcaaggaagaaactcgtggccttactgaggaaacggccgctaaagataccactattaacgagctaaagaataatatggcccaagttagaaagatagctaaaaagtacaagattcaatgcgaagaccaaactaaagaaataagcgcgctgaaacagcaaaatgaacagcacgaatctgatcaagtgagcagtgctgaaaagcaagaactgttggaaagccagcgttcagaattagaagaaaagataaatgaattagaaagaaaccataaagaractgttgaccagttaaatcaagaagtttcttctacaacggagcaaatagagaattaccgaaaggaaattgatactctgaagcaaaccagtcaagagaaggaagaaagatttaaggcgctgtttaagaacgccaaggaa

SEQ ID NO: 44 - DNA, 1073 bp, Artificial Sequence
  hairpin forming sequence
tgattaaaaaagcaacttgatgaggctaaagagtctgttgaaacggagaggcagaaaacggagatctctgttgtatcckctgctaaacatgcagaagtacttcgtaaattagaaactcttaatgccatcaccgacagcaatagagctytaagacaggaacgagatactctagctgcccaattagcagacctccaaagcaaagcagaaacattcgaaactgaagtcgaacctttgcaggagaagaacagagagctcacaacaaaggctgaccaattacaaagtgaaaatatatcattacgtgccgaatgtaccagatggagacagagggctaatatgcttattgaaaagacgaacargaacaagtacccgaagattggaagaagttgcagacagaacgggagaccttatccaaacagttgaagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagtcaactgtttggataaggtctcccgttctgtctgcaacttcttccaatcttcgggtacttgttcytgttcgtcttttcaataagcatattagccctctgtctccatctggtacattcggcacgtaatgatatattttcactttgtaattggtcagcctttgttgtgagctctctgttcttctcctgcaaaggttcgacttcagtttcgaatgtttctgctttgctttggaggtctgctaattgggcagctagagtatctcgttcctgtcttaragctctattgctgtcggtgatggcattaagagtttctaatttacgaagtacttctgcatgtttagcagmggatacaacagagatctccgttttctgcctctccgtttcaacagactctttagcctcatcaagttgcttttttaatca

SEQ ID NO: 45 - DNA, 3383 bp, Diabrotica virgifera
gggaagggacggtaaccggtcgtattcaccgcgcccaaggaacgatagtggtgaaaaacccagtgaaaaattgtaaaatcggcgtgtagattgtgcgggtaatgcgtttacatctatgagacctctacagacaaattctacatgaatattaggagtggacaatatattttctatgtatgcaaaccgatgtggttgtgattttccgagcaccattggatagttttataggtgattggtttaggacagctgtaaagctttggaaagaggagtaaatttgttttgtgtcgaacagtatgaacgaactggattctcttaggcaagaagctgaaaccctcaaaaatgctattagagatgctcgcaaagcggcttgtgacacatctttggtacaggctacctccagcctggagcccattggtcgagtgcagatgcggactagacgtactctcagaggccatttggccaagatctacgctatgcactggggctcagactcaaggaatcttgtctcagcatcacaagatggaaaacttatcgtatgggacagtcacaccacaaataaggtacatgcaatccccctcagatcatcatgggtgatgacatgtgcttatgcaccatctggaaattttgtcgcctgtggaggtttagataatatatgctctatctatagcctaaagactagagaaggcaatgttcgggtcagccgagaattgcccggtcatactggttatctatcttgctgtcgtttcctcgatgacaaccaaatcgtgactagttcaggagatatgtcttgtgccctatgggatattgagactgggcagcaagtcgcctctttcttaggacacacaggagatgtaatgtctctttccttgtcacctgacatgcgtacatttgtatcaggagcatgtgatgcttctgcgaagctgtgggatattcgagaaggtttgtgcaaacagacattccccggtcacgagtcagacatcaacgctgttacgttcttccccaacggtttcgcctttgccacaggtagcgacgatgctacatgcagactgttcgacatcagggcggaccaagaattggctatgtatagccacgataacatcatttgcggcatcactagcgtcgcttttagcaaatctggtagattacttctagctggatatgacgatttcaattgtaatgtgtgggactctatgaaaacagaaagagcaggtatattagctggtcacgataatcgtgttagttgtttaggagttacagataatggaatggcagtggggacaggttcgtgggacagtttcctacgcatttggaattaagatagtgatgccaaaaaacgttaaatttctcacctgcatcacaatatctctttacattgcatttattcaaatcgaaacttttttattttaagtgtactatatatatagttatattttttcaatattctttgcttttttattatcttatttctgttatataaatagcataaaatgtgtctagctttgtgatgtattttctttaatttttaagtcgttattttttgtgacaattctacaaattttaggcgctggcccatgtatgttgtgtatagtatttaatatcattttaagttaataaatcagtattgtttatgttgatgatttgatgatgtaggtgagaaataagaggatattggttgttcagtgcgtacgccaaaatagcgtaccgtttttcattttggttagttccaagtacaaacatatgaccaagctagttaaaacaagtaccgtactttatcctgaactgtttttatccagttgagccagtctctcaaatcactctcttttaacaaaataagggccagctgcgtaaaaacttcctttggcgaagaacaaactgaaaaaaatctgcttctgaacaaacaaaaaatagcagatatgataaaactagtcttacaaattttaaccaaatgttgaaagtgtgccataatctcaacttagcgtcacaaacaaacaaaaaaagaccaatgatagagttgattgtggcgaggttcctttgattgaacaacaggcaggttacgaatcaaattgtttgagatactgcctataaaaattttggaaagaacagtgttccatttttgctaatactttttttatcaacactttttttatttacctcagtgttttttctatgacatcgcagttctgtacatatatgttcctctgatgatgttttttcgtcttgttttttagaaatctttaaggttttatcatcccccacatgactatatagcttctgcttgaatgtaagtggtatgaacatttttttaaaaagctaaatttcacttatttttttgatttttgattgcacaagcataatacggtgtatctcccgtgatattttcaacgtctttttataaaatttcataccgtatcgcggggcatattgccattaaaactaagtgtataattttaaattatttattcgagtgtgaacgtcagtgaatatgagaacaaacaatcgtgtgcgttgggagactttggggtagggttcgcgttaagataagctacgaaatcacaaataccacaatgtcactgttggcaatttattaaataattcttaattggaaatacacatccatccaagtattaagggctcaaaattgtttgcagtttcatttgatgcactgtgttgcatactttttccttgtacatttctgaacaaatttttaattgacattgtaacggtcttagaaaactgaatagtggatttcctacatactcaacattactggttcaactaaattacctctgttgactgccacctatggtcaagataagctttaacatatcatcaatacctgattgatgttttcccttgattcattacattttcttgcattacgagccctaacaacttgttctaacaaacagaaatttattgaaaatgttcgcctttacaaccttgcttattgggttaaagaacgagatcagcaatacattggagtctatcattaatgataaaatattgaaatggcttggtttctatgaactttaaattaaatgttgtaatcaacaaatggcaaaacaagaagaaatctctttcacaatcaagaaacggaaacttgaatacctttgtcatattatgatacatgataaatgtcatatactccaactgataatacagggtaaaatagaaagcaaacgcggacccggaagaagaagacactcatggcttcaaaacttacgccaaaggtttggactaacatcgatcgagttatttaaaaacgccgcaaacaaaattagaattgctatgatgatagccaacgtccgcaacggacaaggcacacgaagaataagaagagttgtaatcaagatctctgttggactttctttcaaaactacgct

SEQ ID NO: 46 - protein, 344 aa, Diabrotica virgifera
VSNSMNELDSLRQEAETLKNAIRDARKAACDTSLVQATSSLEPIGRVQMRTRRTLRGHLAKIYAMHWGSDSRNLVSASQDGKLIVWDSHTTNKVHAIPLRSSWVMTCAYAPSGNFVACGGLDNICSIYSLKTREGNVRVSRELPGHTGYLSCCRFLDDNQIVTSSGDMSCALWDIETGQQVASFLGHTGDVMSLSLSPDMRTFVSGACDASAKLWDIREGLCKQTFPGHESDINAVTFFPNGFAFATGSDDATCRLFDIRADQELAMYSHDNIICGITSVAFSKSGRLLLAGYDDFNCNVWDSMKTERAGILAGHDNRVSCLGVTDNGMAVGTGSWDSFLRIWN

SEQ ID NO: 47 - DNA, 469 bp, Diabrotica virgifera
gtatgaacgaactggattctcttaggcaagaagctgaaaccctcaaaaatgctattagagatgctcgcaaagcggcttgtgacacatctttggtacaggctacctccagcctggagcccattggtcgagtgcagatgcggactagacgtactctcagaggccatttggccaagatctacgctatgcactggggctcagactcaaggaatcttgtctcagcatcacaagatggaaaacttatcgtatgggacagtcacaccacaaataaggtacatgcaatccccctcagatcatcatgggtgatgacatgtgcttatgcaccatctggaaattttgtcgcctgtggaggtttagataatatatgctctatctatagcctaaagactagagaaggcaatgttcgggtcagccgagaattgcccggtcatactggttatctatcttgctgtcgtttcctcgatgacaacca

SEQ ID NO: 48 - DNA, 547 bp, Diabrotica virgifera
agttcaggagatatgtcttgtgccctatgggatattgagactgggcagcaagtcgcctctttcttaggacacacaggagatgtaatgtctctttccttgtcacctgacatgcgtacatttgtatcaggagcatgtgatgcttctgcgaagctgtgggatattcgagaaggtttgtgcaaacagacattccccggtcacgagtcagacatcaacgctgttacgttcttccccaacggtttcgcctttgccacaggtagcgacgatgctacatgcagactgttcgacatcagggcggaccaagaattggctatgtatagccacgataacatcatttgcggcatcactagcgtcgcttttagcaaatctggtagattacttctagctggatatgacgatttcaattgtaatgtgtgggactctatgaaaacagaaagagcaggtatattagctggtcacgataatcgtgttagttgtttaggagttacagataatggaatggcagtggggacaggttcgtgggacagtttcctacgcatttggaattaag

SEQ ID NO: 49 - DNA, 1171 bp, Artificial Sequence
  hairpin forming sequence
gtatgaacgaactggattctcttaggcaagaagctgaaaccctcaaaaatgctattagagatgctcgcaaagcggcttgtgacacatctttggtacaggctacctccagcctggagcccattggtcgagtgcagatgcggactagacgtactctcagaggccatttggccaagatctacgctatgcactggggctcagactcaaggaatcttgtctcagcatcacaagatggaaaacttatcgtatgggacagtcacaccacaaataaggtacatgcaatccccctcagatcatcatgggtgatgacatgtgcttatgcaccatctggaaattttgtcgcctgtggaggtttagataatatatgctctatctatagcctaaagactagagaaggcaatgttcgggtcagccgagaattgcccggtcatactggttatctatcttgctgtcgtttcctcgatgacaaccaagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagtggttgtcatcgaggaaacgacagcaagatagataaccagtatgaccgggcaattctcggctgacccgaacattgccttctctagtctttaggctatagatagagcatatattatctaaacctccacaggcgacaaaatttccagatggtgcataagcacatgtcatcacccatgatgatctgagggggattgcatgtaccttatttgtggtgtgactgtcccatacgataagttttccatcttgtgatgctgagacaagattccttgagtctgagccccagtgcatagcgtagatcttggccaaatggcctctgagagtacgtctagtccgcatctgcactcgaccaatgggctccaggctggaggtagcctgtaccaaagatgtgtcacaagccgctttgcgagcatctctaatagcatttttgagggtttcagcttcttgcctaagagaatccagttcgttcatac

SEQ ID NO: 50 - DNA, 2122 bp, Diabrotica virgifera
ggcaaatcggtccagatgaccggacatatttgttgtgtgccgtgaacaatttgttgaggaaaaatggcagagccagaattaaatgtagacagtttaatacaaagattattagaagtaagagggatgagaccgggcaaatcggtccagatgaccgagtccgaagtgcgcggtctctgcctcaaatcccgcgagatcttcctgcagcagccgatcctgctggagctcgaggcgccgctcaagatctgcggcgacatccacggtcaatacaccgacctgctgcgtctcttcgagtacgggggtttcccccctgaagccaactatctgtttctcggagactacgtcgatcgcggcaagcagtccttggagacgatatgtctactgttagcgtacaagattaagtatccagagaattttttcctgctcagaggtaaccacgaatgcgcctccatcaacaggatttatgggttctatgacgaatgtaaaagaaggtacaatataaaattatggaagacattcacggactgcttcaattgcttacccatttccgccattatcgatgaaaagattttctgctgtcacggtggactgagtccagatttgcaaggaatggaacaaatcaggagaataatgaggccgacagatgtaccagacaccggtcttctttgtgaccttctttggtccgatccagacaaagacgtccaagggtggggcgagaacgatcgaggtgtttccttcaccttcggagcagacgtcgtaagcaaattcctgaaccgacacgatctcgatttgatctgccgcgcccaccaagttgtcgaagatggctacgagttcttcgccaagcgacagctcgtcactttgttctccgctcccaactattgcggcgagttcgacaatgctggagggatgatgagcgtggacgagacgttgatgtgttcgtttcagattctcaaaccgtcagaaaagaaggcaaagtaccaataccaggggatgaattcaggccgcccatccacaccgcaacgaaatccccctaacaaaaataagaagtagatcactaacttatttctagcccccctccttcccttccctctacacctccgccctatccttacactactttttgtattcatcttatctgtcgacgatgcatttgtaatgagtagtttctctaagcaatgccccccggaacagaataaaaaatggtttaagcatgtcaagaactattattgtatattatttaatgttttattataatttatatattgtattatgtgactgattgtttttttttcaggtttttttttgttgagtgctgactgaattgctttgtattatttttcttttgcgttatcccggcttggatttgtcatttgagatgatcatttcagctgtattgtttccttaaatcgtctacaaaaatttgtaaaaatcgaacactacagattctgctgatactcaaatcttagaaatttagtgtatgtagctcccactgatgacactcattgccaattgtcatcaatcgattacttttacgatttaaaaaagcaaagaaatttataaaaggacatataacttcaatttcgatgattctttcgagtttaacggtggaaatataacagaaagtgggagtatccctctcccactgacgccactgtcttgtcaaatgtcaccaatgcggcagcttgttatttgtcattttctacactgactgattatcttaactgactaatctatgcctaaatttacaattattcgtgttaagtgtgtacccacattgtacataaacactttcttttaaaattcaccgaggagcactgtaaatccacatactaactacttaaaagacagtctcctgaattttcaaccatagtgaaaggttttcaatcgtacaatcatcaataaatttaagtttgaatgtttttgttcttttttcttaccgcaatcgaagtgaaatgacaattatatgtgtgctttctactgttttaagtcgataaaaggacaaagttccaattattagtcgtgtgaacaaatatttttattagatttcttagagtacgctagaaaatctaattttgaagtgtacagggcactatattttgatccccctgtaaactgcttcatttac

SEQ ID NO: 51 - protein, 327 aa, Diabrotica virgifera
MAEPELNVDSLIQRLLEVRGMRPGKSVQMTESEVRGLCLKSREIFLQQPILLELEAPLKICGDIHGQYTDLLRLFEYGGFPPEANYLFLGDYVDRGKQSLETICLLLAYKIKYPENFFLLRGNHECASINRIYGFYDECKRRYNIKLWKTFTDCFNCLPISAIIDEKIFCCHGGLSPDLQGMEQIRRIMRPTDVPDTGLLCDLLWSDPDKDVQGWGENDRGVSFTFGADVVSKFLNRHDLDLICRAHQVVEDGYEFFAKRQLVTLFSAPNYCGEFDNAGGMMSVDETLMCSFQILKPSEKKAKYQYQGMNSGRPSTPQRNPPNKNKK

SEQ ID NO: 52 - DNA, 473 bp, Diabrotica virgifera
ggtggactgagtccagatttgcaaggaatggaacaaatcaggagaataatgaggccgacagatgtaccagacaccggtcttctttgtgaccttctttggtccgatccagacaaagacgtccaagggtggggcgagaacgatcgaggtgtttccttcaccttcggagcagacgtcgtaagcaaattcctgaaccgacacgatctcgatttgatctgccgcgcccaccaagttgtcgaagatggctacgagttcttcgccaagcgacagctcgtcactttgttctccgctcccaactattgcggcgagttcgacaatgctggagggatgatgagcgtggacgagacgttgatgtgttcgtttcagattctcaaaccgtcagaaaagaaggcaaagtaccaataccaggggatgaattcaggccgcccatccacaccgcaacgaaatccccctaacaaaaataagaagtagatcac

SEQ ID NO: 53 - DNA, 1179 bp, Artificial Sequence
  hairpin forming sequence
ggtggactgagtccagatttgcaaggaatggaacaaatcaggagaataatgaggccgacagatgtaccagacaccggtcttctttgtgaccttctttggtccgatccagacaaagacgtccaagggtggggcgagaacgatcgaggtgtttccttcaccttcggagcagacgtcgtaagcaaattcctgaaccgacacgatctcgatttgatctgccgcgcccaccaagttgtcgaagatggctacgagttcttcgccaagcgacagctcgtcactttgttctccgctcccaactattgcggcgagttcgacaatgctggagggatgatgagcgtggacgagacgttgatgtgttcgtttcagattctcaaaccgtcagaaaagaaggcaaagtaccaataccaggggatgaattcaggccgcccatccacaccgcaacgaaatccccctaacaaaaataagaagtagatcacagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagaggtgatctacttcttatttttgttagggggatttcgttgcggtgtggatgggcggcctgaattcatcccctggtattggtactttgccttcttttctgacggtttgagaatctgaaacgaacacatcaacgtctcgtccacgctcatcatccctccagcattgtcgaactcgccgcaatagttgggagcggagaacaaagtgacgagctgtcgcttggcgaagaactcgtagccatcttcgacaacttggtgggcgcggcagatcaaatcgagatcgtgtcggttcaggaatttgcttacgacgtctgctccgaaggtgaaggaaacacctcgatcgttctcgccccacccttggacgtctttgtctggatcggaccaaagaaggtcacaaagaagaccggtgtctggtacatctgtcggcctcattattctcctgatttgttccattccttgcaaatctggactcagtccacc

SEQ ID NO: 54 - DNA, 3472 bp, Diabrotica virgifera
ttataatgtcatcaatttcaccttcccgctaaagcaaatactattttttgttaattctacgacttattgtatccttgctagtaaaaacaattgaattttcaatgttttccgaatttaataccaaattaaatatttaaatcaatgcatttgaaggatttcaatttacaaaatagtttttaaagtgacagttgacaagtcgatttgacagaaggcagtaaatttgtctagactgccgtgaaattacatgtggtgtagaaccgttttgtgaagccggttaattttctagaacaaataaatacaattcttggcttttggaaggcgtaaaatagtttcaaaatgcatcaggaggccatacaactcatccaggtcctagaaaaaacagtttcaccggataaaaatgaattggaacaagcgtcatcgtttctcgaacaagctgcagtgactaacctactagaatttatcaaaactctatcagatatattaagacatggcggaaatagccctgtggcccgaatggccgcaggtttgcagttaaaaaatcaactcacctctaaagactccaacataaagaccacctaccaacaacggtggttagcttttccagaggacgtccgggggtatattaaaaataacgttgtgtgcgcacttggaaccgaaaccagcagaccatcatccgcagctcagtgcgtagcatacgtcgcagtaacagaactaccacaagggcaatggccagatctcatagcaacgttggtcaacaatgtttgccatgcaaattccacagaaatgcaaaaggaagccacattagaagctatcggttatatatgccaagaaatcgactcggacgtattagtatctcaatcaaacgacatcttaacagccatagttcatggaatgcgagcctcggaaccgagcaaccacgttcgtttagcagctacacaggccctactaaactctctagaattcacaaaggccaactttgaccaaccgacggaacggaactatatcatggaagtcgtctgtgaagccacgcagtctccagattcacagatcagagtggccgctttgcagtgtctagttaagattatgtctttgtattatcaacacatggaaccttatatggctcctgctctattccctatcactctagaagccatgaagtcggaaaacgatgctgtttctctacagggtatcgaattctggtctaacgttagtgatgaagaggttgatttggccatcgaagataccgaagccactgaagcaggtcgaccacctacaagatgttcacggcactacgccaaaggtgccctacaatatatagtaccgattctgctgcaaaaattaacgaaacaggaagaactggacgacgaagacgattggaatccttgcaaagcagccggcgtttgcttaatgcttctcgccacgtgttgcgaagacgaaatcgttccacacgttctgcccttcatcaaagaaaatattaagtctgataattggcgattccgagatgcttctttgatggctttcggatctattttaggtggtttggagagtactactttgagaccgcttgttgaacaggcgatgccgaccttgatagatctgatgtacgacaatagcgttatcgtaagagacacagccgcctggaacttcggcagaatttgcgaaatcattccggaggcggccatcaacgaaaccttcctcaaaccgctattggaaagcttgatcacaggcctcaaggccgaacctcgtgttgccgccaatgtctgttgggcgttctctggattggtggaggcagcttacgaaaatgccgacgttaatgaagaaaccggtactccagatacttacgtcttgtcacaatacttcgagttcattatacaaagactgcttgaaacaacggatcgacccgatggcgcccaacacaatttacgacctgcagcctacgaagccctcatggaaatggtgaaaaactcacccaaagattgctatgtcaccgtgcaaaagactacaatggtcattttggacagattacatcaggtcttacagatggaaactcacataagcagccacaacgatagatcgcaattcaacgatctccagagccttttgtgtgccactcttcagtcagtactcagaaaagttaccccagaagatgcgcctcacatctcagacgccataatgaccgccatgttgaccatgttcaattccaattcatgtaagagtggaggtgttcaagaagacgctattatggccgtctcgacgctggtcgaagtcctgggagaaaacttcataaaatacatggacgccttcaaaccgttcctctacctcggattgaagaaccaccaagaataccaagtgtgcgttacggcagtgggcttaaccggcgacatcttcagagctctcaaactgaaagcgctaccatactgtgatgaaataatgaccttattactagaaaatctccgcgatcagtcagtccatcgtagtgtgaagcctgagatcttgggcgtgttcggagacatcggtctcagcatcggctccgagttcaagaagtacctcgaggtggttctgtccactttggctcaggcttcgcaggcgcaagtcgaccgcaacgacttcgacatgatcgattatcttaacgacctaagagagggagttttagaagcttataccggcatcatacaagggcttaagggtgacgggcccactccacatcctgacgttttgatacttgaaccccacattccatttatcgttcaatttattactgtcgtagcacaagatacggaacattctgatagtattatagctgccgcagctggtattgtaggtgatatgtgtaatgttttcggagctccaatggttcaattcttagatctggacccaattaatgaaatgttagcacaaggtagacgtagtcgagtaaataggactaagactttggccaattgggccgttaaagaattaaagaagctgaaggccgtaaacaccgcggctgctgctagctgattcttcttggctgtttagaaacaccaaagagagatctacatcatcataaaaaaattttcatgttgatttttattcaaaatttacaaaaaaaactcgttttaaattatgtttgattaattattaaaaaatatattacatacatacgtggccggtctcaggtatattttaaaaaatcaagtcaagaatcatcatccaacatgagaatttgtcataataactttcagattaactgatactgagtgatcatctatctgtgcagtgcaaataaaggaactccggaaccgtttggtgatataaaatagcaaaatgaacgcttgtaaatagtgtctgttctagtgcgcgtcagcagacctttcagctcaagactgatgaaaagtgatggcatttcagtatcacttggtgcgtcagttcatcatttttaagtaatgtaacatgataaaaataaagaattttttctatatttaaaaaaaaa

SEQ ID NO: 55 - protein, 887 aa, Diabrotica virgifera
MHQEAIQLIQVLEKTVSPDKNELEQASSFLEQAAVTNLLEFIKTLSDILRHGGNSPVARMAAGLQLKNQLTSKDSNIKTTYQQRWLAFPEDVRGYIKNNVVCALGTETSRPSSAAQCVAYVAVTELPQGQWPDLIATLVNNVCHANSTEMQKEATLEAIGYICQEIDSDVLVSQSNDILTAIVHGMRASEPSNHVRLAATQALLNSLEFTKANFDQPTERNYIMEVVCEATQSPDSQIRVAALQCLVKIMSLYYQHMEPYMAPALFPITLEAMKSENDAVSLQGIEFWSNVSDEEVDLAIEDTEATEAGRPPTRCSRHYAKGALQYIVPILLQKLTKQEELDDEDDWNPCKAAGVCLMLLATCCEDEIVPHVLPFIKENIKSDNWRFRDASLMAFGSILGGLESTTLRPLVEQAMPTLIDLMYDNSVIVRDTAAWNFGRICEIIPEAAINETFLKPLLESLITGLKAEPRVAANVCWAFSGLVEAAYENADVNEETGTPDTYVLSQYFEFIIQRLLETTDRPDGAQHNLRPAAYEALMEMVKNSPKDCYVTVQKTTMVILDRLHQVLQMETHISSHNDRSQFNDLQSLLCATLQSVLRKVTPEDAPHISDAIMTAMLTMFNSNSCKSGGVQEDAIMAVSTLVEVLGENFIKYMDAFKPFLYLGLKNHQEYQVCVTAVGLTGDIFRALKLKALPYCDEIMTLLLENLRDQSVHRSVKPEILGVFGDIGLSIGSEFKKYLEVVLSTLAQASQAQVDRNDFDMIDYLNDLREGVLEAYTGIIQGLKGDGPTPHPDVLILEPHIPFIVQFITVVAQDTEHSDSIIAAAAGIVGDMCNVFGAPMVQFLDLDPINEMLAQGRRSRVNRTKTLANWAVKELKKLKAVNTAAAAS

SEQ ID NO: 56 - DNA, 491 bp, Diabrotica virgifera
cgattctgctgcaaaaattaacgaaacaggaagaactggacgacgaagacgattggaatccttgcaaagcagccggcgtttgcttaatgcttctcgccacgtgttgcgaagacgaaatcgttccacacgttctgcccttcatcaaagaaaatattaagtctgataattggcgattccgagatgcttctttgatggctttcggatctattttaggtggtttggagagtactactttgagaccgcttgttgaacaggcgatgccgaccttgatagatctgatgtacgacaatagcgttatcgtaagagacacagccgcctggaacttcggcagaatttgcgaaatcattccggaggcggccatcaacgaaaccttcctcaaaccgctattagaaagcttgatcacaggcctcaaggccgaacctcgtgttgccgccaatgtctgttgggcgttctctggattggtggaggcagcttacgaaaatgccgacgtt

SEQ ID NO: 57 - DNA, 604 bp, Diabrotica virgifera
aatgaagaaaccggtactccagatacttacgtcttgtcacaatacttcgagttcattatacaaagactgcttgaaacaacggatcgacccgatggcgcccaacacaatttacgacctgcagcctacgaagccctcatggaaatggtgaaaaactcacccaaagattgctatgtcaccgtgcaaaagactacaatggtcattttggacagattacatcaggtcttacagatggaaactcacataagcagccacaacgatagatcgcaattcaacgatctccagagccttttgtgtgccactcttcagtcagtactcagaaaagttaccccagaagatgcgcctcacatctcagacgccataatgaccgccatgttgaccatgttcaattccaattcatgtaagagtggaggtgttcaagaagacgctattatggccgtctcgacgctggtcgaagtcctgggagaaaacttcataaaatacatggacgccttcaaaccgttcctctacctcggattgaagaaccaccaagaataccaagtgtgcgttacggcagtgggcttaaccggcgacatcttcagagctctcaaactgaaagcgctaccat

SEQ ID NO: 58 - DNA, 1215 bp, Artificial Sequence
  hairpin forming sequence
cgattctgctgcaaaaattaacgaaacaggaagaactggacgacgaagacgattggaatccttgcaaagcagccggcgtttgcttaatgcttctcgccacgtgttgcgaagacgaaatcgttccacacgttctgcccttcatcaaagaaaatattaagtctgataattggcgattccgagatgcttctttgatggctttcggatctattttaggtggtttggagagtactactttgagaccgcttgttgaacaggcgatgccgaccttgatagatctgatgtacgacaatagcgttatcgtaagagacacagccgcctggaacttcggcagaatttgcgaaatcattccggaggcggccatcaacgaaaccttcctcaaaccgctattagaaagcttgatcacaggcctcaaggccgaacctcgtgttgccgccaatgtctgttgggcgttctctggattggtggaggcagcttacgaaaatgccgacgttagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagaacgtcggcattttcgtaagctgcctccaccaatccagagaacgcccaacagacattggcggcaacacgaggttcggccttgaggcctgtgatcaagctttctaatagcggtttgaggaaggtttcgttgatggccgcctccggaatgatttcgcaaattctgccgaagttccaggcggctgtgtctcttacgataacgctattgtcgtacatcagatctatcaaggtcggcatcgcctgttcaacaagcggtctcaaagtagtactctccaaaccacctaaaatagatccgaaagccatcaaagaagcatctcggaatcgccaattatcagacttaatattttctttgatgaagggcagaacgtgtggaacgatttcgtcttcgcaacacgtggcgagaagcattaagcaaacgccggctgctttgcaaggattccaatcgtcttcgtcgtccagttcttcctgtttcgttaatttttgcagcagaatcg

SEQ ID NO: 59 - DNA, 4030 bp, Diabrotica virgifera
cccacctctagtttatacattagtaaatgtcaaatttattaagaaaatgtcactgatgtgatagttctttagttttattgtagaaaaaattaaaaacttaaaaatttagttaagacagttgtatatggacatgaatgaagctttctccctaacatgtgaacgtgcgtaaacggttcccgcgcactgcgtgctcctagtattttcatggctccatggcatatccgccatgatggctttatgctcaacacgctcaacatggaaagtttcaaagttgttttatcgtatttgtgactgagtgcgtgtaaataaaaacaatgggaacgtactaaagtaaacgaagaattgtttagtgccctcctcgaacggcccgagggaaagggagctagtttttaggaggttttatgttggccttgtcgagtgttttgtgctgtcaacaaatatgtcgaagctttcatttagggcgagggccctggatgctagcaaacccatgcctatatacatggctgaggaactcccggatcttcctgattattcagcgatcaatcgggcagttcctcaaatgccatcaggaatgcaaaaggaggaggaatgtgaacaccatcttcaacgtgcaattgttgctggactcatcattcctactcccgaagtttccgaattgccagacaaggagttctatgaaaaggtctatccagcaaattataagcaaccccgacagcttatccacatgcaaccattcacaatggaacaggatattcccgattatgatatggactcggatgacgagaggtggcttcaagctcagacgcaaaaattggacctaagtccagtcaagtttgaagagatgatggacaggttagaaaagagcagtggtcaaactgttgtcaccttaaacgaggccaaggcgcttctcaaagaagacgacgatctcataatagcagtatttgattattggctgaataaacgcctgaaaacacaacatcctttgattttgaccgtaaaaacggaaataagatctggtactgcagctaacaacccatatctggcatttaggcgacgaacagagaagatgcagacaaggaaaaaccgcaaaaacgatgaagcttcctatgaaaagatgctaaaactgagaagggatttatacagggctgtgacactgttagaacttgtaaagaggcgagaaaaaatcaagcgagagtatgtacatcttacggtagaggtatttgagaaacgcttccaagccaaggatttcagtggtgctgtaatggcagaagcttcggctatcaaatcatccagacctgcatttacaccaatatttcacaatcactatccaaatcaaagttgggcgaataagtctatacttaaagatgaggttataccacgaagagaaaaacgacagtacaaaaaacgcaaacacaaatcgctgggtaatcgatctggaggttatggagttgattctttgggcggtatgtcttctgatgatgaggtgaatatgtcacagttatctccagaaccagaagaggctgaagatgaaagtcagtttgcattcaaaaggaacaagctatgttcttatcataggccactttcgcatgaaggcaactggaggtgggcgtcaaaggaagagaatggttcagcagacaaacggttccgatttacgttaacatccctctcgaatccgcgtcggtgtataggcttcgctcgaaggagggttggtagaggcggtagaataattctcgatcgaatttcaacaaattacgacgatttctggaggacattagatttctctataacggaaccggatagagaggctggtacgagtagagttgtcgaggaggagccagccctagttcaagaaactgatattaaaagtgaagttagaagtgaagttagtgttagtaacagtgcacggataagtgatagtagtgtgattagtgatagtattaaggtggaaattaagaaggagccggaggatgtgcaagaggagggtgttacacaactggaacctgtgtatactaaagaagagaatgatgatatggtcgattttcttcgatcacttaggagggattggttacattttcgacctaaaacgccaccacctgactatgaagttccctgtgatatgcttcattctgaggagacattcttcgatcccagtgccaacaccttctcgatggagatacagacattggatgcacctagttctacctttctggatacctctacgtttgtctctgatcctttcacgctcgaccagctggacttggattcaaggcagctcctacctgctgcatccactctcaatacgcttatacctagtgttagtagtgatagtaacgaaaacgacaatttcttgtcaagtgattcttctagtagtgatagtgactttagaactattggttgttcgaattttaaagtaaatgatctaggtttgaatgacagtaatgttactacaagttctacaatgacgaaaagtcagcaaacaaaagttcaatattccagtacctctgctagtcctaaagtaaatagtgtgaatacaacatcctcttcgagtggggaagattttaaaccaaccagaggcaactctgactcttctttgcttgggaataccaatggattgttgagtcagttttgctttgatgtacctccttctagaacaaaaaataataaattacaatacgcttactctggagcaatgggatatccaaatgcttcttcaggtaccctaagtcttcacacttctgcgcatacccttacgcttaatagtcatagtgccaacatattggatataccattgactaatgacacagaaacaaataaaatagattcagaagccactggggggcctgtatcaagtaacaataagtcaaaaagtatagtacgaaaaaataacattataatggaggtaacgtgatgatcattttggcgcctgctatcctcgtggattgcgggatgaggaatcagtttttgagtacagggtgttgtattacacaaaacataaaaaactcagcatatatgcctgcatgatttcggtgttaagttattgatttatataatatagaaactagaactcatgtatcacatgctaagaggagttgagagaaatatttaaatggtttttctctgctttcaaatgatttttattgttgcaaattatagtacaaaaatattagtatataatgcaagtatataagaaaatatgttataaacaaatatcaaaagcatcatattaaatcacgtagtaatatttcaggtgttgatcaaaaatgcagtatgtgtcgaaagtacagatgataaagccttaaaagtagctgatgcacaaactaacatcatttctgaaagattttacatttattttacatcaaataatcatagtggacaaaattttttaatatgtattcttgaaaaaaaaattatacaattgttaataagaaagcgtatcaactagttgtcttttttaagcaaactagcaagtttacatacaaatttgaaaagatgaattatattagatacttgtaaaaatgataattctagccatacagctcacaatactttcaaaacttggtttgactcaaacaaaaacttgatacaaaaactcaaaaggtttgtttatgcagcattttcaaacgcatcaaaaactgagaagtttgcacagtaaacatttggctttcataaatcaaacagtttgatcaaatgttcgtacagtatttgcactcaatcgattgcaaaatttcatgttggatggtatcaaatttgtaacgtgttcaattgtgtttttagggctattctcaaatctttactacaaagaattacatatttcattttgataaagttggctcattctttttcagtttagacttgtatttcggtagtctacttttttattttctgttattagtgttttaacgaaaattaaacttgtgtgggaagaaagaatgcccagagttgggtattattttgggcaatgaaaaaattaagggttaatatgaattcagaattatgaaggtttccttggtagatagagttacgaacaatg

SEQ ID NO: 60 - protein, 822 aa, Diabrotica virgifera
MSKLSFRARALDASKPMPIYMAEELPDLPDYSAINRAVPQMPSGMQKEEECEHHLQRAIVAGLIIPTPEVSELPDKEFYEKVYPANYKQPRQLIHMQPFTMEQDIPDYDMDSDDERWLQAQTQKLDLSPVKFEEMMDRLEKSSGQTVVTLNEAKALLKEDDDLIIAVFDYWLNKRLKTQHPLILTVKTEIRSGTAANNPYLAFRRRTEKMQTRKNRKNDEASYEKMLKLRRDLYRAVTLLELVKRREKIKREYVHLTVEVFEKRFQAKDFSGAVMAEASAIKSSRPAFTPIFHNHYPNQSWANKSILKDEVIPRREKRQYKKRKHKSLGNRSGGYGVDSLGGMSSDDEVNMSQLSPEPEEAEDESQFAFKRNKLCSYHRPLSHEGNWRWASKEENGSADKRFRFTLTSLSNPRRCIGFARRRVGRGGRIILDRISTNYDDFWRTLDFSITEPDREAGTSRVVEEEPALVQETDIKSEVRSEVSVSNSARISDSSVISDSIKVEIKKEPEDVQEEGVTQLEPVYTKEENDDMVDFLRSLRRDWLHFRPKTPPPDYEVPCDMLHSEETFFDPSANTFSMEIQTLDAPSSTFLDTSTFVSDPFTLDQLDLDSRQLLPAASTLNTLIPSVSSDSNENDNFLSSDSSSSDSDFRTIGCSNFKVNDLGLNDSNVTTSSTMTKSQQTKVQYSSTSASPKVNSVNTTSSSSGEDFKPTRGNSDSSLLGNTNGLLSQFCFDVPPSRTKNNKLQYAYSGAMGYPNASSGTLSLHTSAHTLTLNSHSANILDIPLTNDTETNKIDSEATGGPVSSNNKSKSIVRKNNIIMEVT

SEQ ID NO: 61 - DNA, 225 bp, Diabrotica virgifera
atgtcgaagctttcatttagggcgagggccctggatgctagcaaacccatgcctatatacatggctgaggaactcccggatcttcctgattattcagcgatcaatcgggcagttcctcaaatgccatcaggaatgcaaaaggaggaggaatgtgaacaccatcttcaacgtgcaattgttgctggactcatcattcctactcccgaagtttccgaattgccaggt

SEQ ID NO: 62 - DNA, 395 bp, Diabrotica virgifera
tccaaatcaaagttgggcgaataagtctatacttaaagatgaggttataccacgaagagaaaaacgacagtacaaaaaacgcaaacacaaatcgctgggtaatcgatctggaggttatggagttgattctttgggcggtatgtcttctgatgatgaggtgaatatgtcacagttatctccagaaccagaagaggctgaagatgaaagtcagtttgcattcaaaaggaacaagctatgttcttatcataggccactttcgcatgaaggcaactggaggtgggcgtcaaaggaagagaatggttcagcagacaaacggttccgatttacgttaacatccctctcgaatccgcgtcggtgtataggcttcgctcgaaggagggttggtagaggcggct

SEQ ID NO: 63 - DNA, 683 bp, Artificial Sequence
  hairpin forming sequence
atgtcgaagctttcatttagggcgagggccctggatgctagcaaacccatgcctatatacatggctgaggaactcccggatcttcctgattattcagcgatcaatcgggcagttcctcaaatgccatcaggaatgcaaaaggaggaggaatgtgaacaccatcttcaacgtgcaattgttgctggactcatcattcctactcccgaagtttccgaattgccaggtagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagacctggcaattcggaaacttcgggagtaggaatgatgagtccagcaacaattgcacgttgaagatggtgttcacattcctcctccttttgcattcctgatggcatttgaggaactgcccgattgatcgctgaataatcaggaagatccgggagttcctcagccatgtatataggcatgggtttgctagcatccagggccctcgccctaaatgaaagcttcgacat

SEQ ID NO: 64 - DNA, 2196 bp, Diabrotica virgifera
aaactattcataaattagtacaatcaaaaacattgcttcgaccctgtacttcaagcacgacgttcaaagctcgctgagggtgcgaattggcatcgttgcgctccgtaagaacctgtactgacttctcgaagtgacaaacactattggcatctcgtcctaagcaatacgccctctagatactcgtttctgaggttcacagctaaaacaacatgtttttgtgaggttaagtttacatattttatttctcaaaattttaaatttttttataaatattgaataatatggaactgtttgttgttaacaggcatgatgatggacagcaagaagaaattcataacgaacaggataaattagataaggtcctcaagaagattcagaaaaataaaaagttacgtcaaaaacagaaagaaaagatatctcaagttaaacaagccaaacagcgaacattacaatcaagggaaatcaaacgaaaaataaaagcttctatcataaaagttcctgataatataaatgatgattttctggataaaactgtagaaaatttagttattacacaaaccagtgataatattgacgaatcaaaacacaaaaaacgcaaattaggaccagaatcactggaaggatttacggttctaggtgcagaaaatgttgcaaataaagttaaagtaaaaagagttcttccatcatggttatcaaatccaacagttatatcagtaaacttacaagacctacaaaccaaagtttctgatctaaaacaattagacaaaggaattcgtaaattactcaaagcaaataatatacaatatttcttccctgttcaagcagaagtaattccatggctactagaaacttctagacattctgatgtgatattccctagagatatttgtgtatcagccccaactggaagtggtaaaacattagcatatgttttacctattgttcaagcattaaaaaagtacactgtgaagagaataagagctttggtcattttaccaacacaggacttggctacgcaggtgtttaagacatttaaaacctattcccaggatacaaatatagatgtgtgtttaataagtggaactaactcttttgctgtagaacaatcacagttgatcattgagaataaagctttcggagcagtcagtaaaattgatatcttggtatgtacagctggaagacttgtagatcacttaaaagtaactaaaggatttagtttacgaaatttggagtttttagttatcgatgaggcagatagagtgcttgaaaatgttcagaacgattggctgtatcacttggaaaaacatatttatcaagaggcaactaatggacaaacgagaaaagttctcaacctctgtactttagaacaagcacggcctccacagaaactcctattctcagctacattgtcacaagatccggaaaaattacagaaactttccctatttcagcccaaactctttacttcaattgtagaaactgacaatgcagaagctgccccaacttccgttgcagatactttcataggaaagtacacaactccgaaagaattaactgaaaaatacgttgttacaagtgcggagttaaagccgctgtttttgtacgaactaatcaaattggagaaattaacgaactctatcgtctttacgcactctgtcgaaagtgcgcataggctagcaattttattaaaatctctgtttaaagaggaattaaaggtcgatgaaatatcttctaatctacaaggaaagaacagatcaaaacttattgaaaagtttgcagcaggtgatattgatttgttagtatgtacagatgccctggctagaggaatagaccttcctcatatacaatgtgttatttcttattctgcccccaaatacctcaagacatacattcatcgtgctggtagaactgctagagcaggggaacctggtttggctgttgccttattaaataaaccgcaagtatcaaagtttaaatctatgttgaatcaagcaaatcatacgaacattcaagagcttgttattcctgaagataatttggaacccctcggtgaaaagtacaaagaatctttacaagaattgaaaaaagtcgtagacaatgaagaaaaattagatttggaaaagacattaagtgctaaacgaacaaagaaaattaagcgaaaaaggaataattcaattactagtaatattagtaaat

SEQ ID NO: 65 - protein, 638 aa, Diabrotica virgifera
MELFVVNRHDDGQQEEIHNEQDKLDKVLKKIQKNKKLRQKQKEKISQVKQAKQRTLQSREIKRKIKASIIKVPDNINDDFLDKTVENLVITQTSDNIDESKHKKRKLGPESLEGFTVLGAENVANKVKVKRVLPSWLSNPTVISVNLQDLQTKVSDLKQLDKGIRKLLKANNIQYFFPVQAEVIPWLLETSRHSDVIFPRDICVSAPTGSGKTLAYVLPIVQALKKYTVKRIRALVILPTQDLATQVFKTFKTYSQDTNIDVCLISGTNSFAVEQSQLIIENKAFGAVSKIDILVCTAGRLVDHLKVTKGFSLRNLEFLVIDEADRVLENVQNDWLYHLEKHIYQEATNGQTRKVLNLCTLEQARPPQKLLFSATLSQDPEKLQKLSLFQPKLFTSIVETDNAEAAPTSVADTFIGKYTTPKELTEKYVVTSAELKPLFLYELIKLEKLTNSIVFTHSVESAHRLAILLKSLFKEELKVDEISSNLQGKNRSKLIEKFAAGDIDLLVCTDALARGIDLPHIQCVISYSAPKYLKTYIHRAGRTARAGEPGLAVALLNKPQVSKFKSMLNQANHTNIQELVIPEDNLEPLGEKYKESLQELKKVVDNEEKLDLEKTLSAKRTKKIKRKRNNSITSNISK

SEQ ID NO: 66 - DNA, 454 bp, Diabrotica virgifera
agatgtgtgtttaataagtggaactaactcttttgctgtagaacaatcacagttgatcattgagaataaagctttcggagcagtcagtaaaattgatatcttggtatgtacagctggaagacttgtagatcacttaaaagtaactaaaggatttagtttacgaaatttggagtttttagttatcgatgaggcagatagagtgcttgaaaatgttcagaacgattggctgtatcacttggaaaaacatatttatcaagaggcaactaatggacaaacgagaaaagttctcaacctctgtactttagaacaagcacggcctccacagaaactcctattctcagccacattgtcacaagatccggaaaaattacagaaactttccctatttcagcccaaactctttacttcaattgttgaaactgacaataacagaagctgtcccaactcccgtgtt

SEQ ID NO: 67 - DNA, 1077 bp, Artificial Sequence
  hairpin forming sequence
ttaccaaaaagtcgccaaaatttattttttttcagcaactatcacagatacgttagagaaattaaaggaagtcagcaataaagatatgttctattatgaagctccttccatctcagatgctgttactgttgaacagttagaacaaaagwatgtgttatgtcctaaagatgttaaagatgcttatctagctcaagtgattagggagtttagagccgaaaatgaagatggcaacattatgatttttacagatacttgcaaaaattgccaagtactctcaatgaccttaaacgatgtaggatttgaaaatgtatctctgcatgcgatgattccacagcaacagcgcctagcagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagacctgaggaatctggcggcatgtttggattgggaggtagcactcaacataggaatatttggtacaacaactaggtcatctggactacctttagatgcttctgggtcgaaatggtaaattttctgtagattaaatgtaagggttccgttgtcgtgtattgaaatattttttctctcccatctttctctgtatacatatggacccaattcgttgacgataggtttcgatccattattaaggaattcatcagcgttggtaacgttgtagatataaagccttatcttgggctctactggaggtttggcccaccaactgaacgtttgtgtaccatttactaatttaatttgtgaatttattattaaattaacccagctactgaagaaaacagcgattattattccaaaaagtacacatcctagtgatatcgctacgacgaaccaccatttccttaggaaagcacttgatagtttagcacataacttttcccttcctttcat

SEQ ID NO: 68 - DNA, 3593 bp, Diabrotica virgifera
aggtgtaacgtacgttgttcttcgatcttacagaaccaaagttagagttgcgggtaccggaaccatacgacgatcgtacgcgtgaactatttactaggctccggagtagtaccgctgttagtactttatttttaaaaagtggaccgcgagtgcaaagacttctttgacatcgaagtaagataatctggaaccgacgatcgtgtaatgaagcggagcgaataacttctatacggttacctacctctaatagtgttttgtgaatctcaatagttttacgtagttaaagacaatttaggtaaacaaaatgaaaggaagggaaaagttatgtgctaaactatcaagtgctttcctaaggaaatggtggttcgtcgtagcgatatcactaggatgtgtactttttggaataataatcgctgttttcttcagtagctgggttaatttaataataaattcacaaattaaattagtaaatggtacacaaacgttcagttggtgggccaaacctccagtagagcccaagataaggctttatatctacaacgttaccaacgctgatgaattccttaataatggatcgaaacctatcgtcaacgaattgggtccatatgtatacagagaaagatgggagagaaaaaatatttcaatacacgacaacggaacccttacatttaatctacagaaaatttaccatttcgacccagaagcatctaaaggtagtccagatgacctagttgttgtaccaaatattcctatgttgagtgctacctcccaatccaaacatgccgccagattcctcaggttggccatggccagtattatggatattctcaagattaaacccttcgtagaagtatcagttgaacagttgctgtggggttacgaagatcctctacttaaattggctaaagacgttgttcctaaagaacagaaattgccctatgaagaatttggtcttctttataagaaaaatggtacttctccagataatattacgattttcacaggagtagaagacataacaaagtacggaattatcgaaagtgttaacggaaaaactaatttaagtcactacggtacagatcaatgtaacagtcttgaagcatctgacggatctatctttcccccacatatgactaagaacaccactttgtacatttttgacaaggatctgtgtcgaagattgcctttagtatttgacaaagaggttctagcaagtagcaatgaagtacctgcttatagatttaccccacctaaaaatgtattcggaagtgtagaggaaaatccagagaatgcctgcttctgtcctcagggtccaccatgcacaccgtctggattcttcaacgtatcgctatgtcaatttgactcaccagttttactatcttttcctcatttctacctcgctgatgacaaatatagaactgcattggaaggagtgtccccacctgatccagaaaagcacgccttctacatggatgttcaacctgaaatgggctcagcgatgtctgctaaagctcgcatccaaatcaacttggctgtttctcaagtaatagacataaaacaagttgccacattcccagacatcattttccctatcttgtggttcgaagagggcttggattctcttccagcagagatgaccggtctcatgaaaatcgctactacagttccaccaatagccaagatcgtcatatcagtctcactgtttgtcattggaatagttctactcattatcgccatctggcggctacttagaggtgcgaataggcttagttcgcttcacctagcgcctggacatgtgggacaatcaacaaagaccaaggatgtcccaatggcgaatgttccaaaatactaagacatattagataatagagacggtggttaaaaatcgttttgaaaggtggttcctggttcatgaaaaaagacatttacattatacctactgttgttttgggacagagatatacttattaaagttttttactatgctgttctcctttaatatttttgaaaattttagggctatagaggtttggaccaggaaaatccttttttatttacattgaatatcataaaattcgataaaatagttcttaacttatttgcaaaatatatttaattaatatattaaataaagattaaatatatatttataaatattttttaaactatatgcggatagtgttcacctttttttctgttacttgtactcacgaatttaccacaagttaccgcaagaaaaacatttttattcctaaaatccgatattgttgttttaaaaaataatttttgacaagtgcttccctatcgaacttaaaacgttcgtaataaaagtgttattatacattataatatgagctgcttttacctgataaaacatttaaattttataattaaatttagttaactaaattacaataattaaataatttaaaatattgattttcaaaagtgtttgaaagcttcgagatgatgaaaagaactggttattacattttatttataattattcaaaagtgaaaaaagattcgattaagagaataattcgtcaagattagatgcgataagactgtgctgtagtttttcaaatagctaattctatactcttgctaataagatgagccattttcagaaggatgaactataagctatgatgtttgtattttatattgatttaattataagatttggaacaacaaattcaagcaatttttttggaagtgtgagttggaagttgtagttagttcagacatgtcaaaatttcgtattatcaatcatatattttaattttgtagcaattttttatctggtcttctagttgatgataaatgttctacgtgaaaaatcgcagaacacgaataataacaccaacaaaatttacaacgtaatatcaattaaaataagcttgaatttaagcggtttattgattacaataaacataacactacatgatgaaacataacataataattgttagataatacgagctttagaaaaaagacattttcacatcaccaaaaaagtttcacttgcctcatatgtattattagcaaatttaataatttactaaataagaaaaccttaaacttgaactatatataattaaaatatcacaaatacttaaaaaacaacacaaataactgacttacaggttaaaaataattgcattgcagattagtgtctctctttgtctactgctaactgcgttcaatgtgatgccgatagaaggaagactaataatgaaatatgtatgtgtactattcgggaataatttgtttactgagtaaataaacaaagtttttcttccctttattaaaaatatgaatacccctgggacatttagcctgggtaatttccagtcccgagaaagtttctgatggcagatcgtatttatatatttttttatttatttttattaaaaaggcatgccttaccgcattcacttcgatgacaaggtatccgctgcggcatgcgctaaggaatgcgtttagcacacgctaaaggcacgtagtttggacataacaattgtttcataaccttaaaacacaaaagaggcaagtactcacttcatttacttgtaataattattgcacaag

SEQ ID NO: 69 - protein, 515 aa, Diabrotica virgifera
MKGREKLCAKLSSAFLRKWWFVVAISLGCVLFGIIIAVFFSSWVNLIINSQIKLVNGTQTFSWWAKPPVEPKIRLYIYNVTNADEFLNNGSKPIVNELGPYVYRERWERKNISIHDNGTLTFNLQKIYHFDPEASKGSPDDLVVVPNIPMLSATSQSKHAARFLRLAMASIMDILKIKPFVEVSVEQLLWGYEDPLLKLAKDVVPKEQKLPYEEFGLLYKKNGTSPDNITIFTGVEDITKYGIIESVNGKTNLSHYGTDQCNSLEASDGSIFPPHMTKNTTLYIFDKDLCRRLPLVFDKEVLASSNEVPAYRFTPPKNVFGSVEENPENACFCPQGPPCTPSGFFNVSLCQFDSPVLLSFPHFYLADDKYRTALEGVSPPDPEKHAFYMDVQPEMGSAMSAKARIQINLAVSQVIDIKQVATFPDIIFPILWFEEGLDSLPAEMTGLMKIATTVPPIAKIVISVSLFVIGIVLLIIAIWRLLRGANRLSSLHLAPGHVGQSTKTKDVPMANVPKY

SEQ ID NO: 70 - DNA, 496 bp, Diabrotica virgifera
atgaaaggaagggaaaagttatgtgctaaactatcaagtgctttcctaaggaaatggtggttcgtcgtagcgatatcactaggatgtgtactttttggaataataatcgctgttttcttcagtagctgggttaatttaataataaattcacaaattaaattagtaaatggtacacaaacgttcagttggtgggccaaacctccagtagagcccaagataaggctttatatctacaacgttaccaacgctgatgaattccttaataatggatcgaaacctatcgtcaacgaattgggtccatatgtatacagagaaagatgggagagaaaaaatatttcaatacacgacaacggaacccttacatttaatctacagaaaatttaccatttcgacccagaagcatctaaaggtagtccagatgacctagttgttgtaccaaatattcctatgttgagtgctacctcccaatccaaacatgccgccagattcctcaggt

SEQ ID NO: 71 - DNA, 1225 bp, Artificial Sequence
  hairpin forming sequence
atgaaaggaagggaaaagttatgtgctaaactatcaagtgctttcctaaggaaatggtggttcgtcgtagcgatatcactaggatgtgtactttttggaataataatcgctgttttcttcagtagctgggttaatttaataataaattcacaaattaaattagtaaatggtacacaaacgttcagttggtgggccaaacctccagtagagcccaagataaggctttatatctacaacgttaccaacgctgatgaattccttaataatggatcgaaacctatcgtcaacgaattgggtccatatgtatacagagaaagatgggagagaaaaaatatttcaatacacgacaacggaacccttacatttaatctacagaaaatttaccatttcgacccagaagcatctaaaggtagtccagatgacctagttgttgtaccaaatattcctatgttgagtgctacctcccaatccaaacatgccgccagattcctcaggtagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagacctgaggaatctggcggcatgtttggattgggaggtagcactcaacataggaatatttggtacaacaactaggtcatctggactacctttagatgcttctgggtcgaaatggtaaattttctgtagattaaatgtaagggttccgttgtcgtgtattgaaatattttttctctcccatctttctctgtatacatatggacccaattcgttgacgataggtttcgatccattattaaggaattcatcagcgttggtaacgttgtagatataaagccttatcttgggctctactggaggtttggcccaccaactgaacgtttgtgtaccatttactaatttaatttgtgaatttattattaaattaacccagctactgaagaaaacagcgattattattccaaaaagtacacatcctagtgatatcgctacgacgaaccaccatttccttaggaaagcacttgatagtttagcacataacttttcccttcctttcat

SEQ ID NO: 72 - DNA, 2453 bp, Diabrotica virgifera
aagtttgtcaaagaagtcgataaaggaaacacatgagaattggaattaggagtgaaaagtgatatttattgtggtttaggctgaattaaattaaatataagtgtgtcagactaacgcccaaaatgaggttatatttgggatttggagtgctgcttcttttagctgccgttttgggagctaaagaagagaaaaaggataaagaagatgttggaacagtcatcggaattgatttgggaacaacgtattcatgtgtgggtgtatataaaaatggtagagtagaaatcatcgccaatgaccaaggtaaccgtatcaccccctcgtatgttgccttcactgccgatggtgagcgtctcatcggagatgctgccaagaatcaacttacaacaaaccctgaaaacactgtttttgatgccaagcgtctcattggtcgtgacttcacagaccatacagttcaacatgacttgaagctcttccccttcaaagtcattgaaaagaaccagaagccacatattcaagtagaaaccagccagggaactaaggtctttgcccctgaagaaatttctgctatggttttgggaaagatgaaggaaacagcagaagcttatttaggcaagaaggttacccatgctgttgtaacagtacctgcttatttcaatgatgcccaacgtcaagccaccaaagatgctggtaccattgctggtcttaatgtcatgagaatcatcaatgaacctactgccgcagctattgcctatggtcttgataagaaggaaggagaaaagaatgtattagtatttgatttgggtggtggtacttttgatgtgtcccttttgactattgacaatggagtatttgaagtagttgctaccaatggagacactcacttgggtggtgaagatttcgatcaaagagttatggatcatttcatcaagttgtacaagaagaagaagggcaaggatatcagaaaagacaacagagccgtccagaaattgagaagagaagttgaaaaggccaagagagctttgtcttccagtcaccaaaacaggattgaaattgaatccttctttgaaggtgatgatttctctgaaactctcaccagagccaaatttgaagaattaaacatggatcttttccgttctaccatgaaaccagtacagaaagtattagaagatgctgacatgaacaagaaagatgtagatgaaattgtacttgtaggtggttcaactcgtattcccaaagtacaacaattggtcaaagaattctttggaggcaaagaaccttcaagaggtatcaaccctgatgaagctgttgcttatggtgctgccgttcaagctggtgtcctctctggagaacaagatactgatgccattgtactattggatgtcaatcctttgaccatgggtattgaaactgtaggaggtgtcatgaccaagttgatcccaagaaacactgtcatacccacaaagaaatctcagatcttctctactgcatctgataaccaacacactgtcaccatccaagtatacgaaggagaacgtcctatgaccaaggacaatcacttgttgggtaaatttgatttgaccggtatcccaccagcacctagaggagtaccacagattgaagttactttcgaaattgatgccaacggtatcttgcaagtttctgctgaagacaaaggaactggaaacagggagaagattgttatcaccaacgatcagaacagattgacacctgaagacattgaccgtatgatcaacgatgccgagaaattcgctgatgaagacaagaaacttaaagaaagagtagaagccagaaacgaattggaaagctacgcatactcgctcaagaaccaaattaacgacaaagacaagttgggagctaagttgtcagatgacgaaaagaccaaaatggaagaagcaatcgatgagaagatcaaatggttggaagacaatcaagatactgattcagaagactacaagaaacagaagaaggaattggaagatgtcgtgcagcccatcatcgctaaattgtaccagagcacaggtggcgccccacctccaccctcaagtgaggatgatgatgaacttaaagatgaattgtgaggtgaattgtaggccaaaaagactgaaaaaacttgtgtggatgcgtgagaatgcggacgatcgtgtgttatttaatttccgtgactgttatccctttttttgtaatagacttttttgtaaattttccggtttgctcattgaatgggtgaacttatttatttattattttgcgtgttaggtacaggcgtatgattgtgatatatattttttgtattaagtagtaaatgcattgtttttgtatattgtacacctgtatttgactattctcaattttcctgtaatttttcaataaaattacttagaaaaaataaaaaaaaaaaaaaaaaaaaaaaaataaacgcagtagagtagaca

SEQ ID NO: 73 - protein, 658 aa, Diabrotica virgifera
MRLYLGFGVLLLLAAVLGAKEEKKDKEDVGTVIGIDLGTTYSCVGVYKNGRVEIIANDQGNRITPSYVAFTADGERLIGDAAKNQLTTNPENTVFDAKRLIGRDFTDHTVQHDLKLFPFKVIEKNQKPHIQVETSQGTKVFAPEEISAMVLGKMKETAEAYLGKKVTHAVVTVPAYFNDAQRQATKDAGTIAGLNVMRIINEPTAAAIAYGLDKKEGEKNVLVFDLGGGTFDVSLLTIDNGVFEVVATNGDTHLGGEDFDQRVMDHFIKLYKKKKGKDIRKDNRAVQKLRREVEKAKRALSSSHQNRIEIESFFEGDDFSETLTRAKFEELNMDLFRSTMKPVQKVLEDADMNKKDVDEIVLVGGSTRIPKVQQLVKEFFGGKEPSRGINPDEAVAYGAAVQAGVLSGEQDTDAIVLLDVNPLTMGIETVGGVMTKLIPRNTVIPTKKSQIFSTASDNQHTVTIQVYEGERPMTKDNHLLGKFDLTGIPPAPRGVPQIEVTFEIDANGILQVSAEDKGTGNREKIVITNDQNRLTPEDIDRMINDAEKFADEDKKLKERVEARNELESYAYSLKNQINDKDKLGAKLSDDEKTKMEEAIDEKIKWLEDNQDTDSEDYKKQKKELEDVVQPIIAKLYQSTGGAPPPPSSEDDDELKDEL

SEQ ID NO: 74 - DNA, 478 bp, Diabrotica virgifera
atgaggttatatttgggatttggagtgctgcttcttttagctgccgttttgggagctaaagaagagaaaaaggataaagaagatgttggaacagtcatcggaattgatttgggaacaacgtattcatgtgtgggtgtatataaaaatggtagagtagaaatcatcgccaatgaccaaggtaaccgtatcaccccctcgtatgttgccttcactgccgatggtgagcgtctcatcggagatgctgccaagaatcaacttacaacaaaccctgaaaacactgtttttgatgccaagcgtctcattggtcgtgacttcacagaccatacagttcaacatgacttgaagctcttccccttcaaagtcattgaaaagaaccagaagccacatattcaagtagaaaccagccagggaactaaggtctttgcccctgaagaaatttctgctatggttttgggaaagatgaaggaaacagcagaag

SEQ ID NO: 75 - DNA, 1181 bp, Artificial Sequence
  hairpin forming sequence
atgaggttatatttgggatttggagtgctgcttcttttagctgccgttttgggagctaaagaagagaaaaaggataaagaagatgttggaacagtcatcggaattgatttgggaacaacgtattcatgtgtgggtgtatataaaaatggtagagtagaaatcatcgccaatgaccaaggtaaccgtatcaccccctcgtatgttgccttcactgccgatggtgagcgtctcatcggagatgctgccaagaatcaacttacaacaaaccctgaaaacactgtttttgatgccaagcgtctcattggtcgtgacttcacagaccatacagttcaacatgacttgaagctcttccccttcaaagtcattgaaaagaaccagaagccacatattcaagtagaaaccagccagggaactaaggtctttgcccctgaagaaatttctgctatggttttgggaaagatgaaggaaacagcagaaggactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttacttctgctgtttccttcatctttcccaaaaccatagcagaaatttcttcaggggcaaagaccttagttccctggctggtttctacttgaatatgtggcttctggttcttttcaatgactttgaaggggaagagcttcaagtcatgttgaactgtatggtctgtgaagtcacgaccaatgagacgcttggcatcaaaaacagtgttttcagggtttgttgtaagttgattcttggcagcatctccgatgagacgctcaccatcggcagtgaaggcaacatacgagggggtgatacggttaccttggtcattggcgatgatttctactctaccatttttatatacacccacacatgaatacgttgttcccaaatcaattccgatgactgttccaacatcttctttatcctttttctcttctttagctcccaaaacggcagctaaaagaagcagcactccaaatcccaaatataacctcat

SEQ ID NO: 76 - DNA, 2240 bp, Diabrotica virgifera
  misc_feature (2227)..(2227): n is a, c, g, or t
atcgatttgattgcagtcattgctgtagtgcacacgtaatttaagtatttatttaagaattgtctgcaaataattaattataagatggctaaagccccagctgtaggtattgatttaggaaccacatactcctgtgtaggagttttccaacatggcaaagttgaaattattgccaacgaccaaggtaacagaaccacaccatcatatgtggccttcacagacacagaacgtctcatcggagatgctgccaagaaccaggtagccatgaaccccaataacacaatttttgatgccaaacgtcttattgggcgtcgctttgatgacagtgctgtacagtctgacatgaaacattggccatttgaagtagtaaatgatgcaggtaaaccaaagattaaagttgcatacaagggcgaagacaaatccttctacccagaagaagtcagttccatggtccttacaaaaatgaaggaaacagcagaagcatacttaggaaagaatgtcaccaacgctgtcatcacagtacctgcatatttcaatgactcacaacgtcaagctaccaaagatgctggaaccattgctggactccaggtattacgtattattaacgaacccactgctgctgccatcgcctatggtttagacaaaaagggccaaggtgaaagaaacgtcctcattttcgatctgggtggtggtacttttgatgtatcaatcttaacaattgaagatggtatctttgaagtcaaatcaactgctggagacactcacttgggaggtgaagacttcgacaacagaatggtcaatcactttgttggagaattcaagaggaaatacaagaaggaccttaccacaaacaaacgtgccctccgtcgtttgagaacaagctgtgaaagagctaagcgtaccctttcctcatcaacccaagccagcattgaaatcgattctctatttgagggtattgacttctacacctcaatcaccagggctagatttgaagaactgaacgctgatttgttcagatctaccatggaacctgtagaaaaggccatcagggatgccaagatggacaaatctcaagttcacgatattgtgttggttggtggatctacccgtattccaaaggtacaaaaactgttgcaagatttcttcaatggtaaagaattgaacaagtcgatcaaccccgatgaagctgttgcttatggtgcagccgtccaagccgccatcttgcacggagataagtctgaggaagtacaggacttgctcttgctcgatgtcactccactttcattgggtattgaaactgccggaggtgttatgactgccctcatcaaacgtaacactaccattcccaccaagcaaacccagaccttcactacttactctgacaaccaacccggagtacttattcaggtatatgaaggtgaacgtgccatgaccaaggacaacaacctcttgggaaaattcgaacttaccggcattcctcctgcgccccgtggtgtccctcagattgaagtaaccttcgacattgatgccaacggtatcttgaacgtcacagctattgaaaaatccaccaacaaagaaaacaagatcaccatcaccaatgataaaggtcgtctcagcaaagaagacattgaaagaatggtcaacgatgcagaaaaataccgtaacgaagatgataagcaaaaggctaccatctctgccaagaacgtcctcgaatcatactgtttcaacatcaagagcaccatggaagatgacaaaatcaaggacaagatcagcgaaagtgacaaaactaccgtcatggagaaatgcaacgaggttattgcttggttggatgctaaccaattagccgacaaggaagaatatgaacacaaacaaaaggagctcgaaaacatctgcaaccccatcatcaccaagttgtaccaaggtgccggtggagctccaggtggcatgcccggtggtttcccaggtggagcagctccaggagctgcaggcgccgctggaggtgctggaccaaccattgaagaagtcgattaaactattcattccattttttaaacttttgttaaccgttgtgcagtattacttgcagacaaagctttttaaaattactttttttacacaatttttcaattgttagacaacgtttttggtaacagttcaaaaattactgcaataattatgttcgttcatgaataaaataaaaagtcaaatccgaaaaaaaaaaaaaaanaaaaaaaaaaaaa

SEQ ID NO: 77 - protein, 648 aa, Diabrotica virgifera
MAKAPAVGIDLGTTYSCVGVFQHGKVEIIANDQGNRTTPSYVAFTDTERLIGDAAKNQVAMNPNNTIFDAKRLIGRRFDDSAVQSDMKHWPFEVVNDAGKPKIKVAYKGEDKSFYPEEVSSMVLTKMKETAEAYLGKNVTNAVITVPAYFNDSQRQATKDAGTIAGLQVLRIINEPTAAAIAYGLDKKGQGERNVLIFDLGGGTFDVSILTIEDGIFEVKSTAGDTHLGGEDFDNRMVNHFVGEFKRKYKKDLTTNKRALRRLRTSCERAKRTLSSSTQASIEIDSLFEGIDFYTSITRARFEELNADLFRSTMEPVEKAIRDAKMDKSQVHDIVLVGGSTRIPKVQKLLQDFFNGKELNKSINPDEAVAYGAAVQAAILHGDKSEEVQDLLLLDVTPLSLGIETAGGVMTALIKRNTTIPTKQTQTFTTYSDNQPGVLIQVYEGERAMTKDNNLLGKFELTGIPPAPRGVPQIEVTFDIDANGILNVTAIEKSTNKENKITITNDKGRLSKEDIERMVNDAEKYRNEDDKQKATISAKNVLESYCFNIKSTMEDDKIKDKISESDKTTVMEKCNEVIAWLDANQLADKEEYEHKQKELENICNPIITKLYQGAGGAPGGMPGGFPGGAAPGAAGAAGGAGPTIEEVD

SEQ ID NO: 78 - DNA, 417 bp, Diabrotica virgifera
gatggctaaagccccagctgtaggtattgatttaggaaccacatactcctgtgtaggagttttccaacatggcaaagttgaaattattgccaacgaccaaggtaacagaaccacaccatcatatgtggccttcacagacacagaacgtctcatcggagatgctgccaagaaccaggtagccatgaaccccaataacacaatttttgatgccaaacgtcttattgggcgtcgctttgatgacagtgctgtacagtctgacatgaaacattggccatttgaagtagtaaatgatgcaggtaaaccaaagattaaagttgcatacaagggcgaagacaaatccttctacccagaagaagtcagttccatggtccttacaaaaatgaaggaaacagcagaagcatacttaggaaagaatgt

SEQ ID NO: 79 - DNA, 1059 bp, Artificial Sequence
  hairpin forming sequence
gatggctaaagccccagctgtaggtattgatttaggaaccacatactcctgtgtaggagttttccaacatggcaaagttgaaattattgccaacgaccaaggtaacagaaccacaccatcatatgtggccttcacagacacagaacgtctcatcggagatgctgccaagaaccaggtagccatgaaccccaataacacaatttttgatgccaaacgtcttattgggcgtcgctttgatgacagtgctgtacagtctgacatgaaacattggccatttgaagtagtaaatgatgcaggtaaaccaaagattaaagttgcatacaagggcgaagacaaatccttctacccagaagaagtcagttccatggtccttacaaaaatgaaggaaacagcagaagcatacttaggaaagaatgtgactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttaacattctttcctaagtatgcttctgctgtttccttcatttttgtaaggaccatggaactgacttcttctgggtagaaggatttgtcttcgcccttgtatgcaactttaatctttggtttacctgcatcatttactacttcaaatggccaatgtttcatgtcagactgtacagcactgtcatcaaagcgacgcccaataagacgtttggcatcaaaaattgtgttattggggttcatggctacctggttcttggcagcatctccgatgagacgttctgtgtctgtgaaggccacatatgatggtgtggttctgttaccttggtcgttggcaataatttcaactttgccatgttggaaaactcctacacaggagtatgtggttcctaaatcaatacctacagctggggctttagccatc

SEQ ID NO: 80 - DNA, 2826 bp, Diabrotica virgifera
gtgacggtgattgtaggaccataacatagaagagtgggaccataaagagaaaaatagaagaatgtattttgacaatagttaggttaattaacaaaaatagatatttggcgggaattagaagctttctacagtttttattaccgttttattgataatataatttcaaatttagatattaaaacgttgaaaagtatctttaaaaatctcaagtcttaaaaatggtgaaaccgttgaataaattttatgttttaaaaagaggaggccgaagagaagaggtccatattgataaaatcacatcacgtattcagaaattatgttatggcttgaatgccgattatgtcgatccagttagtattaccttgaaagtggttaatggcctatacccaggagttactactgcagaattagatgcactggctgcagaaactgctgctaccctcagcgaagaccatcctgattatgctactctagcagctaggatcgctatatctaatcttcagaaagaaacgaaaaaattatttagtgatgtaatagatgacctttacaatgtattcgacgaaaacctaggagaaaaatcaccaataatttcagaagaacttcacaaaataatagcagaaaattcagaacgtctaaattcatgtatgatatacgatagggatttttcctatgattatttcgctttcaaagaactggaaaaatcatatctattaagaataaacggcaaagttgtggagagaccacaacatatgttaatgagagtagctgtaggtattcataaggaaaacattgatgatgcaatcacaacttacaacctattgtccgaaagattttatatccactctacaaatactctactgttagcttctacacccaaacctcaactatgttctagcattgtcatgatgatgccagatgatagcatagaaggaatctttaactgcatcagactttgtggattggtttccaaatacaccggtactgtaggtctaaacattcagaatataagagctaaaggtacttatatagcgggtactaatggtgtctcaaatggtttagttcctatgttacgagtttttaataacatggcatgctacgtagatcaacttagcaccaataaaccttctgagatagctgtgtatattgaaccttggcatgctgatattctcgaatttttgaacctaagtaaacccggtggtaaagaagaagtgagaacaagagatttatcatatgctttgtgggtaccagatttgtttatgaaaagggtagaatctaacgggaaatggtctctaatgtgcccccatcaatctccaggtcttactgattcttatggagataaatttgaacagttatacgaaaaatatgagaaagaggggaaatatataaaacaaatacaagctcaagaattgtggagggcgattattgttcttcaagcagagacaggtggcccggttatgatgtacaaggatgtatgcaacagaaaaagtaatcaaaaacacataggaacaattaggggtggaagttacagtggtgaagtagttgcatatacatcagatgaagaaatagtagcatgtccccaagcttctatcgctgtaaatatgtttgtcagtcccgacagaaagagtttcgacttccaaaagcttaaagaagttaccaaagttgtaaccaagaatctagacaaaatcattgatgttagcttctatcctgtgcctgaagaaaagaagtcaaacttaaaccatagagccattggcataggtgttcaaggtttggctgatactttcatattgatgcgtatgccctgtgatagtgaagaggcgcgaaaattaaataagcagatattcgaaactttatattatggagctctagaggcaagttctaatttggcagagttgaatggaccttacccaatgtacgagggcagtcctgtcagcaaaggtgttcttcaatatgatttgtgggacgtaaaaccatccgatctatgggattggcaatcacttaaagaaacaatcgctaaaactggtgtgcgtaattccttattaattgcacagatgtcgacaccgattctatccaggttgtttgcaaatagcgaatccaccgacctctacagatcttttctatacaccagacgagtctcctcaggagagtttcctgtagtcaatccttatttactgagagatttaacagaaaaagatatatgggatgacaaggtgaaagaatcaattgtcagtaacgcgggatccattcagaacatcccagaaatcccggatgctttgaaacagatatataaaactacttgggaagtatcacagaaggttgttttgaacatggccatcgatagaggagcgttcatcgaccaaagtcaaagtttaaatatacatatgatgaatccaaactacggaagcctaagttcaatgcatttctatggatggaatagcggacttaaaactggacttaataagttaagaaccaagtctgctcctaaagtttataccaaattgggcgacaaacaaaaaattgaggttgtatccaaagaagacgatttagaagaaatgaagaggaaacagcgggaagaggaagaaaagaatatggctagtttggtatgttctctacaaaatcgtgaagcttgtgacatgtgcggagcttgagttaatttatgttcttaagttttattagtattttatttatatcagcaagattatcttgatttatacatatctgatagaattgtaagtttttcttttattatttttattttttgaaatgtttgtatttaggatataagattttcaatgtaaaaatataaattttttatcctttattaaaaaaaaa

SEQ ID NO: 81 - protein, 807 aa, Diabrotica virgifera
MVKPLNKFYVLKRGGRREEVHIDKITSRIQKLCYGLNADYVDPVSITLKVVNGLYPGVTTAELDALAAETAATLSEDHPDYATLAARIAISNLQKETKKLFSDVIDDLYNVFDENLGEKSPIISEELHKIIAENSERLNSCMIYDRDFSYDYFAFKELEKSYLLRINGKVVERPQHMLMRVAVGIHKENIDDAITTYNLLSERFYIHSTNTLLLASTPKPQLCSSIVMMMPDDSIEGIFNCIRLCGLVSKYTGTVGLNIQNIRAKGTYIAGTNGVSNGLVPMLRVFNNMACYVDQLSTNKPSEIAVYIEPWHADILEFLNLSKPGGKEEVRTRDLSYALWVPDLFMKRVESNGKWSLMCPHQSPGLTDSYGDKFEQLYEKYEKEGKYIKQIQAQELWRAIIVLQAETGGPVMMYKDVCNRKSNQKHIGTIRGGSYSGEVVAYTSDEEIVACPQASIAVNMFVSPDRKSFDFQKLKEVTKVVTKNLDKIIDVSFYPVPEEKKSNLNHRAIGIGVQGLADTFILMRMPCDSEEARKLNKQIFETLYYGALEASSNLAELNGPYPMYEGSPVSKGVLQYDLWDVKPSDLWDWQSLKETIAKTGVRNSLLIAQMSTPILSRLFANSESTDLYRSFLYTRRVSSGEFPVVNPYLLRDLTEKDIWDDKVKESIVSNAGSIQNIPEIPDALKQIYKTTWEVSQKVVLNMAIDRGAFIDQSQSLNIHMMNPNYGSLSSMHFYGWNSGLKTGLNKLRTKSAPKVYTKLGDKQKIEVVSKEDDLEEMKRKQREEEEKNMASLVCSLQNREACDMCGA

SEQ ID NO: 82 - DNA, 291 bp, Diabrotica virgifera
atggtgaaaccgttgaataaattttatgttttaaaaagaggaggccgaagagaagaggtccatattgataaaatcacatcacgtattcagaaattatgttatggcttgaatgccgattatgtcgatccagttagtattaccttgaaagtggttaatggcctatacccaggagttactactgcagaattagatgcactggctgcagaaactgctgctaccctcagcgaagaccatcctgattatgctactctagcagctaggatcgctatatctaatcttcagaaagaaacg

SEQ ID NO: 83 - DNA, 483 bp, Diabrotica virgifera
atgatatacgatagggatttttcctatgattatttcgctttcaaagaactggaaaaatcatatctattaagaataaacggcaaagttgtggagagaccacaacatatgttaatgagagtagctgtaggtattcataaggaaaacattgatgatgcaatcacaacttacaacctattgtccgaaagattttatatccactctacaaatactctactgttagcttctacacccaaacctcaactatgttctagcattgtcatgatgatgccagatgatagcatagaaggaatctttaactgcatcagactttgtggattggtttccaaatacaccggtactgtaggtctaaacattcagaatataagagctaaaggtacttatatagcgggtactaatggtgtctcaaatggtttagttcctatgttacgagtttttaataacatggcatgctacgtagatcaacttagcaccaataaaccttct

SEQ ID NO: 84 - DNA, 444 bp, Diabrotica virgifera
atgtacgagggcagtcctgtcagcaaaggtgttcttcaatatgatttgtgggacgtaaaaccatccgatctatgggattggcaatcacttaaagaaacaatcgctaaaactggtgtgcgtaattccttattaattgcacagatgtcgacaccgattctatccaggttgtttgcaaatagcgaatccaccgacctctacagatcttttctatacaccagacgagtctcctcaggagagtttcctgtagtcaatccttatttactgagagatttaacagaaaaagatatatgggatgacaaggtgaaagaatcaattgtcagtaacgcgggatccattcagaacatcccagaaatcccggatgctttgaaacagatatataaaactacttgggaagtatcacagaaggttgttttgaacatggccatcgatagaggagcgttcatc

SEQ ID NO: 85 - DNA, 807 bp, Artificial Sequence
  hairpin forming sequence
atggtgaaaccgttgaataaattttatgttttaaaaagaggaggccgaagagaagaggtccatattgataaaatcacatcacgtattcagaaattatgttatggcttgaatgccgattatgtcgatccagttagtattaccttgaaagtggttaatggcctatacccaggagttactactgcagaattagatgcactggctgcagaaactgctgctaccctcagcgaagaccatcctgattatgctactctagcagctaggatcgctatatctaatcttcagaaagaaacggactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttacgtttctttctgaagattagatatagcgatcctagctgctagagtagcataatcaggatggtcttcgctgagggtagcagcagtttctgcagccagtgcatctaattctgcagtagtaactcctgggtataggccattaaccactttcaaggtaatactaactggatcgacataatcggcattcaagccataacataatttctgaatacgtgatgtgattttatcaatatggacctcttctcttcggcctcctctttttaaaacataaaatttattcaacggtttcaccat

SEQ ID NO: 86 - DNA, 3351 bp, Diabrotica virgifera
gttgtaatttagtgtgtgagcgagagtggtgttgtgtaccttcacaacccacttaaaattgtgaaataatttatttaaaaatacaaaaaaatataaaaaaaactaataaaaaataacaaaaaaatcgcaaaatagtgtaaaaaagtaaaataacaaacgtaatgaatccaggcgccccaaattaccccatggcgtctttgtacgttggggatttacactctgatattactgaggccatgctttttgagaaattttcaactgcgggccctgttctttcgattcgcgtttgcagagacttgattacacgtagatccttgggttatgcgtacgtaaatttccagcaacctgctgatgctgagcgtgcgctggatacaatgaactttgacttgattaaaggacgtcccatcagaataatgtggtctcaacgagatccatcattgagaaaatctggtgttggtaatgtttttattaagaatttggatcgttccattgacaacaaggctatgtatgatactttctcagctttcggtaatattcttagttgtaaggtagctcaagatgaaaacggctctagtaaaggatatggatttgttcattttgaaactgaagaagctgcaaataaatccattgaaaaggtaaatggtatgttgttgaatggaaaaaaagtatatgttggtcgctttattccacgtaaagaaagagaaaaggaactgggagaaaaggccaagcttttcacaaatgtatatgttaaaaattttggtgaagattttgctgaggaacagttaaaggttatgtttgaaaagtatggtaaaattaccagctacaaaataatgagtaaagatgatggtaaatctaaaggctttggctttgttgcctttgaaaatccagaggcagccgaaacagcggtggaagctttgaatggaaaagaattgatggaaggaaaacctttgtatgtaggaagagcccaaaagaaagctgagcgccagcaagaactaaaacgtcgatttgaagcccttaaaatggaaagacttaatcgttatcagggtgtaaatctctatgttaaaaacttggatgatacaatagacgacgaacgtctccgtaaagaattctctccgttcggaaccataacttctgccaaagtaatgatggaagaaggtagaagcaagggttttggtttcgtctgcttttcctccccggaagaagctacaaaagcagtaacagagatgaacggcagaattgtcggtacgaagcctttatatgttgccttagctcagcgtaaagaagatcgcaaagcacatttaacatctcagtacatgcagcgcatggccaatatgcgcatgcaccagatgggtcagttcattcaaccaggtgcgtctagcggctatttcgttccaaccattcctaccgcacagagattctatggaccagcccagatggctcagattcgtaccagtcccagatggcctgcccaaacacctgtaagaccaggtgctcaaggaggagctactgcctacactggcatgccaaacacttacagagcggctactcgtccaccaaaccagtcaacaacaatgcgtagcaacatcagtgtacctagaccaattactggacaacaacctcaaagtatgcaaggtcgtccacttgctggacaaggtggagttgtaccttccgctggccgcacagcaaacttcaaatatacttcgaacatgagaaatccacctcaatccttgggaggtataccaggaactgcggctcctgtacagcaagctgtacacatccagggtcaagagccacttacagcaacaatgttggctgctgctcccccacaggaacaaaaacagatgttgggtgaaagattgtttccacttatccaacgcatgtatgcagatatggcaggtaaaatcactggtatgttgttggaaattgacaatactgagttgttgcacatgttggaacaccaggagtcgcttaagaataaagtggaagaagcagttgctgtactacaagcccatcaagccaaacaggctgctactcagattaaaaaagattagtagttggtcgtaggcgactgtagaagggatataatcgtttaagaatataatatgtatcctgtaattatgtattgttgattttcatattttactttatatctcaacgtctgaaaaggcataagttattgagaagttaaagctatgagtaggcccttatgtgttcttacgcctatgaacacgttgttacatgtaaaacaaattgtgtatgaaaaatttcttcttcagatgtcattttaatgggaaactgctagcattcgtttatatattaaatatattgtatataataattattattataaatataataataattgttgcaaatttagcaattaatattttgtgaattttcaatgcaacttagcagaacttccatgtgtattttagttttttcatattattatttattttatattttcatgttataaattacccgttgtctgaaaagacgagggattgagatcattgttattgattattgattgtgcttgattattgattgattattaatattacccttccttacatcgcctatgaacaggatactatcaaataaataaaataaatataaaaattgcaaatagtcctatgacaaaaaagtaaaaaaatctatacttaacaatcccataataaagatttcttttcaattggaaactgtaacattcgtttgtattttcgcattgtttgcaattgttattttgatcattttcaatacaatttagtataagtttagtaatctaattttatatttttactgttttcatattgtgacaaattaatttctaaaagggcataaaaattttccaatatgacccttcttcgttcttacgttgcctttgaaaagaatactatcaaattaaacaaaaataattataaaaattgcaaagagtcctatgaccaaaaacgtaaaaaagattatctctatttaataatcaattcgtttgtatttttgcttatttacaatttttattttgatctattcaatggaattgtaagtctgacaatgctttatggaccattttgatggaagaaagcgttaatatattatttcagatatttttcgacaatttccttcattgtttatatccttcaatacatgaattttatggtttgtggactatcatgaatttttttatcgtttgtctcttagttttttaatttgatacgattatgactcacgtgtgttgcaaactagagatacggaatgagtataagtaaaactattttgtattcacatgccataaaattgatatataaatatattcgaattcaataaatcagttaaatcaaaaaaaaaaaaaaa

SEQ ID NO: 87 - protein, 629 aa, Diabrotica virgifera
MNPGAPNYPMASLYVGDLHSDITEAMLFEKFSTAGPVLSIRVCRDLITRRSLGYAYVNFQQPADAERALDTMNFDLIKGRPIRIMWSQRDPSLRKSGVGNVFIKNLDRSIDNKAMYDTFSAFGNILSCKVAQDENGSSKGYGFVHFETEEAANKSIEKVNGMLLNGKKVYVGRFIPRKEREKELGEKAKLFTNVYVKNFGEDFAEEQLKVMFEKYGKITSYKIMSKDDGKSKGFGFVAFENPEAAETAVEALNGKELMEGKPLYVGRAQKKAERQQELKRRFEALKMERLNRYQGVNLYVKNLDDTIDDERLRKEFSPFGTITSAKVMMEEGRSKGFGFVCFSSPEEATKAVTEMNGRIVGTKPLYVALAQRKEDRKAHLTSQYMQRMANMRMHQMGQFIQPGASSGYFVPTIPTAQRFYGPAQMAQIRTSPRWPAQTPVRPGAQGGATAYTGMPNTYRAATRPPNQSTTMRSNISVPRPITGQQPQSMQGRPLAGQGGVVPSAGRTANFKYTSNMRNPPQSLGGIPGTAAPVQQAVHIQGQEPLTATMLAAAPPQEQKQMLGERLFPLIQRMYADMAGKITGMLLEIDNTELLHMLEHQESLKNKVEEAVAVLQAHQAKQAATQIKKD

SEQ ID NO: 88 - DNA, 340 bp, Diabrotica virgifera
cgtaatgaatccaggcgccccaaattaccccatggcgtctttgtacgttggggatttacactctgatattactgaggccatgctttttgagaaattttcaactgcgggccctgttctttcgattcgcgtttgcagagacttgattacacgtagatccttgggttatgcgtacgtaaatttccagcaacctgctgatgctgagcgtgcgctggatacaatgaactttgacttgattaaaggacgtcccatcagaataatgtggtctcaacgagatccatcattgagaaaatctggtgttggtaatgtttttattaagaatttggatcgttccattgacaac

SEQ ID NO: 89 - DNA, 905 bp, Artificial Sequence
  hairpin forming sequence
cgtaatgaatccaggcgccccaaattaccccatggcgtctttgtacgttggggatttacactctgatattactgaggccatgctttttgagaaattttcaactgcgggccctgttctttcgattcgcgtttgcagagacttgattacacgtagatccttgggttatgcgtacgtaaatttccagcaacctgctgatgctgagcgtgcgctggatacaatgaactttgacttgattaaaggacgtcccatcagaataatgtggtctcaacgagatccatcattgagaaaatctggtgttggtaatgtttttattaagaatttggatcgttccattgacaacgactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttagttgtcaatggaacgatccaaattcttaataaaaacattaccaacaccagattttctcaatgatggatctcgttgagaccacattattctgatgggacgtcctttaatcaagtcaaagttcattgtatccagcgcacgctcagcatcagcaggttgctggaaatttacgtacgcataacccaaggatctacgtgtaatcaagtctctgcaaacgcgaatcgaaagaacagggcccgcagttgaaaatttctcaaaaagcatggcctcagtaatatcagagtgtaaatccccaacgtacaaagacgccatggggtaatttggggcgcctggattcattacg

SEQ ID NO: 90 - DNA, 1974 bp, Diabrotica virgifera
actaaatcgaatcacttatcagatgaaaatagaaacaccttcttcacaaaagatttacacaaagtggaaggggtattgagaccaaatcagaagtgacagttcttttaatttcctctaattttcactaaaaaatgagttttttccccgaaaataagcgtattaaaaattttcaaataatgacagtacaaatgtagtgcaagaaaattataaaagtgtgttgccgaggtacttactgaccagtataactatcaacctcatgtaaacattgtgattgtgaaggaacattttcgatcagtctattttctttattatggaaaaacataatgaaagtattgaattttctttcttggctttaaaatgttgggaaatctacaaagaactgtaaaaactcggtacaatcaatcaagagaagccgatgttaatattttatgggtcagtgttttagtgctagcagagcccaggatgcacctcaacctaaacatggtatcaaaacgtatttagtccagcaagcatccgaaaccgctttttccgacagtaaaaacgagcagaccattgacagtgtaatggctacatctttttctaatatgaatattacaaatcctatcaaaggacttgttagtaaaagaagaaatagatataagaaagatggtttcaatctggatctgacctatatcaccgacaatataattgcaatggggtatccggcctctaagatagaaggagtttaccgtaatcatattgatgacgttgtaaagttcctcgacaagaagcatcccgaccactattacatttacaatctttgctccgaacgtagctacgacaagatgaaattccacaatagagtcaaaatatttcccttcgacgatcacaacccccctaaaatcgattcgatccaaccgttttgtaacgacgtccacgaatggttatcgatagacaagaagaatgtggctgtggtacattgcaaggctggcaaaggcaggaccgggaccatgatctgttgctatctcctccacagccgaatgttctccaacgctgaatcggctctgaactattacggacaaactaggactcaagataagaaaggagtcacgattcccagtcaagtgcgatatgtacagtactacgagacgttattaaaacaacacctattttataccccagtttctatgtatataaaagaatttatacttaatccggtgccgaattttactggaggacaaggttctctgtctctcactatttcccatcaaactcttttgaaagaaggtgataaatacacaccaaaacttcaaaaattacggaagaatgattggtatgaagtgaaaaagagtggatctcctttttcgataaagttggactattgtctacctctgaaaggggatataaaagttgagttttttagtaaaactatgatgaggaaagagaagttattccaattttggttcaatacgttttttatatgcaattctcttaatggatttctgccgaggaacgggagtagtgaagaagtttgttacgtggtagattttgaaaagaacgaacttgatattgttaataaaaaggacaagcaaaacaaaatgtttcacgcagattttaagcttactttacatcttcaaaggattccgagagacgattgttgtccgacgtcgtacgaacgacattctcaaacacaagatacgccaagtgaaagttcggcagaatcatctgatgattatacgaacgaagacgaagactgggacagcggagaaggcaattccctcatacccaaaaacgaaccctgtgattctactaaaagttgatcctcaattattactaattttctgtataaagtttgttagattgtacattaatatgtacagagcatcctggattaggtgtttctacaggaaattgccgaaatagcagttgttggtcgaaagctattgtccgaggcatctcagttttgcaatttcttgggtaaataatgtgatatac

SEQ ID NO: 91 - protein, 456 aa, Diabrotica virgifera
MGQCFSASRAQDAPQPKHGIKTYLVQQASETAFSDSKNEQTIDSVMATSFSNMNITNPIKGLVSKRRNRYKKDGFNLDLTYITDNIIAMGYPASKIEGVYRNHIDDVVKFLDKKHPDHYYIYNLCSERSYDKMKFHNRVKIFPFDDHNPPKIDSIQPFCNDVHEWLSIDKKNVAVVHCKAGKGRTGTMICCYLLHSRMFSNAESALNYYGQTRTQDKKGVTIPSQVRYVQYYETLLKQHLFYTPVSMYIKEFILNPVPNFTGGQGSLSLTISHQTLLKEGDKYTPKLQKLRKNDWYEVKKSGSPFSIKLDYCLPLKGDIKVEFFSKTMMRKEKLFQFWFNTFFICNSLNGFLPRNGSSEEVCYVVDFEKNELDIVNKKDKQNKMFHADFKLTLHLQRIPRDDCCPTSYERHSQTQDTPSESSAESSDDYTNEDEDWDSGEGNSLIPKNEPCDSTKS

SEQ ID NO: 92 - DNA, 441 bp, Diabrotica virgifera
atgggtcagtgttttagtgctagcagagcccaggatgcacctcaacctaaacatggtatcaaaacgtatttagtccagcaagcatccgaaaccgctttttccgacagtaaaaacgagcagaccattgacagtgtaatggctacatctttttctaatatgaatattacaaatcctatcaaaggacttgttagtaaaagaagaaatagatataagaaagatggtttcaatctggatctgacctatatcaccgacaatataattgcaatggggtatccggcctctaagatagaaggagtttaccgtaatcatattgatgacgttgtaaagttcctcgacaagaagcatcccgaccactattacatttacaatctttgctccgaacgtagctacgacaagatgaaattccacaatagagtcaaaatatttcccttcgacgatcac

SEQ ID NO: 93 - DNA, 1115 bp, Artificial Sequence
  hairpin forming sequence
atgggtcagtgttttagtgctagcagagcccaggatgcacctcaacctaaacatggtatcaaaacgtatttagtccagcaagcatccgaaaccgctttttccgacagtaaaaacgagcagaccattgacagtgtaatggctacatctttttctaatatgaatattacaaatcctatcaaaggacttgttagtaaaagaagaaatagatataagaaagatggtttcaatctggatctgacctatatcaccgacaatataattgcaatggggtatccggcctctaagatagaaggagtttaccgtaatcatattgatgacgttgtaaagttcctcgacaagaagcatcccgaccactattacatttacaatctttgctccgaacgtagctacgacaagatgaaattccacaatagagtcaaaatatttcccttcgacgatcacagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagaggtgatcgtcgaagggaaatattttgactctattgtggaatttcatcttgtcgtagctacgttcggagcaaagattgtaaatgtaatagtggtcgggatgcttcttgtcgaggaactttacaacgtcatcaatatgattacggtaaactccttctatcttagaggccggataccccattgcaattatattgtcggtgatataggtcagatccagattgaaaccatctttcttatatctatttcttcttttactaacaagtcctttgataggatttgtaatattcatattagaaaaagatgtagccattacactgtcaatggtctgctcgtttttactgtcggaaaaagcggtttcggatgcttgctggactaaatacgttttgataccatgtttaggttgaggtgcatcctgggctctgctagcactaaaacactgacccat

SEQ ID NO: 94 - DNA, 1019 bp, Diabrotica virgifera
ggatggagtattctttgatcgaaccatcatagaaattcaaacatgacatggaacaaaatgtcctcccctatgtcaaaaaagtgagggaaaccgtttaaacataaccccaaatgatgctacaaaagtagatgaactggtaatattcgtaatatttaattattgcagtgtataatctgacttgttgtcttgttggcgtaaatttaaattaactacaggtaaaatgagtatgaaacgaggagcactaatcgtaatagaaggtgtagatcgctcaggaaaatcaacccaatgtaaaaagttgatccacgctctagagaaaaagaaaattgaatccaagttgatcgcttttccagaccgtagtaccttaacgggaaaacttattgacgagtacttaaagaataaagactgtaaactcaatgatcaagccatacacctcttgttttccgctaatcgatgggaaaatgtggagaaaattaagaatctgttatttgatggggttaccctaattatcgataggtattcttattctgggatagtcttttcatcggtgaaaaaaaatatgtctatggaatggtgtcagcatcctgagaacggccttcctaaaccagatctagtgcttttgctaactttaagtctagaagaaatgcaatcaaggccgggttttgggaatgaaaggtacgaaaatatggaatttcagagaaacgtggcaaacatgtacaaccagctctacgatgaagatgacaattgggttagaattgatgctgctgggtctataaataaagtccataataaaattttagaaacttgtttaaggaaaatagacgaagttgggacaaaacagctaaaaactttaaattttaataaaggcagttaaaataatagatctactaggtgaatttataaatatatttaaattatattaattattttttatatattgtattagtatttagtaattgcacatatgaggagtaattgtatatatacggatgttaattattaacatgatgtcaactataaac

SEQ ID NO: 95 - protein, 216 aa, Diabrotica virgifera
MSMKRGALIVIEGVDRSGKSTQCKKLIHALEKKKIESKLIAFPDRSTLTGKLIDEYLKNKDCKLNDQAIHLLFSANRWENVEKIKNLLFDGVTLIIDRYSYSGIVFSSVKKNMSMEWCQHPENGLPKPDLVLLLTLSLEEMQSRPGFGNERYENMEFQRNVANMYNQLYDEDDNWVRIDAAGSINKVHNKILETCLRKIDEVGTKQLKTLNFNKGS

SEQ ID NO: 96 - DNA, 549 bp, Diabrotica virgifera
atgagtatgaaacgaggagcactaatcgtaatagaaggtgtagatcgctcaggaaaatcaacccaatgtaaaaagttgatccacgctctagagaaaaagaaaattgaatccaagttgatcgcttttccagaccgtagtaccttaacgggaaaacttattgacgagtacttaaagaataaagactgtaaactcaatgatcaagccatacacctcttgttttccgctaatcgatgggaaaatgtggagaaaattaagaatctgttatttgatggggttaccctaattatcgataggtattcttattctgggatagtcttttcatcggtgaaaaaaaatatgtctatggaatggtgtcagcatcctgagaacggccttcctaaaccagatctagtgcttttgctaactttaagtctagaagaaatgcaatcaaggccgggttttgggaatgaaaggtacgaaaatatggaatttcagagaaacgtggcaaacatgtacaaccagctctacgatgaagatgacaattgggttagaattgatgctgctgggtct

SEQ ID NO: 97 - DNA, 1323 bp, Artificial Sequence
  Artificial sequence
atgagtatgaaacgaggagcactaatcgtaatagaaggtgtagatcgctcaggaaaatcaacccaatgtaaaaagttgatccacgctctagagaaaaagaaaattgaatccaagttgatcgcttttccagaccgtagtaccttaacgggaaaacttattgacgagtacttaaagaataaagactgtaaactcaatgatcaagccatacacctcttgttttccgctaatcgatgggaaaatgtggagaaaattaagaatctgttatttgatggggttaccctaattatcgataggtattcttattctgggatagtcttttcatcggtgaaaaaaaatatgtctatggaatggtgtcagcatcctgagaacggccttcctaaaccagatctagtgcttttgctaactttaagtctagaagaaatgcaatcaaggccgggttttgggaatgaaaggtacgaaaatatggaatttcagagaaacgtggcaaacatgtacaaccagctctacgatgaagatgacaattgggttagaattgatgctgctgggtctagagggatccaggcctaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggtatttaaataccggtccatggagagtgggaggctgattgatacctgtatttaagatgattacaacgtctttgaattcttctaatttataacctgtataaaattccaatgttggagtccagtggctgatacgttgcatcctaagtgctatataaagagcagctgatgccaatttggaatccctaatagtgactgtctcgtaattcattaatgagtattctagaataaatctagctaatgtcaaaacaggcattgttattttcgcacacctggcatatcttctcagaaatctgtaactaataggtattcccagatcgaatcctattactttaaacaaattgatctccatcctaataagttccctcttggtataagcgccatcgcaaatatacaagaaatcgtcaagcataggtggaatcctttcgtcgtatttactggctatcaacatggctgcggcaccgactaattgtaaagtttcctttcctactgtcattttactgaggtacatatcaactaatttaactcccaaataaagagtctcatgattgagttcgaaactttcttgaat

SEQ ID NO: 98 - DNA, 24 bp, Artificial Sequence
  Promotor oligonucleotide
ttaatacgactcactatagggaga

SEQ ID NO: 99 - DNA, 503 bp, Artificial Sequence
  Partial coding region
caccatgggctccagcggcgccctgctgttccacggcaagatcccctacgtggtggagatggagggcaatgtggatggccacaccttcagcatccgcggcaagggctacggcgatgccagcgtgggcaaggtggatgcccagttcatctgcaccaccggcgatgtgcccgtgccctggagcaccctggtgaccaccctgacctacggcgcccagtgcttcgccaagtacggccccgagctgaaggatttctacaagagctgcatgcccgatggctacgtgcaggagcgcaccatcaccttcgagggcgatggcaatttcaagacccgcgccgaggtgaccttcgagaatggcagcgtgtacaatcgcgtgaagctgaatggccagggcttcaagaaggatggccacgtgctgggcaagaatctggagttcaatttcaccccccactgcctgtacatctggggcgatcaggccaatcacggcctgaagagcgccttcaagatct

SEQ ID NO: 100 - DNA, 225 bp, Solanum tuberosum
gactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggtta

SEQ ID NO: 101 - DNA, 218 bp, Diabrotica virgifera
tagctctgatgacagagcccatcgagtttcaagccaaacagttgcataaagctatcagcggattgggaactgatgaaagtacaatmgtmgaaattttaagtgtmcacaacaacgatgagattataagaatttcccaggcctatgaaggattgtaccaacgmtcattggaatctgatatcaaaggagatacctcaggaacattaaaaaagaattattag

SEQ ID NO: 102 - DNA, 424 bp, Diabrotica virgifera
  misc_feature (393)..(393): n is a, c, g, or t
  misc_feature (394)..(394): n is a, c, g, or t
  misc_feature (395)..(395): n is a, c, g, or t
ttgttacaagctggagaacttctctttgctggaaccgaagagtcagtatttaatgctgtattctgtcaaagaaataaaccacaattgaatttgatattcgacaaatatgaagaaattgttgggcatcccattgaaaaagccattgaaaacgagttttcaggaaatgctaaacaagccatgttacaccttatccagagcgtaagagatcaagttgcatatttggtaaccaggctgcatgattcaatggcaggcgtcggtactgacgatagaactttaatcagaattgttgtttcgagatctgaaatcgatctagaggaaatcaaacaatgctatgaagaaatctacagtaaaaccttggctgataggatagcggatgacacatctggcgactannnaaaagccttattagccgttgttggttaag

SEQ ID NO: 103 - DNA, 397 bp, Diabrotica virgifera
agatgttggctgcatctagagaattacacaagttcttccatgattgcaaggatgtactgagcagaatagtggaaaaacaggtatccatgtctgatgaattgggaagggacgcaggagctgtcaatgcccttcaacgcaaacaccagaacttcctccaagacctacaaacactccaatcgaacgtccaacaaatccaagaagaatcagctaaacttcaagctagctatgccggtgatagagctaaagaaatcaccaacagggagcaggaagtggtagcagcctgggcagccttgcagatcgcttgcgatcagagacacggaaaattgagcgatactggtgatctattcaaattctttaacttggtacgaacgttgatgcagtggatggacgaatggac

SEQ ID NO: 104 - DNA, 490 bp, Diabrotica virgifera
gcagatgaacaccagcgagaaaccaagagatgttagtggtgttgaattgttgatgaacaaccatcagacactcaaggctgagatcgaagccagagaagacaactttacggcttgtatttctttaggaaaggaattgttgagccgtaatcactatgctagtgctgatattaaggataaattggtcgcgttgacgaatcaaaggaatgctgtactacagaggtgggaagaaagatgggagaacttgcaactcatcctcgaggtataccaattcgccagagatgcggccgtcgccgaagcatggttgatcgcacaagaaccttacttgatgagccaagaactaggacacaccattgacgacgttgaaaacttgataaagaaacacgaagcgttcgaaaaatcggcagcggcgcaagaagagagattcagtgctttggagagactgacgacgttcgaattgagagaaataaagaggaaacaagaagctgcccag

SEQ ID NO: 105 - DNA, 330 bp, Diabrotica virgifera
agtgaaatgttagcaaatataacatccaagtttcgtaattgtacttgctcagttagaaaatattctgtagtttcactatcttcaaccgaaaatagaataaatgtagaacctcgcgaacttgcctttcctccaaaatatcaagaacctcgacaagtttggttggagagtttagatacgatagacgacaaaaaattgggtattcttgagctgcatcctgatgtttttgctactaatccaagaatagatattatacatcaaaatgttagatggcaaagtttatatagatatgtaagctatgctcatacaaagtcaagatttgaagtgagaggt

SEQ ID NO: 106 - DNA, 320 bp, Diabrotica virgifera
caaagtcaagatttgaagtgagaggtggaggtcgaaaaccgtggccgcaaaagggattgggacgtgctcgacatggttcaattagaagtccactttggagaggtggaggagttgttcatggaccaaaatctccaacccctcatttttacatgattccattctacacccgtttgctgggtttgactagcgcactttcagtaaaatttgcccaagatgacttgcacgttgtggatagtctagatctgccaactgacgaacaaagttatatagaagagctggtcaaaagccgcttttgggggtccttcttgttttatttgtag

SEQ ID NO: 107 - DNA, 47 bp, Artificial Sequence
  Primer oligonucleotide
ttaatacgactcactatagggagacgtagtgtcaaaagcaagtttac

SEQ ID NO: 108 - DNA, 23 bp, Artificial Sequence
  Primer oligonucleotide
gcgaacttatgttgatcttgata

SEQ ID NO: 109 - DNA, 23 bp, Artificial Sequence
  Primer oligonucleotide
cgtagtgtcaaaagcaagtttac

SEQ ID NO: 110 - DNA, 47 bp, Artificial Sequence
  Primer oligonucleotide
ttaatacgactcactatagggagagcgaacttatgttgatcttgata

SEQ ID NO: 111 - DNA, 44 bp, Artificial Sequence
  Primer oligonucleotide
ttaatacgactcactatagggagagttgtgtggcctctggtaga

SEQ ID NO: 112 - DNA, 21 bp, Artificial Sequence
  Primer oligonucleotide
tctaactcgtagtaatcaggc

SEQ ID NO: 113 - DNA, 20 bp, Artificial Sequence
  Primer oligonucleotide
gttgtgtggcctctggtaga

SEQ ID NO: 114 - DNA, 45 bp, Artificial Sequence
  Primer oligonucleotide
ttaatacgactcactatagggagatctaactcgtagtaatcaggc

SEQ ID NO: 115 - DNA, 47 bp, Artificial Sequence
  Primer oligonucleotide
ttaatacgactcactatagggagatcttgtcatgatgacagtattcg

SEQ ID NO: 116 - DNA, 26 bp, Artificial Sequence
  Primer oligonucleotide
ccatctggtcctaatttttttttgca

SEQ ID NO: 117 - DNA, 23 bp, Artificial Sequence
  Primer oligonucleotide
tcttgtcatgatgacagtattcg

SEQ ID NO: 118 - DNA, 50 bp, Artificial Sequence
  Primer oligonucleotide
ttaatacgactcactatagggagaccatctggtcctaatttttttttgca

SEQ ID NO: 119 - DNA, 46 bp, Artificial Sequence
  Primer oligonucleotide
ttaatacgactcactatagggagacgctgtcgctaaagatttaaag

SEQ ID NO: 120 - DNA, 26 bp, Artificial Sequence
  Primer oligonucleotide
ctttattattttccgctgttaaatag

SEQ ID NO: 121 - DNA, 22 bp, Artificial Sequence
  Primer oligonucleotide
cgctgtcgctaaagatttaaag

SEQ ID NO: 122 - DNA, 50 bp, Artificial Sequence
  Primer oligonucleotide
ttaatacgactcactatagggagactttattattttccgctgttaaatag

SEQ ID NO: 123 - DNA, 54 bp, Artificial Sequence
  Primer oligonucleotide
ttaatacgactcactatagggagaaggttgaaaaaacaaagttgttgttacttc

SEQ ID NO: 124 - DNA, 25 bp, Artificial Sequence
  Primer oligonucleotide
ctactagctctttggcccaacagag

SEQ ID NO: 125 - DNA, 30 bp, Artificial Sequence
  Primer oligonucleotide
aggttgaaaaaacaaagttgttgttacttc

SEQ ID NO: 126 - DNA, 48 bp, Artificial Sequence
  Primer oligonucleotide
taatacgactcactatagggagactactagctctttggcccaacagag

SEQ ID NO: 127 - DNA, 50 bp, Artificial Sequence
  Primer oligonucleotide
ttaatacgactcactatagggagacggtgtagaaagattaatggaatgtg

SEQ ID NO: 128 - DNA, 26 bp, Artificial Sequence
  Primer oligonucleotide
tcacaggctaactttttgttggagtt

SEQ ID NO: 129 - DNA, 26 bp, Artificial Sequence
  Primer oligonucleotide
cggtgtagaaagattaatggaatgtg

SEQ ID NO: 130 - DNA, 50 bp, Artificial Sequence
  Primer oligonucleotide
ttaatacgactcactatagggagatcacaggctaactttttgttggagtt

SEQ ID NO: 131 - DNA, 48 bp, Artificial Sequence
  Primer oligonucleotide
ttaatacgactcactatagggagacaatgacgcgatcagaagataaag

SEQ ID NO: 132 - DNA, 29 bp, Artificial Sequence
  Primer oligonucleotide
tcagtcaaaactgcaaagaaatattgtaa

SEQ ID NO: 133 - DNA, 24 bp, Artificial Sequence
  Primer oligonucleotide
caatgacgcgatcagaagataaag

SEQ ID NO: 134 - DNA, 53 bp, Artificial Sequence
  Primer oligonucleotide
ttaatacgactcactatagggagatcagtcaaaactgcaaagaaatattgtaa

SEQ ID NO: 135 - DNA, 49 bp, Artificial Sequence
  Primer oligonucleotide
ttaatacgactcactatagggagaacaagtagtatcatcggtatcggaa

SEQ ID NO: 136 - DNA, 19 bp, Artificial Sequence
  Primer oligonucleotide
ggaaaattcagcgggaagc

SEQ ID NO: 137 - DNA, 25 bp, Artificial Sequence
  Primer oligonucleotide
acaagtagtatcatcggtatcggaa

SEQ ID NO: 138 - DNA, 43 bp, Artificial Sequence
  Primer oligonucleotide
ttaatacgactcactatagggagaggaaaattcagcgggaagc

SEQ ID NO: 139 - DNA, 54 bp, Artificial Sequence
  Primer oligonucleotide
ttaatacgactcactatagggagagtattcggttttacacaaaaatgaaaatgc

SEQ ID NO: 140 - DNA, 30 bp, Artificial Sequence
  Primer oligonucleotide
actataggctgtaatttttccagatccaga

SEQ ID NO: 141 - DNA, 30 bp, Artificial Sequence
  Primer oligonucleotide
gtattcggttttacacaaaaatgaaaatgc

SEQ ID NO: 142 - DNA, 54 bp, Artificial Sequence
  Primer oligonucleotide
ttaatacgactcactatagggagaactataggctgtaatttttccagatccaga

SEQ ID NO: 143 - DNA, 46 bp, Artificial Sequence
  Primer oligonucleotide
ttaatacgactcactatagggagaatgatgccaataggaggagctc

SEQ ID NO: 144 - DNA, 28 bp, Artificial Sequence
  Primer oligonucleotide
gtttgtgtcctgaaatggtttttcaaac

SEQ ID NO: 145 - DNA, 22 bp, Artificial Sequence
  Primer oligonucleotide
atgatgccaataggaggagctc

SEQ ID NO: 146 - DNA, 52 bp, Artificial Sequence
  Primer oligonucleotide
ttaatacgactcactatagggagagtttgtgtcctgaaatggtttttcaaac

SEQ ID NO: 147 - DNA, 49 bp, Artificial Sequence
  Primer oligonucleotide
ttaatacgactcactatagggagagaagcaaattgtaacaaaccaaacg

SEQ ID NO: 148 - DNA, 27 bp, Artificial Sequence
  Primer oligonucleotide
cttcttgtacaacagttgttttttcag

SEQ ID NO: 149 - DNA, 25 bp, Artificial Sequence
  Primer oligonucleotide
gaagcaaattgtaacaaaccaaacg

SEQ ID NO: 150 - DNA, 51 bp, Artificial Sequence
  Primer oligonucleotide
ttaatacgactcactatagggagacttcttgtacaacagttgttttttcag

SEQ ID NO: 151 - DNA, 46 bp, Artificial Sequence
  Primer oligonucleotide
ttaatacgactcactatagggagacctactgaagctgtgcaattcc

SEQ ID NO: 152 - DNA, 29 bp, Artificial Sequence
  Primer oligonucleotide
agcgttgtaatactttataacgacattct

SEQ ID NO: 153 - DNA, 22 bp, Artificial Sequence
  Primer oligonucleotide
cctactgaagctgtgcaattcc

SEQ ID NO: 154 - DNA, 53 bp, Artificial Sequence
  Primer oligonucleotide
ttaatacgactcactatagggagaagcgttgtaatactttataacgacattct

SEQ ID NO: 155 - DNA, 52 bp, Artificial Sequence
  Primer oligonucleotide
ttaatacgactcactatagggagaacctgaaaagcttagatttttcaaattc

SEQ ID NO: 156 - DNA, 25 bp, Artificial Sequence
  Primer oligonucleotide
tgtgattttggtcttgtgtaacagg

SEQ ID NO: 157 - DNA, 28 bp, Artificial Sequence
  Primer oligonucleotide
acctgaaaagcttagatttttcaaattc

SEQ ID NO: 158 - DNA, 49 bp, Artificial Sequence
  Primer oligonucleotide
ttaatacgactcactatagggagatgtgattttggtcttgtgtaacagg

SEQ ID NO: 159 - DNA, 48 bp, Artificial Sequence
  Primer oligonucleotide
ttaatacgactcactatagggagaccatgttttcgcaatctcaagtag

SEQ ID NO: 160 - DNA, 22 bp, Artificial Sequence
  Primer oligonucleotide
gtcgcattctttgccggtgaat

SEQ ID NO: 161 - DNA, 24 bp, Artificial Sequence
  Primer oligonucleotide
ccatgttttcgcaatctcaagtag

SEQ ID NO: 162 - DNA, 46 bp, Artificial Sequence
  Primer oligonucleotide
ttaatacgactcactatagggagagtcgcattctttgccggtgaat

SEQ ID NO: 163 - DNA, 45 bp, Artificial Sequence
  Primer oligonucleotide
ttaatacgactcactatagggagaatggctgaccaactcaccgaa

SEQ ID NO: 164 - DNA, 26 bp, Artificial Sequence
  Primer oligonucleotide
ctcttcgtaattgacttgaccatcac

SEQ ID NO: 165 - DNA, 21 bp, Artificial Sequence
  Primer oligonucleotide
atggctgaccaactcaccgaa

SEQ ID NO: 166 - DNA, 50 bp, Artificial Sequence
  Primer oligonucleotide
ttaatacgactcactatagggagactcttcgtaattgacttgaccatcac

SEQ ID NO: 167 - DNA, 51 bp, Artificial Sequence
  Primer oligonucleotide
ttaatacgactcactatagggagatgattaaaaaagcaacttgatgaggct

SEQ ID NO: 168 - DNA, 24 bp, Artificial Sequence
  Primer oligonucleotide
tcaactgtttggataaggtctccc

SEQ ID NO: 169 - DNA, 27 bp, Artificial Sequence
  Primer oligonucleotide
tgattaaaaaagcaacttgatgaggct

SEQ ID NO: 170 - DNA, 48 bp, Artificial Sequence
  Primer oligonucleotide
ttaatacgactcactatagggagatcaactgtttggataaggtctccc

SEQ ID NO: 171 - DNA, 48 bp, Artificial Sequence
  Primer oligonucleotide
ttaatacgactcactatagggagaagtgccgaaataattaggttcaag

SEQ ID NO: 172 - DNA, 23 bp, Artificial Sequence
  Primer oligonucleotide
ttccttggcgttcttaaacagcg

SEQ ID NO: 173 - DNA, 24 bp, Artificial Sequence
  Primer oligonucleotide
agtgccgaaataattaggttcaag

SEQ ID NO: 174 - DNA, 47 bp, Artificial Sequence
  Primer oligonucleotide
ttaatacgactcactatagggagattccttggcgttcttaaacagcg

SEQ ID NO: 175 - DNA, 50 bp, Artificial Sequence
  Primer oligonucleotide
ttaatacgactcactatagggagagtatgaacgaactggattctcttagg

SEQ ID NO: 176 - DNA, 20 bp, Artificial Sequence
  Primer oligonucleotide
tggttgtcatcgaggaaacg

SEQ ID NO: 177 - DNA, 26 bp, Artificial Sequence
  Primer oligonucleotide
gtatgaacgaactggattctcttagg

SEQ ID NO: 178 - DNA, 44 bp, Artificial Sequence
  Primer oligonucleotide
ttaatacgactcactatagggagatggttgtcatcgaggaaacg

SEQ ID NO: 179 - DNA, 49 bp, Artificial Sequence
  Primer oligonucleotide
ttaatacgactcactatagggagaagttcaggagatatgtcttgtgccc

SEQ ID NO: 180 - DNA, 25 bp, Artificial Sequence
  Primer oligonucleotide
cttaattccaaatgcgtaggaaact

SEQ ID NO: 181 - DNA, 25 bp, Artificial Sequence
  Primer oligonucleotide
agttcaggagatatgtcttgtgccc

SEQ ID NO: 182 - DNA, 49 bp, Artificial Sequence
  Primer oligonucleotide
ttaatacgactcactatagggagacttaattccaaatgcgtaggaaact

SEQ ID NO: 183 - DNA, 46 bp, Artificial Sequence
  Primer oligonucleotide
ttaatacgactcactatagggagaggtggactgagtccagatttgc

SEQ ID NO: 184 - DNA, 28 bp, Artificial Sequence
  Primer oligonucleotide
gtgatctacttcttatttttgttagggg

SEQ ID NO: 185 - DNA, 22 bp, Artificial Sequence
  Primer oligonucleotide
ggtggactgagtccagatttgc

SEQ ID NO: 186 - DNA, 52 bp, Artificial Sequence
  Primer oligonucleotide
ttaatacgactcactatagggagagtgatctacttcttatttttgttagggg

SEQ ID NO: 187 - DNA, 47 bp, Artificial Sequence
  Primer oligonucleotide
ttaatacgactcactatagggagacgattctgctgcaaaaattaacg

SEQ ID NO: 188 - DNA, 21 bp, Artificial Sequence
  Primer oligonucleotide
aacgtcggcattttcgtaagc

SEQ ID NO: 189 - DNA, 23 bp, Artificial Sequence
  Primer oligonucleotide
cgattctgctgcaaaaattaacg

SEQ ID NO: 190 - DNA, 45 bp, Artificial Sequence
  Primer oligonucleotide
ttaatacgactcactatagggagaaacgtcggcattttcgtaagc

SEQ ID NO: 191 - DNA, 47 bp, Artificial Sequence
  Primer oligonucleotide
ttaatacgactcactatagggagaaatgaagaaaccggtactccaga

SEQ ID NO: 192 - DNA, 22 bp, Artificial Sequence
  Primer oligonucleotide
atggtagcgctttcagtttgag

SEQ ID NO: 193 - DNA, 23 bp, Artificial Sequence
  Primer oligonucleotide
aatgaagaaaccggtactccaga

SEQ ID NO: 194 - DNA, 46 bp, Artificial Sequence
  Primer oligonucleotide
ttaatacgactcactatagggagaatggtagcgctttcagtttgag

SEQ ID NO: 195 - DNA, 46 bp, Artificial Sequence
  Primer oligonucleotide
ttaatacgactcactatagggagaatgtcgaagctttcatttaggg

SEQ ID NO: 196 - DNA, 21 bp, Artificial Sequence
  Primer oligonucleotide
acctggcaattcggaaacttc

SEQ ID NO: 197 - DNA, 22 bp, Artificial Sequence
  Primer oligonucleotide
atgtcgaagctttcatttaggg

SEQ ID NO: 198 - DNA, 45 bp, Artificial Sequence
  Primer oligonucleotide
ttaatacgactcactatagggagaacctggcaattcggaaacttc

SEQ ID NO: 199 - DNA, 44 bp, Artificial Sequence
  Primer oligonucleotide
ttaatacgactcactatagggagatccaaatcaaagttgggcga

SEQ ID NO: 200 - DNA, 19 bp, Artificial Sequence
  Primer oligonucleotide
agccgcctctaccaaccct

SEQ ID NO: 201 - DNA, 20 bp, Artificial Sequence
  Primer oligonucleotide
tccaaatcaaagttgggcga

SEQ ID NO: 202 - DNA, 43 bp, Artificial Sequence
  Primer oligonucleotide
ttaatacgactcactatagggagaagccgcctctaccaaccct

SEQ ID NO: 203 - DNA, 53 bp, Artificial Sequence
  Primer oligonucleotide
ttaatacgactcactatagggagaagatgtgtgtttaataagtggaactaact

SEQ ID NO: 204 - DNA, 19 bp, Artificial Sequence
  Primer oligonucleotide
aacacgggagttgggacag

SEQ ID NO: 205 - DNA, 29 bp, Artificial Sequence
  Primer oligonucleotide
agatgtgtgtttaataagtggaactaact

SEQ ID NO: 206 - DNA, 43 bp, Artificial Sequence
  Primer oligonucleotide
ttaatacgactcactatagggagaaacacgggagttgggacag

SEQ ID NO: 207 - DNA, 50 bp, Artificial Sequence
  Primer oligonucleotide
ttaatacgactcactatagggagaatgaaaggaagggaaaagttatgtgc

SEQ ID NO: 208 - DNA, 18 bp, Artificial Sequence
  Primer oligonucleotide
acctgaggaatctggcgg

SEQ ID NO: 209 - DNA, 26 bp, Artificial Sequence
  Primer oligonucleotide
atgaaaggaagggaaaagttatgtgc

SEQ ID NO: 210 - DNA, 42 bp, Artificial
  Primer oligonucleotide
ttaatacgactcactatagggagaacctgaggaatctggcgg

SEQ ID NO: 211 - DNA, 43 bp, Artificial Sequence
  Primer oligonucleotide
ttaatacgactcactatagggagagatggctaaagccccagct

SEQ ID NO: 212 - DNA, 50 bp, Artificial Sequence
  Primer oligonucleotide
ttaatacgactcactatagggagaacattctttcctaagtatgcttctgc

SEQ ID NO: 213 - DNA, 49 bp, Artificial Sequence
  Primer oligonucleotide
ttaatacgactcactatagggagaatgaggttatatttgggatttggag

SEQ ID NO: 214 - DNA, 49 bp, Artificial Sequence
  Primer oligonucleotide
ttaatacgactcactatagggagacttctgctgtttccttcatctttcc

SEQ ID NO: 215 - DNA, 52 bp, Artificial Sequence
  Primer oligonucleotide
ttaatacgactcactatagggagaatggtgaaaccgttgaataaattttatg

SEQ ID NO: 216 - DNA, 52 bp, Artificial Sequence
  Primer oligonucleotide
ttaatacgactcactatagggagacgtttctttctgaagattagatatagcg

SEQ ID NO: 217 - DNA, 52 bp, Artificial Sequence
  Primer oligonucleotide
ttaatacgactcactatagggagaatgatatacgatagggatttttcctatg

SEQ ID NO: 218 - DNA, 51 bp, Artificial Sequence
  Primer oligonucleotide
ttaatacgactcactatagggagaagaaggtttattggtgctaagttgatc

SEQ ID NO: 219 - DNA, 46 bp, Artificial Sequence
  Primer oligonucleotide
ttaatacgactcactatagggagaatgtacgagggcagtcctgtca

SEQ ID NO: 220 - DNA, 46 bp, Artificial Sequence
  Primer oligonucleotide
ttaatacgactcactatagggagagatgaacgctcctctatcgatg

SEQ ID NO: 221 - DNA, 42 bp, Artificial Sequence
  Primer oligonucleotide
ttaatacgactcactatagggagacgtaatgaatccaggcgc

SEQ ID NO: 222 - DNA, 49 bp, Artificial Sequence
  Primer oligonucleotide
ttaatacgactcactatagggagagttgtcaatggaacgatccaaattc

SEQ ID NO: 223 - DNA, 48 bp, Artificial Sequence
  Primer oligonucleotide
ttaatacgactcactatagggagaatgggtcagtgttttagtgctagc

SEQ ID NO: 224 - DNA, 49 bp, Artificial Sequence
  Primer oligonucleotide
ttaatacgactcactatagggagagtgatcgtcgaagggaaatattttg

SEQ ID NO: 225 - DNA, 47 bp, Artificial Sequence
  Primer oligonucleotide
ttaatacgactcactatagggagaatgagtatgaaacgaggagcact

SEQ ID NO: 226 - DNA, 48 bp, Artificial Sequence
  Primer oligonucleotide
ttaatacgactcactatagggagaagacccagcagcatcaattctaac

SEQ ID NO: 227 - DNA, 47 bp, Artificial Sequence
  Primer oligonucleotide
ttaatacgactcactatagggagacaccatgggctccagcggcgccc

SEQ ID NO: 228 - DNA, 23 bp, Artificial Sequence
  Primer oligonucleotide
agatcttgaaggcgctcttcagg

SEQ ID NO: 229 - DNA, 23 bp, Artificial Sequence
  Primer oligonucleotide
caccatgggctccagcggcgccc

SEQ ID NO: 230 - DNA, 47 bp, Artificial Sequence
  Primer oligonucleotide
ttaatacgactcactatagggagaagatcttgaaggcgctcttcagg

SEQ ID NO: 231 - DNA, 46 bp, Artificial Sequence
  Primer oligonucleotide
ttaatacgactcactatagggagagctccaacagtggttccttatc

SEQ ID NO: 232 - DNA, 29 bp, Artificial Sequence
  Primer oligonucleotide
ctaataattcttttttaatgttcctgagg

SEQ ID NO: 233 - DNA, 22 bp, Artificial Sequence
  Primer oligonucleotide
gctccaacagtggttccttatc

SEQ ID NO: 234 - DNA, 53 bp, Artificial Sequence
  Primer oligonucleotide
ttaatacgactcactatagggagactaataattcttttttaatgttcctgagg

SEQ ID NO: 235 - DNA, 48 bp, Artificial Sequence
  Primer oligonucleotide
ttaatacgactcactatagggagattgttacaagctggagaacttctc

SEQ ID NO: 236 - DNA, 24 bp, Artificial Sequence
  Primer oligonucleotide
cttaaccaacaacggctaataagg

SEQ ID NO: 237 - DNA, 24 bp, Artificial Sequence
  Primer oligonucleotide
ttgttacaagctggagaacttctc

SEQ ID NO: 238 - DNA, 48 bp, Artificial Sequence
  Primer oligonucleotide
ttaatacgactcactatagggagacttaaccaacaacggctaataagg

SEQ ID NO: 239 - DNA, 47 bp, Artificial Sequence
  Primer oligonucleotide
ttaatacgactcactatagggagaagatgttggctgcatctagagaa

SEQ ID NO: 240 - DNA, 22 bp, Artificial Sequence
  Primer oligonucleotide
gtccattcgtccatccactgca

SEQ ID NO: 241 - DNA, 23 bp, Artificial Sequence
  Primer oligonucleotide
agatgttggctgcatctagagaa

SEQ ID NO: 242 - DNA, 46 bp, Artificial Sequence
  Primer oligonucleotide
ttaatacgactcactatagggagagtccattcgtccatccactgca

SEQ ID NO: 243 - DNA, 46 bp, Artificial Sequence
  Primer oligonucleotide
ttaatacgactcactatagggagagcagatgaacaccagcgagaaa

SEQ ID NO: 244 - DNA, 22 bp, Artificial Sequence
  Primer oligonucleotide
ctgggcagcttcttgtttcctc

SEQ ID NO: 245 - DNA, 22 bp, Artificial Sequence
  Primer oligonucleotide
gcagatgaacaccagcgagaaa

SEQ ID NO: 246 - DNA, 46 bp, Artificial Sequence
  Primer oligonucleotide
ttaatacgactcactatagggagactgggcagcttcttgtttcctc

SEQ ID NO: 247 - DNA, 51 bp, Artificial Sequence
  Primer oligonucleotide
ttaatacgactcactatagggagaagtgaaatgttagcaaatataacatcc

SEQ ID NO: 248 - DNA, 26 bp, Artificial Sequence
  Primer oligonucleotide
acctctcacttcaaatcttgactttg

SEQ ID NO: 249 - DNA, 27 bp, Artificial Sequence
  Primer oligonucleotide
agtgaaatgttagcaaatataacatcc

SEQ ID NO: 250 - DNA, 50 bp, Artificial Sequence
  Primer oligonucleotide
ttaatacgactcactatagggagaacctctcacttcaaatcttgactttg

SEQ ID NO: 251 - DNA, 50 bp, Artificial Sequence
  Primer oligonucleotide
ttaatacgactcactatagggagacaaagtcaagatttgaagtgagaggt

SEQ ID NO: 252 - DNA, 25 bp, Artificial Sequence
  Primer oligonucleotide
ctacaaataaaacaagaaggacccc

SEQ ID NO: 253 - DNA, 26 bp, Artificial Sequence
  Primer oligonucleotide
caaagtcaagatttgaagtgagaggt

SEQ ID NO: 254 - DNA, 49 bp, Artificial Sequence
  Primer oligonucleotide
ttaatacgactcactatagggagactacaaataaaacaagaaggacccc

SEQ ID NO: 255 - DNA, 22 bp, Artificial Sequence
  Primer oligonucleotide
  misc_feature (22)..(22): n is a, c, g, or t
ttttttttttttttttttttvn

SEQ ID NO: 256 - DNA, 20 bp, Artificial Sequence
  Primer oligonucleotide
ttgtgatgttggtggcgtat

SEQ ID NO: 257 - DNA, 24 bp, Artificial Sequence
  Primer oligonucleotide
tgttaaataaaaccccaaagatcg

SEQ ID NO: 258 - DNA, 21 bp, Artificial Sequence
  Primer oligonucleotide
tgagggtaatgccaactggtt

SEQ ID NO: 259 - DNA, 24 bp, Artificial Sequence
  Primer oligonucleotide
gcaatgtaaccgagtgtctctcaa

SEQ ID NO: 260 - DNA, 32 bp, Artificial Sequence
  Probe oligonucleotide
tttttggcttagagttgatggtgtactgatga

SEQ ID NO: 261 - DNA, 25 bp, Artificial Sequence
  Primer oligonucleotide
gtatgtttctgcttctacctttgat

SEQ ID NO: 262 - DNA, 29 bp, Artificial Sequence
  Primer oligonucleotide
ccatgttttggtcatatattagaaaagtt

SEQ ID NO: 263 - DNA, 34 bp, Artificial Sequence
  Probe oligonucleotide
agtaatatagtatttcaagtatttttttcaaaat

SEQ ID NO: 264 - DNA, 20 bp, Artificial Sequence
  Primer oligonucleotide
tgttcggttccctctaccaa

SEQ ID NO: 265 - DNA, 22 bp, Artificial Sequence
  Primer oligonucleotide
caacatccatcaccttgactga

SEQ ID NO: 266 - DNA, 24 bp, Artificial Sequence
  Probe oligonucleotide
cacagaaccgtcgcttcagcaaca

SEQ ID NO: 267 - DNA, 18 bp, Artificial Sequence
  Primer oligonucleotide
tggcggacgacgacttgt

SEQ ID NO: 268 - DNA, 19 bp, Artificial Sequence
  Primer oligonucleotide
aaagtttggaggctgccgt

SEQ ID NO: 269 - DNA, 26 bp, Artificial Sequence
  Probe oligonucleotide
cgagcagaccgccgtgtacttctacc

SEQ ID NO: 270 - DNA, 19 bp, Artificial Sequence
  Primer oligonucleotide
cttagctggataacgccac

SEQ ID NO: 271 - DNA, 19 bp, Artificial Sequence
  Primer oligonucleotide
gaccgtaaggcttgatgaa

SEQ ID NO: 272 - DNA, 21 bp, Artificial Sequence
  Probe oligonucleotide
cgagattctccgcgctgtaga

SEQ ID NO: 273 - DNA, 151 bp, Escherichia coli
gaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaag

SEQ ID NO: 274 - DNA, 69 bp, Artificial Sequence
  Partial coding region
tgttcggttccctctaccaagcacagaaccgtcgcttcagcaacacctcagtcaaggtgatggatgttg

SEQ ID NO: 275 - DNA, 4233 bp, Zea mays
agcctggtgtttccggaggagacagacatgatccctgccgttgctgatccgacgacgctggacggcgggggcgcgcgcaggccgttgctcccggagacggaccctcgggggcgtgctgccgccggcgccgagcagaagcggccgccggctacgccgaccgttctcaccgccgtcgtctccgccgtgctcctgctcgtcctcgtggcggtcacagtcctcgcgtcgcagcacgtcgacgggcaggctgggggcgttcccgcgggcgaagatgccgtcgtcgtcgaggtggccgcctcccgtggcgtggctgagggcgtgtcggagaagtccacggccccgctcctcggctccggcgcgctccaggacttctcctggaccaacgcgatgctggcgtggcagcgcacggcgttccacttccagccccccaagaactggatgaacggttagttggacccgtcgccatcggtgacgacgcgcggatcgtttttttcttttttcctctcgttctggctctaacttggttccgcgtttctgtcacggacgcctcgtgcacatggcgatacccgatccgccggccgcgtatatctatctacctcgaccggcttctccagatccgaacggtaagttgttggctccgatacgatcgatcacatgtgagctcggcatgctgcttttctgcgcgtgcatgcggctcctagcattccacgtccacgggtcgtgacatcaatgcacgatataatcgtatcggtacagagatattgtcccatcagctgctagctttcgcgtattgatgtcgtgacattttgcacgcaggtccgctgtatcacaagggctggtaccacctcttctaccagtggaacccggactccgcggtatggggcaacatcacctggggccacgccgtctcgcgcgacctcctccactggctgcacctaccgctggccatggtgcccgatcacccgtacgacgccaacggcgtctggtccgggtcggcgacgcgcctgcccgacggccggatcgtcatgctctacacgggctccacggcggagtcgtcggcgcaggtgcagaacctcgcggagccggccgacgcgtccgacccgctgctgcgggagtgggtcaagtcggacgccaacccggtgctggtgccgccgccgggcatcgggccgacggacttccgcgacccgacgacggcgtgtcggacgccggccggcaacgacacggcgtggcgggtcgccatcgggtccaaggaccgggaccacgcggggctggcgctggtgtaccggacggaggacttcgtgcggtacgacccggcgccggcgctgatgcacgccgtgccgggcaccggcatgtgggagtgcgtggacttctacccggtggccgcgggatcaggcgccgcggcgggcagcggggacgggctggagacgtccgcggcgccgggacccggggtgaagcacgtgctcaaggctagcctcgacgacgacaagcacgactactacgcgatcggcacctacgacccggcgacggacacctggacccccgacagcgcggaggacgacgtcgggatcggcctccggtacgactatggcaagtactacgcgtcgaagaccttctacgaccccgtccttcgccggcgggtgctctgggggtgggtcggcgagaccgacagcgagcgcgcggacatcctcaagggctgggcatccgtgcaggtacgtctcagggtttgaggctagcatggcttcaatcttgctggcatcgaatcattaatgggcagatattataacttgataatctgggttggttgtgtgtggtggggatggtgacacacgcgcggtaataatgtagctaagctggttaaggatgagtaatggggttgcgtataaacgacagctctgctaccattacttctgacacccgattgaaggagacaacagtaggggtagccggtagggttcgtcgacttgccttttcttttttcctttgttttgttgtggatcgtccaacacaaggaaaataggatcatccaacaaacatggaagtaatcccgtaaaacatttctcaaggaaccatctagctagacgagcgtggcatgatccatgcatgcacaaacactagataggtctctgcagctgtgatgttcctttacatataccaccgtccaaactgaatccggtctgaaaattgttcaagcagagaggccccgatcctcacacctgtacacgtccctgtacgcgccgtcgtggtctcccgtgatcctgccccgtcccctccacgcggccacgcctgctgcagcgctctgtacaagcgtgcaccacgtgagaatttccgtctactcgagcctagtagttagacgggaaaacgagaggaagcgcacggtccaagcacaacactttgcgcgggcccgtgacttgtctccggttggctgagggcgcgcgacagagatgtatggcgccgcggcgtgtcttgtgtcttgtcttgcctatacaccgtagtcagagactgtgtcaaagccgtccaacgacaatgagctaggaaacgggttggagagctgggttcttgccttgcctcctgtgatgtctttgccttgcatagggggcgcagtatgtagctttgcgttttacttcacgccaaaggatactgctgatcgtgaattattattattatatatatatcgaatatcgatttcgtcgctctcgtggggttttattttccagactcaaacttttcaaaaggcctgtgttttagttcttttcttccaattgagtaggcaaggcgtgtgagtgtgaccaacgcatgcatggatatcgtggtagactggtagagctgtcgttaccagcgcgatgcttgtatatgtttgcagtattttcaaatgaatgtctcagctagcgtacagttgaccaagtcgacgtggagggcgcacaacagacctctgacattattcactttttttttaccatgccgtgcacgtgcagtcaatccccaggacggtcctcctggacacgaagacgggcagcaacctgctccagtggccggtggtggaggtggagaacctccggatgagcggcaagagcttcgacggcgtcgcgctggaccgcggatccgtcgtgcccctcgacgtcggcaaggcgacgcaggtgacgccgcacgcagcctgctgcagcgaacgaactcgcgcgttgccggcccgcggccagctgacttagtttctctggctgatcgaccgtgtgcctgcgtgcgtgcagttggacatcgaggctgtgttcgaggtggacgcgtcggacgcggcgggcgtcacggaggccgacgtgacgttcaactgcagcaccagcgcaggcgcggcgggccggggcctgctcggcccgttcggccttctcgtgctggcggacgacgacttgtccgagcagaccgccgtgtacttctacctgctcaagggcacggacggcagcctccaaactttcttctgccaagacgagctcaggtatgtatgttatgacttatgaccatgcatgcatgcgcatttcttagctaggctgtgaagcttcttgttgagttgtttcacagatgcttaccgtctgctttgtttcgtatttcgactaggcatccaaggcgaacgatctggttaagagagtatacgggagcttggtccctgtgctagatggggagaatctctcggtcagaatactggtaagtttttacagcgccagccatgcatgtgttggccagccagctgctggtactttggacactcgttcttctcgcactgctcattattgcttctgatctggatgcactacaaattgaaggttgaccactccatcgtggagagctttgctcaaggcgggaggacgtgcatcacgtcgcgagtgtaccccacacgagccatctacgactccgcccgcgtcttcctcttcaacaacgccacacatgctcacgtcaaagcaaaatccgtcaagatctggcagctcaactccgcctacatccggccatatccggcaacgacgacttctctatgactaaattaagtgacggacagataggcgatattgcatacttgcatcatgaactcatttgtacaacagtgattgtttaatttatttgctgccttccttatccttcttgtgaaactatatggtacacacatgtatcattaggtctagtagtgttgttgcaaagacacttagacaccagaggttccaggagtatcagagataaggtataagagggagcagggagcag

SEQ ID NO: 276 - DNA, 1150 bp, Zea mays
caacggggcagcactgcactgcactgcaactgcgaatttccgtcagcttggagcggtccaagcgccctgcgaagcaaactacgccgatggcttcggcggcggcgtgggagggtccgacggccgcggagctgaagacagcgggggcggaggtgattcccggcggcgtgcgagtgaaggggtgggtcatccagtcccacaaaggccctatcctcaacgccgcctctctgcaacgctttgaagatgaacttcaaacaacacatttacctgagatggtttttggagagagtttcttgtcacttcaacatacacaaactggcatcaaatttcattttaatgcgcttgatgcactcaaggcatggaagaaagaggcactgccacctgttgaggttcctgctgcagcaaaatggaagttcagaagtaagccttctgaccaggttatacttgactacgactatacatttacgacaccatattgtgggagtgatgctgtggttgtgaactctggcactccacaaacaagtttagatggatgcggcactttgtgttgggaggatactaatgatcggattgacattgttgccctttcagcaaaagaacccattcttttctacgacgaggttatcttgtatgaagatgagttagctgacaatggtatctcatttcttactgtgcgagtgagggtaatgccaactggttggtttctgcttttgcgtttttggcttagagttgatggtgtactgatgaggttgagagacactcggttacattgcctgtttggaaacggcgacggagccaagccagtggtacttcgtgagtgctgctggagggaagcaacatttgctactttgtctgcgaaaggatatccttcggactctgcagcgtacgcggacccgaaccttattgcccataagcttcctattgtgacgcagaagacccaaaagctgaaaaatcctacctgactgacacaaaggcgccctaccgcgtgtacatcatgactgtcctgtcctatcgttgccttttgtgtttgccacatgttgtggatgtacgtttctatgacgaaacaccatagtccatttcgcctgggccgaacagagatagctgattgtcatgtcacgtttgaattagaccattccttagccctttttccccc

SEQ ID NO: 277 - DNA, 471 bp, Artificial Sequence
  Artificial sequence
atgtcatctggagcacttctctttcatgggaagattccttacgttgtggagatggaagggaatgttgatggccacacctttagcatacgtgggaaaggctacggagatgcctcagtgggaaaggactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttactttcccactgaggcatctccgtagcctttcccacgtatgctaaaggtgtggccatcaacattcccttccatctccacaacgtaaggaatcttcccatgaaagagaagtgctccagatgacat

SEQ ID NO: 278 - DNA, 1010 bp, Diabrotica virgifera
attgtaatgctttttgttggcattagatagcgtgtttataataaagaagcaataagcgccaaatattcaattcttgagcctctcgaaccgcagtattcatttctaagacatatttatccgttttaaagaagtttgtaatacaaaacagaagagctgctgttgttttacttaaaatatatgtagggtggaagatagtgttaggttaactaatgaagttgagcccaaaatagtgacacttgcgtttttaggactaataccaccaataccgtaatgatgccaataggaggagctcctccggtgcacggctctagtagtttatcacaaagtaatacctcacaaaatgctagtttaacacctacaggtggatccagcaaatccagtagtagcttgaagccaaattatgcccttaaatttacgctagctggacatacaaaagcagtgtcttctgttaaatttagccctaatggagaatggttagccagctcatctgcagataaacttgtaaaaatttggggagcatatgacgggaagtttgaaaaaccatttcaggacacaaactaggaataagtgatgtagcatggtccagtgatagcagactactagtgtctggtagtgatgataaaacgctcaaaatatgggagctatcctcaggtaaatgtctaaagactctcaaagggcacagcaactacgtattctgctgcaactttaatcctcaatccaaccttatcgtttctggttcttttgacgagtctgtcagaatatgggatgtgagaacaggaaaatgtcttaagacactgccagcacattcagaccctgttagtgcagtacatttcaacagagacggatctcttattgtcagtagcagttacgatggactttgtcgtatttgggacacagcctcgggtcagtgtttgaaaactctcattgacgatgacaatccaccagtatcctttgtcaaattttcaccaaacggcaagtatatattggctgctacattagataacacactc

SEQ ID NO: 279 - DNA, 545 bp, Diabrotica virgifera
tgagtgaagttgcgggtgttacttggagaaactaacatcgttgacaaccatggcggataaagaaaagaaggtcaagaagaagaagaaggaagaggcgaagcaccagctgccgctcctgcccccgccgaaaccaggtcctcctcaaagagttcctccaagaaagccaaaaggtctggttccaatgtcttctccatgttttcgcaatctcaagtagctgaattcaaagaagctttccaacttatggaccacgacaaagatggcatcatctctaagagcgatttgagggccaccttcgatgctgttggtaaactcgccagtgagaaagagctcgacgagatgatcaacgaagcacccggaccaatcaacttcactcaattgttgggattgttcggtacccgtatggctgattccggcggtactgacgatgatgaagttgtcgtcaaggctttcagatcctttgacgaaaacggcaccattgacggagacagattccgccatgccctcatgacctggggagaaaaattcaccggcaaagaatgcgacga

(19) United States
(12) Patent Application Publication
(10) Pub. No.: US 2021/0277413 A1
(43) Pub. Date: Sep. 9, 2021

(54) NUCLEIC ACID MOLECULES THAT CONFER RESISTANCE TO COLEOPTERAN PESTS

(71) Applicant: Dow AgroSciences LLC, Indianapolis, IN (US)

(72) Inventors: Kenneth E. Narva, Zionsville, IN (US); Huarong Li, Zionsville, IN (US); Chaoxian Geng, Zionsville, IN (US); Ignacio Mario Larrinua, Indianapolis, IN (US); Navin Elango, Indianapolis, IN (US); Aaron T. Woosley, Fishers, IN (US); Monica B. Olson, Lebanon, IN (US); Matthew J. Henry, Indianapolis, IN (US); Murugesan Rangasamy, Zionsville, IN (US); Kanika Arora, West New York, NJ (US); Premchand Gandra, Zionsville, IN (US); Sarah E. Worden, Indianapolis, IN (US)

(21) Appl. No.: 15/770,379

(22) PCT Filed: Oct. 20, 2016
(86) PCT No.: PCT/US16/57848

§ 371 Date: Apr. 23, 2018

Related U.S. Application Data
(60) Provisional application No. 62/247,391, filed on Oct. 28, 2015.

(51) Int. Cl. C12N 15/82; C07K 14/435

(52) U.S. Cl. CPC C12N 15/8286; C12N 15/8218; C07K 14/43563

(57) ABSTRACT

This disclosure concerns nucleic acid molecules and methods of use thereof for control of coleopteran pests through RNA interference-mediated inhibition of target coding and transcribed non-coding sequences in coleopteran pests. The disclosure also concerns methods for making transgenic plants that express nucleic acid molecules useful for the control of coleopteran pests, and the plant cells and plants obtained thereby.

### CROSS-REFERENCE TO RELATED APPLICATIONS

This applications claims priority from, and the benefit of PCT/US2016/057848, filed Oct. 20, 2016, and U.S. Provisional Application 62/247,391, filed on Oct. 28, 2015. The entire contents of these applications are hereby incorporated by reference into this application.

### REFERENCE TO SEQUENCE LISTING SUBMITTED ELECTRONICALLY

The official copy of the sequence listing is submitted electronically via EFS-Web as an ASCII formatted sequence listing with a file named “71329-WO-PCT_20170117_R_MIR_Seq_Listing_ST25”, created on Jan. 17, 2017, and having the file size of 277 kilobytes and is filed concurrently with the specification. The sequence listing contained in this ASCII formatted document is part of the specification, and is incorporated herein by reference in its entirety. This is the replacement file submitted in response to the invitation dated Dec. 16, 2016 for the stated reason that the initial file contained several raw sequence listing errors. It merely corrected the defects and no new matter has been added.

### TECHNICAL FIELD OF THE DISCLOSURE

The present invention relates generally to genetic control of plant damage caused by coleopteran pests. In particular embodiments, the present invention relates to identification of target coding and non-coding sequences, and the use of recombinant DNA technologies for post-transcriptionally repressing or inhibiting expression of target coding and non-coding sequences in the cells of a coleopteran pest to provide a plant protective effect.

### BACKGROUND

The western corn rootworm (WCR), Diabrotica virgifera virgifera LeConte, is one of the most devastating corn rootworm species in North America and is a particular concern in corn-growing areas of the Midwestern United States. The northern corn rootworm (NCR), Diabrotica barberi Smith and Lawrence, is a closely-related species that co-inhabits much of the same range as WCR. There are several other related subspecies of Diabrotica that are significant pests in the Americas: the Mexican corn rootworm (MCR), D. virgifera zeae Krysan and Smith; the southern corn rootworm (SCR), D. undecimpunctata howardi Barber; D. balteata LeConte; D. undecimpunctata tenella; D. speciosa Germar; and D. u. undecimpunctata Mannerheim. Twenty-seven years ago, annual economic losses due to rootworm were estimated at $1 billion in lost revenue each year; since then, the average acreage, yield, and price of maize have all increased substantially, such that the current economic impact of corn rootworm is likely to be much higher.

Both WCR and NCR eggs are deposited in the soil during the summer. The insects remain in the egg stage throughout the winter. The eggs are oblong, white, and less than 0.004 inches (0.010 cm) in length. The larvae hatch in late May or early June, with the precise timing of egg hatching varying from year to year due to temperature differences and location. The newly hatched larvae are white worms that are less than 0.125 inches (0.3175 cm) in length. Once hatched, the larvae begin to feed on corn roots. Corn rootworms go through three larval instars. After feeding for several weeks, the larvae molt into the pupal stage. They pupate in the soil, and then emerge from the soil as adults in July and August. Adult rootworms are about 0.25 inches (0.635 cm) in length.

Corn rootworm larvae complete development on corn and several other species of grasses. Larvae reared on yellow foxtail emerge later and have a smaller head capsule size as adults than larvae reared on corn (Ellsbury et al. (2005) Environ. Entomol. 34:627-634). WCR adults feed on corn silk, pollen, and kernels on exposed ear tips. If WCR adults emerge before corn reproductive tissues are present, they may feed on leaf tissue, thereby slowing plant growth and occasionally killing the host plant. However, the adults will quickly shift to preferred silks and pollen when they become available. NCR adults also feed on reproductive tissues of the corn plant, but in contrast rarely feed on corn leaves.

Most of the rootworm damage in corn is caused by larval feeding. Newly hatched rootworms initially feed on fine corn root hairs and burrow into root tips. As the larvae grow larger, they feed on and burrow into primary roots. When corn rootworms are abundant, larval feeding often results in the pruning of roots all the way to the base of the corn stalk. Severe root injury interferes with the roots' ability to transport water and nutrients into the plant, reduces plant growth, and results in reduced grain production, thereby often drastically reducing overall yield. Severe root injury also often results in lodging of corn plants, which makes harvest more difficult and further decreases yield. Furthermore, feeding by adults on the corn reproductive tissues can result in pruning of silks at the ear tip. If this “silk clipping” is severe enough during pollen shed, pollination may be disrupted.

Control of corn rootworms may be attempted by crop rotation, chemical insecticides, biopesticides (e.g., the spore-forming gram-positive bacterium, Bacillus thuringiensis (Bt)), transgenic plants that express Bt toxins, or a combination thereof. Crop rotation suffers from the significant disadvantage of placing unwanted restrictions upon the use of farmland. Moreover, oviposition of some rootworm species may occur in soybean fields, thereby mitigating the effectiveness of crop rotation practiced with corn and soybean.

Chemical insecticides are the most heavily relied upon strategy for achieving corn rootworm control. Chemical insecticide use, though, is an imperfect corn rootworm control strategy; over $1 billion may be lost in the United States each year due to corn rootworm when the costs of the chemical insecticides are added to the costs of the rootworm damage that may occur despite the use of the insecticides. High populations of larvae, heavy rains, and improper application of the insecticide(s) may all result in inadequate corn rootworm control. Furthermore, the continual use of insecticides may select for insecticide-resistant rootworm strains, as well as raise significant environmental concerns due to the toxicity of many of them to non-target species.

RNA interference (RNAi) is a process utilizing endogenous cellular pathways, whereby an interfering RNA (iRNA) molecule (e.g., a dsRNA molecule) that is specific for all, or any portion of adequate size, of a target gene sequence results in the degradation of the mRNA encoded thereby. In recent years, RNAi has been used to perform gene “knockdown” in a number of species and experimental systems; for example, Caenorhabitis elegans, plants, insect embryos, and cells in tissue culture. See, e.g., Fire et al. (1998) Nature 391:806-811; Martinez et al. (2002) Cell 110:563-574; McManus and Sharp (2002) Nature Rev. Genetics 3:737-747.

RNAi accomplishes degradation of mRNA through an endogenous pathway including the DICER protein complex. DICER cleaves long dsRNA molecules into short fragments of approximately 20 nucleotides, termed small interfering RNA (siRNA). The siRNA is unwound into two single-stranded RNAs: the passenger strand and the guide strand. The passenger strand is degraded, and the guide strand is incorporated into the RNA-induced silencing complex (RISC).

U.S. Pat. No. 7,612,194 and U.S. Patent Publication Nos. 2007/0050860, 2010/0192265, and 2011/0154545 disclose a library of 9112 expressed sequence tag (EST) sequences isolated from D. v. virgifera LeConte pupae. It is suggested in U.S. Pat. No. 7,612,194 and U.S. Patent Publication No. 2007/0050860 to operably link to a promoter a nucleic acid molecule that is complementary to one of several particular partial sequences of D. v. virgifera vacuolar-type H⁺-ATPase (V-ATPase) disclosed therein for the expression of anti-sense RNA in plant cells. U.S. Patent Publication No. 2010/0192265 suggests operably linking a promoter to a nucleic acid molecule that is complementary to a particular partial sequence of a D. v. virgifera gene of unknown and undisclosed function (the partial sequence is stated to be 58% identical to C56C10.3 gene product in C. elegans) for the expression of anti-sense RNA in plant cells. U.S. Patent Publication No. 2011/0154545 suggests operably linking a promoter to a nucleic acid molecule that is complementary to two particular partial sequences of D. v. virgifera coatomer beta subunit genes for the expression of anti-sense RNA in plant cells. Further, U.S. Pat. No. 7,943,819 discloses a library of 906 expressed sequence tag (EST) sequences isolated from D. v. virgifera LeConte larvae, pupae, and dissected midguts, and suggests operably linking a promoter to a nucleic acid molecule that is complementary to a particular partial sequence of a D. v. virgifera charged multivesicular body protein 4b gene for the expression of double-stranded RNA in plant cells.

No further suggestion is provided in U.S. Pat. No. 7,612,194, and U.S. Patent Publication Nos. 2007/0050860, 2010/0192265 and 2011/0154545 to use any particular sequence of the more than nine thousand sequences listed therein for RNA interference, other than the several particular partial sequences of V-ATPase and the particular partial sequences of genes of unknown function. Furthermore, none of U.S. Pat. No. 7,612,194, and U.S. Patent Publication Nos. 2007/0050860 and 2010/0192265, and 2011/0154545 provides any guidance as to which other of the over nine thousand sequences provided would be lethal, or even otherwise useful, in species of corn rootworm when used as dsRNA or siRNA. U.S. Pat. No. 7,943,819 provides no suggestion to use any particular sequence of the more than nine hundred sequences listed therein for RNA interference, other than the particular partial sequence of a charged multivesicular body protein 4b gene. Furthermore, U.S. Pat. No. 7,943,819 provides no guidance as to which other of the over nine hundred sequences provided would be lethal, or even otherwise useful, in species of corn rootworm when used as dsRNA or siRNA. U.S. Patent Application Publication No. U.S. 2013/040173 and PCT Application Publication No. WO 2013/169923 describe the use of a sequence derived from a Diabrotica virgifera Snf7 gene for RNA interference in maize. (Also disclosed in Bolognesi et al. (2012) PLos ONE 7(10): e47534. doi:10.1371/journal.pone.0047534).

The overwhelming majority of sequences complementary to corn rootworm DNAs (such as the foregoing) are not lethal in species of corn rootworm when used as dsRNA or siRNA. For example, Baum et al. (2007, Nature Biotechnology 25:1322-1326), describe the effects of inhibiting several WCR gene targets by RNAi. These authors reported that the 8 of 26 target genes they tested were not able to provide experimentally significant coleopteran pest mortality at a very high iRNA (e.g., dsRNA) concentration of more than 520 ng/cm².

The authors of U.S. Pat. No. 7,612,194 and U.S. Patent Publication No. 2007/0050860 made the first report of in planta RNAi in corn plants targeting the western corn rootworm. Baum et al. (2007) Nat. Biotechnol. 25(11):1322-6. These authors describe a high-throughput in vivo dietary RNAi system to screen potential target genes for developing transgenic RNAi maize. Of an initial gene pool of 290 targets, only 14 exhibited larval control potential. One of the most effective double-stranded RNAs (dsRNA) targeted a gene encoding vacuolar ATPase subunit A (V-ATPase), resulting in a rapid suppression of corresponding endogenous mRNA and triggering a specific RNAi response with low concentrations of dsRNA. Thus, these authors documented for the first time the potential for in planta RNAi as a possible pest management tool, while simultaneously demonstrating that effective targets could not be accurately identified a priori, even from a relatively small set of candidate genes.

### SUMMARY OF THE DISCLOSURE

Disclosed herein are nucleic acid molecules (e.g., target genes, DNAs, dsRNAs, siRNAs, shRNAs, miRNAs, and hpRNAs), and methods of use thereof, for the control of coleopteran pests, including, for example, D. v. virgifera LeConte (western corn rootworm, “WCR”); D. barberi Smith and Lawrence (northern corn rootworm, “NCR”); D. u. howardi Barber (southern corn rootworm, “SCR”); D. v. zeae Krysan and Smith (Mexican corn rootworm, “MCR”); D. balteata LeConte; D. u. tenella; D. speciosa Germar; and D. u. undecimpunctata Mannerheim. In particular examples, exemplary nucleic acid molecules are disclosed that may be homologous to at least a portion of one or more native nucleic acid sequences in a coleopteran pest.

In these and further examples, the native nucleic acid sequence may be a target gene, the product of which may be, for example and without limitation: involved in a metabolic process; involved in a reproductive process; or involved in larval development. In some examples, post-translational inhibition of the expression of a target gene by a nucleic acid molecule comprising a sequence homologous thereto may be lethal in coleopteran pests, or result in reduced growth and/or reproduction. In specific examples, a gene consisting of chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94) may be selected as a target gene for post-transcriptional silencing. In particular examples, the target genes useful for post-transcriptional inhibition are the novel genes referred to herein as chitin synthase, outer membrane translocase, double parked, discs overgrown, ctf4, rpl9, serpin protease inhibitor I4, myosin 3 LC, megator, g-protein beta subunit, flap wing, female sterile 2 ketel, enhancer of polycomb, dead box 73D, cg7000, heat shock protein 70-331, heat shock protein 70-12300, rnr1, elav, pten, and cdc8. Isolated nucleic acid molecules comprising a nucleotide sequence of chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), and cdc8 (SEQ ID NO:94); the complement of chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), and cdc8 (SEQ ID NO:94); and fragments of any of the foregoing are therefore disclosed herein.

Also disclosed are nucleic acid molecules comprising a nucleotide sequence that encodes a polypeptide that is at least 85% identical to an amino acid sequence within a target gene product (for example, the product of a gene referred to as CHITIN SYNTHASE, OUTER MEMBRANE TRANSLOCASE, DOUBLE PARKED, DISCS OVERGROWN, CTF4, RPL9, SERPIN PROTEASE INHIBITOR I4, MYOSIN 3 LC, MEGATOR, G-PROTEIN BETA SUBUNIT, FLAP WING, FEMALE STERILE 2 KETEL, ENHANCER OF POLYCOMB, DEAD BOX 73D, CG7000, HEAT SHOCK PROTEIN 70-331, HEAT SHOCK PROTEIN 70-12300, RNR1, ELAV, PTEN, or CDC8). For example, a nucleic acid molecule may comprise a nucleotide sequence encoding a polypeptide that is at least 85% identical to an amino acid sequence of SEQ ID NO:2, (CHITIN SYNTHASE protein); SEQ ID NO:9 (OUTER MEMBRANE TRANSLOCASE protein); SEQ ID NO:14, (DOUBLE PARKED protein); SEQ ID NO:18, (DISCS OVERGROWN protein); SEQ ID NO:22, (CTF4 protein); SEQ ID NO:27, (RPL9 protein); SEQ ID NO:31, (SERPIN PROTEASE INHIBITOR I4 protein); SEQ ID NO:36, (MYOSIN 3 LC protein); SEQ ID NO:41, (MEGATOR protein); SEQ ID NO:46, (G-PROTEIN BETA SUBUNIT protein); SEQ ID NO:51, (FLAP WING protein); SEQ ID NO:55, (FEMALE STERILE 2 KETEL protein); SEQ ID NO:60, (ENHANCER OF POLYCOMB protein); SEQ ID NO:65, (DEAD BOX 73D protein); SEQ ID NO:69, (CG7000 protein); SEQ ID NO:73, (HEAT SHOCK PROTEIN 70-331 protein); SEQ ID NO:77, (HEAT SHOCK PROTEIN 70-12300 protein); SEQ ID NO:81, (RNR1 protein); SEQ ID NO:87, (ELAV protein); SEQ ID NO:91, (PTEN protein); or SEQ ID NO:95 (CDC8 protein). In particular examples, a nucleic acid molecule comprises a nucleotide sequence encoding a polypeptide that is at least 85% identical to an amino acid sequence within a product of CHITIN SYNTHASE, OUTER MEMBRANE TRANSLOCASE, DOUBLE PARKED, DISCS OVERGROWN, CTF4, RPL9, SERPIN PROTEASE INHIBITOR I4, MYOSIN 3 LC, MEGATOR, G-PROTEIN BETA SUBUNIT, FLAP WING, FEMALE STERILE 2 KETEL, ENHANCER OF POLYCOMB, DEAD BOX 73D, CG7000, HEAT SHOCK PROTEIN 70-331, HEAT SHOCK PROTEIN 70-12300, RNR1, ELAV, PTEN, or CDC8. Further disclosed are nucleic acid molecules comprising a nucleotide sequence that is the reverse complement of a nucleotide sequence that encodes a polypeptide at least 85% identical to an amino acid sequence within a target gene product.

Also disclosed are cDNA sequences that may be used for the production of iRNA (e.g., dsRNA, siRNA, miRNA, and hpRNA) molecules that are complementary to all or part of a coleopteran pest target gene, for example: chitin synthase, outer membrane translocase, double parked, discs overgrown, ctf4, rpl9, serpin protease inhibitor I4, myosin 3 LC, megator, g-protein beta subunit, flap wing, female sterile 2 ketel, enhancer of polycomb, dead box 73D, cg7000, heat shock protein 70-331, heat shock protein 70-12300, rnr1, elav, pten, and cdc8. In particular embodiments, dsRNAs, siRNAs, miRNAs, shRNAs, and/or hpRNAs may be produced in vitro or in vivo by a genetically-modified organism, such as a plant or bacterium. In particular examples, cDNA molecules are disclosed that may be used to produce iRNA molecules that are complementary to all or part of chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), and cdc8 (SEQ ID NO:94).

Further disclosed are means for inhibiting expression of an essential gene in a coleopteran pest, and means for providing coleopteran pest resistance to a plant. A means for inhibiting expression of an essential gene in a coleopteran pest is a single- or double-stranded RNA molecule comprising at least one of the genes/segments listed in Table 1, or the complement thereof.

TABLE 1
Coleopteran target sequences for RNAi
control of a coleopteran pest.

| SEQ ID NO: | Gene/Segment |
|---|---|
| 3 | chitin synthase Region 1 (Reg1) |
| 4 | chitin synthase Region 2 (Reg2) |
| 5 | chitin synthase Region 3 (Reg3) |
| 6 | chitin synthase Region 4 (Reg4) |
| 10 | outer membrane translocase Region 1 (Reg1) |
| 11 | outer membrane translocase Region 2 (Reg2) |
| 15 | double parked Region 1 (Reg1) |
| 19 | discs overgrown Region 1 (Reg1) |
| 23 | ctf4 Region 1 (Reg1) |
| 24 | ctf4 Variant 1 (Var1) |
| 28 | rpl9 Region 1 (Reg1) |
| 32 | serpin protease inhibitor I4 Region 1 (Reg1) |
| 33 | serpin protease inhibitor I4 Region 2 (Reg2) |
| 37 | myosin 3 LC Region 1 (Reg1) |
| 38 | myosin 3 LC Region 2 (Reg2) |
| 42 | megator Region 1 (Reg1) |
| 43 | megator Region 2 (Reg2) |
| 47 | g-protein beta subunit Region 1 (Reg1) |
| 48 | g-protein beta subunit Region 2 (Reg2) |
| 52 | flap wingRegion 1 (Reg1) |
| 56 | female sterile (2) ketel Region 1 (Reg1) |
| 57 | female sterile (2) ketel Region 2 (Reg2) |
| 61 | enhancer of polycomb Region 1 (Reg1) |
| 62 | enhancer of polycomb Region 2 (Reg2) |
| 66 | dead box 73D Region 1 (Reg1) |
| 70 | cg7000 Region 1 (Reg1) |
| 74 | heat shock protein 70-331 Region 1 (Reg1) |
| 78 | heat shock protein 70-12300 Region 1 (Reg1) |
| 82 | rnr1 Region 1 (Reg1) |
| 83 | rnr1 Region 2 (Reg2) |
| 84 | rnr1 Region 3 (Reg3) |
| 88 | elav Region 1 (Reg1) |
| 92 | pten Region 1 (Reg1) |
| 96 | cdc8 Region 1 (Reg1) |

Functional equivalents of means for inhibiting expression of an essential gene in a coleopteran pest include single- or double-stranded RNA molecules that are substantially homologous to all or part of a WCR gene selected from the list comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94. A means for providing coleopteran pest resistance to a plant is a DNA molecule comprising a nucleic acid sequence encoding a means for inhibiting expression of an essential gene in a coleopteran pest operably linked to a promoter, wherein the DNA molecule is capable of being integrated into the genome of a maize plant.

Disclosed are methods for controlling a population of a coleopteran pest, comprising providing to a coleopteran pest an iRNA (e.g., dsRNA, siRNA, miRNA, and hpRNA) molecule that functions upon being taken up by the coleopteran pest to inhibit a biological function within the coleopteran pest, wherein the iRNA molecule comprises all or part of a nucleotide sequence selected from the group consisting of: SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; the complement of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; a native coding sequence of a Diabrotica organism (e.g., WCR) comprising all or part of any of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; the complement of a native coding sequence of a Diabrotica organism comprising all or part of any of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; a native non-coding sequence of a Diabrotica organism that is transcribed into a native RNA molecule comprising all or part of any of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; and the complement of a native non-coding sequence of a Diabrotica organism that is transcribed into a native RNA molecule comprising all or part of any of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97.

In particular examples, methods are disclosed for controlling a population of a coleopteran pest, comprising providing to a coleopteran pest an iRNA (e.g., dsRNA, siRNA, miRNA, and hpRNA) molecule that functions upon being taken up by the coleopteran pest to inhibit a biological function within the coleopteran pest, wherein the iRNA molecule comprises a nucleotide sequence selected from the group consisting of: all or part of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; the complement of all or part of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; all or part of a native coding sequence of a Diabrotica organism (e.g., WCR) comprising SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; all or part of the complement of a native coding sequence of a Diabrotica organism comprising SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; all or part of a native non-coding sequence of a Diabrotica organism that is transcribed into a native RNA molecule comprising SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; and all or part of the complement of a native non-coding sequence of a Diabrotica organism that is transcribed into a native RNA molecule comprising SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97.

Also disclosed herein are methods wherein dsRNAs, siRNAs, miRNAs, shRNAs, and/or hpRNAs may be provided to a coleopteran pest in a diet-based assay, or in genetically-modified plant cells expressing the dsRNAs, siRNAs, miRNAs, shRNAs, and/or hpRNAs. In these and further examples, the dsRNAs, siRNAs, miRNAs, shRNAs, and/or hpRNAs may be ingested by coleopteran pest larvae. Ingestion of dsRNAs, siRNA, miRNAs, shRNAs, and/or hpRNAs of the invention may then result in RNAi in the larvae, which in turn may result in silencing of a gene essential for viability of the coleopteran pest and leading ultimately to larval mortality. Thus, methods are disclosed wherein nucleic acid molecules comprising exemplary nucleic acid sequence(s) useful for control of coleopteran pests are provided to a coleopteran pest. In particular examples, the coleopteran pest controlled by use of nucleic acid molecules of the invention may be WCR, NCR, SCR, MCR, D. balteata, D. u. tenella, D. speciosa, and/or D. u. undecimpunctata. The foregoing and other features will become more apparent from the following Detailed Description of several embodiments, which proceeds with reference to the accompanying FIGURE.

### BRIEF DESCRIPTION OF THE FIGURES

FIG. 1 and FIG. 2 present depictions of the strategies used to provide specific templates for dsRNA production.

### SEQUENCE LISTING

The nucleic acid sequences listed in the accompanying sequence listing are shown using standard letter abbreviations for nucleotide bases, as defined in 37 C.F.R. § 1.822. Only one strand of each nucleic acid sequence is shown, but the complementary strand and reverse complementary strand are understood as included by any reference to the displayed strand. In the accompanying sequence listing:

SEQ ID NO:1 shows a DNA sequence comprising chitin synthase.

SEQ ID NO:2 shows an amino acid sequence of a CHITIN SYNTHASE protein.

SEQ ID NO:3 shows a DNA sequence of chitin synthase Region 1 (Reg1) that was used for in vitro dsRNA synthesis (T7 promoter sequences at 5′ and 3′ ends not shown).

SEQ ID NO:4 shows a DNA sequence of chitin synthase Region 2 (Reg2) that was used for in vitro dsRNA synthesis (T7 promoter sequences at 5′ and 3′ ends not shown).

SEQ ID NO:5 shows a DNA sequence of chitin synthase Region 3 (Reg3) that was used for in vitro dsRNA synthesis (T7 promoter sequences at 5′ and 3′ ends not shown).

SEQ ID NO:6 shows a DNA sequence of chitin synthase Region 4 (Reg4) that was used for in vitro dsRNA synthesis (T7 promoter sequences at 5′ and 3′ ends not shown).

SEQ ID NO:7 presents a chitin synthase hairpin-RNA-forming sequence.

Upper case bases are chitin synthase sense strand, underlined lower case bases comprise an ST-LS1 intron, non-underlined lower case bases are chitin synthase antisense strand.

CTAAGTTGTGTGGCCTCTGGTAGAAAACAGGAAAATATTGTATATCATTC
CTATAGCTCTCAGTCTAATATCTGTTGGATGGTGGGAAAATTTTCTCTCG
GAGAAAACTCCAATTCCCTGGGTAAAAAAATTTGCAAAGGCAAAAAAACA
ATTTAAAACAGATGTTTATTTTACATATGCCTTAATCACCCCCATCAAAT
GTTTGATCTTCCTTTTATCTGCAGTAACAATTATATGGATAAGAGAAGGA
GATGTTGGCTTCTTGTTCGATAAATTTGGAGATGCATTTAGTAGTCACTC
GTTTAATGTTACACAGATCGAACCAGTGGTAGGAACATCCAACATTAATT
ACACgactagtaccggttgggaaaggtatgtttctgcttctacctttgat
atatatataataattatcactaattagtagtaatatagtatttcaagtat
tatttcaaaataaaagaatgtagtatatagctattgatactgtagatata
agtgtgtatatataatttataactatctaatatatgaccaaaacatggtg
atgtgcaggagatccgcggttagtgtaattaatgaggatgacctaccact
ggttcgatctgtgtaacattaaacgagtgactactaaatgcatctccaaa
tttatcgaacaagaagccaacatctccactcttatccatataattgttac
tgcagataaaaggaagatcaaacatttgatgggggtgattaaggcatatg
taaaataaacatctgattaaattgattatgcattgcaaattatttaccca
gggaattggagattctccgagagaaaattacccaccatccaacagatatt
agactgagagctataggaatgatatacaatattacctgattctaccagag
gccacacaacagagct

SEQ ID NO:8 shows a DNA sequence comprising outer membrane translocase (omt).

SEQ ID NO:9 shows an amino acid sequence of a OUTER MEMBRANE TRANSLOCASE (OMT) protein.

SEQ ID NO:10 shows a DNA sequence of outer membrane translocase (omt) Region 1 (Reg1) that was used for in vitro dsRNA synthesis (T7 promoter sequences at 5′ and 3′ ends not shown).

SEQ ID NO:11 shows a DNA sequence of outer membrane translocase (omt) Region 2 (Reg2) that was used for in vitro dsRNA synthesis (T7 promoter sequences at 5′ and 3′ ends not shown).

SEQ ID NO:12 presents a outer membrane translocase (omt) hairpin-RNA-forming sequence. Upper case bases are outer membrane translocase sense strand, underlined lower case bases comprise an ST-LS1 intron, non-underlined lower case bases are outer membrane translocase antisense strand.

CTAAGGAGTTCCTTGGTTTCTTGAAATACTTGATTCTGAAGATGTAAAAC
AAGTGTCTGCCGCAGAGCACTGTATGCAATCTATTATAAACGCATTCTCT
GGCATGGAAAATCGACCAGACACTAAACCTAAACAGGAATTGATAGAAAG
TAACAAGAAACAAATAGATACATTATTGACTTGCTTAGTATATTCTATTA
ATAACAGAGTGATTTCAGGACTAGCTAGGGATGCATTGATAGAACTCATT
ATGAGGAATATTCACTATGATCAGCTCTGTTGGGCCAAAGAGCTAGTAGA
TATAGGCGGTGTAGAAAGATTAATGGAATGTGCTAGTGAATTGGAGGAGT
ACAAtagttagttgagactagtaccggttgggaaaggtatgtttctgctt
ctaccatgatatatatataataattatcactaattagtagtaatatagta
tttcaagtattatttcaaaataaaagaatgtagtatatagctattgcttt
tctgtagatataagtgtgtatatataatttataactatctaatatatgac
caaaacatggtgatgtgcaggagatccgcggttatcaactaactattgta
ctcctccaattcactagcacattccattaatctactacaccgcctatatc
tactagctctaggcccaacagagctgatcatagtgaatattcctcataat
gagactatcaatgcatccctagctagtcctgaaatcactctgttattaat
agaatatactaagcaagtcaataatgtatctatttgatcagttactacta
tcaattcctgataggatagtgtctggtcgattaccatgccagagaatgcg
atataatagattgcatacagtgctctgcggcagacacttgattacatcac
agaatcaagtatttcaagaaaccaaggaactccagagct

SEQ ID NO:13 shows a DNA sequence comprising double parked.

SEQ ID NO:14 shows an amino acid sequence of a DOUBLE PARKED protein.

SEQ ID NO:15 shows a DNA sequence of double parked Region 1 (Reg1) that was used for in vitro dsRNA synthesis (T7 promoter sequences at 5′ and 3′ ends not shown).

SEQ ID NO:16 presents a double parked hairpin-RNA-forming sequence. Upper case bases are double parked sense strand, underlined lower case bases comprise an ST-LS1 intron, non-underlined lower case bases are double parked antisense strand.

CTAATAGTTAGTTGAGACGCGATCAGAAGATAAAGAAAAAGAACTTAAGG
TTTACTCGAGACTCCCCGAACTGGCACGTTTAACAAGAAATGTATTTGTA
TCCGAAAAGAAGAACGTACTACAACTAGACATCCTCGTCGATAAGTTAGG
AAACTGTTATAGATCACATTTAACCAAACTAGAAATGGAAGAACACTTAA
GGATTCTTTCAAAAGAATTTCCCACGTGGTTGGTGTTCCATGTTGTAAGG
AATTGCGTGTATGTGAAGATAGCCAAAAGTGATGATTTAAGTTTGATAAT
TAATAAGTTGGAGAGTATAGTTAAACAAAAAAGTAGAACTTTAACATTTT
ATATTACAATATTTCTTTGCAGTTTTGACTGAgactagtaccggagggaa
aggtatgatctgcactaccatgatatatatataataattatcactaatta
gtagtaatatagtatttcaagtattatttcaaaataaaagaatgtagtat
atagctattgcttactgtagatataagtgtgtatatataatttataacta
tctaatatatgaccaaaacatggtgatgtgcaggagatccgcggttatca
gtcaaaactgcaaagaaatattgtaatataaaatgttaaagactacttat
tgataactatactctccaacttattaattatcaaacttaaatcatcactt
aggctatatcacatacacgcaattccttacaacatggaacaccaaccacg
tgggaaattcttttgaaagaatccttaagtgttcttccatttctagtttg
gttaaatgtgatctataacagtttcctaacttatcgacgaggatgtctag
ttgtagtacgttcttcttttcggatacaaatacatttcttgttaaacgtg
ccagttcggggagtctcgagtaaaccttaagttctttttctttatcttct
gatcgcgtcattgtagttagttgaagagct

SEQ ID NO:17 shows a DNA sequence comprising discs overgrown.

SEQ ID NO:18 shows an amino acid sequence of a DISCS OVERGROWN protein.

SEQ ID NO:19 shows a DNA sequence of discs overgrown Region 1 (Reg1) that was used for in vitro dsRNA synthesis (T7 promoter sequences at 5′ and 3′ ends not shown).

SEQ ID NO:20 presents a discs overgrown hairpin-RNA-forming sequence.

Upper case bases are discs overgrown sense strand, underlined lower case bases comprise an ST-LS1 intron, non-underlined lower case bases are discs overgrown antisense strand.

CTAAACAAGTAGTATCATCGGTATCGGAAAGCCTGATTATGTGGTCGGAG
GAAAATATCGTCTACTTCGTAAAATCGGTAGTGGATCTTTTGGTGACATA
TACCTTGGAATAAATATTACGAATGGAGAGGAAGTAGCAGTAAAACTTGA
ACCAATTAGAGCAAGGCATCCACAGCTTTTATATGAAAGTAAACTATATA
AAATTCTTCATGGAGGCATAGGAATACCACATATCAGATATTATGGACAA
GAAAAAGAATATAATGTCCTTGTCATGGACCTGCTCGGTCCGTCGTTGGA
AGACCTCTTCAATTTTTGTACCCGCAGATTCACCATTAAAACCGTACTCA
TGTTGGCgactagtaccggttgggaaaggtatgtttctgcttctaccttt
gatatatatataataattatcactaattagtagtaatatagtatttcaag
tatttttttcaaaataaaagaatgtagtatatagctattgcttttctgta
gatataagtgtgtatatataatttataactatctaatatatgaccaaaac
atggtgatgtgcaggagatccgcggttagccaacatgagtacggattaat
ggtgaatctgcgggtacaaaaattgaagaggtatccaacgacggaccgag
caggtccatgacaaggacattatattcttatcagtccataatatctgata
tgtggtattcctatgcctccatgaagaatatatatagatactacatataa
aagctgtggatgccagctctaattggacaagattactgctacttcctctc
cattcgtaatatttattccaaggtatatgtcaccaaaagatccactaccg
atatacgaagtagacgatattacctccgaccacataatcaggctaccgat
accgatgatactacttgacagtagagct

SEQ ID NO:21 shows a DNA sequence comprising ctf4.

SEQ ID NO:22 shows an amino acid sequence of a CTF4 protein.

SEQ ID NO:23 shows a DNA sequence of ctf4 Region 1 (Reg1) that was used for in vitro dsRNA synthesis (T7 promoter sequences at 5′ and 3′ ends not shown).

SEQ ID NO:24 shows a DNA sequence of ctf4 Variant 1 (Var1) that was used for in vitro dsRNA synthesis (T7 promoter sequences at 5′ and 3′ ends not shown).

SEQ ID NO:25 presents a ctf4 hairpin-RNA-forming sequence. Upper case bases are ctf4 sense strand, underlined lower case bases comprise an ST-LS1 intron, non-underlined lower case bases are ctf4 antisense strand.

GTTGAAACAGCCAAAGCTGAACAAACTTTTGAAACTCGAGGAAAATTTAC
TTTAAGCATAGCTTATAGCCCAGATGGAAAATACATAGCAAGTGGAGCTA
TTGAAGGCATTATTAATATATTTGATGTAGCAGCCAACAAACTGTTGCAC
ACATTAGAAGGTCACGCTATGCCCATCCGTTCTCTTTGCTTTTCTCCTGA
CTCCCAACTACTGCTAACAGGATCTGATGATGGGTATATGAAACTTTATG
ATGTGCGAGATCAAAAAACCCGTCTGATAGGAACATTATCAGGCCATGCT
TCTTGGGTTCTTAGTGTAGCATTTTCTCCTGATGGCAAGAACTTTGTTTC
TGGCAGTTCCGATAAAACGGTAAAAGTTTGGGATGTCGCTACCAGACAGT
GTCTTCATACATTTAATGAACATACAGATCAGGTATGGGGCGTGACATAT
AACCCAGATAGTAATAAAATTCTTTCAGTATCTGAAGATAAAAGTATCAA
TGTATATAGTTGTCCTGagagggatccaggcctaggtatgatctgcacta
ccatgatatatatataataattatcactaattagtagtaatatagtattt
caagtatattacaaaataaaagaatgtagtatatagctattgcattctgt
agatataagtgtgtataattaatttataactatctaatatatgaccaaaa
catggtgatgtgcaggtatttaaataccggtccatggagagcaggacaac
tatatacattgatactatatcacagatactgaaagaatatattactatct
gggttatatgtcacgccccatacctgatctgtatgacattaaatgtatga
agacactgtctggtagcgacatcccaaactataccgattatcggaactgc
cagaaacaaagacttgccatcaggagaaaatgctacactaagaacccaag
aagcatggcctgataatgacctatcagacgggattagatctcgcacatca
taaagatcatatacccatcatcagatcctgttagcagtagagggagtcag
gagaaaagcaaagagaacggatgggcatagcgtgaccactaatgtgtgca
acagatgaggctgctacatcaaatatattaataatgccacaatagctcca
cttgctatgtattaccatctgggctataagctatgcttaaagtaaattac
ctcgagtacaaaagatgacagctaggctgatcaac

SEQ ID NO:26 shows a DNA sequence comprising rpl9.

SEQ ID NO:27 shows an amino acid sequence of a RPL9 protein.

SEQ ID NO:28 shows a DNA sequence of rpl9 Region 1 (Reg1) that was used for in vitro dsRNA synthesis (T7 promoter sequences at 5′ and 3′ ends not shown).

SEQ ID NO:29 presents a rpl9 hairpin-RNA-forming sequence. Upper case bases are rpl9 sense strand, underlined lower case bases comprise an ST-LS1 intron, non-underlined lower case bases are rpl9 antisense strand.

GAAGCAAATTGTAACAAACCAAACGGTAAAAATTCCTGAGGGAATTACAG
TAACCACTAAGTCAAGGGTAGTAACTGTAAAAGGACCCCGAGGTAATCTG
AAGAGATCCTTCAAACATCTTACCCTGGACATTCGAATGATCAACCCCAG
GTTACTGAAAGTAGAGAAATGGTTCGGTACCAAGAAGGAATTAGCTGCTG
TCAGAACAGTATGCTCTCATGTTGAAAACATGTTAAAGGGTGTTACAAAA
GGATACCAATACAAGATGAGAGCAGCATATGCTCACTTTCCCATTAACTG
TGTCACCACTGAAGGAAACACAGTTATCGAAATTAGAAATTTCTTGGGAG
AAAAATACATCAGAAGAGTAAAGATGGCCCCAGGTGTTACAGTAGTAAAC
TCCACCAAACAAAAGGATGAACTTATCATTGAAGGAAACTCATTGGAGGA
TGTTTCAAAGTCAGCTGCCCTTATTCAACAATCTACAACAGTTAAAAATA
AGGATATCCGTAAATTCTTAGATGGGCTTTATGTATCTGAAAAAACAACT
GTTGTACAAGAAGagagggatccaggcctaggtatgatctgatctaccat
gatatatatataataattatcactaattagtagtaatatagtatttcaag
tattatttcaaaataaaagaatgtagtatatagctattgatactgtagat
ataagtgtgtatatataatttataacttactaatatatgaccaaaacatg
gtgatgtgcaggtatttaaataccggtccatggagagatcagtacaacag
ttgattacagatacataaagcccatctaagaatttacggatatccttatt
ataactgagtagattgagaataagggcagctgactagaaacatcctccaa
tgagatcatcaatgataagttcatcatttgatggtggagatactactgta
acacctggggccatattactatctgatgtattatctcccaagaaatacta
atttcgataactgtgatcatcagtggtgacacagttaatgggaaagtgag
catatgctgctctcatcagtattggtatccttagtaacaccctttaacat
gattcaacatgagagcatactgttctgacagcagctaattcatcaggtac
cgaaccatactctactacagtaacctggggagatcattcgaatgtccagg
gtaagatgatgaaggatctatcagattacctcggggtccattacagttac
tacccttgacttagtggttactgtaattccctcaggaatttttaccgttt
ggtttgttacaatttgcttc

SEQ ID NO:30 shows a DNA sequence comprising serpin protease inhibitor I4.

SEQ ID NO:31 shows an amino acid sequence of a SERPIN PROTEASE INHIBITOR I4 protein.

SEQ ID NO:32 shows a DNA sequence of serpin protease inhibitor 4 Region 1 (Reg1) that was used for in vitro dsRNA synthesis (T7 promoter sequences at 5′ and 3′ ends not shown).

SEQ ID NO:33 shows a DNA sequence of serpin protease inhibitor 4 Region 2 (Reg2) that was used for in vitro dsRNA synthesis (T7 promoter sequences at 5′ and 3′ ends not shown).

SEQ ID NO:34 presents a serpin protease inhibitor I4 hairpin-RNA-forming sequence. Upper case bases are serpin protease inhibitor I4 sense strand, underlined lower case bases comprise an ST-LS1 intron, non-underlined lower case bases are serpin protease inhibitor I4 antisense strand.

CCTACTGAAGCTGTGCAATTCCAAAATACTCGCCCAAGTAGACCAAATAA
TGAAATTGATCAATCCAATAATGTACAGTTCCAACAGCCTCGTCCAAATA
GACCAAGTGTTGAAAGTCAACGACCCGACAATGTACAATTCCAACAGAAT
CGCCCAAATAGACCAAATACTGAAAATGAACAGCCCAGCAATGTACAAGG
TCTTAATTTGAATCCAAATCTGGACCCCGACTACTATCAACCTCCAGATG
TAGAAATGGATGAGATTGCATATAGATTCAATCTGTTTGATATTGACTTA
TTATATTCCTTTTCTGATTTATCCACGAACGTATTAATATCTCCCGCTAG
CATCAAAACGACATTAGCAATGATTTTAGAAGGAGCAGAAGGAACTTGTG
CAGAAGAAATAAGTGAAGCACTGAGGATACCTGATATTAATCAAAAAGGT
GTCAGAAGTGTTCTTGTAGAACTTCTAAATAATCTTAATGAAAGATCTAC
GCCAAATAGTGTCCTAGAAAGCCATAATGCCATATTTGTCTCTGAAAAAC
ATCGACTGGTTGATAATTATAAGAATGTCGTTATAAAGTATTACAACGCT
agagggatccaggcctaggtatgtttctgcttctacctttgatatatata
taataattatcactaattagtagtaatatagtatttcaagtatttttttc
aaaataaaagaatgtagtatatagctattgcttactgtagatataagtgt
gtatatataatttataactatctaatatatgaccaaaacatggtgatgtg
caggtatttaaataccggtccatggagagagcgagtaatactttataacg
acattcttataattatcaaccagtcgatgatttcagagacaaatatggca
ttatggctactaggacactataggcgtagatattcattaagattatttag
aagactacaagaacacactgacaccatagattaatatcaggtatcctcag
tgcttcacttatttcactgcacaagaccactgctccactaaaatcattgc
taatgtcgattgatgctagcgggagatattaatacgttcgtggataaatc
agaaaaggaatataataagtcaatatcaaacagattgaatctatatgcaa
tctcatccatactacatctggaggagatagtagtcggggtccagatagga
ttcaaattaagaccagtacattgctgggctgacattacagtatttggtct
atagggcgattctgaggaattgtacattgtcgggtcgttgactacaacac
ttggtctatttggacgaggctgaggaactgtacattattggattgatcaa
tttcattatttggtctacttgggcgagtattttggaattgcacagcttca
gtagg

SEQ ID NO:35 shows a DNA sequence comprising myosin 3LC.

SEQ ID NO:36 shows an amino acid sequence of a Myosin 3LC protein.

SEQ ID NO:37 shows a DNA sequence of myosin 3LC Region 1 (Reg1) that was used for in vitro dsRNA synthesis (T7 promoter sequences at 5′ and 3′ ends not shown).

SEQ ID NO:38 shows a DNA sequence of myosin 3LC Region 2 (Reg2) that was used for in vitro dsRNA synthesis (T7 promoter sequences at 5′ and 3′ ends not shown).

SEQ ID NO:39 presents a myosin 3LC hairpin-RNA-forming sequence. Upper case bases are myosin 3LC sense strand, underlined lower case bases comprise an ST-LS1 intron, non-underlined lower case bases are myosin 3LC antisense strand.

CCATGTTTTCGCAATCTCAAGTAGCTGAATTCAAAGAAGCTTTCCAACTT
ATGGACCACGACAAAGATGGCATCATCTCTAAGAGCGATTTGAGGGCCAC
CTTCGATGCTGTTGGTAAACTCGCCAGTGAGAAAGAGCTCGACGAGATGA
TCAACGAAGCACCCGGACCAATCAACTTCACTCAATTGTTGGGATTGTTC
GGTACCCGTATGGCTGATTCCGGCGGTACTGACGATGATGAAGTTGTCGT
CAAGGCTTTCAGATCCTTTGACGAAAACGGCACCATTGACGGAGACAGAT
TCCGCCATGCCCTCATGACCTGGGGAGAAAAATTCACCGGCAAAGAATGC
GACagagggatccaggcctaggtatgtttctgcttctacctttgatatat
atataataattatcactaattagtagtaatatagtatttcaagtattatt
tcaaaataaaagaatgtagtatatagctattgatactgtagatataagtg
tgtatattttaatttataacttttctaatatatgaccaaaacatggtgat
gtgcaggtatttaaataccggtccatggagaggtcgcattctttgccggt
gaatttactccccaggtcatgagggcatggcggaatctgtctccgtcaat
ggtgccgattcgtcaaaggatctgaaagccagacgacaacttcatcatcg
tcagtaccgccggaatcagccatacgggtaccgaacaatcccaacaattg
agtgaagttgattggtccgggtgcttcgttgatcatctcgtcgagctatt
ctcactggcgagataccaacagcatcgaaggtggccctcaaatcgctctt
agagatgatgccatctagtcgtggtccataagaggaaagatattgaattc
agctacttgagattgcgaaaacatgg

SEQ ID NO:40 shows a DNA sequence comprising megator.

SEQ ID NO:41 shows an amino acid sequence of a MEGATOR protein.

SEQ ID NO:42 shows a DNA sequence of megator Region 1 (Reg1) that was used for in vitro dsRNA synthesis (T7 promoter sequences at 5′ and 3′ ends not shown).

SEQ ID NO:43 shows a DNA sequence of megator Region 2 (Reg2) that was used for in vitro dsRNA synthesis (T7 promoter sequences at 5′ and 3′ ends not shown).

SEQ ID NO:44 presents a megator hairpin-RNA-forming sequence. Upper case bases are megator sense strand, underlined lower case bases comprise an ST-LS1 intron, non-underlined lower case bases are megator antisense strand.

TGATTAAAAAAGCAACTTGATGAGGCTAAAGAGTCTGTTGAAACGGAGAG
GCAGAAAACGGAGATCTCTGTTGTATCCKCTGCTAAACATGCAGAAGTAC
TTCGTAAATTAGAAACTCTTAATGCCATCACCGACAGCAATAGAGCTYTA
AGACAGGAACGAGATACTCTAGCTGCCCAATTAGCAGACCTCCAAAGCAA
AGCAGAAACATTCGAAACTGAAGTCGAACCTTTGCAGGAGAAGAACAGAG
AGCTCACAACAAAGGCTGACCAATTACAAAGTGAAAATATATCATTACGT
GCCGAATGTACCAGATGGAGACAGAGGGCTAATATGCTTATTGAAAAGAC
GAACARGAACAAGTACCCGAAGATTGGAAGAAGTTGCAGACAGAACGGGA
GACCTTATCCAAACAGTTGAagagggatccaggcctaggtatgatctgat
ctaccatgatatatatataataattatcactaattagtagtaatatagta
tttcaagtattatttcaaaataaaagaatgtagtatatagctattgctta
ctgtagatataagtgtgtatatataatttataactatctaatatatgacc
aaaacatggtgatgtgcaggtatttaaataccggtccatggagagtcaac
tgtaggataaggtctcccgactgtctgcaacttcaccaatcacgggtact
tgacytgacgtatttcaataagcatattagccctctgtctccatctggta
cattcggcacgtaatgatatattacactagtaattggtcagcattgagtg
agctctctgacttctcctgcaaaggttcgacttcagtttcgaatgtttct
gctttgctttggaggtctgctaattgggcagctagagtatctcgttcctg
tcttaragctctattgctgtcggtgatggcattaagagtttctaatttac
gaagtacttctgcatgtttagcagmggatacaacagagatctccgttttc
tgcctctccgatcaacagactattagcctcatcaagagcttattaatca

SEQ ID NO:45 shows a DNA sequence comprising g-protein beta subunit.

SEQ ID NO:46 shows an amino acid sequence of a G-PROTEIN BETA SUBUNIT protein.

SEQ ID NO:47 shows a DNA sequence of g-protein beta subunit Region 1 (Reg1) that was used for in vitro dsRNA synthesis (T7 promoter sequences at 5′ and 3′ ends not shown).

SEQ ID NO:48 shows a DNA sequence of g-protein beta subunit Region 2 (Reg2) that was used for in vitro dsRNA synthesis (T7 promoter sequences at 5′ and 3′ ends not shown).

SEQ ID NO:49 presents a g-protein beta subunit hairpin-RNA-forming sequence. Upper case bases are G-Protein Beta subunit sense strand, underlined lower case bases comprise an ST-LS1 intron, non-underlined lower case bases are g-protein beta subunit antisense strand.

GTATGAACGAACTGGATTCTCTTAGGCAAGAAGCTGAAACCCTCAAAAAT
GCTATTAGAGATGCTCGCAAAGCGGCTTGTGACACATCTTTGGTACAGGC
TACCTCCAGCCTGGAGCCCATTGGTCGAGTGCAGATGCGGACTAGACGTA
CTCTCAGAGGCCATTTGGCCAAGATCTACGCTATGCACTGGGGCTCAGAC
TCAAGGAATCTTGTCTCAGCATCACAAGATGGAAAACTTATCGTATGGGA
CAGTCACACCACAAATAAGGTACATGCAATCCCCCTCAGATCATCATGGG
TGATGACATGTGCTTATGCACCATCTGGAAATTTTGTCGCCTGTGGAGGT
TTAGATAATATATGCTCTATCTATAGCCTAAAGACTAGAGAAGGCAATGT
TCGGGTCAGCCGAGAATTGCCCGGTCATACTGGTTATCTATCTTGCTGTC
GTTTCCTCGATGACAACCAagagggatccaggcctaggtatgtttctgct
tctacctttgatatatatataataattatcactaattagtagtaatatag
tatttcaagtattatttcaaaataaaagaatgtagtatatagctattgca
ttctgtagtttataagtgtgtatattttaatttataacttttctaatata
tgaccaaaacatggtgatgtgcaggtatttaaataccggtccatggagag
tggttgtcatcgaggaaacgacagcaagatagataaccagtatgaccggg
caattctcggctgacccgaacattgccttctctagtctttaggctataga
tagagcatatattatctaaacctccacaggcgacaaaataccagatggtg
cataagcacatgtcatcacccatgatgatctgagggggattgcatgtacc
ttatagtggtgtgactgtcccatacgataagattccatcagtgatgctga
gacaagattccttgagtctgagccccagtgcatagcgtagatcttggcca
aatggcctctgagagtacgtctagtccgcatctgcactcgaccaatgggc
tccaggctggaggtagcctgtaccaaagatgtgtcacaagccgctagcga
gcatctctaatagcatattgagggatcagcacttgcctaagagaatccag
ttcgttcatac

SEQ ID NO:50 shows a DNA sequence comprising flap wing.

SEQ ID NO:51 shows an amino acid sequence of a FLAP WING protein.

SEQ ID NO:52 shows a DNA sequence of flap wing Region 1 (Reg1) that was used for in vitro dsRNA synthesis (T7 promoter sequences at 5′ and 3′ ends not shown).

SEQ ID NO:53 presents a flap wing hairpin-RNA-forming sequence. Upper case bases are flap wing sense strand, underlined lower case bases comprise an ST-LS1 intron, non-underlined lower case bases are flap wing antisense strand.

GGTGGACTGAGTCCAGATTTGCAAGGAATGGAACAAATCAGGAGAATAAT
GAGGCCGACAGATGTACCAGACACCGGTCTTCTTTGTGACCTTCTTTGGT
CCGATCCAGACAAAGACGTCCAAGGGTGGGGCGAGAACGATCGAGGTGTT
TCCTTCACCTTCGGAGCAGACGTCGTAAGCAAATTCCTGAACCGACACGA
TCTCGATTTGATCTGCCGCGCCCACCAAGTTGTCGAAGATGGCTACGAGT
TCTTCGCCAAGCGACAGCTCGTCACTTTGTTCTCCGCTCCCAACTATTGC
GGCGAGTTCGACAATGCTGGAGGGATGATGAGCGTGGACGAGACGTTGAT
GTGTTCGTTTCAGATTCTCAAACCGTCAGAAAAGAAGGCAAAGTACCAAT
ACCAGGGGATGAATTCAGGCCGCCCATCCACACCGCAACGAAATCCCCCT
AACAAAAATAAGAAGTAGATCACagagggatccaggcctaggtatgtttc
tgcttctaccatgatatatatataataattatcactaattagtagtaata
tagtatttcaagtattatttcaaaataaaagaatgtagtatatagctatt
gcttttctgtagtttataagtgtgtatattttaatttataacttttctaa
tatatgaccaaaacatggtgatgtgcaggtatttaaataccggtccatgg
agaggtgatctacttcttatttttgttagggggatttcgttgcggtgtgg
atgggcggcctgaattcatcccctggtattggtactttgccttatactga
cggatgagaatctgaaacgaacacatcaacgtctcgtccacgctcatcat
ccctccagcattgtcgaactcgccgcaatagagggagcggagaacaaagt
gacgagctgtcgcaggcgaagaactcgtagccatatcgacaacttggtgg
gcgcggcagatcaaatcgagatcgtgtcggacaggaatttgcttacgacg
tctgctccgaaggtgaaggaaacacctcgatcgactcgccccacccagga
cgtctagtctggatcggaccaaagaaggtcacaaagaagaccggtgtctg
gtacatctgtcggcctcattattctcctgatttgaccattccttgcaaat
ctggactcagtccacc

SEQ ID NO:54 shows a DNA sequence comprising female sterile 2 ketel.

SEQ ID NO:55 shows an amino acid sequence of a FEMALE STERILE 2 KETEL protein.

SEQ ID NO:56 shows a DNA sequence of female sterile 2 ketel Region 1 (Reg1) that was used for in vitro dsRNA synthesis (T7 promoter sequences at 5′ and 3′ ends not shown).

SEQ ID NO:57 shows a DNA sequence of female sterile 2 ketel Region 2 (Reg2) that was used for in vitro dsRNA synthesis (T7 promoter sequences at 5′ and 3′ ends not shown).

SEQ ID NO:58 presents a female sterile 2 ketel hairpin-RNA-forming sequence. Upper case bases are female sterile 2 ketel sense strand, underlined lower case bases comprise an ST-LS1 intron, non-underlined lower case bases are female sterile 2 ketel antisense strand.

CGATTCTGCTGCAAAAATTAACGAAACAGGAAGAACTGGACGACGAAGAC
GATTGGAATCCTTGCAAAGCAGCCGGCGTTTGCTTAATGCTTCTCGCCAC
GTGTTGCGAAGACGAAATCGTTCCACACGTTCTGCCCTTCATCAAAGAAA
ATATTAAGTCTGATAATTGGCGATTCCGAGATGCTTCTTTGATGGCTTTC
GGATCTATTTTAGGTGGTTTGGAGAGTACTACTTTGAGACCGCTTGTTGA
ACAGGCGATGCCGACCTTGATAGATCTGATGTACGACAATAGCGTTATCG
TAAGAGACACAGCCGCCTGGAACTTCGGCAGAATTTGCGAAATCATTCCG
GAGGCGGCCATCAACGAAACCTTCCTCAAACCGCTATTAGAAAGCTTGAT
CACAGGCCTCAAGGCCGAACCTCGTGTTGCCGCCAATGTCTGTTGGGCGT
TCTCTGGATTGGTGGAGGCAGCTTACGAAAATGCCGACGTTagagggatc
caggcctaggtatgtttctgcttctacctttgatatatatataataatta
tcactaattagtagtaatatagtatttcaagtatttttttcaaaataaaa
gaatgtagtatatagctattgcttactgtagatataagtgtgtatatata
atttataactatctaatatatgaccaaaacatggtgatgtgcaggtattt
aaataccggtccatggagagaacgtcggcattacgtaagctgcctccacc
aatccagagaacgcccaacagacattggcggcaacacgaggacggccttg
aggcctgtgatcaagctactaatagcggatgaggaaggatcgttgatggc
cgcctccggaatgatttcgcaaattctgccgaagttccaggcggctgtgt
ctcttacgataacgctattgtcgtacatcagatctatcaaggtcggcatc
gcctgacaacaagcggtctcaaagtagtactctccaaaccacctaaaata
gatccgaaagccatcaaagaagcatctcggaatcgccaattatcagactt
aatattactagatgaagggcagaacgtgtggaacgatttcgtatcgcaac
acgtggcgagaagcattaagcaaacgccggctgctagcaaggattccaat
cgtatcgtcgtccagacttcctgatcgttaatattgcagcagaatcg

SEQ ID NO:59 shows a DNA sequence comprising enhancer of polycomb.

SEQ ID NO:60 shows an amino acid sequence of an ENHANCER OF POLYCOMB protein.

SEQ ID NO:61 shows a DNA sequence of enhancer of polycomb Region 1 (Reg1) that was used for in vitro dsRNA synthesis (T7 promoter sequences at 5′ and 3′ ends not shown).

SEQ ID NO:62 shows a DNA sequence of enhancer of polycomb Region 2 (Reg2) that was used for in vitro dsRNA synthesis (T7 promoter sequences at 5′ and 3′ ends not shown).

SEQ ID NO:63 presents a enhancer of polycomb hairpin-RNA-forming sequence. Upper case bases are enhancer of polycomb sense strand, underlined lower case bases comprise an ST-LS1 intron, non-underlined lower case bases are enhancer of polycomb antisense strand.

ATGTCGAAGCTTTCATTTAGGGCGAGGGCCCTGGATGCTAGCAAACCCAT
GCCTATATACATGGCTGAGGAACTCCCGGATCTTCCTGATTATTCAGCGA
TCAATCGGGCAGTTCCTCAAATGCCATCAGGAATGCAAAAGGAGGAGGAA
TGTGAACACCATCTTCAACGTGCAATTGTTGCTGGACTCATCATTCCTAC
TCCCGAAGTTTCCGAATTGCCAGGTagagggatccaggcctaggtatgat
ctgatctaccatgatatatatataataattatcactaattagtagtaata
tagtatttcaagtattatttcaaaataaaagaatgtagtatatagctatt
gatactgtagatataagtgtgtatatataatttataactatctaatatat
gaccaaaacatggtgatgtgcaggtatttaaataccggtccatggagaga
cctggcaattcggaaacttcgggagtaggaatgatgagtccagcaacaat
tgcacgttgaagatggtgttcacattcctcctccttttgcattcctgatg
gcatttgaggaactgcccgattgatcgctgaataatcaggaagatccggg
agacctcagccatgtatataggcatgggatgctagcatccagggccctcg
ccctaaatgaaagcttcgacat

SEQ ID NO:64 shows a DNA sequence comprising dead box 73D.

SEQ ID NO:65 shows an amino acid sequence of a DEAD BOX 73D peptide.

SEQ ID NO:66 shows a DNA sequence of dead box 73D Region 1 (Reg1) that was used for in vitro dsRNA synthesis (T7 promoter sequences at 5′ and 3′ ends not shown).

SEQ ID NO:67 presents a dead box 73D hairpin-RNA-forming sequence. Upper case bases are dead box 73D sense strand, underlined lower case bases comprise an ST-LS1 intron, non-underlined lower case bases are dead box 73D antisense strand.

AGATGTGTGTTTAATAAGTGGAACTAACTCTTTTGCTGTAGAACAATCAC
AGTTGATCATTGAGAATAAAGCTTTCGGAGCAGTCAGTAAAATTGATATC
TTGGTATGTACAGCTGGAAGACTTGTAGATCACTTAAAAGTAACTAAAGG
ATTTAGTTTACGAAATTTGGAGTTTTTAGTTATCGATGAGGCAGATAGAG
TGCTTGAAAATGTTCAGAACGATTGGCTGTATCACTTGGAAAAACATATT
TATCAAGAGGCAACTAATGGACAAACGAGAAAAGTTCTCAACCTCTGTAC
TTTAGAACAAGCACGGCCTCCACAGAAACTCCTATTCTCAGCCAagaggg
atccaggcctaggtatgtttctgcttctacctttgatatatatataataa
ttatcactaattagtagtaatatagtatttcaagtatttttttcaaaata
aaagaatgtagtatatagctattgcttttctgtagtttataagtgtgtat
attttaatttataacttttctaatatatgaccaaaacatggtgatgtgca
ggtatttaaataccggtccatggagagtggctgagaataggagtttctgt
ggaggccgtgcttgttctaaagtacagaggttgagaacttttctcgtttg
tccattagttgcctcttgataaatatgtttttccaagtgatacagccaat
cgttctgaacattttcaagcactctatctgcctcatcgataactaaaaac
tccaaatttcgtaaactaaatcctttagttacttttaagtgatctacaag
tcttccagctgtacataccaagatatcaattttactgactgctccgaaag
ctttattctcaatgatcaactgtgattgttctacagcaaaagagttagtt
ccacttattaaacacacatct

SEQ ID NO:68 shows a DNA sequence comprising cg7000.

SEQ ID NO:69 shows an amino acid sequence of a CG7000 protein.

SEQ ID NO:70 shows a DNA sequence of cg7000 Region 1 (Reg1) that was used for in vitro dsRNA synthesis (T7 promoter sequences at 5′ and 3′ ends not shown).

SEQ ID NO:71 presents a cg7000 hairpin-RNA-forming sequence. Upper case bases are cg7000 sense strand, underlined lower case bases comprise an ST-LS1 intron, non-underlined lower case bases are cg7000 antisense strand.

ATGAAAGGAAGGGAAAAGTTATGTGCTAAACTATCAAGTGCTTTCCTAAG
GAAATGGTGGTTCGTCGTAGCGATATCACTAGGATGTGTACTTTTTGGAA
TAATAATCGCTGTTTTCTTCAGTAGCTGGGTTAATTTAATAATAAATTCA
CAAATTAAATTAGTAAATGGTACACAAACGTTCAGTTGGTGGGCCAAACC
TCCAGTAGAGCCCAAGATAAGGCTTTATATCTACAACGTTACCAACGCTG
ATGAATTCCTTAATAATGGATCGAAACCTATCGTCAACGAATTGGGTCCA
TATGTATACAGAGAAAGATGGGAGAGAAAAAATATTTCAATACACGACAA
CGGAACCCTTACATTTAATCTACAGAAAATTTACCATTTCGACCCAGAAG
CATCTAAAGGTAGTCCAGATGACCTAGTTGTTGTACCAAATATTCCTATG
TTGAGTGCTACCTCCCAATCCAAACATGCCGCCAGATTCCTCAGGTagag
ggatccaggcctaggtatgtttctgcttctacctttgatatatatataat
aattatcactaattagtagtaatatagtatttcaagtatttttttcaaaa
taaaagaatgtagtatatagctattgcttttctgtagtttataagtgtgt
atattttaatttataacttttctaatatatgaccaaaacatggtgatgtg
caggtatttaaataccggtccatggagagacctgaggaatctggcggcat
gtttggattgggaggtagcactcaacataggaatatttggtacaacaact
aggtcatctggactacctttagatgcttctgggtcgaaatggtaaatttt
ctgtagattaaatgtaagggttccgttgtcgtgtattgaaatattttttc
tctcccatctttctctgtatacatatggacccaattcgttgacgataggt
ttcgatccattattaaggaattcatcagcgttggtaacgttgtagatata
aagccttatcttgggctctactggaggtttggcccaccaactgaacgttt
gtgtaccatttactaatttaatttgtgaatttattattaaattaacccag
ctactgaagaaaacagcgattattattccaaaaagtacacatcctagtga
tatcgctacgacgaaccaccatttccttaggaaagcacttgatagtttag
cacataacttttcccttcctttcat

SEQ ID NO:72 shows a DNA sequence comprising heat shock protein 70-331.

SEQ ID NO:73 shows an amino acid sequence of a HEAT SHOCK PROTEIN 70-331 protein.

SEQ ID NO:74 shows a DNA sequence of heat shock protein 70-331 Region 1 (Reg1) that was used for in vitro dsRNA synthesis (T7 promoter sequences at 5′ and 3′ ends not shown).

SEQ ID NO:75 presents a heat shock protein 70-331 hairpin-RNA-forming sequence. Upper case bases are heat shock protein 70-331 sense strand, underlined lower case bases comprise an ST-LS1 intron, non-underlined lower case bases are heat shock protein 70-331 antisense strand.

ATGAGGTTATATTTGGGATTTGGAGTGCTGCTTCTTTTAGCTGCCGTTTT
GGGAGCTAAAGAAGAGAAAAAGGATAAAGAAGATGTTGGAACAGTCATCG
GAATTGATTTGGGAACAACGTATTCATGTGTGGGTGTATATAAAAATGGT
AGAGTAGAAATCATCGCCAATGACCAAGGTAACCGTATCACCCCCTCGTA
TGTTGCCTTCACTGCCGATGGTGAGCGTCTCATCGGAGATGCTGCCAAGA
ATCAACTTACAACAAACCCTGAAAACACTGTTTTTGATGCCAAGCGTCTC
ATTGGTCGTGACTTCACAGACCATACAGTTCAACATGACTTGAAGCTCTT
CCCCTTCAAAGTCATTGAAAAGAACCAGAAGCCACATATTCAAGTAGAAA
CCAGCCAGGGAACTAAGGTCTTTGCCCCTGAAGAAATTTCTGCTATGGTT
TTGGGAAAGATGAAGGAAACAGCAGAAGgactagtaccggttgggaaagg
tatgtttctgcttctacctttgatatatatataataattatcactaatta
gtagtaatatagtatttcaagtatttttttcaaaataaaagaatgtagta
tatagctattgcttttctgtagtttataagtgtgtatattttaatttata
acttttctaatatatgaccaaaacatggtgatgtgcaggttgatccgcgg
ttacttctgctgtttccttcatctttcccaaaaccatagcagaaatttct
tcaggggcaaagaccttagttccctggctggtttctacttgaatatgtgg
cttctggttcttttcaatgactttgaaggggaagagcttcaagtcatgtt
gaactgtatggtctgtgaagtcacgaccaatgagacgcttggcatcaaaa
acagtgttttcagggtttgttgtaagttgattcttggcagcatctccgat
gagacgctcaccatcggcagtgaaggcaacatacgagggggtgatacggt
taccaggtcattggcgatgatttctactctaccatttttatatacaccca
cacatgaatacgttgttcccaaatcaattccgatgactgttccaacatct
tctttatcctttttctcttctttagctcccaaaacggcagctaaaagaag
cagcactccaaatcccaaatataacctcat

SEQ ID NO:76 shows a DNA sequence comprising heat shock protein 70-12300.

SEQ ID NO:77 shows an amino acid sequence of a HEAT SHOCK PROTEIN 70-12300 protein.

SEQ ID NO:78 shows a DNA sequence of heat shock protein 70-12300 Region 1 (Reg1) that was used for in vitro dsRNA synthesis (T7 promoter sequences at 5′ and 3′ ends not shown).

SEQ ID NO:79 presents a heat shock protein 70-12300 hairpin-RNA-forming sequence. Upper case bases are heat shock protein 70-12300 sense strand, underlined lower case bases comprise an ST-LS1 intron, non-underlined lower case bases are heat shock protein 70-12300 antisense strand.

GATGGCTAAAGCCCCAGCTGTAGGTATTGATTTAGGAACCACATACTCCT
GTGTAGGAGTTTTCCAACATGGCAAAGTTGAAATTATTGCCAACGACCAA
GGTAACAGAACCACACCATCATATGTGGCCTTCACAGACACAGAACGTCT
CATCGGAGATGCTGCCAAGAACCAGGTAGCCATGAACCCCAATAACACAA
TTTTTGATGCCAAACGTCTTATTGGGCGTCGCTTTGATGACAGTGCTGTA
CAGTCTGACATGAAACATTGGCCATTTGAAGTAGTAAATGATGCAGGTAA
ACCAAAGATTAAAGTTGCATACAAGGGCGAAGACAAATCCTTCTACCCAG
AAGAAGTCAGTTCCATGGTCCTTACAAAAATGAAGGAAACAGCAGAAGCA
TACTTAGGAAAGAATGTgactagtaccggttgggaaaggtatgtttctgc
ttctacctttgatatatatataataattatcactaattagtagtaatata
gtatttcaagtatttttttcaaaataaaagaatgtagtatatagctattg
cttttctgtagtttataagtgtgtatattttaatttataacttttctaat
atatgaccaaaacatggtgatgtgcaggagatccgcggttaacattcttt
cctaagtatgcttctgctgtttccttcatttttgtaaggaccatggaact
gacttcttctgggtagaaggatttgtcttcgcccttgtatgcaactttaa
tctttggtttacctgcatcatttactacttcaaatggccaatgtttcatg
tcagactgtacagcactgtcatcaaagcgacgcccaataagacgtttggc
atcaaaaattgtgttattggggttcatggctacctggttcttggcagcat
ctccgatgagacgttctgtgtctgtgaaggccacatatgatggtgtggtt
ctgttaccttggtcgttggcaataatttcaactttgccatgttggaaaac
tcctacacaggagtatgtggttcctaaatcaatacctacagctggggctt
tagccatc

SEQ ID NO:80 shows a DNA sequence comprising rnr1.

SEQ ID NO:81 shows an amino acid sequence of a RNR1 protein.

SEQ ID NO:82 shows a DNA sequence of rnr1 Region 1 (Reg1) that was used for in vitro dsRNA synthesis (T7 promoter sequences at 5′ and 3′ ends not shown).

SEQ ID NO:83 shows a DNA sequence of rnr1 Region 2 (Reg2) that was used for in vitro dsRNA synthesis (T7 promoter sequences at 5′ and 3′ ends not shown).

SEQ ID NO:84 shows a DNA sequence of rnr1 Region 3 (Reg3) that was used for in vitro dsRNA synthesis (T7 promoter sequences at 5′ and 3′ ends not shown).

SEQ ID NO:85 presents a rnr1 hairpin-RNA-forming sequence. Upper case bases are rnr1 sense strand, underlined lower case bases comprise an ST-LS1 intron, non-underlined lower case bases are rnr1 antisense strand.

ATGGTGAAACCGTTGAATAAATTTTATGTTTTAAAAAGAGGAGGCCGAAG
AGAAGAGGTCCATATTGATAAAATCACATCACGTATTCAGAAATTATGTT
ATGGCTTGAATGCCGATTATGTCGATCCAGTTAGTATTACCTTGAAAGTG
GTTAATGGCCTATACCCAGGAGTTACTACTGCAGAATTAGATGCACTGGC
TGCAGAAACTGCTGCTACCCTCAGCGAAGACCATCCTGATTATGCTACTC
TAGCAGCTAGGATCGCTATATCTAATCTTCAGAAAGAAACGgactagtac
cggttgggaaaggtatgtttctgcttctacctttgatatatatataataa
ttatcactaattagtagtaatatagtatttcaagtatttttttcaaaata
aaagaatgtagtatatagctattgcttttctgtagtttataagtgtgtat
atttaatttataacttttctaatatatgaccaaaacatggtgatgtgcag
gttgatccgcggttacgtttctttctgaagattagatatagcgatcctag
ctgctagagtagcataatcaggatggtcttcgctgagggtagcagcagtt
tctgcagccagtgcatctaattctgcagtagtaactcctgggtataggcc
attaaccactttcaaggtaatactaactggatcgacataatcggcattca
agccataacataatttctgaatacgtgatgtgattttatcaatatggacc
tcttctcttcggcctcctctttttaaaacataaaatttattcaacggttt
caccat

SEQ ID NO:86 shows a DNA sequence comprising elav.

SEQ ID NO:87 shows an amino acid sequence of a ELAV protein.

SEQ ID NO:88 shows a DNA sequence of elav Region 1 (Reg1) that was used for in vitro dsRNA synthesis (T7 promoter sequences at 5′ and 3′ ends not shown).

SEQ ID NO:89 presents a elav hairpin-RNA-forming sequence. Upper case bases are elav sense strand, underlined lower case bases comprise an ST-LS1 intron, non-underlined lower case bases are elav antisense strand.

CGTAATGAATCCAGGCGCCCCAAATTACCCCATGGCGTCTTTGTACGTTG
GGGATTTACACTCTGATATTACTGAGGCCATGCTTTTTGAGAAATTTTCA
ACTGCGGGCCCTGTTCTTTCGATTCGCGTTTGCAGAGACTTGATTACACG
TAGATCCTTGGGTTATGCGTACGTAAATTTCCAGCAACCTGCTGATGCTG
AGCGTGCGCTGGATACAATGAACTTTGACTTGATTAAAGGACGTCCCATC
AGAATAATGTGGTCTCAACGAGATCCATCATTGAGAAAATCTGGTGTTGG
TAATGTTTTTATTAAGAATTTGGATCGTTCCATTGACAACgactagtacc
ggttgggaaaggtatgtttctgcttctacctttgatatatatataataat
tatcactaattagtagtaatatagtatttcaagtatttttttcaaaataa
aagaatgtagtatatagctattgcttttctgtagtttataagtgtgtata
ttttaatttataacttttctaatatatgaccaaaacatggtgatgtgcag
gttgatccgcggttagttgtcaatggaacgatccaaattcttaataaaaa
cattaccaacaccagattttctcaatgatggatctcgttgagaccacatt
attctgatgggacgtcctttaatcaagtcaaagttcattgtatccagcgc
acgctcagcatcagcaggttgctggaaatttacgtacgcataacccaagg
atctacgtgtaatcaagtctctgcaaacgcgaatcgaaagaacagggccc
gcagttgaaaatttctcaaaaagcatggcctcagtaatatcagagtgtaa
atccccaacgtacaaagacgccatggggtaatttggggcgcctggattca
ttacg

SEQ ID NO:90 shows a DNA sequence comprising pten.

SEQ ID NO:91 shows an amino acid sequence of a PTEN protein.

SEQ ID NO:92 shows a DNA sequence of pten Region 1 (Reg1) that was used for in vitro dsRNA synthesis (T7 promoter sequences at 5′ and 3′ ends not shown).

SEQ ID NO:93 presents a pten hairpin-RNA-forming sequence. Upper case bases are pten sense strand, underlined lower case bases comprise an ST-LS1 intron, non-underlined lower case bases are pten antisense strand.

ATGGGTCAGTGTTTTAGTGCTAGCAGAGCCCAGGATGCACCTCAACCTAA
ACATGGTATCAAAACGTATTTAGTCCAGCAAGCATCCGAAACCGCTTTTT
CCGACAGTAAAAACGAGCAGACCATTGACAGTGTAATGGCTACATCTTTT
TCTAATATGAATATTACAAATCCTATCAAAGGACTTGTTAGTAAAAGAAG
AAATAGATATAAGAAAGATGGTTTCAATCTGGATCTGACCTATATCACCG
ACAATATAATTGCAATGGGGTATCCGGCCTCTAAGATAGAAGGAGTTTAC
CGTAATCATATTGATGACGTTGTAAAGTTCCTCGACAAGAAGCATCCCGA
CCACTATTACATTTACAATCTTTGCTCCGAACGTAGCTACGACAAGATGA
AATTCCACAATAGAGTCAAAATATTTCCCTTCGACGATCACagagggatc
caggcctaggtatgtttctgcttctacctttgatatatatataataatta
tcactaattagtagtaatatagtatttcaagtatttttttcaaaataaaa
gaatgtagtatatagctattgcttttctgtagtttataagtgtgtatatt
ttaatttataacttttctaatatatgaccaaaacatggtgatgtgcaggt
atttaaataccggtccatggagaggtgatcgtcgaagggaaatattttga
ctctattgtggaatttcatcttgtcgtagctacgttcggagcaaagattg
taaatgtaatagtggtcgggatgcttcttgtcgaggaactttacaacgtc
atcaatatgattacggtaaactccttctatcttagaggccggatacccca
ttgcaattatattgtcggtgatataggtcagatccagattgaaaccatct
ttcttatatctatttcttcttttactaacaagtcctttgataggatttgt
aatattcatattagaaaaagatgtagccattacactgtcaatggtctgct
cgtttttactgtcggaaaaagcggtttcggatgcttgctggactaaatac
gttttgataccatgtttaggttgaggtgcatcctgggctctgctagcact
aaaacactgacccat

SEQ ID NO:94 shows a DNA sequence comprising cdc8.

SEQ ID NO:95 shows an amino acid sequence of a CDC8 protein.

SEQ ID NO:96 shows a DNA sequence of cdc8 Region 1 (Reg1) that was used for in vitro dsRNA synthesis (T7 promoter sequences at 5′ and 3′ ends not shown).

SEQ ID NO:97 presents a cdc8 hairpin-RNA-forming sequence. Upper case bases are cdc8 sense strand, underlined lower case bases comprise an ST-LS1 intron, non-underlined lower case bases are cdc8 antisense strand.

ATGAGTATGAAACGAGGAGCACTAATCGTAATAGAAGGTGTAGATCGCTC
AGGAAAATCAACCCAATGTAAAAAGTTGATCCACGCTCTAGAGAAAAAGA
AAATTGAATCCAAGTTGATCGCTTTTCCAGACCGTAGTACCTTAACGGGA
AAACTTATTGACGAGTACTTAAAGAATAAAGACTGTAAACTCAATGATCA
AGCCATACACCTCTTGTTTTCCGCTAATCGATGGGAAAATGTGGAGAAAA
TTAAGAATCTGTTATTTGATGGGGTTACCCTAATTATCGATAGGTATTCT
TATTCTGGGATAGTCTTTTCATCGGTGAAAAAAAATATGTCTATGGAATG
GTGTCAGCATCCTGAGAACGGCCTTCCTAAACCAGATCTAGTGCTTTTGC
TAACTTTAAGTCTAGAAGAAATGCAATCAAGGCCGGGTTTTGGGAATGAA
AGGTACGAAAATATGGAATTTCAGAGAAACGTGGCAAACATGTACAACCA
GCTCTACGATGAAGATGACAATTGGGTTAGAATTGATGCTGCTGGGTCTa
gagggatccaggcctaggtatgtttctgcttctacctttgatatatatat
aataattatcactaattagtagtaatatagtatttcaagtatttttttca
aaataaaagaatgtagtatatagctattgcttttctgtagtttataagtg
tgtatattttaatttataacttttctaatatatgaccaaaacatggtgat
gtgcaggtatttaaataccggtccatggagagtgggaggctgattgatac
ctgtatttaagatgattacaacgtctttgaattcttctaatttataacct
gtataaaattccaatgttggagtccagtggctgatacgttgcatcctaag
tgctatataaagagcagctgatgccaatttggaatccctaatagtgactg
tctcgtaattcattaatgagtattctagaataaatctagctaatgtcaaa
acaggcattgttattttcgcacacctggcatatcttctcagaaatctgta
actaataggtattcccagatcgaatcctattactttaaacaaattgatct
ccatcctaataagaccctcttggtataagcgccatcgcaaatatacaaga
aatcgtcaagcataggtggaatcctttcgtcgtatttactggctatcaac
atggctgcggcaccgactaattgtaaagtttcctttcctactgtcatttt
actgaggtacatatcaactaatttaactcccaaataaagagtctcatgat
tgagttcgaaactttcttgaat

SEQ ID NO:98 shows a DNA sequence of a T7 phage promoter.

SEQ ID NO:99 shows a DNA sequence comprising a YFP coding region segment that was used for in vitro dsRNA synthesis (T7 promoter sequences at 5′ and 3′ ends not shown).

SEQ ID NO:100 shows a DNA sequence of an ST-LS1 Intron.

SEQ ID NO:101 shows a DNA sequence of annexin region1.

SEQ ID NO:102 shows a DNA sequence of annexin region 2.

SEQ ID NO:103 shows a DNA sequence of beta spectrin 2 region 1.

SEQ ID NO:104 shows a DNA sequence of beta spectrin 2 region 2.

SEQ ID NO:105 shows a DNA sequence of mtRP-L4 region 1.

SEQ ID NO:106 shows a DNA sequence of mtRP-L4 region 2.

SEQ ID NOs:107 to 122 show primers used to amplify portions of a chitin synthase sequence comprising chitin synthase Reg1, chitin synthase Reg2, chitin synthase Reg3, and chitin synthase Reg4.

SEQ ID NOs:123 to 130 show primers used to amplify portions of an outer membrane translocase sequence comprising outer membrane translocase Reg1 and outer membrane translocase Reg2.

SEQ ID NOs:131 to 134 show primers used to amplify portions of a double parked sequence comprising double parked Reg1.

SEQ ID NOs:135 to 138 show primers used to amplify portions of a discs overgrown sequence comprising discs overgrown reg1.

SEQ ID NOs:139 to 146 show primers used to amplify portions of a ctf4 sequence comprising ctf4 Reg 1 and ctf4 Var1.

SEQ ID NOs:147 to 150 show primers used to amplify portions of a rpl9 sequence comprising rpl9 Reg1.

SEQ ID NOs:151 to 158 show primers used to amplify portions of a serpin protease inhibitor I4 sequence comprising serpin protease inhibitor I4 Reg1 and serpin protease inhibitor I4 Reg2.

SEQ ID NOs:159 to 166 show primers used to amplify portions of a myosin 3LC sequence comprising myosin 3LC Reg1 and myosin 3LC Reg2.

SEQ ID NOs:167 to 174 show primers used to amplify portions of a megator sequence comprising megator Reg1 and megator Reg2.

SEQ ID NOs:175 to 182 show primers used to amplify portions of a g-protein beta subunit sequence comprising g-protein beta subunit Reg1 and g-protein beta subunit Reg2.

SEQ ID NOs:183 to 186 show primers used to amplify portions of a flap wing sequence comprising flap wing Reg1.

SEQ ID NOs:187 to 194 show primers used to amplify portions of a discs overgrown sequence comprising discs overgrown Reg1.

SEQ ID NOs:195 to 202 show primers used to amplify portions of a enhancer of polycomb sequence comprising enhancer of polycomb Reg1 and enhancer of polycomb Reg2.

SEQ ID NOs:203 to 206 show primers used to amplify portions of a deadbox 73D sequence comprising deadbox 73D reg1.

SEQ ID NOs:207 to 210 show primers used to amplify portions of a cg7000 sequence comprising cg7000 Reg1.

SEQ ID NOs:211 to 214 show primers used to amplify portions of a heat shock protein 70-4 sequence comprising heat shock protein 70-331 and heat shock protein 70-12300.

SEQ ID NOs:215 to 220 show primers used to amplify portions of a rnr1 sequence comprising rnr1 Reg1, rnr1 Reg2 and rnr1 Reg3.

SEQ ID NOs:221 to 222 show primers used to amplify portions of a elav sequence comprising elav Reg1.

SEQ ID NOs:223 to 224 show primers used to amplify portions of a pten sequence comprising pten Reg1.

SEQ ID NOs:225 to 226 show primers used to amplify portions of a cdc8 sequence comprising cdc8 Reg1.

SEQ ID NOs:227 to 254 show primers used to amplify gene regions of YFP, annexin, beta spectrin 2, and mtRP-L4 for dsRNA synthesis.

SEQ ID NO:255 shows a DNA sequence of oligonucleotide T20NV.

SEQ ID NOs:256 to 263 show sequences of primers and probes used to measure maize transcript levels.

SEQ ID NOs:264 to 272 show sequences of primers and probes used for gene copy number analyses.

SEQ ID NO:273 shows a DNA sequence of a portion of a SpecR coding region used for binary vector backbone detection.

SEQ ID NO:274 shows a DNA sequence of a portion of an AAD1 coding region used for genomic copy number analysis.

SEQ ID NO:275 shows a DNA sequence of a maize invertase gene.

SEQ ID NO:276 shows a maize DNA sequence encoding a TIP41-like protein.

SEQ ID NO:277 presents a YFP hairpin-RNA-forming sequence v2 as found in pDAB110853. Upper case bases are YFP sense strand, underlined bases comprise an ST-LS1 intron, lower case, non-underlined bases are YFP antisense strand.

ATGTCATCTGGAGCACTTCTCTTTCATGGGAAGATTCCTTACGTTGTGGA
GATGGAAGGGAATGTTGATGGCCACACCTTTAGCATACGTGGGAAAGGCT
ACGGAGATGCCTCAGTGGGAAAGgactagtaccggttgggaaaggtatgt
ttctgcttctacctttgatatatatataataattatcactaattagtagt
aatatagtatttcaagtatttttttcaaaataaaagaatgtagtatatag
ctattgcttttctgtagtttataagtgtgtatattttaatttataacttt
tctaatatatgaccaaaacatggtgatgtgcaggttgatccgcggttact
ttcccactgaggcatctccgtagcctttcccacgtatgctaaaggtgtgg
ccatcaacattcccttccatctccacaacgtaaggaatcttcccatgaaa
gagaagtgctccagatgacat

SEQ ID NO:278 shows a DNA sequence of ctf4 var2.

SEQ ID NO:279 shows a DNA sequence of myosin 3LC var1.

### DETAILED DESCRIPTION

### I. Overview of Several Embodiments

Disclosed herein are methods and compositions for genetic control of coleopteran pest infestations. Methods for identifying one or more gene(s) essential to the lifecycle of a coleopteran pest for use as a target gene for RNAi-mediated control of a coleopteran pest population are also provided. DNA plasmid vectors encoding one or more dsRNA molecules may be designed to suppress one or more target gene(s) essential for growth, survival, development, and/or reproduction. In some embodiments, methods are provided for post-transcriptional repression of expression or inhibition of a target gene via nucleic acid molecules that are complementary to a coding or non-coding sequence of the target gene in a coleopteran pest. In these and further embodiments, a coleopteran pest may ingest one or more dsRNA, siRNA, miRNA, shRNA, and/or hpRNA molecules transcribed from all or a portion of a nucleic acid molecule that is complementary to a coding or non-coding sequence of a target gene, thereby providing a plant-protective effect.

Thus, some embodiments involve sequence-specific inhibition of expression of target gene products, using dsRNA, siRNA, miRNA, shRNA, and/or hpRNA that is complementary to coding and/or non-coding sequences of the target gene(s) to achieve at least partial control of a coleopteran pest. Disclosed is a set of isolated and purified nucleic acid molecules comprising a nucleotide sequence, for example, as set forth in any of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, SEQ ID NOs:95 to 97, and fragments thereof. In some embodiments, a stabilized dsRNA molecule may be expressed from this sequence, fragments thereof, or a gene comprising one of these sequences, for the post-transcriptional silencing or inhibition of a target gene. In certain embodiments, isolated and purified nucleic acid molecules comprise all or part of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94. In other embodiments, isolated and purified nucleic acid molecules comprise all or part of sequences as listed in Table 1.

Some embodiments involve a recombinant host cell (e.g., a plant cell) having in its genome at least one recombinant DNA sequence encoding at least one iRNA (e.g., dsRNA) molecule(s). In particular embodiments, the dsRNA molecule(s) may be produced when ingested by a coleopteran pest to post-transcriptionally silence or inhibit the expression of a target gene in the coleopteran pest. The recombinant DNA sequence may comprise, for example, one or more of any of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; fragments of any of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; or a partial sequence of a gene comprising one or more of SEQ ID NO:1, SEQ ID NOs:3 to 7, SEQ ID NO:8, SEQ ID NOs:10 to 12, SEQ ID NO:13, SEQ ID NOs:15 to 16, SEQ ID NO:17, SEQ ID NOs:19 to 20, SEQ ID NO:21, SEQ ID NOs:23 to 25, SEQ ID NO:26, SEQ ID NOs:28 to 29, SEQ ID NO:30, SEQ ID NOs:32 to 34, SEQ ID NO:35, SEQ ID NOs:37 to 39, SEQ ID NO:40, SEQ ID NOs:42 to 44, SEQ ID NO:45, SEQ ID NOs:47 to 49, SEQ ID NO:50, SEQ ID NOs:52 to 53, SEQ ID NO:54, SEQ ID NOs:56 to 58, SEQ ID NO:59, SEQ ID NOs:61 to 63, SEQ ID NO:64, SEQ ID NOs:66 to 67, SEQ ID NO:68, SEQ ID NOs:70 to 71, SEQ ID NO:72, SEQ ID NOs:74 to 75, SEQ ID NO:76, SEQ ID NOs:78 to 79, SEQ ID NO:80, SEQ ID NOs:82 to 85, SEQ ID NO:86, SEQ ID NOs:88 to 89, SEQ ID NO:90, SEQ ID NOs:92 to 93, SEQ ID NO:94, and SEQ ID NOs:95 to 97; or complements thereof.

Particular embodiments involve a recombinant host cell having in its genome a recombinant DNA sequence encoding at least one iRNA (e.g., dsRNA) molecule(s) comprising all or part of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94. When ingested by a coleopteran pest, the iRNA molecule(s) may silence or inhibit the expression of a target gene comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, in the coleopteran pest, and thereby result in cessation of growth, development, reproduction, and/or feeding in the coleopteran pest.

In some embodiments, a recombinant host cell having in its genome at least one recombinant DNA sequence encoding at least one dsRNA molecule may be a transformed plant cell. Some embodiments involve transgenic plants comprising such a transformed plant cell. In addition to such transgenic plants, progeny plants of any transgenic plant generation, transgenic seeds, and transgenic plant products, are all provided, each of which comprises recombinant DNA sequence(s). In particular embodiments, a dsRNA molecule of the invention may be expressed in a transgenic plant cell. Therefore, in these and other embodiments, a dsRNA molecule of the invention may be isolated from a transgenic plant cell. In particular embodiments, the transgenic plant is a plant selected from the group comprising corn (Zea mays) and plants of the family Poaceae.

Some embodiments involve a method for modulating the expression of a target gene in a coleopteran pest cell. In these and other embodiments, a nucleic acid molecule may be provided, wherein the nucleic acid molecule comprises a nucleotide sequence encoding a dsRNA molecule. In particular embodiments, a nucleotide sequence encoding a dsRNA molecule may be operatively linked to a promoter, and may also be operatively linked to a transcription termination sequence. In particular embodiments, a method for modulating the expression of a target gene in a coleopteran pest cell may comprise: (a) transforming a plant cell with a vector comprising a nucleotide sequence encoding a dsRNA molecule; (b) culturing the transformed plant cell under conditions sufficient to allow for development of a plant cell culture comprising a plurality of transformed plant cells; (c) selecting for a transformed plant cell that has integrated the vector into its genome; and (d) determining that the selected transformed plant cell comprises the dsRNA molecule encoded by the nucleotide sequence of the vector. A plant may be regenerated from a plant cell that has the vector integrated in its genome and comprises the dsRNA molecule encoded by the nucleotide sequence of the vector.

Thus, also disclosed is a transgenic plant comprising a vector having a nucleotide sequence encoding a dsRNA molecule integrated in its genome, wherein the transgenic plant comprises the dsRNA molecule encoded by the nucleotide sequence of the vector. In particular embodiments, expression of a dsRNA molecule in the plant is sufficient to modulate the expression of a target gene in a cell of a coleopteran pest that contacts the transformed plant or plant cell, for example, by feeding on the transformed plant, a part of the plant (e.g., root) or plant cell. Transgenic plants disclosed herein may display resistance and/or enhanced tolerance to coleopteran pest infestations. Particular transgenic plants may display resistance and/or enhanced tolerance to one or more coleopteran pests selected from the group consisting of: WCR; NCR; SCR; MCR; D. balteata LeConte; D. u. tenella; D. speciosa Germar; and D. u. undecimpunctata Mannerheim.

Also disclosed herein are methods for delivery of control agents, such as an iRNA molecule, to a coleopteran pest. Such control agents may cause, directly or indirectly, an impairment in the ability of the coleopteran pest to feed, grow or otherwise cause damage in a host. In some embodiments, a method is provided comprising delivery of a stabilized dsRNA molecule to a coleopteran pest to suppress at least one target gene in the coleopteran pest, thereby reducing or eliminating plant damage by a coleopteran pest. In some embodiments, a method of inhibiting expression of a target gene in a coleopteran pest may result in the cessation of growth, development, reproduction, and/or feeding in the coleopteran pest. In some embodiments, the method may eventually result in death of the coleopteran pest.

In some embodiments, compositions (e.g., a topical composition) are provided that comprise an iRNA (e.g., dsRNA) molecule of the invention for use in plants, animals, and/or the environment of a plant or animal to achieve the elimination or reduction of a coleopteran pest infestation. In particular embodiments, the composition may be a nutritional composition or food source to be fed to the coleopteran pest. Some embodiments comprise making the nutritional composition or food source available to the coleopteran pest. Ingestion of a composition comprising iRNA molecules may result in the uptake of the molecules by one or more cells of the coleopteran pest, which may in turn result in the inhibition of expression of at least one target gene in cell(s) of the coleopteran pest. Ingestion of or damage to a plant or plant cell by a coleopteran pest may be limited or eliminated in or on any host tissue or environment in which the coleopteran pest is present by providing one or more compositions comprising an iRNA molecule of the invention in the host of the coleopteran pest.

By combining target RNAi with other useful RNAi targets (e.g., ROP (U.S. patent application Publication Ser. No. 14/577,811), RNAPII (U.S. patent application Ser. No. 14/577,854), RNA polymerase I (U.S. Patent Application No. 62/133,214), RNA polymerase II-33 (U.S. Patent Application No. 62/133,210), ncm (U.S. Patent Application No. 62/095,487), Dre4 (U.S. patent application Ser. No. 14/705,807), COPI alpha (U.S. Patent Application No. 62/063,199), COPI beta (U.S. Patent Application No. 62/063,203), COPI gamma (U.S. Patent Application No. 62/063,192), COPI delta (U.S. Patent Application No. 62/063,216), Caf1-180 (U.S. Patent Application Publication No. 2012/0174258), VatpaseC (U.S. Patent Application Publication No. 2012/0174259), Rho1 (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), and RNA polymerase II215 (U.S. Patent Application No. 62/133,202)), the potential to affect multiple target sequences, for example, in larval rootworms, may increase opportunities to develop sustainable approaches to insect pest management involving RNAi technologies.

RNAi baits are formed when the dsRNA is mixed with food or an attractant or both. When the pests eat the bait, they also consume the dsRNA. Baits may take the form of granules, gels, flowable powders, liquids, or solids. In another embodiment, useful RNAi targets may be incorporated into a bait formulation such as that described in U.S. Pat. No. 8,530,440 which is hereby incorporated by reference. Generally, with baits, the baits are placed in or around the environment of the insect pest, for example, WCR can come into contact with, and/or be attracted to, the bait.

The compositions and methods disclosed herein may be used together in combinations with other methods and compositions for controlling damage by coleopteran pests. For example, an iRNA molecule as described herein for protecting plants from coleopteran pests may be used in a method comprising the additional use of one or more chemical agents effective against a coleopteran pest, biopesticides effective against a coleopteran pest, crop rotation, or recombinant genetic techniques that exhibit features different from the features of the RNAi-mediated methods and RNAi compositions of the invention (e.g., recombinant production of proteins in plants that are harmful to a coleopteran pest (e.g., Bt toxins)).

### II. Abbreviations

dsRNA double-stranded ribonucleic acid
GI growth inhibition
NCBI National Center for Biotechnology Information
gDNA genomic Deoxyribonucleic Acid
iRNA inhibitory ribonucleic acid
ORF open reading frame
RNAi ribonucleic acid interference
miRNA micro ribonucleic acid
siRNA small inhibitory ribonucleic acid
hpRNA hairpin ribonucleic acid
UTR untranslated region
WCR western corn rootworm (Diabrotica virgifera virgifera LeConte)
NCR northern corn rootworm (Diabrotica barberi Smith and Lawrence)
MCR Mexican corn rootworm (Diabrotica virgifera zeae Krysan and Smith)
PCR Polymerase chain reaction
RISC RNA-induced Silencing Complex
SCR southern corn rootworm (Diabrotica undecimpunctata howardi Barber)
YFP yellow fluorescent protein
SEM standard error of the mean

### III. Terms

In the description and tables which follow, a number of terms are used. In order to provide a clear and consistent understanding of the specification and claims, including the scope to be given such terms, the following definitions are provided:

Coleopteran pest: As used herein, the term “coleopteran pest” refers to insects of the genus Diabrotica, which feed upon corn and other true grasses. In particular examples, a coleopteran pest is selected from the list comprising D. v. virgifera LeConte (WCR); D. barberi Smith and Lawrence (NCR); D. u. howardi (SCR); D. v. zeae (MCR); D. balteata LeConte; D. u. tenella; D. speciosa Germar; and D. u. undecimpunctata Mannerheim.

Contact (with an organism): As used herein, the term “contact with” or “uptake by” an organism (e.g., a coleopteran pest), with regard to a nucleic acid molecule, includes internalization of the nucleic acid molecule into the organism, for example and without limitation: ingestion of the molecule by the organism (e.g., by feeding); contacting the organism with a composition comprising the nucleic acid molecule; and soaking of organisms with a solution comprising the nucleic acid molecule.

Contig: As used herein the term “contig” refers to a DNA sequence that is reconstructed from a set of overlapping DNA segments derived from a single genetic source.

Corn plant: As used herein, the term “corn plant” refers to a plant of the species, Zea mays (maize).

Encoding a dsRNA: As used herein, the term “encoding a dsRNA” includes a gene whose RNA transcription product is capable of forming an intramolecular dsRNA structure (e.g., a hairpin) or intermolecular dsRNA structure (e.g., by hybridizing to a target RNA molecule).

Expression: As used herein, “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, northern (RNA) blot, RT-PCR, western (immuno-) blot, or in vitro, in situ, or in vivo protein activity assay(s).

Genetic material: As used herein, the term “genetic material” includes all genes and nucleic acid molecules, such as DNA and RNA.

Inhibition: As used herein, the term “inhibition”, when used to describe an effect on a coding sequence (for example, a gene), refers to a measurable decrease in the cellular level of mRNA transcribed from the coding sequence and/or peptide, polypeptide, or protein product of the coding sequence. In some examples, expression of a coding sequence may be inhibited such that expression is approximately eliminated. “Specific inhibition” refers to the inhibition of a target coding sequence without consequently affecting expression of other coding sequences (e.g., genes) in the cell wherein the specific inhibition is being accomplished.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

Nucleic acid molecule: As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. By convention, the nucleotide sequence of a nucleic acid molecule is read from the 5′ to the 3′ end of the molecule. The “complement” of a nucleotide sequence refers to the sequence, from 5′ to 3′, of the nucleobases which form base pairs with the nucleobases of the nucleotide sequence (i.e., A-T/U, and G-C). The “reverse complement” of a nucleic acid sequence refers to the sequence, from 3′ to 5′, of the nucleobases which form base pairs with the nucleobases of the nucleotide sequence.

“Nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), shRNA (small hairpin RNA), hpRNA (hairpin RNA), tRNA (transfer RNA, whether charged or discharged with a corresponding acylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment”, or more generally “segment”, will be understood by those in the art as a functional term that includes both genomic sequences, ribosomal RNA sequences, transfer RNA sequences, messenger RNA sequences, operon sequences, and smaller engineered nucleotide sequences that encode or may be adapted to encode, peptides, polypeptides, or proteins.

Oligonucleotide: An oligonucleotide is a short nucleic acid polymer. Oligonucleotides may be formed by cleavage of longer nucleic acid segments, or by polymerizing individual nucleotide precursors. Automated synthesizers allow the synthesis of oligonucleotides up to several hundred bases in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of DNA and RNA (reverse transcribed into a cDNA) sequences. In PCR, the oligonucleotide is typically referred to as a “primer”, which allows a DNA polymerase to extend the oligonucleotide and replicate the complementary strand.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding sequence”, “structural nucleotide sequence”, or “structural nucleic acid molecule” refers to a nucleotide sequence that is ultimately translated into a polypeptide, via transcription and mRNA, when placed under the control of appropriate regulatory sequences. With respect to RNA, the term “coding sequence” refers to a nucleotide sequence that is translated into a peptide, polypeptide, or protein. The boundaries of a coding sequence are determined by a translation start codon at the 5′-terminus and a translation stop codon at the 3-terminus. Coding sequences include, but are not limited to: genomic DNA; cDNA; EST; and recombinant nucleotide sequences.

Genome: As used herein, the term “genome” refers to chromosomal DNA found within the nucleus of a cell, and also refers to organelle DNA found within subcellular components of the cell. In some embodiments of the invention, a DNA molecule may be introduced into a plant cell such that the DNA molecule is integrated into the genome of the plant cell. In these and further embodiments, the DNA molecule may be either integrated into the nuclear DNA of the plant cell, or integrated into the DNA of the chloroplast or mitochondrion of the plant cell. The term “genome” as it applies to bacteria refers to both the chromosome and plasmids within the bacterial cell. In some embodiments of the invention, a DNA molecule may be introduced into a bacterium such that the DNA molecule is integrated into the genome of the bacterium. In these and further embodiments, the DNA molecule may be either chromosomally-integrated or located as or in a stable plasmid.

Sequence identity: The term “sequence identity” or “identity”, as used herein in the context of two nucleic acid or polypeptide sequences, refers to the residues in the two sequences that are the same when aligned for maximum correspondence over a specified comparison window.

As used herein, the term “percentage of sequence identity” may refer to the value determined by comparing two optimally aligned sequences (e.g., nucleic acid sequences or polypeptide sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-244; Higgins and Sharp (1989) CABIOS 5:151-153; Corpet et al. (1988) Nucleic Acids Res. 16:10881-10890; Huang et al. (1992) Comp. Appl. Biosci. 8:155-165; Pearson et al. (1994) Methods Mol. Biol. 24:307-331; Tatiana et al. (1999) FEMS Microbiol. Lett. 174:247-250. A detailed consideration of sequence alignment methods and homology calculations can be found in, e.g., Altschul et al. (1990) J. Mol. Biol. 215:403-410.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. Nucleic acid sequences with even greater similarity to the reference sequences will show increasing percentage identity when assessed by this method.

Specifically hybridizable/Specifically complementary: As used herein, the terms “Specifically hybridizable” and “Specifically complementary” are terms that indicate a sufficient degree of complementarity such that stable and specific binding occurs between the nucleic acid molecule and a target nucleic acid molecule. Hybridization between two nucleic acid molecules involves the formation of an anti-parallel alignment between the nucleic acid sequences of the two nucleic acid molecules. The two molecules are then able to form hydrogen bonds with corresponding bases on the opposite strand to form a duplex molecule that, if it is sufficiently stable, is detectable using methods well known in the art. A nucleic acid molecule need not be 100% complementary to its target sequence to be specifically hybridizable. However, the amount of sequence complementarity that must exist for hybridization to be specific is a function of the hybridization conditions used.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization will determine the stringency of hybridization. The ionic strength of the wash buffer and the wash temperature also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11, and updates; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, N Y, 1993; and Ausubel et al., Eds., Current Protocols in Molecular Biology, Chapter 2, Greene Publishing and Wiley-Interscience, N Y, 1995, and updates.

As used herein, “stringent conditions” encompass conditions under which hybridization will occur only if there is more than 80% sequence match between the hybridization molecule and a homologous sequence within the target nucleic acid molecule. “Stringent conditions” include further particular levels of stringency. Thus, as used herein, “moderate stringency” conditions are those under which molecules with more than 80% sequence match (i.e. having less than 20% mismatch) will hybridize; conditions of “high stringency” are those under which sequences with more than 90% match (i.e. having less than 10% mismatch) will hybridize; and conditions of “very high stringency” are those under which sequences with more than 95% match (i.e. having less than 5% mismatch) will hybridize.

The following are representative, non-limiting hybridization conditions.

High Stringency condition (detects sequences that share at least 90% sequence identity): Hybridization in 5×SSC buffer at 65° C. for 16 hours; wash twice in 2×SSC buffer at room temperature for 15 minutes each; and wash twice in 0.5×SSC buffer at 65° C. for 20 minutes each.

Moderate Stringency condition (detects sequences that share at least 80% sequence identity): Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.

Non-stringent control condition (sequences that share at least 50% sequence identity will hybridize): Hybridization in 6×SSC buffer at room temperature to 55° C. for 16-20 hours; wash at least twice in 2×-3×SSC buffer at room temperature to 55° C. for 20-30 minutes each.

As used herein, the term “substantially homologous” or “substantial homology”, with regard to a contiguous nucleic acid sequence, refers to contiguous nucleotide sequences that are borne by nucleic acid molecules that hybridize under stringent conditions to a nucleic acid molecule having the reference nucleic acid sequence. For example, nucleic acid molecules having sequences that are substantially homologous to a reference nucleic acid sequence of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94 are those nucleic acid molecules that hybridize under stringent conditions (e.g., the Moderate Stringency conditions set forth, supra) to nucleic acid molecules having the reference nucleic acid sequence of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94. Substantially homologous sequences may have at least 80% sequence identity. For example, substantially homologous sequences may have from about 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

As used herein, the term “ortholog” refers to a gene in two or more species that has evolved from a common ancestral nucleotide sequence, and may retain the same function in the two or more species.

As used herein, two nucleic acid sequence molecules are said to exhibit “complete complementarity” when every nucleotide of a sequence read in the 5′ to 3′ direction is complementary to every nucleotide of the other sequence when read in the 3′ to 5′ direction. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

Operably linked: A first nucleotide sequence is operably linked with a second nucleic acid sequence when the first nucleic acid sequence is in a functional relationship with the second nucleic acid sequence. When recombinantly produced, operably linked nucleic acid sequences are generally contiguous, and, where necessary, two protein-coding regions may be joined in the same reading frame (e.g., in a translationally fused ORF). However, nucleic acids need not be contiguous to be operably linked.

The term, “operably linked”, when used in reference to a regulatory sequence and a coding sequence, means that the regulatory sequence affects the expression of the linked coding sequence. “Regulatory sequences”, or “control elements”, refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

Promoter: As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissues are referred to as “tissue-specific”. A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most tissue or cell types.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that respond to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:10421-10425).

Exemplary constitutive promoters include, but are not limited to: Promoters from plant viruses, such as the 35S promoter from Cauliflower Mosaic Virus (CaMV); promoters from rice actin genes; ubiquitin promoters; pEMU; MAS; maize H3 histone promoter; and the ALS promoter, XbaI/NcoI fragment 5′ to the Brassica napus ALS3 structural gene (or a nucleotide sequence similar to said XbaI/NcoI fragment) (U.S. Pat. No. 5,659,026).

Additionally, any tissue-specific or tissue-preferred promoter may be utilized in some embodiments of the invention. Plants transformed with a nucleic acid molecule comprising a coding sequence operably linked to a tissue-specific promoter may produce the product of the coding sequence exclusively, or preferentially, in a specific tissue. Exemplary tissue-specific or tissue-preferred promoters include, but are not limited to: A seed-preferred promoter, such as that from the phaseolin gene; a leaf-specific and light-induced promoter such as that from cab or rubisco; an anther-specific promoter such as that from LAT52; a pollen-specific promoter such as that from Zm13; and a microspore-preferred promoter such as that from apg.

Transformation: As used herein, the term “transformation” or “transduction” refers to the transfer of one or more nucleic acid molecule(s) into a cell. A cell is “transformed” by a nucleic acid molecule transduced into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-793); lipofection (Felgner et al. (1987) Proc. Natl. Acad. Sci. USA 84:7413-7417); microinjection (Mueller et al. (1978) Cell 15:579-585); Agrobacterium-mediated transfer (Fraley et al. (1983) Proc. Natl. Acad. Sci. USA 80:4803-4807); direct DNA uptake; and microprojectile bombardment (Klein et al. (1987) Nature 327:70).

Transgene: An exogenous nucleic acid sequence. In some examples, a transgene may be a sequence that encodes one or both strand(s) of a dsRNA molecule that comprises a nucleotide sequence that is complementary to a nucleic acid molecule found in a coleopteran pest. In further examples, a transgene may be an antisense nucleic acid sequence, wherein expression of the antisense nucleic acid sequence inhibits expression of a target nucleic acid sequence. In still further examples, a transgene may be a gene sequence (e.g., a herbicide-resistance gene), a gene encoding an industrially or pharmaceutically useful compound, or a gene encoding a desirable agricultural trait. In these and other examples, a transgene may contain regulatory sequences operably linked to a coding sequence of the transgene (e.g., a promoter).

Vector: A nucleic acid molecule as introduced into a cell, for example, to produce a transformed cell. A vector may include nucleic acid sequences that permit it to replicate in the host cell, such as an origin of replication. Examples of vectors include, but are not limited to: a plasmid; cosmid; bacteriophage; or virus that carries exogenous DNA into a cell. A vector may also be an RNA molecule. A vector may also include one or more genes, antisense sequences, and/or selectable marker genes and other genetic elements known in the art. A vector may transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coating, etc.).

Yield: A stabilized yield of about 100% or greater relative to the yield of check varieties in the same growing location growing at the same time and under the same conditions. In particular embodiments, “improved yield” or “improving yield” means a cultivar having a stabilized yield of 105% to 115% or greater relative to the yield of check varieties in the same growing location containing significant densities of coleopteran pests that are injurious to that crop growing at the same time and under the same conditions.

Unless specifically indicated or implied, the terms “a”, “an”, and “the” signify “at least one” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin's Genes X, Jones & Bartlett Publishers, 2009 (ISBN 10 0763766321); Krebs et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

### IV. Nucleic Acid Molecules Comprising a Coleopteran Pest Sequence

A. Overview

Described herein are nucleic acid molecules useful for the control of coleopteran pests. Described nucleic acid molecules include target sequences (e.g., native genes, and non-coding sequences), dsRNAs, siRNAs, shRNAs, hpRNAs, and miRNAs. For example, dsRNA, siRNA, miRNA and/or hpRNA molecules are described in some embodiments that may be specifically complementary to all or part of one or more native nucleic acid sequences in a coleopteran pest. In these and further embodiments, the native nucleic acid sequence(s) may be one or more target gene(s), the product of which may be, for example and without limitation: involved in a metabolic process; involved in a reproductive process; or involved in larval development. Nucleic acid molecules described herein, when introduced into a cell comprising at least one native nucleic acid sequence(s) to which the nucleic acid molecules are specifically complementary, may initiate RNAi in the cell, and consequently reduce or eliminate expression of the native nucleic acid sequence(s). In some examples, reduction or elimination of the expression of a target gene by a nucleic acid molecule comprising a sequence specifically complementary thereto may be lethal in coleopteran pests, or result in reduced growth and/or reproduction.

In some embodiments, at least one target gene in a coleopteran pest may be selected, wherein the target gene comprises a nucleotide sequence comprising chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94). In particular examples, a target gene in a coleopteran pest is selected, wherein the target gene comprises a novel nucleotide sequence comprising chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94).

In some embodiments, a target gene may be a nucleic acid molecule comprising a nucleotide sequence that encodes a polypeptide comprising a contiguous amino acid sequence that is at least 85% identical (e.g., about 90%, about 95%, about 96%, about 97%, about 98%, about 99%, about 100%, or 100% identical) to the amino acid sequence of a protein product of chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94). A target gene may be any nucleic acid sequence in a coleopteran pest, the post-transcriptional inhibition of which has a deleterious effect on the coleopteran pest, or provides a protective benefit against the coleopteran pest to a plant. In particular examples, a target gene is a nucleic acid molecule comprising a nucleotide sequence that encodes a polypeptide comprising a contiguous amino acid sequence that is at least 85% identical, about 90% identical, about 95% identical, about 96% identical, about 97% identical, about 98% identical, about 99% identical, about 100% identical, or 100% identical to the amino acid sequence of a protein product of novel nucleotide sequence chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94).

Provided according to the invention are nucleotide sequences, the expression of which results in an RNA molecule comprising a nucleotide sequence that is specifically complementary to all or part of a native RNA molecule that is encoded by a coding sequence in a coleopteran pest. In some embodiments, after ingestion of the expressed RNA molecule by a coleopteran pest, down-regulation of the coding sequence in cells of the coleopteran pest may be obtained. In particular embodiments, down-regulation of the coding sequence in cells of the coleopteran pest may result in a deleterious effect on the growth, viability, proliferation, and/or reproduction of the coleopteran pest.

In some embodiments, target sequences include transcribed non-coding RNA sequences, such as 5′UTRs; 3′UTRs; spliced leader sequences; intron sequences; outron sequences (e.g., 5′UTR RNA subsequently modified in trans splicing); donatron sequences (e.g., non-coding RNA required to provide donor sequences for trans splicing); and other non-coding transcribed RNA of target coleopteran pest genes. Such sequences may be derived from both mono-cistronic and poly-cistronic genes.

Thus, also described herein in connection with some embodiments are iRNA molecules (e.g., dsRNAs, siRNAs, shRNAs, miRNAs and hpRNAs) that comprise at least one nucleotide sequence that is specifically complementary to all or part of a target sequence in a coleopteran pest. In some embodiments an iRNA molecule may comprise nucleotide sequence(s) that are complementary to all or part of a plurality of target sequences; for example, 2, 3, 4, 5, 6, 7, 8, 9, 10, or more target sequences. In particular embodiments, an iRNA molecule may be produced in vitro or in vivo by a genetically-modified organism, such as a plant or bacterium. Also disclosed are cDNA sequences that may be used for the production of dsRNA molecules, siRNA molecules, miRNA molecules, shRNA molecules, and/or hpRNA molecules that are specifically complementary to all or part of a target sequence in a coleopteran pest. Further described are recombinant DNA constructs for use in achieving stable transformation of particular host targets. Transformed host targets may express effective levels of dsRNA, siRNA, miRNA, shRNA, and/or hpRNA molecules from the recombinant DNA constructs. Therefore, also described is a plant transformation vector comprising at least one nucleotide sequence operably linked to a heterologous promoter functional in a plant cell, wherein expression of the nucleotide sequence(s) results in an RNA molecule comprising a nucleotide sequence that is specifically complementary to all or part of a target sequence in a coleopteran pest.

In some embodiments, nucleic acid molecules useful for the control of coleopteran pests may include: all or part of a native nucleic acid sequence isolated from Diabrotica comprising chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94); nucleotide sequences that when expressed result in an RNA molecule comprising a nucleotide sequence that is specifically complementary to all or part of a native RNA molecule that is encoded by chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94); iRNA molecules (e.g., dsRNAs, siRNAs, shRNAs, miRNAs and hpRNAs) that comprise at least one nucleotide sequence that is specifically complementary to all or part of chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94); cDNA sequences that may be used for the production of dsRNA molecules, siRNA molecules, miRNA molecules, shRNA molecules, and/or hpRNA molecules that are specifically complementary to all or part of chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), or cdc8 (SEQ ID NO:94); and recombinant DNA constructs for use in achieving stable transformation of particular host targets, wherein a transformed host target comprises one or more of the foregoing nucleic acid molecules.

B. Nucleic Acid Molecules

The present invention provides, inter alia, iRNA (e.g., dsRNA, siRNA, miRNA, shRNA, and hpRNA) molecules that inhibit target gene expression in a cell, tissue, or organ of a coleopteran pest; and DNA molecules capable of being expressed as an iRNA molecule in a cell or microorganism to inhibit target gene expression in a cell, tissue, or organ of a coleopteran pest.

Some embodiments of the invention provide an isolated nucleic acid molecule comprising at least one (e.g., one, two, three, or more) nucleotide sequence(s) selected from the group consisting of: SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a fragment of at least 19 contiguous nucleotides of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a fragment of at least 19 contiguous nucleotides of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; and the complement of a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94. In particular embodiments, contact with or uptake by a coleopteran pest of the isolated nucleic acid sequence inhibits the growth, development, reproduction and/or feeding of the coleopteran pest.

In some embodiments, a nucleic acid molecule of the invention may comprise at least one (e.g., one, two, three, or more) DNA sequence(s) capable of being expressed as an iRNA molecule in a cell or microorganism to inhibit target gene expression in a cell, tissue, or organ of a coleopteran pest. Such DNA sequence(s) may be operably linked to a promoter sequence that functions in a cell comprising the DNA molecule to initiate or enhance the transcription of the encoded RNA capable of forming a dsRNA molecule(s). In one embodiment, at least one (e.g., one, two, three, or more) DNA sequence(s) may be derived from a nucleotide sequence comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94. Derivatives of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94 including fragments of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94. In some embodiments, such a fragment may comprise, for example, at least about 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, SEQ ID NO:94, or a complement thereof. Thus, such a fragment may comprise, for example, 19, 20, 21, 22, 23, 24, 25, 26, 27, 28, 29, or 30 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, SEQ ID NO:94, or a complement thereof. In these and further embodiments, such a fragment may comprise, for example, more than about 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, SEQ ID NO:94, or a complement thereof. Thus, a fragment of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94 may comprise, for example, 19, 20, 21, about 25, (e.g., 22, 23, 24, 25, 26, 27, 28, and 29), about 30, about 40, (e.g., 35, 36, 37, 38, 39, 40, 41, 42, 43, 44, and 45), about 50, about 60, about 70, about 80, about 90, about 100, about 110, about 120, about 130, about 140, about 150, about 160, about 170, about 180, about 190, about 200 or more contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, SEQ ID NO:94, or a complement thereof.

Some embodiments comprise introducing partial- or fully-stabilized dsRNA molecules into a coleopteran pest to inhibit expression of a target gene in a cell, tissue, or organ of the coleopteran pest. When expressed as an iRNA molecule (e.g., dsRNA, siRNA, miRNA, shRNA, and hpRNA) and taken up by a coleopteran pest, nucleic acid sequences comprising one or more fragments of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94 may cause one or more of death, growth inhibition, reduction in brood size, cessation of infection, and/or cessation of feeding by a coleopteran pest. For example, in some embodiments, a dsRNA molecule comprising a nucleotide sequence including about 19 to about 300 nucleotides that are substantially homologous to a coleopteran pest target gene sequence and comprising one or more fragments of a nucleotide sequence comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94 is provided. Expression of such a dsRNA molecule may, for example, lead to mortality and/or growth inhibition in a coleopteran pest that takes up the dsRNA molecule.

In certain embodiments, dsRNA molecules provided by the invention comprise nucleotide sequences complementary to a target gene comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, and/or nucleotide sequences complementary to a fragment of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, the inhibition of which target gene in a coleopteran pest results in the reduction or removal of a protein or nucleotide sequence agent that is essential for the coleopteran pest's growth, development, or other biological function. A selected nucleotide sequence may exhibit from about 80% to about 100% sequence identity to SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, a contiguous fragment of the nucleotide sequence set forth in SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, or the complement of either of the foregoing. For example, a selected nucleotide sequence may exhibit about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; or about 100% sequence identity to SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, a contiguous fragment of the nucleotide sequence set forth in SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, or the complement of any of the foregoing.

In some embodiments, a DNA molecule capable of being expressed as an iRNA molecule in a cell or microorganism to inhibit target gene expression may comprise a single nucleotide sequence that is specifically complementary to all or part of a native nucleic acid sequence found in one or more target coleopteran pest species, or the DNA molecule can be constructed as a chimera from a plurality of such specifically complementary sequences.

In some embodiments, a nucleic acid molecule may comprise a first and a second nucleotide sequence separated by a “spacer sequence”. A spacer sequence may be a region comprising any sequence of nucleotides that facilitates secondary structure formation between the first and second nucleotide sequences, where this is desired. In one embodiment, the spacer sequence is part of a sense or antisense coding sequence for mRNA. The spacer sequence may alternatively comprise any combination of nucleotides or homologues thereof that are capable of being linked covalently to a nucleic acid molecule.

For example, in some embodiments, the DNA molecule may comprise a nucleotide sequence coding for one or more different RNA molecules, wherein each of the different RNA molecules comprises a first nucleotide sequence and a second nucleotide sequence, wherein the first and second nucleotide sequences are complementary to each other. The first and second nucleotide sequences may be connected within an RNA molecule by a spacer sequence. The spacer sequence may constitute part of the first nucleotide sequence or the second nucleotide sequence. Expression of an RNA molecule comprising the first and second nucleotide sequences may lead to the formation of a dsRNA molecule of the present invention, by specific base-pairing of the first and second nucleotide sequences. The first nucleotide sequence or the second nucleotide sequence may be substantially identical to a nucleic acid sequence native to a coleopteran pest (e.g., a target gene, or transcribed non-coding sequence), a derivative thereof, or a complementary sequence thereto.

dsRNA nucleic acid molecules comprise double strands of polymerized ribonucleotide sequences, and may include modifications to either the phosphate-sugar backbone or the nucleoside. Modifications in RNA structure may be tailored to allow specific inhibition. In one embodiment, dsRNA molecules may be modified through a ubiquitous enzymatic process so that siRNA molecules may be generated. This enzymatic process may utilize an RNase III enzyme, such as DICER in eukaryotes, either in vitro or in vivo. See Elbashir et al. (2001) Nature 411:494-498; and Hamilton and Baulcombe (1999) Science 286(5441):950-952. DICER or functionally-equivalent RNase III enzymes cleave larger dsRNA strands and/or hpRNA molecules into smaller oligonucleotides (e.g., siRNAs), each of which is about 19-25 nucleotides in length. The siRNA molecules produced by these enzymes have 2 to 3 nucleotide 3′ overhangs, and 5′ phosphate and 3′ hydroxyl termini. The siRNA molecules generated by RNase III enzymes are unwound and separated into single-stranded RNA in the cell. The siRNA molecules then specifically hybridize with RNA sequences transcribed from a target gene, and both RNA molecules are subsequently degraded by an inherent cellular RNA-degrading mechanism. This process may result in the effective degradation or removal of the RNA sequence encoded by the target gene in the target organism. The outcome is the post-transcriptional silencing of the targeted gene. In some embodiments, siRNA molecules produced by endogenous RNase III enzymes from heterologous nucleic acid molecules may efficiently mediate the down-regulation of target genes in coleopteran pests.

In some embodiments, a nucleic acid molecule of the invention may include at least one non-naturally occurring nucleotide sequence that can be transcribed into a single-stranded RNA molecule capable of forming a dsRNA molecule in vivo through intermolecular hybridization. Such dsRNA sequences typically self-assemble, and can be provided in the nutrition source of a coleopteran pest to achieve the post-transcriptional inhibition of a target gene. In these and further embodiments, a nucleic acid molecule of the invention may comprise two different non-naturally occurring nucleotide sequences, each of which is specifically complementary to a different target gene in a coleopteran pest. When such a nucleic acid molecule is provided as a dsRNA molecule to a coleopteran pest, the dsRNA molecule inhibits the expression of at least two different target genes in the coleopteran pest.

C. Obtaining Nucleic Acid Molecules

A variety of native sequences in coleopteran pests may be used as target sequences for the design of nucleic acid molecules of the invention, such as iRNAs and DNA molecules encoding iRNAs. Selection of native sequences is not, however, a straight-forward process. Only a small number of native sequences in the coleopteran pest will be effective targets. For example, it cannot be predicted with certainty whether a particular native sequence can be effectively down-regulated by nucleic acid molecules of the invention, or whether down-regulation of a particular native sequence will have a detrimental effect on the growth, viability, proliferation, and/or reproduction of the coleopteran pest. The vast majority of native coleopteran pest sequences, such as ESTs isolated therefrom (for example, as listed in U.S. Pat. Nos. 7,612,194 and 7,943,819), do not have a detrimental effect on the growth, viability, proliferation, and/or reproduction of the coleopteran pest, such as WCR or NCR. Neither is it predictable which of the native sequences which may have a detrimental effect on a coleopteran pest are able to be used in recombinant techniques for expressing nucleic acid molecules complementary to such native sequences in a host plant and providing the detrimental effect on the coleopteran pest upon feeding without causing harm to the host plant.

In some embodiments, nucleic acid molecules of the invention (e.g., dsRNA molecules to be provided in the host plant of a coleopteran pest) are selected to target cDNA sequences that encode proteins or parts of proteins essential for coleopteran pest survival, such as amino acid sequences involved in metabolic or catabolic biochemical pathways, cell division, reproduction, energy metabolism, digestion, host plant recognition, and the like. As described herein, ingestion of compositions by a target organism containing one or more dsRNAs, at least one segment of which is specifically complementary to at least a substantially identical segment of RNA produced in the cells of the target pest organism, can result in the death or other inhibition of the target. A nucleotide sequence, either DNA or RNA, derived from a coleopteran pest can be used to construct plant cells resistant to infestation by the coleopteran pests. The host plant of the coleopteran pest (e.g., Z. mays), for example, can be transformed to contain one or more of the nucleotide sequences derived from the coleopteran pest as provided herein. The nucleotide sequence transformed into the host may encode one or more RNAs that form into a dsRNA sequence in the cells or biological fluids within the transformed host, thus making the dsRNA available if/when the coleopteran pest forms a nutritional relationship with the transgenic host. This may result in the suppression of expression of one or more genes in the cells of the coleopteran pest, and ultimately death or inhibition of its growth or development.

Thus, in some embodiments, a gene is targeted that is essentially involved in the growth, development and reproduction of a coleopteran pest. Other target genes for use in the present invention may include, for example, those that play important roles in coleopteran pest viability, movement, migration, growth, development, infectivity, establishment of feeding sites and reproduction. A target gene may therefore be a housekeeping gene or a transcription factor. Additionally, a native coleopteran pest nucleotide sequence for use in the present invention may also be derived from a homolog (e.g., an ortholog), of a plant, viral, bacterial or insect gene, the function of which is known to those of skill in the art, and the nucleotide sequence of which is specifically hybridizable with a target gene in the genome of the target coleopteran pest. Methods of identifying a homolog of a gene with a known nucleotide sequence by hybridization are known to those of skill in the art.

In some embodiments, the invention provides methods for obtaining a nucleic acid molecule comprising a nucleotide sequence for producing an iRNA (e.g., dsRNA, siRNA, miRNA, shRNA, and hpRNA) molecule. One such embodiment comprises: (a) analyzing one or more target gene(s) for their expression, function, and phenotype upon dsRNA-mediated gene suppression in a coleopteran pest; (b) probing a cDNA or gDNA library with a probe comprising all or a portion of a nucleotide sequence or a homolog thereof from a targeted coleopteran pest that displays an altered (e.g., reduced) growth or development phenotype in a dsRNA-mediated suppression analysis; (c) identifying a DNA clone that specifically hybridizes with the probe; (d) isolating the DNA clone identified in step (b); (e) sequencing the cDNA or gDNA fragment that comprises the clone isolated in step (d), wherein the sequenced nucleic acid molecule comprises all or a substantial portion of the RNA sequence or a homolog thereof; and (f) chemically synthesizing all or a substantial portion of a gene sequence, or a siRNA or miRNA or shRNA or hpRNA or mRNA or dsRNA.

In further embodiments, a method for obtaining a nucleic acid fragment comprising a nucleotide sequence for producing a substantial portion of an iRNA (e.g., dsRNA, siRNA, miRNA, shRNA, and hpRNA) molecule includes: (a) synthesizing first and second oligonucleotide primers specifically complementary to a portion of a native nucleotide sequence from a targeted coleopteran pest; and (b) amplifying a cDNA or gDNA insert present in a cloning vector using the first and second oligonucleotide primers of step (a), wherein the amplified nucleic acid molecule comprises a substantial portion of a siRNA or miRNA or hpRNA or shRNA or mRNA or dsRNA molecule.

Nucleic acids of the invention can be isolated, amplified, or produced by a number of approaches. For example, an iRNA (e.g., dsRNA, siRNA, miRNA, shRNA, and hpRNA) molecule may be obtained by PCR amplification of a target nucleic acid sequence (e.g., a target gene or a target transcribed non-coding sequence) derived from a gDNA or cDNA library, or portions thereof. DNA or RNA may be extracted from a target organism, and nucleic acid libraries may be prepared therefrom using methods known to those ordinarily skilled in the art. gDNA or cDNA libraries generated from a target organism may be used for PCR amplification and sequencing of target genes. A confirmed PCR product may be used as a template for in vitro transcription to generate sense and antisense RNA with minimal promoters. Alternatively, nucleic acid molecules may be synthesized by any of a number of techniques (See, e.g., Ozaki et al. (1992) Nucleic Acids Research, 20: 5205-5214; and Agrawal et al. (1990) Nucleic Acids Research, 18: 5419-5423), including use of an automated DNA synthesizer (for example, a P. E. Biosystems, Inc. (Foster City, Calif.) model 392 or 394 DNA/RNA Synthesizer), using standard chemistries, such as phosphoramidite chemistry. See, e.g., Beaucage et al. (1992) Tetrahedron, 48: 2223-2311; U.S. Pat. Nos. 4,415,732, 4,458,066, 4,725,677, 4,973,679, and 4,980,460. Alternative chemistries resulting in non-natural backbone groups, such as phosphorothioate, phosphoramidate, and the like, can also be employed.

An RNA, dsRNA, siRNA, miRNA, shRNA, or hpRNA molecule of the present invention may be produced chemically or enzymatically by one skilled in the art through manual or automated reactions, or in vivo in a cell comprising a nucleic acid molecule comprising a sequence encoding the RNA, dsRNA, siRNA, miRNA, shRNA, or hpRNA molecule. RNA may also be produced by partial or total organic synthesis—any modified ribonucleotide can be introduced by in vitro enzymatic or organic synthesis. An RNA molecule may be synthesized by a cellular RNA polymerase or a bacteriophage RNA polymerase (e.g., T3 RNA polymerase, T7 RNA polymerase, and SP6 RNA polymerase). Expression constructs useful for the cloning and expression of nucleotide sequences are known in the art. See, e.g., U.S. Pat. Nos. 5,593,874, 5,693,512, 5,698,425, 5,712,135, 5,789,214, and 5,804,693. RNA molecules that are synthesized chemically or by in vitro enzymatic synthesis may be purified prior to introduction into a cell. For example, RNA molecules can be purified from a mixture by extraction with a solvent or resin, precipitation, electrophoresis, chromatography, or a combination thereof. Alternatively, RNA molecules that are synthesized chemically or by in vitro enzymatic synthesis may be used with no or a minimum of purification, for example, to avoid losses due to sample processing. The RNA molecules may be dried for storage or dissolved in an aqueous solution. The solution may contain buffers or salts to promote annealing, and/or stabilization of dsRNA molecule duplex strands.

In embodiments, a dsRNA molecule may be formed by a single self-complementary RNA strand or from two complementary RNA strands. dsRNA molecules may be synthesized either in vivo or in vitro. An endogenous RNA polymerase of the cell may mediate transcription of the one or two RNA strands in vivo, or cloned RNA polymerase may be used to mediate transcription in vivo or in vitro. Post-transcriptional inhibition of a target gene in a coleopteran pest may be host-targeted by specific transcription in an organ, tissue, or cell type of the host (e.g., by using a tissue-specific promoter); stimulation of an environmental condition in the host (e.g., by using an inducible promoter that is responsive to infection, stress, temperature, and/or chemical inducers); and/or engineering transcription at a developmental stage or age of the host (e.g., by using a developmental stage-specific promoter). RNA strands that form a dsRNA molecule, whether transcribed in vitro or in vivo, may or may not be polyadenylated, and may or may not be capable of being translated into a polypeptide by a cell's translational apparatus.

D. Recombinant Vectors and Host Cell Transformation

In some embodiments, the invention also provides a DNA molecule for introduction into a cell (e.g., a bacterial cell, a yeast cell, or a plant cell), wherein the DNA molecule comprises a nucleotide sequence that, upon expression to RNA and ingestion by a coleopteran pest, achieves suppression of a target gene in a cell, tissue, or organ of the coleopteran pest. Thus, some embodiments provide a recombinant nucleic acid molecule comprising a nucleic acid sequence capable of being expressed as an iRNA (e.g., dsRNA, siRNA, miRNA, shRNA, and hpRNA) molecule in a plant cell to inhibit target gene expression in a coleopteran pest. In order to initiate or enhance expression, such recombinant nucleic acid molecules may comprise one or more regulatory sequences, which regulatory sequences may be operably linked to the nucleic acid sequence capable of being expressed as an iRNA. Methods to express a gene suppression molecule in plants are known, and may be used to express a nucleotide sequence of the present invention. See, e.g., International PCT Publication No. WO06/073727; and U.S. Patent Publication No. 2006/0200878 A1.

In specific embodiments, a recombinant DNA molecule of the invention may comprise a nucleic acid sequence encoding a dsRNA molecule. Such recombinant DNA molecules may encode dsRNA molecules capable of inhibiting the expression of endogenous target gene(s) in a coleopteran pest cell upon ingestion. In many embodiments, a transcribed RNA may form a dsRNA molecule that may be provided in a stabilized form; e.g., as a hairpin and stem and loop structure.

In these and further embodiments, one strand of a dsRNA molecule may be formed by transcription from a nucleotide sequence which is substantially homologous to a nucleotide sequence consisting of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of SEQ ID NO:1; a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a fragment of at least 19 contiguous nucleotides of a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; the complement of a fragment of at least 19 contiguous nucleotides of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94; and the complement of a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94.

In particular embodiments, a recombinant DNA molecule encoding a dsRNA molecule may comprise at least two nucleotide sequence segments within a transcribed sequence, such sequences arranged such that the transcribed sequence comprises a first nucleotide sequence segment in a sense orientation, and a second nucleotide sequence segment (comprising the complement of the first nucleotide sequence segment) is in an antisense orientation, relative to at least one promoter, wherein the sense nucleotide sequence segment and the antisense nucleotide sequence segment are linked or connected by a spacer sequence segment of from about five (˜5) to about one thousand (˜1000) nucleotides. The spacer sequence segment may form a loop between the sense and antisense sequence segments. The sense nucleotide sequence segment or the antisense nucleotide sequence segment may be substantially homologous to the nucleotide sequence of a target gene (e.g., a gene comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94) or fragment thereof. In some embodiments, however, a recombinant DNA molecule may encode a dsRNA molecule without a spacer sequence. In embodiments, a sense coding sequence and an antisense coding sequence may be different lengths.

Sequences identified as having a deleterious effect on coleopteran pests or a plant-protective effect with regard to coleopteran pests may be readily incorporated into expressed dsRNA molecules through the creation of appropriate expression cassettes in a recombinant nucleic acid molecule of the invention. For example, such sequences may be expressed as a hairpin with stem and loop structure by taking a first segment corresponding to a target gene sequence (e.g., SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, and fragments thereof); linking this sequence to a second segment spacer region that is not homologous or complementary to the first segment; and linking this to a third segment, wherein at least a portion of the third segment is substantially complementary to the first segment. Such a construct forms a stem and loop structure by intramolecular base-pairing of the first segment with the third segment, wherein the loop structure forms and comprises the second segment. See, e.g., U.S. Patent Publication Nos. 2002/0048814 and 2003/0018993; and International PCT Publication Nos. WO94/01550 and WO98/05770. A dsRNA molecule may be generated, for example, in the form of a double-stranded structure such as a stem-loop structure (e.g., hairpin), whereby production of siRNA targeted for a native coleopteran pest sequence is enhanced by co-expression of a fragment of the targeted gene, for instance on an additional plant expressible cassette, that leads to enhanced siRNA production, or reduces methylation to prevent transcriptional gene silencing of the dsRNA hairpin promoter.

Embodiments of the invention include introduction of a recombinant nucleic acid molecule of the present invention into a plant (i.e., transformation) to achieve coleopteran pest-inhibitory levels of expression of one or more iRNA molecules. A recombinant DNA molecule may, for example, be a vector, such as a linear or a closed circular plasmid. The vector system may be a single vector or plasmid, or two or more vectors or plasmids that together contain the total DNA to be introduced into the genome of a host. In addition, a vector may be an expression vector. Nucleic acid sequences of the invention can, for example, be suitably inserted into a vector under the control of a suitable promoter that functions in one or more hosts to drive expression of a linked coding sequence or other DNA sequence. Many vectors are available for this purpose, and selection of the appropriate vector will depend mainly on the size of the nucleic acid to be inserted into the vector and the particular host cell to be transformed with the vector. Each vector contains various components depending on its function (e.g., amplification of DNA or expression of DNA) and the particular host cell with which it is compatible.

To impart coleopteran pest resistance to a transgenic plant, a recombinant DNA may, for example, be transcribed into an iRNA molecule (e.g., an RNA molecule that forms a dsRNA molecule) within the tissues or fluids of the recombinant plant. An iRNA molecule may comprise a nucleotide sequence that is substantially homologous and specifically hybridizable to a corresponding transcribed nucleotide sequence within a coleopteran pest that may cause damage to the host plant species. The coleopteran pest may contact the iRNA molecule that is transcribed in cells of the transgenic host plant, for example, by ingesting cells or fluids of the transgenic host plant that comprise the iRNA molecule. Thus, expression of a target gene is suppressed by the iRNA molecule within coleopteran pests that infest the transgenic host plant. In some embodiments, suppression of expression of the target gene in the target coleopteran pest may result in the plant being resistant to attack by the pest.

In order to enable delivery of iRNA molecules to a coleopteran pest in a nutritional relationship with a plant cell that has been transformed with a recombinant nucleic acid molecule of the invention, expression (i.e., transcription) of iRNA molecules in the plant cell is required. Thus, a recombinant nucleic acid molecule may comprise a nucleotide sequence of the invention operably linked to one or more regulatory sequences, such as a heterologous promoter sequence that functions in a host cell, such as a bacterial cell wherein the nucleic acid molecule is to be amplified, and a plant cell wherein the nucleic acid molecule is to be expressed.

Promoters suitable for use in nucleic acid molecules of the invention include those that are inducible, viral, synthetic, or constitutive, all of which are well known in the art. Non-limiting examples describing such promoters include U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. No. 6,232,526 (maize A3 promoter); U.S. Pat. No. 6,177,611 (constitutive maize promoters); U.S. Pat. Nos. 5,322,938, 5,352,605, 5,359,142, and 5,530,196 (CaMV 35S promoter); U.S. Pat. No. 6,433,252 (maize L3 oleosin promoter); U.S. Pat. No. 6,429,357 (rice actin 2 promoter, and rice actin 2 intron); U.S. Pat. No. 6,294,714 (light-inducible promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bidirectional promoters); U.S. Pat. No. 6,635,806 (gamma-coixin promoter); and U.S. Patent Publication No. 2009/757,089 (maize chloroplast aldolase promoter). Additional promoters include the nopaline synthase (NOS) promoter (Ebert et al. (1987) Proc. Natl. Acad. Sci. USA 84(16):5745-5749) and the octopine synthase (OCS) promoters (which are carried on tumor-inducing plasmids of Agrobacterium tumefaciens); the caulimovirus promoters such as the cauliflower mosaic virus (CaMV) 19S promoter (Lawton et al. (1987) Plant Mol. Biol. 9:315-324); the CaMV 35S promoter (Odell et al. (1985) Nature 313:810-812; the figwort mosaic virus 35S-promoter (Walker et al. (1987) Proc. Natl. Acad. Sci. USA 84(19):6624-6628); the sucrose synthase promoter (Yang and Russell (1990) Proc. Natl. Acad. Sci. USA 87:4144-4148); the R gene complex promoter (Chandler et al. (1989) Plant Cell 1:1175-1183); the chlorophyll a/b binding protein gene promoter; CaMV 35S (U.S. Pat. Nos. 5,322,938, 5,352,605, 5,359,142, and 5,530,196); FMV 35S (U.S. Pat. Nos. 5,378,619 and 6,051,753); a PC1SV promoter (U.S. Pat. No. 5,850,019); the SCP1 promoter (U.S. Pat. No. 6,677,503); and AGRtu.nos promoters (GenBank™ Accession No. V00087; Depicker et al. (1982) J. Mol. Appl. Genet. 1:561-573; Bevan et al. (1983) Nature 304:184-187).

In particular embodiments, nucleic acid molecules of the invention comprise a tissue-specific promoter, such as a root-specific promoter. Root-specific promoters drive expression of operably-linked coding sequences exclusively or preferentially in root tissue. Examples of root-specific promoters are known in the art. See, e.g., U.S. Pat. Nos. 5,110,732; 5,459,252 and 5,837,848; and Opperman et al. (1994) Science 263:221-3; and Hirel et al. (1992) Plant Mol. Biol. 20:207-18. In some embodiments, a nucleotide sequence or fragment for coleopteran pest control according to the invention may be cloned between two root-specific promoters oriented in opposite transcriptional directions relative to the nucleotide sequence or fragment, and which are operable in a transgenic plant cell and expressed therein to produce RNA molecules in the transgenic plant cell that subsequently may form dsRNA molecules, as described, supra. The iRNA molecules expressed in plant tissues may be ingested by a coleopteran pest so that suppression of target gene expression is achieved.

Additional regulatory sequences that may optionally be operably linked to a nucleic acid molecule of interest include 5′UTRs that function as a translation leader sequence located between a promoter sequence and a coding sequence. The translation leader sequence is present in the fully-processed mRNA, and it may affect processing of the primary transcript, and/or RNA stability. Examples of translation leader sequences include maize and petunia heat shock protein leaders (U.S. Pat. No. 5,362,865), plant virus coat protein leaders, plant rubisco leaders, and others. See, e.g., Turner and Foster (1995) Molecular Biotech. 3(3):225-36. Non-limiting examples of 5′UTRs include GmHsp (U.S. Pat. No. 5,659,122); PhDnaK (U.S. Pat. No. 5,362,865); AtAnt1; TEV (Carrington and Freed (1990) J. Virol. 64:1590-7); and AGRtunos (GenBank™ Accession No. V00087; and Bevan et al. (1983) Nature 304:184-7).

Additional regulatory sequences that may optionally be operably linked to a nucleic acid molecule of interest also include 3′ non-translated sequences, 3′ transcription termination regions, or poly-adenylation regions. These are genetic elements located downstream of a nucleotide sequence, and include polynucleotides that provide polyadenylation signal, and/or other regulatory signals capable of affecting transcription or mRNA processing. The polyadenylation signal functions in plants to cause the addition of polyadenylate nucleotides to the 3′ end of the mRNA precursor. The polyadenylation sequence can be derived from a variety of plant genes, or from T-DNA genes. A non-limiting example of a 3′ transcription termination region is the nopaline synthase 3′ region (nos 3′; Fraley et al. (1983) Proc. Natl. Acad. Sci. USA 80:4803-7). An example of the use of different 3′ nontranslated regions is provided in Ingelbrecht et al., (1989) Plant Cell 1:671-80. Non-limiting examples of polyadenylation signals include one from a Pisum sativum RbcS2 gene (Ps.RbcS2-E9; Coruzzi et al. (1984) EMBO J. 3:1671-9) and AGRtu.nos (GenBank™ Accession No. E01312).

Some embodiments may include a plant transformation vector that comprises an isolated and purified DNA molecule comprising at least one of the above-described regulatory sequences operatively linked to one or more nucleotide sequences of the present invention. When expressed, the one or more nucleotide sequences result in one or more RNA molecule(s) comprising a nucleotide sequence that is specifically complementary to all or part of a native RNA molecule in a coleopteran pest. Thus, the nucleotide sequence(s) may comprise a segment encoding all or part of a ribonucleotide sequence present within a targeted coleopteran pest RNA transcript, and may comprise inverted repeats of all or a part of a targeted coleopteran pest transcript. A plant transformation vector may contain sequences specifically complementary to more than one target sequence, thus allowing production of more than one dsRNA for inhibiting expression of two or more genes in cells of one or more populations or species of target coleopteran pests. Segments of nucleotide sequence specifically complementary to nucleotide sequences present in different genes can be combined into a single composite nucleic acid molecule for expression in a transgenic plant. Such segments may be contiguous or separated by a spacer sequence.

In some embodiments, a plasmid of the present invention already containing at least one nucleotide sequence(s) of the invention can be modified by the sequential insertion of additional nucleotide sequence(s) in the same plasmid, wherein the additional nucleotide sequence(s) are operably linked to the same regulatory elements as the original at least one nucleotide sequence(s). In some embodiments, a nucleic acid molecule may be designed for the inhibition of multiple target genes. In some embodiments, the multiple genes to be inhibited can be obtained from the same coleopteran pest species, which may enhance the effectiveness of the nucleic acid molecule. In other embodiments, the genes can be derived from different coleopteran pests, which may broaden the range of coleopteran pests against which the agent(s) is/are effective. When multiple genes are targeted for suppression or a combination of expression and suppression, a polycistronic DNA element can be fabricated.

A recombinant nucleic acid molecule or vector of the present invention may comprise a selectable marker that confers a selectable phenotype on a transformed cell, such as a plant cell. Selectable markers may also be used to select for plants or plant cells that comprise a recombinant nucleic acid molecule of the invention. The marker may encode biocide resistance, antibiotic resistance (e.g., kanamycin, Geneticin (G418), bleomycin, hygromycin, etc.), or herbicide tolerance (e.g., glyphosate, etc.). Examples of selectable markers include, but are not limited to: a neo gene which codes for kanamycin resistance and can be selected for using kanamycin, G418, etc.; a bar gene which codes for bialaphos resistance; a mutant EPSP synthase gene which encodes glyphosate tolerance; a nitrilase gene which confers resistance to bromoxynil; a mutant acetolactate synthase (ALS) gene which confers imidazolinone or sulfonylurea tolerance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, streptomycin and tetracycline, and the like. Examples of such selectable markers are illustrated in, e.g., U.S. Pat. Nos. 5,550,318; 5,633,435; 5,780,708 and 6,118,047.

A recombinant nucleic acid molecule or vector of the present invention may also include a screenable marker. Screenable markers may be used to monitor expression. Exemplary screenable markers include a 0-glucuronidase or uidA gene (GUS) which encodes an enzyme for which various chromogenic substrates are known (Jefferson et al. (1987) Plant Mol. Biol. Rep. 5:387-405); an R-locus gene, which encodes a product that regulates the production of anthocyanin pigments (red color) in plant tissues (Dellaporta et al. (1988) “Molecular cloning of the maize R-nj allele by transposon tagging with Ac.” In 18^{th} Stadler Genetics Symposium, P. Gustafson and R. Appels, eds. (New York: Plenum), pp. 263-82); a β-lactamase gene (Sutcliffe et al. (1978) Proc. Natl. Acad. Sci. USA 75:3737-41); a gene which encodes an enzyme for which various chromogenic substrates are known (e.g., PADAC, a chromogenic cephalosporin); a luciferase gene (Ow et al. (1986) Science 234:856-9); an xylE gene that encodes a catechol dioxygenase that can convert chromogenic catechols (Zukowski et al. (1983) Gene 46(2-3):247-55); an amylase gene (Ikatu et al. (1990) Bio/Technol. 8:241-2); a tyrosinase gene which encodes an enzyme capable of oxidizing tyrosine to DOPA and dopaquinone which in turn condenses to melanin (Katz et al. (1983) J. Gen. Microbiol. 129:2703-14); and an α-galactosidase.

In some embodiments, recombinant nucleic acid molecules, as described, supra, may be used in methods for the creation of transgenic plants and expression of heterologous nucleic acids in plants to prepare transgenic plants that exhibit reduced susceptibility to coleopteran pests. Plant transformation vectors can be prepared, for example, by inserting nucleic acid molecules encoding iRNA molecules into plant transformation vectors and introducing these into plants.

Suitable methods for transformation of host cells include any method by which DNA can be introduced into a cell, such as by transformation of protoplasts (See, e.g., U.S. Pat. No. 5,508,184), by desiccation/inhibition-mediated DNA uptake (See, e.g., Potrykus et al. (1985) Mol. Gen. Genet. 199:183-8), by electroporation (See, e.g., U.S. Pat. No. 5,384,253), by agitation with silicon carbide fibers (See, e.g., U.S. Pat. Nos. 5,302,523 and 5,464,765), by Agrobacterium-mediated transformation (See, e.g., U.S. Pat. Nos. 5,563,055; 5,591,616; 5,693,512; 5,824,877; 5,981,840; and 6,384,301) and by acceleration of DNA-coated particles (See, e.g., U.S. Pat. Nos. 5,015,580, 5,550,318, 5,538,880, 6,160,208, 6,399,861, and 6,403,865), etc. Techniques that are particularly useful for transforming corn are described, for example, in U.S. Pat. Nos. 5,591,616, 7,060,876 and 7,939,3281. Through the application of techniques such as these, the cells of virtually any species may be stably transformed. In some embodiments, transforming DNA is integrated into the genome of the host cell. In the case of multicellular species, transgenic cells may be regenerated into a transgenic organism. Any of these techniques may be used to produce a transgenic plant, for example, comprising one or more nucleic acid sequences encoding one or more iRNA molecules in the genome of the transgenic plant.

The most widely utilized method for introducing an expression vector into plants is based on the natural transformation system of various Agrobacterium species. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. The Ti (tumor-inducing)-plasmids contain a large segment, known as T-DNA, which is transferred to transformed plants. Another segment of the Ti plasmid, the Vir region, is responsible for T-DNA transfer. The T-DNA region is bordered by terminal repeats. In modified binary vectors, the tumor-inducing genes have been deleted, and the functions of the Vir region are utilized to transfer foreign DNA bordered by the T-DNA border sequences. The T-region may also contain a selectable marker for efficient recovery of transgenic cells and plants, and a multiple cloning site for inserting sequences for transfer such as a dsRNA encoding nucleic acid.

Thus, in some embodiments, a plant transformation vector is derived from a Ti plasmid of A. tumefaciens (See, e.g., U.S. Pat. Nos. 4,536,475, 4,693,977, 4,886,937, and 5,501,967; and European Patent No. EP 0 122 791) or a Ri plasmid of A. rhizogenes. Additional plant transformation vectors include, for example and without limitation, those described by Herrera-Estrella et al. (1983) Nature 303:209-13; Bevan et al. (1983) Nature 304:184-7; Klee et al. (1985) Bio/Technol. 3:637-42; and in European Patent No. EP 0 120 516, and those derived from any of the foregoing. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector.

After providing exogenous DNA to recipient cells, transformed cells are generally identified for further culturing and plant regeneration. In order to improve the ability to identify transformed cells, one may desire to employ a selectable or screenable marker gene, as previously set forth, with the transformation vector used to generate the transformant. In the case where a selectable marker is used, transformed cells are identified within the potentially transformed cell population by exposing the cells to a selective agent or agents. In the case where a screenable marker is used, cells may be screened for the desired marker gene trait.

Cells that survive the exposure to the selective agent, or cells that have been scored positive in a screening assay, may be cultured in media that supports regeneration of plants. In some embodiments, any suitable plant tissue culture media (e.g., MS and N6 media) may be modified by including further substances, such as growth regulators. Tissue may be maintained on a basic medium with growth regulators until sufficient tissue is available to begin plant regeneration efforts, or following repeated rounds of manual selection, until the morphology of the tissue is suitable for regeneration (e.g., typically about 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturation.

To confirm the presence of a nucleic acid molecule of interest (for example, a DNA sequence encoding one or more iRNA molecules that inhibit target gene expression in a coleopteran pest) in the regenerating plants, a variety of assays may be performed. Such assays include, for example: molecular biological assays, such as Southern and northern blotting, PCR, and nucleic acid sequencing; biochemical assays, such as detecting the presence of a protein product, e.g., by immunological means (ELISA and/or immuno blots) or by enzymatic function; plant part assays, such as leaf or root assays; and analysis of the phenotype of the whole regenerated plant.

Integration events may be analyzed, for example, by PCR amplification using, e.g., oligonucleotide primers specific for a nucleic acid molecule of interest. PCR genotyping is understood to include, but not be limited to, polymerase-chain reaction (PCR) amplification of genomic DNA derived from isolated host plant callus tissue predicted to contain a nucleic acid molecule of interest integrated into the genome, followed by standard cloning and sequence analysis of PCR amplification products. Methods of PCR genotyping have been well described (for example, Rios, G. et al. (2002) Plant J. 32:243-53) and may be applied to genomic DNA derived from any plant species (e.g., Z. mays) or tissue type, including cell cultures.

A transgenic plant formed using Agrobacterium-dependent transformation methods typically contains a single recombinant DNA sequence inserted into one chromosome. The single recombinant DNA sequence is referred to as a “transgenic event” or “integration event”. Such transgenic plants are hemizygous for the inserted exogenous sequence. In some embodiments, a transgenic plant homozygous with respect to a transgene may be obtained by sexually mating (selfing) an independent segregant transgenic plant that contains a single exogenous gene sequence to itself, for example a T₀ plant, to produce T₁ seed. One fourth of the T₁ seed produced will be homozygous with respect to the transgene. Germinating T₁ seed results in plants that can be tested for heterozygosity, typically using an SNP assay or a thermal amplification assay that allows for the distinction between heterozygotes and homozygotes (i.e., a zygosity assay).

In particular embodiments, at least 2, 3, 4, 5, 6, 7, 8, 9 or 10 or more different iRNA molecules that have a coleopteran pest-inhibitory effect are produced in a plant cell. The iRNA molecules (e.g., dsRNA molecules) may be expressed from multiple nucleic acid sequences introduced in different transformation events, or from a single nucleic acid sequence introduced in a single transformation event. In some embodiments, a plurality of iRNA molecules are expressed under the control of a single promoter. In other embodiments, a plurality of iRNA molecules are expressed under the control of multiple promoters. Single iRNA molecules may be expressed that comprise multiple nucleic acid sequences that are each homologous to different loci within one or more coleopteran pests (for example, the locus defined by SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94), both in different populations of the same species of coleopteran pest, or in different species of coleopteran pests.

In addition to direct transformation of a plant with a recombinant nucleic acid molecule, transgenic plants can be prepared by crossing a first plant having at least one transgenic event with a second plant lacking such an event. For example, a recombinant nucleic acid molecule comprising a nucleotide sequence that encodes an iRNA molecule may be introduced into a first plant line that is amenable to transformation to produce a transgenic plant, which transgenic plant may be crossed with a second plant line to introgress the nucleotide sequence that encodes the iRNA molecule into the second plant line.

The invention also includes commodity products containing one or more of the sequences of the present invention. Particular embodiments include commodity products produced from a recombinant plant or seed containing one or more of the nucleotide sequences of the present invention. A commodity product containing one or more of the sequences of the present invention is intended to include, but not be limited to, meals, oils, crushed or whole grains or seeds of a plant, or any food or animal feed product comprising any meal, oil, or crushed or whole grain of a recombinant plant or seed containing one or more of the sequences of the present invention. The detection of one or more of the sequences of the present invention in one or more commodity or commodity products contemplated herein is de facto evidence that the commodity or commodity product is produced from a transgenic plant designed to express one or more of the nucleotides sequences of the present invention for the purpose of controlling coleopteran plant pests using dsRNA-mediated gene suppression methods.

In some aspects, seeds and commodity products produced by transgenic plants derived from transformed plant cells are included, wherein the seeds or commodity products comprise a detectable amount of a nucleic acid sequence of the invention. In some embodiments, such commodity products may be produced, for example, by obtaining transgenic plants and preparing food or feed from them. Commodity products comprising one or more of the nucleic acid sequences of the invention includes, for example and without limitation: meals, oils, crushed or whole grains or seeds of a plant, and any food product comprising any meal, oil, or crushed or whole grain of a recombinant plant or seed comprising one or more of the nucleic acid sequences of the invention. The detection of one or more of the sequences of the invention in one or more commodity or commodity products is de facto evidence that the commodity or commodity product is produced from a transgenic plant designed to express one or more of the iRNA molecules of the invention for the purpose of controlling coleopteran pests.

In some embodiments, a transgenic plant or seed comprising a nucleic acid molecule of the invention also may comprise at least one other transgenic event in its genome, including without limitation: a transgenic event from which is transcribed an iRNA molecule targeting a locus in a coleopteran pest other than the one defined by SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94, such as, for example, one or more loci selected from the group consisting of Caf1-180 (U.S. Patent Application Publication No. 2012/0174258), VatpaseC (U.S. Patent Application Publication No. 2012/0174259), Rho1 (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), ROP (U.S. patent application Ser. No. 14/577,811), RNAPII (U.S. patent application Ser. No. 14/577,854), RPS6 (U.S. Patent Application Publication No. 2013/0097730); RNA polymerase I (U.S. Patent Application No. 62/133,214), RNA polymerase II-33 (U.S. Patent Application No. 62/133,210), ncm (U.S. Patent Application No. 62/095,487), Dre4 (U.S. patent application Ser. No. 14/705,807), COPI alpha (U.S. Patent Application No. 62/063,199), COPI beta (U.S. Patent Application No. 62/063,203), COPI gamma (U.S. Patent Application No. 62/063,192), COPI delta (U.S. Patent Application No. 62/063,216), and RNA polymerase II215 (U.S. Patent Application No. 62/133,202); a transgenic event from which is transcribed an iRNA molecule targeting a gene in an organism other than a coleopteran pest (e.g., a plant-parasitic nematode); a gene encoding an insecticidal protein (e.g., a Bacillus thuringiensis insecticidal protein, such as, for example, Cry34Ab1 (U.S. Pat. Nos. 6,127,180, 6,340,593, and 6,624,145), Cry35Ab1 (U.S. Pat. Nos. 6,083,499, 6,340,593, and 6,548,291), a “Cry34/35Ab1” combination in a single event (e.g., maize event DAS-59122-7; U.S. Pat. No. 7,323,556), Cry3A (e.g., U.S. Pat. No. 7,230,167), Cry3B (e.g., U.S. Pat. No. 8,101,826), Cry6A (e.g., U.S. Pat. No. 6,831,062), and combinations thereof (e.g., U.S. Patent Application Nos. 2013/0167268, 2013/0167269, and 2013/0180016); an herbicide tolerance gene (e.g., a gene providing tolerance to glyphosate, glufosinate, dicamba or 2,4-D (e.g., U.S. Pat. No. 7,838,733)); and a gene contributing to a desirable phenotype in the transgenic plant, such as increased yield, altered fatty acid metabolism, or restoration of cytoplasmic male sterility. In particular embodiments, sequences encoding iRNA molecules of the invention may be combined with other insect control or with disease resistance traits in a plant to achieve desired traits for enhanced control of insect damage and plant disease. Combining insect control traits that employ distinct modes-of-action may provide protected transgenic plants with superior durability over plants harboring a single control trait, for example, because of the reduced probability that resistance to the trait(s) will develop in the field.

### V. Target Gene Suppression in a Coleopteran Pest

A. Overview

In some embodiments of the invention, at least one nucleic acid molecule useful for the control of coleopteran pests may be provided to a coleopteran pest, wherein the nucleic acid molecule leads to RNAi-mediated gene silencing in the coleopteran pest. In particular embodiments, an iRNA molecule (e.g., dsRNA, siRNA, miRNA, shRNA, and hpRNA) may be provided to the coleopteran pest. In some embodiments, a nucleic acid molecule useful for the control of coleopteran pests may be provided to a coleopteran pest by contacting the nucleic acid molecule with the coleopteran pest. In these and further embodiments, a nucleic acid molecule useful for the control of coleopteran pests may be provided in a feeding substrate of the coleopteran pest, for example, a nutritional composition. In these and further embodiments, a nucleic acid molecule useful for the control of coleopteran pests may be provided through ingestion of plant material comprising the nucleic acid molecule that is ingested by the coleopteran pest. In certain embodiments, the nucleic acid molecule is present in plant material through expression of a recombinant nucleic acid sequence introduced into the plant material, for example, by transformation of a plant cell with a vector comprising the recombinant nucleic acid sequence and regeneration of a plant material or whole plant from the transformed plant cell.

B. RNAi-Mediated Target Gene Suppression

In embodiments, the invention provides iRNA molecules (e.g., dsRNA, siRNA, miRNA, shRNA, and hpRNA) that may be designed to target essential native nucleotide sequences (e.g., essential genes) in the transcriptome of a coleopteran pest (e.g., WCR or NCR), for example by designing an iRNA molecule that comprises at least one strand comprising a nucleotide sequence that is specifically complementary to the target sequence. The sequence of an iRNA molecule so designed may be identical to the target sequence, or may incorporate mismatches that do not prevent specific hybridization between the iRNA molecule and its target sequence.

iRNA molecules of the invention may be used in methods for gene suppression in a coleopteran pest, thereby reducing the level or incidence of damage caused by the pest on a plant (for example, a protected transformed plant comprising an iRNA molecule). As used herein the term “gene suppression” refers to any of the well-known methods for reducing the levels of protein produced as a result of gene transcription to mRNA and subsequent translation of the mRNA, including the reduction of protein expression from a gene or a coding sequence including post-transcriptional inhibition of expression and transcriptional suppression. Post-transcriptional inhibition is mediated by specific homology between all or a part of an mRNA transcribed from a gene targeted for suppression and the corresponding iRNA molecule used for suppression. Additionally, post-transcriptional inhibition refers to the substantial and measurable reduction of the amount of mRNA available in the cell for binding by ribosomes.

In embodiments wherein an iRNA molecule is a dsRNA molecule, the dsRNA molecule may be cleaved by the enzyme, DICER, into siRNA molecules (approximately 20 nucleotides in length). The double-stranded siRNA molecule generated by DICER activity upon the dsRNA molecule may be separated into two single-stranded siRNAs; the “passenger strand” and the “guide strand”. The passenger strand may be degraded, and the guide strand may be incorporated into RISC. Post-transcriptional inhibition occurs by specific hybridization of the guide strand with a specifically complementary sequence of an mRNA molecule, and subsequent cleavage by the enzyme, Argonaute (catalytic component of RISC).

In embodiments of the invention, any form of iRNA molecule may be used. Those of skill in the art will understand that dsRNA molecules typically are more stable than are single-stranded RNA molecules, during preparation and during the step of providing the iRNA molecule to a cell, and are typically also more stable in a cell.

In particular embodiments, a nucleic acid molecule is provided that comprises a nucleotide sequence, which nucleotide sequence may be expressed in vitro to produce an iRNA molecule that is substantially homologous to a nucleic acid molecule encoded by a nucleotide sequence within the genome of a coleopteran pest. In certain embodiments, the in vitro transcribed iRNA molecule may be a stabilized dsRNA molecule that comprises a stem-loop structure. After a coleopteran pest contacts the in vitro transcribed iRNA molecule, post-transcriptional inhibition of a target gene in the coleopteran pest (for example, an essential gene) may occur.

In some embodiments of the invention, expression of a nucleic acid molecule comprising at least 19 contiguous nucleotides of a nucleotide sequence is used in a method for post-transcriptional inhibition of a target gene in a coleopteran pest, wherein the nucleotide sequence is selected from the group consisting of: SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a fragment of at least 19 contiguous nucleotides of a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a fragment of at least 19 contiguous nucleotides of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; and the complement of a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94. In certain embodiments, expression of a nucleic acid molecule that is at least 80% identical (e.g., 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, and 100%) with any of the foregoing may be used. In these and further embodiments, a nucleic acid molecule may be expressed that specifically hybridizes to an RNA molecule present in at least one cell of a coleopteran pest.

In some embodiments, expression of at least one nucleic acid molecule comprising at least 19 contiguous nucleotides of a nucleotide sequence may be used in a method for post-transcriptional inhibition of a target gene in a coleopteran pest, wherein the nucleotide sequence is selected from the group consisting of: SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a fragment of at least 19 contiguous nucleotides of SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a fragment of at least 19 contiguous nucleotides of a native coding sequence of a Diabrotica organism (e.g., WCR) comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; the complement of a fragment of at least 19 contiguous nucleotides of a native coding sequence of a Diabrotica organism comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94; and the complement of a fragment of at least 19 contiguous nucleotides of a native non-coding sequence of a Diabrotica organism that is transcribed into a native RNA molecule comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, and SEQ ID NO:94. In certain embodiments, expression of a nucleic acid molecule that is at least 80% identical (e.g., 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, and 100%) with any of the foregoing may be used. In these and further embodiments, a nucleic acid molecule may be expressed that specifically hybridizes to an RNA molecule present in at least one cell of a coleopteran pest. In particular examples, such a nucleic acid molecule may comprise a nucleotide sequence comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94.

It is an important feature of some embodiments of the invention that the RNAi post-transcriptional inhibition system is able to tolerate sequence variations among target genes that might be expected due to genetic mutation, strain polymorphism, or evolutionary divergence. The introduced nucleic acid molecule may not need to be absolutely homologous to either a primary transcription product or a fully-processed mRNA of a target gene, so long as the introduced nucleic acid molecule is specifically hybridizable to either a primary transcription product or a fully-processed mRNA of the target gene. Moreover, the introduced nucleic acid molecule may not need to be full-length, relative to either a primary transcription product or a fully processed mRNA of the target gene.

Inhibition of a target gene using the iRNA technology of the present invention is sequence-specific; i.e., nucleotide sequences substantially homologous to the iRNA molecule(s) are targeted for genetic inhibition. In some embodiments, an RNA molecule comprising a nucleotide sequence identical to a portion of a target gene sequence may be used for inhibition. In these and further embodiments, an RNA molecule comprising a nucleotide sequence with one or more insertion, deletion, and/or point mutations relative to a target gene sequence may be used. In particular embodiments, an iRNA molecule and a portion of a target gene may share, for example, at least from about 80%, at least from about 81%, at least from about 82%, at least from about 83%, at least from about 84%, at least from about 85%, at least from about 86%, at least from about 87%, at least from about 88%, at least from about 89%, at least from about 90%, at least from about 91%, at least from about 92%, at least from about 93%, at least from about 94%, at least from about 95%, at least from about 96%, at least from about 97%, at least from about 98%, at least from about 99%, at least from about 100%, and 100% sequence identity. Alternatively, the duplex region of a dsRNA molecule may be specifically hybridizable with a portion of a target gene transcript. In specifically hybridizable molecules, a less than full length sequence exhibiting a greater homology compensates for a longer, less homologous sequence. The length of the nucleotide sequence of a duplex region of a dsRNA molecule that is identical to a portion of a target gene transcript may be at least about 19, 20, 21, 22, 23, 24, 25, 50, 100, 200, 300, 400, 500, or at least about 1000 bases. In some embodiments, a sequence of greater than 20 to 100 nucleotides may be used. In particular embodiments, a sequence of greater than about 200 to 300 nucleotides may be used. In particular embodiments, a sequence of greater than about 500 to 1000 nucleotides may be used, depending on the size of the target gene.

In certain embodiments, expression of a target gene in a coleopteran pest may be inhibited by at least 10%; at least 33%; at least 50%; or at least 80% within a cell of the coleopteran pest, such that a significant inhibition takes place. Significant inhibition refers to inhibition over a threshold that results in a detectable phenotype (e.g., cessation of growth, cessation of feeding, cessation of development, induced mortality, etc.), or a detectable decrease in RNA and/or gene product corresponding to the target gene being inhibited. Although in certain embodiments of the invention inhibition occurs in substantially all cells of the coleopteran pest, in other embodiments inhibition occurs only in a subset of cells expressing the target gene.

In some embodiments, transcriptional suppression in a cell is mediated by the presence of a dsRNA molecule exhibiting substantial sequence identity to a promoter DNA sequence or the complement thereof, to effect what is referred to as “promoter trans suppression”. Gene suppression may be effective against target genes in a coleopteran pest that may ingest or contact such dsRNA molecules, for example, by ingesting or contacting plant material containing the dsRNA molecules. dsRNA molecules for use in promoter trans suppression may be specifically designed to inhibit or suppress the expression of one or more homologous or complementary sequences in the cells of the coleopteran pest. Post-transcriptional gene suppression by antisense or sense oriented RNA to regulate gene expression in plant cells is disclosed in U.S. Pat. Nos. 5,107,065, 5,231,020, 5,283,184, and 5,759,829.

C. Expression of iRNA Molecules Provided to a Coleopteran Pest

Expression of iRNA molecules for RNAi-mediated gene inhibition in a coleopteran pest may be carried out in any one of many in vitro or in vivo formats. The iRNA molecules may then be provided to a coleopteran pest, for example, by contacting the iRNA molecules with the pest, or by causing the pest to ingest or otherwise internalize the iRNA molecules. Some embodiments of the invention include transformed host plants of a coleopteran pest, transformed plant cells, and progeny of transformed plants. The transformed plant cells and transformed plants may be engineered to express one or more of the iRNA molecules, for example, under the control of a heterologous promoter, to provide a pest-protective effect. Thus, when a transgenic plant or plant cell is consumed by a coleopteran pest during feeding, the pest may ingest iRNA molecules expressed in the transgenic plants or cells. The nucleotide sequences of the present invention may also be introduced into a wide variety of prokaryotic and eukaryotic microorganism hosts to produce iRNA molecules. The term “microorganism” includes prokaryotic and eukaryotic species, such as bacteria and fungi.

Modulation of gene expression may include partial or complete suppression of such expression. In another embodiment, a method for suppression of gene expression in a coleopteran pest comprises providing in the tissue of the host of the pest a gene-suppressive amount of at least one dsRNA molecule formed following transcription of a nucleotide sequence as described herein, at least one segment of which is complementary to an mRNA sequence within the cells of the coleopteran pest. A dsRNA molecule, including its modified form such as an siRNA, miRNA, shRNA, or hpRNA molecule, ingested by a coleopteran pest in accordance with the invention, may be at least from about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or 100% identical to an RNA molecule transcribed from a nucleic acid molecule comprising a nucleotide sequence comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45, SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94. Isolated and substantially purified nucleic acid molecules including, but not limited to, non-naturally occurring nucleotide sequences and recombinant DNA constructs for providing dsRNA molecules of the present invention are therefore provided, which suppress or inhibit the expression of an endogenous coding sequence or a target coding sequence in the coleopteran pest when introduced thereto.

Particular embodiments provide a delivery system for the delivery of iRNA molecules for the post-transcriptional inhibition of one or more target gene(s) in a coleopteran plant pest and control of a population of the coleopteran plant pest. In some embodiments, the delivery system comprises ingestion of a host transgenic plant cell or contents of the host cell comprising RNA molecules transcribed in the host cell. In these and further embodiments, a transgenic plant cell or a transgenic plant is created that contains a recombinant DNA construct providing a stabilized dsRNA molecule of the invention. Transgenic plant cells and transgenic plants comprising nucleic acid sequences encoding a particular iRNA molecule may be produced by employing recombinant DNA technologies (which basic technologies are well-known in the art) to construct a plant transformation vector comprising a nucleotide sequence encoding an iRNA molecule of the invention (e.g., a stabilized dsRNA molecule); to transform a plant cell or plant; and to generate the transgenic plant cell or the transgenic plant that contains the transcribed iRNA molecule.

To impart coleopteran pest resistance to a transgenic plant, a recombinant DNA molecule may, for example, be transcribed into an iRNA molecule, such as a dsRNA molecule, an siRNA molecule, an miRNA molecule, a shRNA molecule, or an hpRNA molecule. In some embodiments, an RNA molecule transcribed from a recombinant DNA molecule may form a dsRNA molecule within the tissues or fluids of the recombinant plant. Such a dsRNA molecule may be comprised in part of a nucleotide sequence that is identical to a corresponding nucleotide sequence transcribed from a DNA sequence within a coleopteran pest of a type that may infest the host plant. Expression of a target gene within the coleopteran pest is suppressed by the ingested dsRNA molecule, and the suppression of expression of the target gene in the coleopteran pest results in, for example, cessation of feeding by the coleopteran pest, with an ultimate result being, for example, that the transgenic plant is protected from further damage by the coleopteran pest. The modulatory effects of dsRNA molecules have been shown to be applicable to a variety of genes expressed in pests, including, for example, endogenous genes responsible for cellular metabolism or cellular transformation, including house-keeping genes; transcription factors; molting-related genes; and other genes which encode polypeptides involved in cellular metabolism or normal growth and development.

For transcription from a transgene in vivo or an expression construct, a regulatory region (e.g., promoter, enhancer, silencer, and polyadenylation signal) may be used in some embodiments to transcribe the RNA strand (or strands). Therefore, in some embodiments, as set forth, supra, a nucleotide sequence for use in producing iRNA molecules may be operably linked to one or more promoter sequences functional in a plant host cell. The promoter may be an endogenous promoter, normally resident in the host genome. The nucleotide sequence of the present invention, under the control of an operably linked promoter sequence, may further be flanked by additional sequences that advantageously affect its transcription and/or the stability of a resulting transcript. Such sequences may be located upstream of the operably linked promoter, downstream of the 3′ end of the expression construct, and may occur both upstream of the promoter and downstream of the 3′ end of the expression construct.

Some embodiments provide methods for reducing the damage to a host plant (e.g., a corn plant) caused by a coleopteran pest that feeds on the plant, wherein the method comprises providing in the host plant a transformed plant cell expressing at least one nucleic acid molecule of the invention, wherein the nucleic acid molecule(s) functions upon being taken up by the coleopteran pest to inhibit the expression of a target sequence within the coleopteran pest, which inhibition of expression results in mortality, reduced growth, and/or reduced reproduction of the coleopteran pest, thereby reducing the damage to the host plant caused by the coleopteran pest. In some embodiments, the nucleic acid molecule(s) comprise dsRNA molecules. In these and further embodiments, the nucleic acid molecule(s) comprise dsRNA molecules that each comprise more than one nucleotide sequence that is specifically hybridizable to a nucleic acid molecule expressed in a coleopteran pest cell. In some embodiments, the nucleic acid molecule(s) consist of one nucleotide sequence that is specifically hybridizable to a nucleic acid molecule expressed in a coleopteran pest cell.

In some embodiments, a method for increasing the yield of a corn crop is provided, wherein the method comprises introducing into a corn plant at least one nucleic acid molecule of the invention; cultivating the corn plant to allow the expression of an iRNA molecule comprising the nucleic acid sequence, wherein expression of an iRNA molecule comprising the nucleic acid sequence inhibits coleopteran pest growth and/or coleopteran pest damage, thereby reducing or eliminating a loss of yield due to coleopteran pest infestation. In some embodiments, the iRNA molecule is a dsRNA molecule. In these and further embodiments, the nucleic acid molecule(s) comprise dsRNA molecules that each comprise more than one nucleotide sequence that is specifically hybridizable to a nucleic acid molecule expressed in a coleopteran pest cell. In some embodiments, the nucleic acid molecule(s) consists of one nucleotide sequence that is specifically hybridizable to a nucleic acid molecule expressed in a coleopteran pest cell.

In some embodiments, a method for modulating the expression of a target gene in a coleopteran pest is provided, the method comprising: transforming a plant cell with a vector comprising a nucleic acid sequence encoding at least one nucleic acid molecule of the invention, wherein the nucleotide sequence is operatively-linked to a promoter and a transcription termination sequence; culturing the transformed plant cell under conditions sufficient to allow for development of a plant cell culture including a plurality of transformed plant cells; selecting for transformed plant cells that have integrated the nucleic acid molecule into their genomes; screening the transformed plant cells for expression of an iRNA molecule encoded by the integrated nucleic acid molecule; selecting a transgenic plant cell that expresses the iRNA molecule; and feeding the selected transgenic plant cell to the coleopteran pest. Plants may also be regenerated from transformed plant cells that express an iRNA molecule encoded by the integrated nucleic acid molecule. In some embodiments, the iRNA molecule is a dsRNA molecule. In these and further embodiments, the nucleic acid molecule(s) comprise dsRNA molecules that each comprise more than one nucleotide sequence that is specifically hybridizable to a nucleic acid molecule expressed in a coleopteran pest cell. In some embodiments, the nucleic acid molecule(s) consists of one nucleotide sequence that is specifically hybridizable to a nucleic acid molecule expressed in a coleopteran pest cell.

iRNA molecules of the invention can be incorporated within the seeds of a plant species (e.g., corn), either as a product of expression from a recombinant gene incorporated into a genome of the plant cells, or as incorporated into a coating or seed treatment that is applied to the seed before planting. A plant cell comprising a recombinant gene is considered to be a transgenic event. Also included in embodiments of the invention are delivery systems for the delivery of iRNA molecules to coleopteran pests. For example, the iRNA molecules of the invention may be directly introduced into the cells of a coleopteran pest. Methods for introduction may include direct mixing of iRNA with plant tissue from a host for the coleopteran pest, as well as application of compositions comprising iRNA molecules of the invention to host plant tissue. For example, iRNA molecules may be sprayed onto a plant surface. Alternatively, an iRNA molecule may be expressed by a microorganism, and the microorganism may be applied onto the plant surface, or introduced into a root or stem by a physical means such as an injection. As discussed, supra, a transgenic plant may also be genetically engineered to express at least one iRNA molecule in an amount sufficient to kill the coleopteran pests known to infest the plant. iRNA molecules produced by chemical or enzymatic synthesis may also be formulated in a manner consistent with common agricultural practices, and used as spray-on products for controlling plant damage by a coleopteran pest. The formulations may include the appropriate stickers and wetters required for efficient foliar coverage, as well as UV protectants to protect iRNA molecules (e.g., dsRNA molecules) from UV damage. Such additives are commonly used in the bioinsecticide industry, and are well known to those skilled in the art. Such applications may be combined with other spray-on insecticide applications (biologically based or otherwise) to enhance plant protection from coleopteran pests.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

The following EXAMPLES are provided to illustrate certain particular features and/or aspects. These EXAMPLES should not be construed to limit the disclosure to the particular features or aspects described.

### EXAMPLES

### Example 1

### Insect Diet Bioassays

Sample Preparation and Bioassays.

A number of dsRNA molecules (including those corresponding to chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), and cdc8 (SEQ ID NO:94)) were synthesized and purified using a MEGASCRIPT® RNAi kit. The purified dsRNA molecules were prepared in TE buffer, and all bioassays contained a control treatment consisting of this buffer, which served as a background check for mortality or growth inhibition of WCR (Diabrotica virgifera virgifera LeConte). The concentrations of dsRNA molecules in the bioassay buffer were measured using a NANODROP™ 8000 spectrophotometer (Thermo Scientific, Wilmington, Del.).

Samples were tested for insect activity in bioassays conducted with neonate insect larvae on artificial insect diet. WCR eggs were obtained from Crop Characteristics, Inc. (Farmington, Minn.).

The bioassays were conducted in 128-well plastic trays specifically designed for insect bioassays (C-D International, Pitman, N.J.). Each well contained approximately 1.0 mL of a diet designed for growth of coleopteran insects. A 60 μL aliquot of dsRNA sample was delivered by pipette onto the 1.5 cm² diet surface of each well (40 μL/cm²). dsRNA sample concentrations were calculated as the amount of dsRNA per square centimeter (ng/cm²) of surface area in the well. The treated trays were held in a fume hood until the liquid on the diet surface evaporated or was absorbed into the diet.

Within a few hours of eclosion, individual larvae were picked up with a moistened camel hair brush and deposited on the treated diet (one or two larvae per well). The infested wells of the 128-well plastic trays were then sealed with adhesive sheets of clear plastic, and vented to allow gas exchange. Bioassay trays were held under controlled environmental conditions (28° C., ˜40% Relative Humidity, 16:8 (Light:Dark)) for 9 days, after which time the total number of insects exposed to each sample, the number of dead insects, and the weight of surviving insects were recorded. Average percent mortality and average growth inhibition were calculated for each treatment. Growth inhibition (GI) was calculated as follows:

GI=[1−(TWIT/TNIT)/(TWIBC/TNIBC)]
where TWIT is the Total Weight of live Insects in the Treatment;
TNIT is the Total Number of Insects in the Treatment;
TWIBC is the Total Weight of live Insects in the Background Check (Buffer control); and
TNIBC is the Total Number of Insects in the Background Check (Buffer control).

Statistical analysis was done using JMP™ software (SAS, Cary, N.C.).

LC₅₀ (Lethal Concentration) is defined as the dosage at which 50% of the test insects are killed. GI₅₀ (Growth Inhibition) is defined as the dosage at which the mean growth (e.g. live weight) of the test insects is 50% of the mean value seen in Background Check samples.

Replicated bioassays demonstrated that ingestion of particular samples resulted in a surprising and unexpected mortality and/or growth inhibition of corn rootworm larvae.

### Example 2

### Identification of Candidate Target Genes

Multiple stages of WCR (Diabrotica virgifera virgifera LeConte) development were selected for pooled transcriptome analysis to provide candidate target gene sequences for control by RNAi transgenic plant insect resistance technology.

In one exemplification, total RNA was isolated from about 0.9 g of whole first-instar WCR larvae; (4 to 5 days post-hatch; held at 16° C.), and purified using the following phenol/TRI REAGENT®-based method (Molecular Research Center, Cincinnati, Ohio; Cat. No. TR 118):

Larvae were homogenized at room temperature in a 15 mL homogenizer with 10 mL of TRI REAGENT® until a homogenous suspension was obtained. Following 5 min. incubation at room temperature, the homogenate was dispensed into 1.5 mL microfuge tubes (1 mL per tube), 200 μL of chloroform was added, and the mixture was vigorously shaken for 15 seconds. After allowing the extraction to sit at room temperature for 10 min, the phases were separated by centrifugation at 12,000×g at 4° C. The upper phase (comprising about 0.6 mL) was carefully transferred into another sterile 1.5 mL tube, and an equal volume (0.6 mL) of room temperature isopropanol was added. After incubation at room temperature for 5 to 10 min, the mixture was centrifuged 8 min at 12,000×g (4° C. or 25° C.).

The supernatant was carefully removed and discarded, and the RNA pellet was washed twice by vortexing with 75% ethanol, with recovery by centrifugation for 5 min at 7,500×g (4° C. or 25° C.) after each wash. The ethanol was carefully removed, the pellet was allowed to air-dry for 3 to 5 min, and then was dissolved in nuclease-free sterile water. RNA concentration was determined by measuring the absorbance (A) at 260 nm and 280 nm. A typical extraction from about 0.9 g of larvae yielded over 1 mg of total RNA, with an A₂₆₀/A₂₈₀ ratio of 1.9. The RNA thus extracted was stored at −80° C. until further processed.

RNA quality was determined by running an aliquot through a 1% agarose gel. The agarose gel solution was made using autoclaved 10×TAE buffer (Tris-acetate EDTA; 1× concentration is 0.04 M Tris-acetate, 1 mM EDTA (ethylenediamine tetra-acetic acid sodium salt), pH 8.0) diluted with DEPC (diethyl pyrocarbonate)-treated water in an autoclaved container. 1×TAE was used as the running buffer. Before use, the electrophoresis tank and the well-forming comb were cleaned with RNaseAway™ (INVITROGEN Inc., Carlsbad, Calif.). Two μL of RNA sample were mixed with 8 μL of TE buffer (10 mM Tris HCl pH 7.0; 1 mM EDTA) and 10 μL of RNA sample buffer (Novagen® Catalog No 70606; EMD4 Bioscience, Gibbstown, N.J.). The sample was heated at 70° C. for 3 min, cooled to room temperature, and 5 μL (containing 1 μg to 2 μg RNA) were loaded per well. Commercially available RNA molecular weight markers were simultaneously run in separate wells for molecular size comparison. The gel was run at 60 volts for 2 hr.

A normalized cDNA library was prepared from the larval total RNA by a commercial service provider (Eurofins MWG Operon, Huntsville, Ala.), using random priming.

The normalized larval cDNA library was sequenced at ½ plate scale by GS FLX 454 Titanium™ series chemistry at Eurofins MWG Operon, which resulted in over 600,000 reads with an average read length of 348 bp. 350,000 reads were assembled into over 50,000 contigs. Both the unassembled reads and the contigs were converted into BLASTable databases using the publicly available program, FORMATDB (available from NCBI).

Total RNA and normalized cDNA libraries were similarly prepared from materials harvested at other WCR developmental stages. A pooled transcriptome library for target gene screening was constructed by combining cDNA library members representing the various developmental stages.

Candidate genes for RNAi targeting were selected using information regarding lethal RNAi effects of particular genes in other insects such as Drosophila and Tribolium. These genes were hypothesized to be essential for survival and growth in coleopteran insects. Selected target gene homologs were identified in the transcriptome sequence database as described below. Full-length or partial sequences of the target genes were amplified by PCR to prepare templates for double-stranded RNA (dsRNA) production.

TBLASTN searches using candidate protein coding sequences were run against BLASTable databases containing the unassembled Diabrotica sequence reads or the assembled contigs. Significant hits to a Diabrotica sequence (defined as better than e⁻²⁰ for contigs homologies and better than e⁻¹⁰ for unassembled sequence reads homologies) were confirmed using BLASTX against the NCBI non-redundant database. The results of this BLASTX search confirmed that the Diabrotica homolog candidate gene sequences identified in the TBLASTN search indeed comprised Diabrotica genes, or were the best hit to the non-Diabrotica candidate gene sequence present in the Diabrotica sequences. In most cases, Tribolium candidate genes which were annotated as encoding a protein gave an unambiguous sequence homology to a sequence or sequences in the Diabrotica transcriptome sequences. In a few cases, it was clear that some of the Diabrotica contigs or unassembled sequence reads selected by homology to a non-Diabrotica candidate gene overlapped, and that the assembly of the contigs had failed to join these overlaps. In those cases, Sequencher™ v4.9 (Gene Codes Corporation, Ann Arbor, Mich.) was used to assemble the sequences into longer contigs.

### Example 3

### Amplification of Candidate Target Genes and In Vitro dsRNA Production

Template Preparation by PCR and dsRNA Synthesis

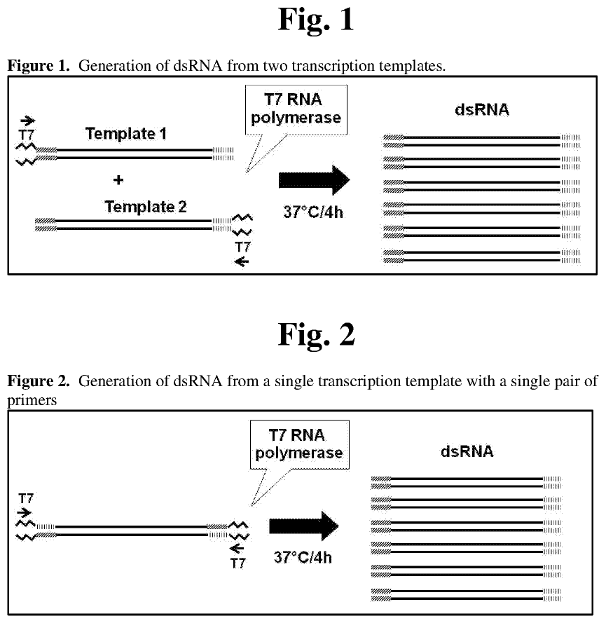

The strategies used to provide specific templates for dsRNA production are shown in FIGURE. 1 and FIG. 2. Primers were designed to amplify portions of coding regions of each target gene by PCR. See Table 2. Where appropriate, a T7 phage promoter sequence (TTAATACGACTCACTATAGGGAGA; SEQ ID NO:98) was incorporated into the 5′ ends of the amplified sense or antisense strands via the primers. See Table 2. Total RNA was extracted from WCR first-instar larvae, and first-strand cDNA was used as template for PCR reactions using opposing primers positioned to amplify all or part of the native target gene sequence. dsRNA was also amplified from the coding region for a yellow fluorescent protein (YFP) (negative control; SEQ ID NO:99).

For some selected target gene regions, two separate PCR amplifications were performed. Template DNAs intended for use in dsRNA synthesis were prepared by PCR using primer pairs in Table 2 and (as PCR template) first-strand cDNA prepared from total RNA isolated from WCR first-instar larvae. (YFP was amplified from a DNA clone.) The first PCR amplification introduced a T7 promoter sequence at the 5′ ends of the amplified sense strands. The second reaction incorporated the T7 promoter sequence at the 5′ ends of the antisense strands. The two PCR amplified fragments for each region of the target genes were then mixed in approximately equal amounts, and the mixture was used as transcription template for dsRNA production. See FIG. 1. Double-stranded RNA was synthesized and purified using an AMBION® MEGAscript® RNAi kit following the manufacturer's instructions (INVITROGEN). The concentrations of dsRNAs were measured using a NANODROP™ 8000 spectrophotometer (THERMO SCIENTIFIC, Wilmington, Del.). Such dsRNAs were each tested in insect diet feeding bioassays as described above. YFP primer sequences for use in the method depicted in FIG. 1 are also listed in Table 2.

For some selected target gene regions, single PCR amplifications were performed. Template DNAs intended for use in dsRNA synthesis were prepared by PCR using primer pairs in Table 2 and (as PCR template) first-strand cDNA prepared from total RNA isolated from WCR first-instar larvae. The PCR amplification introduced a T7 promoter sequence at the 5′ ends of the amplified sense strands and antisense strands. The amplified fragments for each region of the target genes were then used as transcription templates for dsRNA production. See FIG. 2. Double-stranded RNA was synthesized and purified using an Ambion® MEGAscript® RNAi kit following the manufacturer's instructions (Invitrogen). The concentrations of dsRNAs were measured using a NanoDrop™ 8000 spectrophotometer (Thermo Scientific, Wilmington, Del.). Such dsRNAs were each tested in insect diet feeding bioassays as described above.

TABLE 2
Primers and Primer Pairs used to amplify portions of coding regions of exemplary
target genes and YFP negative control gene.

| | | | SEQ | |
|---|---|---|---|---|
| | | Primer | ID | |
| | Gene ID | ID | NO. | Sequence |
| Pair 1 | chitin | chs-F1T7 | 107 | TTAATACGACTCACTATAGGGAGA |
| | synthase | | | CGTAGTGTCAAAAGCAAGTTTAC |
| | (Region 1) | | | |
| | chitin | chs-R1 | 108 | GCGAACTTATGTTGATCTTGATA |
| | synthase | | | |
| | (Region 1) | | | |
| Pair 2 | chitin | chs-F1 | 109 | CGTAGTGTCAAAAGCAAGTTTAC |
| | synthase | | | |
| | (Region 1) | | | |
| | chitin | chs-R1T7 | 110 | TTAATACGACTCACTATAGGGAGA |
| | synthase | | | GCGAACTTATGTTGATCTTGATA |
| | (Region 1) | | | |
| Pair 3 | chitin | chs-F2T7 | 111 | TTAATACGACTCACTATAGGGAGA |
| | synthase | | | GTTGTGTGGCCTCTGGTAGA |
| | (Region 2) | | | |
| | chitin | chs-R2 | 112 | TCTAACTCGTAGTAATCAGGC |
| | synthase | | | |
| | (Region 2) | | | |
| Pair 4 | chitin | chs-F2 | 113 | GTTGTGTGGCCTCTGGTAGA |
| | synthase | | | |
| | (Region 2) | | | |
| | chitin | chs-R2T7 | 114 | TTAATACGACTCACTATAGGGAGAT |
| | synthase | | | CTAACTCGTAGTAATCAGGC |
| | (Region 2) | | | |
| Pair 5 | chitin | chs-F3T7 | 115 | TTAATACGACTCACTATAGGGAGAT |
| | synthase | | | CTTGTCATGATGACAGTATTCG |
| | (Region 3) | | | |
| | chitin | chs-R3 | 116 | CCATCTGGTCCTAATTTTTTTTTGCA |
| | synthase | | | |
| | (Region 3) | | | |
| Pair 6 | chitin | chs-F3 | 117 | TCTTGTCATGATGACAGTATTCG |
| | synthase | | | |
| | (Region 3) | | | |
| | chitin | chs-R3T7 | 118 | TTAATACGACTCACTATAGGGAGA |
| | synthase | | | CCATCTGGTCCTAATTTTTTTTTGCA |
| | (Region 3) | | | |
| Pair 7 | chitin | chs-F4T7 | 119 | TTAATACGACTCACTATAGGGAGA |
| | synthase | | | CGCTGTCGCTAAAGATTTAAAG |
| | (Region 4) | | | |
| | chitin | chs-R4 | 120 | CTTTATTATTTTCCGCTGTTAAATA |
| | synthase | | | G |
| | (Region 4) | | | |
| Pair 8 | chitin | chs-F4 | 121 | CGCTGTCGCTAAAGATTTAAAG |
| | synthase | | | |
| | (Region 4) | | | |
| | chitin | chs-R4T7 | 122 | TTAATACGACTCACTATAGGGAGA |
| | synthase | | | CTTTATTATTTTCCGCTGTTAAATA |
| | (Region 4) | | | G |
| Pair 9 | outer | translocase- | 123 | TTAATACGACTCACTATAGGGAGA |
| | membrane | F1T7 | | AGGTTGAAAAAACAAAGTTGTTGT |
| | translocase | | | TACTTC |
| | (Region 1) | | | |
| | outer | translocase- | 124 | CTACTAGCTCTTTGGCCCAACAGAG |
| | membrane | R1 | | |
| | translocase | | | |
| | (Region 1) | | | |
| Pair 10 | outer | translocase- | 125 | AGGTTGAAAAAACAAAGTTGTTGT |
| | membrane | F1 | | TACTTC |
| | translocase | | | |
| | (Region 1) | | | |
| | outer | translocase- | 126 | TTAATACGACTCACTATAGGGAGA |
| | membrane | R1T7 | | CTACTAGCTCTTTGGCCCAACAGAG |
| | translocase | | | |
| | (Region 1) | | | |
| Pair 11 | outer | translocase- | 127 | TTAATACGACTCACTATAGGGAGA |
| | membrane | F2T7 | | CGGTGTAGAAAGATTAATGGAATG |
| | translocase | | | TG |
| | (Region 2) | | | |
| | outer | translocase- | 128 | TCACAGGCTAACTTTTTGTTGGAGT |
| | membrane | R2 | | T |
| | translocase | | | |
| | (Region 2) | | | |
| Pair 12 | outer | translocase- | 129 | CGGTGTAGAAAGATTAATGGAATG |
| | membrane | F2 | | TG |
| | translocase | | | |
| | (Region 2) | | | |
| | outer | translocase- | 130 | TTAATACGACTCACTATAGGGAGAT |
| | membrane | R2T7 | | CACAGGCTAACTTTTTGTTGGAGTT |
| | translocase | | | |
| | (Region 2) | | | |
| Pair 13 | double parked | double-FT7 | 131 | TTAATACGACTCACTATAGGGAGA |
| | | | | CAATGACGCGATCAGAAGATAAAG |
| | double parked | double-R | 132 | TCAGTCAAAACTGCAAAGAAATAT |
| | | | | TGTAA |
| Pair 14 | double parked | double-F | 133 | CAATGACGCGATCAGAAGATAAAG |
| | double parked | double-RT7 | 134 | TTAATACGACTCACTATAGGGAGAT |
| | | | | CAGTCAAAACTGCAAAGAAATATT |
| | | | | GTAA |
| Pair 15 | disc | discover-FT7 | 135 | TTAATACGACTCACTATAGGGAGA |
| | overgrown | | | ACAAGTAGTATCATCGGTATCGGA |
| | | | | A |
| | disc | discover-R | 136 | GGAAAATTCAGCGGGAAGC |
| | overgrown | | | |
| Pair 16 | disc | discover-F | 137 | ACAAGTAGTATCATCGGTATCGGA |
| | overgrown | | | A |
| | disc | discover- | 138 | TTAATACGACTCACTATAGGGAGA |
| | overgrown | RT7 | | GGAAAATTCAGCGGGAAGC |
| Pair 17 | ctf4 | Ctf4-024- | 139 | TTAATACGACTCACTATAGGGAGA |
| | contig05024 | F1T7 | | GTATTCGGTTTTACACAAAAATGAA |
| | | | | AATGC |
| | ctf4 | Ctf4-024-R1 | 140 | ACTATAGGCTGTAATTTTTCCAGAT |
| | contig05024 | | | CCAGA |
| Pair 18 | ctf4 | Ctf4-024-F1 | 141 | GTATTCGGTTTTACACAAAAATGAA |
| | contig05024 | | | AATGC |
| | ctf4 | Ctf4-024- | 142 | TTAATACGACTCACTATAGGGAGA |
| | contig05024 | R1T7 | | ACTATAGGCTGTAATTTTTCCAGAT |
| | | | | CCAGA |
| Pair 19 | ctf4 Var1 | Ctf4-054- | 143 | TTAATACGACTCACTATAGGGAGA |
| | (c06054) | FT7 | | ATGATGCCAATAGGAGGAGCTC |
| | ctf4 Var1 | Ctf4-054-R | 144 | GTTTGTGTCCTGAAATGGTTTTTCA |
| | (c06054) | | | AAC |
| Pair 20 | ctf4 Var1 | Ctf4-054-F | 145 | ATGATGCCAATAGGAGGAGCTC |
| | (c06054) | | | |
| | ctf4 Var1 | Ctf4-054- | 146 | TTAATACGACTCACTATAGGGAGA |
| | (c06054) | RT7 | | GTTTGTGTCCTGAAATGGTTTTTCA |
| | | | | AAC |
| Pair 21 | rpl9 | rpl9-FT7 | 147 | TTAATACGACTCACTATAGGGAGA |
| | | | | GAAGCAAATTGTAACAAACCAAAC |
| | | | | G |
| | rpl9 | rpl9-R | 148 | CTTCTTGTACAACAGTTGTTTTTTCA |
| | | | | G |
| Pair 22 | rpl9 | rpl9-F | 149 | GAAGCAAATTGTAACAAACCAAAC |
| | | | | G |
| | rpl9 | rpl9-RT7 | 150 | TTAATACGACTCACTATAGGGAGA |
| | | | | CTTCTTGTACAACAGTTGTTTTTTCA |
| | | | | G |
| Pair 23 | serpin | serpin-744- | 151 | TTAATACGACTCACTATAGGGAGA |
| | protease | F1T7 | | CCTACTGAAGCTGTGCAATTCC |
| | inhibitor 14 | | | |
| | (Region 1) | | | |
| | serpin | serpin-744- | 152 | AGCGTTGTAATACTTTATAACGACA |
| | protease | R1 | | TTCT |
| | inhibitor 14 | | | |
| | (Region 1) | | | |
| Pair 24 | serpin | serpin-744- | 153 | CCTACTGAAGCTGTGCAATTCC |
| | protease | F1 | | |
| | inhibitor 14 | | | |
| | (Region 1) | | | |
| | serpin | serpin-744- | 154 | TTAATACGACTCACTATAGGGAGA |
| | protease | R1T7 | | AGCGTTGTAATACTTTATAACGACA |
| | inhibitor 14 | | | TTCT |
| | (Region 1) | | | |
| Pair 25 | serpin | serpin-744- | 155 | TTAATACGACTCACTATAGGGAGA |
| | protease | F2T7 | | ACCTGAAAAGCTTAGATTTTTCAAA |
| | inhibitor 14 | | | TTC |
| | (Region 2) | | | |
| | serpin | serpin-744- | 156 | TGTGATTTTGGTCTTGTGTAACAGG |
| | protease | R2 | | |
| | inhibitor 14 | | | |
| | (Region 2) | | | |
| Pair 26 | serpin | serpin-744- | 157 | ACCTGAAAAGCTTAGATTTTTCAAA |
| | protease | F2 | | TTC |
| | inhibitor 14 | | | |
| | (Region 2) | | | |
| | serpin | serpin-744- | 158 | TTAATACGACTCACTATAGGGAGAT |
| | protease | R2T7 | | GTGATTTTGGTCTTGTGTAACAGG |
| | inhibitor 14 | | | |
| | (Region 2) | | | |
| Pair 27 | myosin 3 LC | Myosin-164- | 159 | TTAATACGACTCACTATAGGGAGA |
| | 19164 | FT7 | | CCATGTTTTCGCAATCTCAAGTAG |
| | myosin 3 LC | Myosin-164- | 160 | GTCGCATTCTTTGCCGGTGAAT |
| | 19164 | R | | |
| Pair 28 | myosin 3 LC | Myosin-164- | 161 | CCATGTTTTCGCAATCTCAAGTAG |
| | 19164 | F | | |
| | myosin 3 LC | Myosin-164- | 162 | TTAATACGACTCACTATAGGGAGA |
| | 19164 | RT7 | | GTCGCATTCTTTGCCGGTGAAT |
| Pair 29 | myosin 3 LC | Myosin-333- | 163 | TTAATACGACTCACTATAGGGAGA |
| | 1333 | FT7 | | ATGGCTGACCAACTCACCGAA |
| | myosin 3 LC | Myosin-333- | 164 | CTCTTCGTAATTGACTTGACCATCA |
| | 31333 | R | | C |
| Pair 30 | myosin 3 LC | Myosin-333- | 165 | ATGGCTGACCAACTCACCGAA |
| | 31333 | F | | |
| | myosin 3 LC | Myosin-333- | 166 | TTAATACGACTCACTATAGGGAGA |
| | 31333 | RT7 | | CTCTTCGTAATTGACTTGACCATCA |
| | | | | C |
| Pair 31 | megator | mega-F1T7 | 167 | TTAATACGACTCACTATAGGGAGAT |
| | (Region1) | | | GATTAAAAAAGCAACTTGATGAGG |
| | | | | CT |
| | megator | mega-R1 | 168 | TCAACTGTTTGGATAAGGTCTCCC |
| | (Region 1) | | | |
| Pair 32 | megator | mega-F1 | 169 | TGATTAAAAAAGCAACTTGATGAG |
| | (Region 1) | | | GCT |
| | megator | mega-R1T7 | 170 | TTAATACGACTCACTATAGGGAGAT |
| | (Region 1) | | | CAACTGTTTGGATAAGGTCTCCC |
| Pair 33 | megator | mega-F2T7 | 171 | TTAATACGACTCACTATAGGGAGA |
| | (Region 2) | | | AGTGCCGAAATAATTAGGTTCAAG |
| | megator | mega-R2 | 172 | TTCCTTGGCGTTCTTAAACAGCG |
| | (Region 2) | | | |
| Pair 34 | megator | mega-F2 | 173 | AGTGCCGAAATAATTAGGTTCAAG |
| | (Region 2) | | | |
| | megator | mega-R2T7 | 174 | TTAATACGACTCACTATAGGGAGAT |
| | (Region 2) | | | TCCTTGGCGTTCTTAAACAGCG |
| Pair 35 | g-protein | G-beta-F1T7 | 175 | TTAATACGACTCACTATAGGGAGA |
| | (Region 1) | | | GTATGAACGAACTGGATTCTCTTAG |
| | | | | G |
| | g-protein | G-beta-R1 | 176 | TGGTTGTCATCGAGGAAACG |
| | (Region 1) | | | |
| Pair 36 | g-protein | G-beta-F1 | 177 | GTATGAACGAACTGGATTCTCTTAG |
| | (Region 1) | | | G |
| | g-protein | G-beta-R1T7 | 178 | TTAATACGACTCACTATAGGGAGAT |
| | (Region 1) | | | GGTTGTCATCGAGGAAACG |
| Pair 37 | g-protein | G-beta-F2T7 | 179 | TTAATACGACTCACTATAGGGAGA |
| | (Region 2) | | | AGTTCAGGAGATATGTCTTGTGCCC |
| | g-protein | G-beta-R2 | 180 | CTTAATTCCAAATGCGTAGGAAACT |
| | (Region 2) | | | |
| Pair 38 | g-protein | G-beta-F2 | 181 | AGTTCAGGAGATATGTCTTGTGCCC |
| | (Region 2) | | | |
| | g-protein | G-beta-R2T7 | 182 | TTAATACGACTCACTATAGGGAGA |
| | (Region 2) | | | CTTAATTCCAAATGCGTAGGAAACT |
| Pair 39 | flap wing | flapwing- | 183 | TTAATACGACTCACTATAGGGAGA |
| | | FT7 | | GGTGGACTGAGTCCAGATTTGC |
| | flap wing | flapwing-R | 184 | GTGATCTACTTCTTATTTTTGTTAGG |
| | | | | GG |
| Pair 40 | flap wing | flapwing-F | 185 | GGTGGACTGAGTCCAGATTTGC |
| | flap wing | flapwing- | 186 | TTAATACGACTCACTATAGGGAGA |
| | | RT7 | | GTGATCTACTTCTTATTTTTGTTAGG |
| | | | | GG |
| Pair 41 | female sterile | female-F1T7 | 187 | TTAATACGACTCACTATAGGGAGA |
| | 2 ketel | | | CGATTCTGCTGCAAAAATTAACG |
| | (Region1) | | | |
| | female sterile | female-R1 | 188 | AACGTCGGCATTTTCGTAAGC |
| | 2 ketel | | | |
| | (Region1) | | | |
| Pair 42 | female sterile | female-F1 | 189 | CGATTCTGCTGCAAAAATTAACG |
| | 2 ketel | | | |
| | (Region1) | | | |
| | female sterile | female-R1T7 | 190 | TTAATACGACTCACTATAGGGAGA |
| | 2 ketel | | | AACGTCGGCATTTTCGTAAGC |
| | (Region1) | | | |
| Pair 43 | female sterile | female-F2T7 | 191 | TTAATACGACTCACTATAGGGAGA |
| | 2 ketel | | | AATGAAGAAACCGGTACTCCAGA |
| | (Region2) | | | |
| | female sterile | Female-R2 | 192 | ATGGTAGCGCTTTCAGTTTGAG |
| | 2 ketel | | | |
| | (Region2) | | | |
| Pair 44 | female sterile | female-F2T7 | 193 | AATGAAGAAACCGGTACTCCAGA |
| | 2 ketel | | | |
| | (Region2) | | | |
| | Female sterile | female-R2T7 | 194 | TTAATACGACTCACTATAGGGAGA |
| | 2 Ketel | | | ATGGTAGCGCTTTCAGTTTGAG |
| | (Region2) | | | |
| Pair 45 | enh. of | Enh- | 195 | TTAATACGACTCACTATAGGGAGA |
| | polycomb | polycomb- | | ATGTCGAAGCTTTCATTTAGGG |
| | (Region 1) | F1T7 | | |
| | enh. of | Enh- | 196 | ACCTGGCAATTCGGAAACTTC |
| | polycomb | polycomb- | | |
| | (Region 1) | R1 | | |
| Pair 46 | enh. of | Enh- | 197 | ATGTCGAAGCTTTCATTTAGGG |
| | polycomb | polycomb-F1 | | |
| | (Region 1) | | | |
| | enh. of | Enh- | 198 | TTAATACGACTCACTATAGGGAGA |
| | polycomb | polycomb- | | ACCTGGCAATTCGGAAACTTC |
| | (Region 1) | R1T7 | | |
| Pair 47 | enh. of | Enh- | 199 | TTAATACGACTCACTATAGGGAGAT |
| | polycomb | polycomb- | | CCAAATCAAAGTTGGGCGA |
| | (Region 2) | F2T7 | | |
| | enh. of | Enh- | 200 | AGCCGCCTCTACCAACCCT |
| | polycomb | polycomb- | | |
| | (Region 2) | R2 | | |
| Pair 48 | enh. of | Enh- | 201 | TCCAAATCAAAGTTGGGCGA |
| | polycomb | polycomb-F2 | | |
| | (Region 2) | | | |
| | enh. of | Enh- | 202 | TTAATACGACTCACTATAGGGAGA |
| | polycomb | polycomb- | | AGCCGCCTCTACCAACCCT |
| | (Region 2) | R2T7 | | |
| Pair 49 | dead box 73D | Deadbox73D- | 203 | TTAATACGACTCACTATAGGGAGA |
| | | F1T7 | | AGATGTGTGTTTAATAAGTGGAACT |
| | | | | AACT |
| | dead box 73D | Deadbox73D- | 204 | AACACGGGAGTTGGGACAG |
| | | R1 | | |
| Pair 50 | dead box 73D | Deadbox73D- | 205 | AGATGTGTGTTTAATAAGTGGAACT |
| | | F1 | | AACT |
| | dead box 73D | Deadbox73D- | 206 | TTAATACGACTCACTATAGGGAGA |
| | | R1T7 | | AACACGGGAGTTGGGACAG |
| Pair 51 | cg7000 | cg7000 - | 207 | TTAATACGACTCACTATAGGGAGA |
| | | 956-FT7 | | ATGAAAGGAAGGGAAAAGTTATGT |
| | | | | GC |
| | cg7000 | cg7000 - | 208 | ACCTGAGGAATCTGGCGG |
| | | 956-R | | |
| Pair 52 | cg7000 | cg7000 - | 209 | ATGAAAGGAAGGGAAAAGTTATGT |
| | | 956-F | | GC |
| | cg7000 | cg7000 - | 210 | TTAATACGACTCACTATAGGGAGA |
| | | 956-RT7 | | ACCTGAGGAATCTGGCGG |
| Pair 53 | heat shock | Hsc70- | 211 | TTAATACGACTCACTATAGGGAGA |
| | protein-12300 | 300FT7 | | GATGGCTAAAGCCCCAGCT |
| | heat shock | Hsc70- | 212 | TTAATACGACTCACTATAGGGAGA |
| | protein-12300 | 300RT7 | | ACATTCTTTCCTAAGTATGCTTCTG |
| | | | | C |
| Pair 54 | heat shock | Hsc70- | 213 | TTAATACGACTCACTATAGGGAGA |
| | protein-331 | 331FT7 | | ATGAGGTTATATTTGGGATTTGGAG |
| | heat shock | Hsc70- | 214 | TTAATACGACTCACTATAGGGAGA |
| | protein-331 | 331RT7 | | CTTCTGCTGTTTCCTTCATCTTTCC |
| Pair 55 | rnr1 | rnr1-F1T7 | 215 | TTAATACGACTCACTATAGGGAGA |
| | (Region 1) | | | ATGGTGAAACCGTTGAATAAATTTT |
| | | | | ATG |
| | rnr1 | rnr1-R1T7 | 216 | TTAATACGACTCACTATAGGGAGA |
| | (Region 1) | | | CGTTTCTTTCTGAAGATTAGATATA |
| | | | | GCG |
| Pair 56 | rnr1 | rnr1-F2T7 | 217 | TTAATACGACTCACTATAGGGAGA |
| | (Region 2) | | | ATGATATACGATAGGGATTTTTCCT |
| | | | | ATG |
| | rnr1 | rnr1-R2T7 | 218 | TTAATACGACTCACTATAGGGAGA |
| | (Region 2) | | | AGAAGGTTTATTGGTGCTAAGTTGA |
| | | | | TC |
| Pair 57 | rnr1 | rnr1-F3T7 | 219 | TTAATACGACTCACTATAGGGAGA |
| | (Region 3) | | | ATGTACGAGGGCAGTCCTGTCA |
| | rnr1 | rnr1-R3T7 | 220 | TTAATACGACTCACTATAGGGAGA |
| | (Region 3) | | | GATGAACGCTCCTCTATCGATG |
| Pair 58 | elav | Elav-F1T7 | 221 | TTAATACGACTCACTATAGGGAGA |
| | | | | CGTAATGAATCCAGGCGC |
| | elav | Elav-RT7 | 222 | TTAATACGACTCACTATAGGGAGA |
| | | | | GTTGTCAATGGAACGATCCAAATTC |
| Pair 59 | pten | Pten-F1T7 | 223 | TTAATACGACTCACTATAGGGAGA |
| | | | | ATGGGTCAGTGTTTTAGTGCTAGC |
| | pten | Pten-RT7 | 224 | TTAATACGACTCACTATAGGGAGA |
| | | | | GTGATCGTCGAAGGGAAATATTTTG |
| Pair 60 | cdc8 | Cdc8-827- | 225 | TTAATACGACTCACTATAGGGAGA |
| | | F1T7 | | ATGAGTATGAAACGAGGAGCACT |
| | cdc8 | Cdc8-827- | 226 | TTAATACGACTCACTATAGGGAGA |
| | | RT7 | | AGACCCAGCAGCATCAATTCTAAC |
| Pair 61 | YFP | YFP-F_T7 | 227 | TTAATACGACTCACTATAGGGAGA |
| | | | | CACCATGGGCTCCAGCGGCGCCC |
| | YFP | YFP-R | 228 | AGATCTTGAAGGCGCTCTTCAGG |
| Pair 62 | YFP | YFP-F | 229 | CACCATGGGCTCCAGCGGCGCCC |
| | YFP | YFP-R_T7 | 230 | TTAATACGACTCACTATAGGGAGA |
| | | | | AGATCTTGAAGGCGCTCTTCAGG |

### Example 4

### Identification of Diabrotica Spp. Candidate Target Gene: Chitin Synthase

A candidate target gene encoding chitin synthase (SEQ ID NO: 1) was identified as a gene that may lead to coleopteran pest mortality, inhibition of growth, inhibition of development, or inhibition of reproduction in WCR. Chitin synthases (CHSs, EC: 2.4.1.16, UDP-N-acetyl-D-glucosamine:chitin 4-beta-N-acetylglucosaminyltranserase) catalyse the polymerization of N-acetyl-D-glucosamine (GlcNAc) into chitin from intracellular pools of UDP-GlcNAc. Chitin is a major component of cuticular exoskeletons and midgut peritrophic membranes of arthropods, including insects. In the model insect Tribolium castaneum (Tc; red flour beetle), expression of a chitin synthase gene (TcCHs1; specialized for synthesis of epidermal cuticle) was knocked down after dsRNA homologous to the gene sequence was injected into the insect. The insect did not survive, and there was disruption of the molting process (larva->larva, larva->pupa, pupa->adult) (Arakane et al., 2005, Insect Molecular Biology 14:453-463). It was further shown that knocking down the expression of chitin synthase in Spodoptera exigua caused abnormality (Chen et al., 2008, Bulletin of Entomological Research 98: 613), and in the oriental migratory locust, Locusta migratoria (Meyen), silencing chitin synthase caused mortality (Zhang et al., 2010, Insect Biochemistry and Molecular Biology 40: 824-833).

A clone of a Diabrotica candidate gene encoding a chitin synthase (SEQ ID NO:1) was used to generate PCR amplicons for dsRNA synthesis. The sequence of SEQ ID NO:1 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. The Diabrotica chitin synthase sequence (SEQ ID NO:1) is somewhat related to a fragment of a chitin synthase gene from human body louse, Pediculus humanus corporis (GENBANK Accession No. XM_002423559). The closest homolog of the Diabrotica chitin synthase amino acid sequence (SEQ ID NO:1) is a Tribolium casetanum CHITIN SYNTHASE1 protein having GENBANK Accession No. NP_001034492 (71% similar; 55% identical over the homology region).

SEQ ID NO:1 presents a 4659 bp DNA sequence that includes an open reading frame that encodes a Diabrotica chitin synthase. SEQ ID NO:2 presents a 1476 amino acid sequence of a Diabrotica CHITIN SYNTHASE protein.

SEQ ID NO:3 shows an exemplary amplified fragment of chitin synthase Region 1 used for in vitro dsRNA synthesis, which was amplified using primer Pair 1 (T7 at 5′ end) and primer Pair 2 (T7 at 3′ end) (Table 2). T7 promoter sequences (SEQ ID NO:98) at the 5′ and 3′ ends are not shown.

SEQ ID NO:4 shows an exemplary amplified fragment of chitin synthase Region 2 used for in vitro dsRNA synthesis, which was amplified using primer Pair 3 (T7 at 5′ end) and primer Pair 4 (T7 at 3′ end) (Table 2). T7 promoter sequences at the 5′ and 3′ ends are not shown.

SEQ ID NO:5 shows an exemplary amplified fragment of chitin synthase Region 3 used for in vitro dsRNA synthesis, which was amplified using primer Pair 5 (T7 at 5′ end) and primer Pair 6 (T7 at 3′ end) (Table 2). T7 promoter sequences at the 5′ and 3′ ends are not shown.

SEQ ID NO:6 shows an exemplary amplified fragment of chitin synthase Region 4 used for in vitro dsRNA synthesis, which was amplified using primer Pair 7 (T7 at 5′ end) and primer Pair 8 (T7 at 3′ end) (Table 2). T7 promoter sequences at the 5′ and 3′ ends are not shown.

SEQ ID NO:7 shows a DNA sequence of a chitin synthase hairpin RNA for expression in corn cells or corn plants. A sense DNA segment (comprising bases 807 to 1157 of SEQ ID NO:1) is separated from a segment comprising the antisense orientation of the sense DNA bases by an ST-LS1 intron segment (SEQ ID NO:100).

Synthetic dsRNA designed to inhibit chitin synthase target gene sequences caused growth inhibition when administered to WCR in diet-based assays. Table 3 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 3
Results of dsRNA diet feeding assays obtained with western
corn rootworm larvae after 9 days of feeding. ANOVA analysis
found significant differences in Mean Growth Inhibition.
Means were separated using the Tukey-Kramer test.

| Sample | Dose | Repli- | Mean % | Mean Growth |
|---|---|---|---|---|
| Name | (ng/cm²) | cations | Mortality* | Inhibition* |
| chitin | 1000 | 4 | 27.88 ± 5.37 (A) | 0.77 ± 0.08 (A) |
| synthase | | | | |
| Region 1 | | | | |
| chitin | 1000 | 4 | 21.02 ± 8.71 (A) | 0.73 ± 0.08 (A) |
| synthase | | | | |
| Region 2 | | | | |
| chitin | 1000 | 4 | 18.37 ± 3.99 (A) | 0.68 ± 0.12 (A) |
| synthase | | | | |
| Region 3 | | | | |
| chitin | 1000 | 4 | 17.03 ± 7.20 (A) | 0.73 ± 0.07 (A) |
| synthase | | | | |
| Region 4 | | | | |
| TE | 0 | 4 | 4.70 ± 2.80 (A) | 0 (B) |
| buffer** | | | | |
| Water | 0 | 2 | 6.67 ± 4.71 (A) | 0 (B) |
| YFP*** | 1000 | 4 | 8.51 ± 3.81 (A) | −0.24 ± 0.27 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from chitin synthase Region 1, chitin synthase Region 2, chitin synthase Region 3, and chitin synthase Region 4 exhibited increased efficacy in this assay over other dsRNAs screened.

### Example 5

### Identification of Diabrotica Spp. Candidate Target Gene: Outer Membrane Translocase

A candidate target gene encoding an outer membrane translocase (SEQ ID NO:8) was identified as a gene that may lead to coleopteran pest mortality, inhibition of growth, inhibition of development, or inhibition of reproduction in WCR. In Drosophila, an outer membrane translocase gene (also known as Tom 34, or unc-45) encodes a protein having functions in chaperone-mediated protein folding, myosin filament assembly, and somatic muscle development (Lee et al., (2011) J. Cell Science 124:699-705, and FLYBASE). Loss-of-function mutations in the Drosophila unc-45 gene cause lethality (Lee et al., ibid.; Spradling et al., (1999) Genetics 153:135-177.)

A clone of a Diabrotica candidate gene encoding an outer membrane translocase (SEQ ID NO:8) was used to generate PCR amplicons for dsRNA synthesis. The sequence of SEQ ID NO:8 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found within a GENBANK search. The closest homolog of the Diabrotica OUTER MEMBRANE TRANSLOCASE amino acid sequence (SEQ ID NO:9) is a Tribolium casetanum protein having GENBANK Accession No. XP_973113 (90% similar; 76% identical over the homology region).

SEQ ID NO:8 presents a 4239 bp DNA sequence that includes an open reading frame that encodes a Diabrotica outer membrane translocase protein. SEQ ID NO:9 presents an 1476 amino acid sequence of a Diabrotica OUTER MEMBRANE TRANSLOCASE.

SEQ ID NO:10 shows an exemplary amplified fragment of outer membrane translocase Region 1 used for in vitro dsRNA synthesis, which was amplified using primer Pair 9 (T7 at 5′ end) and primer Pair 10 (T7 at 3′ end) (Table 2). T7 promoter sequences (SEQ ID NO:98) at the 5′ and 3′ ends are not shown.

SEQ ID NO:11 shows an exemplary amplified fragment of outer membrane translocase Region 2 used for in vitro dsRNA synthesis, which was amplified using primer Pair 11 (T7 at 5′ end) and primer Pair 12 (T7 at 3′ end) (Table 2). T7 promoter sequences at the 5′ and 3′ ends are not shown.

SEQ ID NO:12 shows a DNA sequence of an outer membrane translocase hairpin RNA for expression in corn cells or corn plants. A sense DNA segment (comprising bases 962 to 1151 of SEQ ID NO:8) is separated from a segment comprising the antisense orientation of the sense DNA bases by an ST-LS1 intron segment (SEQ ID NO:100).

Synthetic dsRNA designed to inhibit outer membrane translocase target gene sequences caused mortality and growth inhibition when administered to WCR in diet-based assays. Table 4 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 4
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| outer | 1000 | 6 | 30.17 ± 6.03 (A) | 0.56 ± 0.08 (A) |
| membrane | | | | |
| translocase | | | | |
| Region 1 | | | | |
| outer | 1000 | 6 | 37.17 ± 2.02 (A) | 0.63 ± 0 .07 (A) |
| membrane | | | | |
| translocase | | | | |
| Region 2 | | | | |
| TE** | 0 | 6 | 12.88 ± 4.92 (B) | 0.00 ± 0.00 (B) |
| WATER | 0 | 6 | 6.94 ± 3.16 (B) | 0.00 ± 0.00 (B) |
| YFP*** | 1000 | 6 | 12.32 ± 3.07 (B) | −0.20 ± 0.33 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from outer membrane translocase Region 1 and outer membrane translocase Region 2 exhibited increased efficacy in this assay over other dsRNAs screened.

### Example 6

### Identification of Diabrotica Spp. Candidate Target Gene: Double Parked

A candidate target gene referred to herein as double parked (SEQ ID NO:13) was identified as a gene that may lead to coleopteran pest mortality, inhibition of growth, inhibition of development, or inhibition of reproduction in WCR. In Drosophila, the double parked gene encodes a DNA binding protein with functions in the regulation of DNA replication, mitotic sister chromatid separation, and cytokinesis (FLYBASE). Mutations in the Drosophila double parked gene can cause lethality (Roch et al., (1998) Molecular and General Genetics 257:103-112; Spradling et al., (1999) Genetics 153:135-277; Underwood et al., (1990) Genetics 126:639-650; Whittaker et al., (2000) Genes and Development 14:1765-1776).

A clone of a Diabrotica candidate gene encoding double parked (SEQ ID NO:13) was used to generate PCR amplicons for dsRNA synthesis. The sequence of SEQ ID NO:13 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found within a GENBANK search. The closest homolog of the Diabrotica DOUBLE PARKED amino acid sequence (SEQ ID NO:14) is a Tribolium casetanum protein having GENBANK Accession No. XP_969028 (81% similar; 58% identical over the homology region).

SEQ ID NO:13 presents a 510 bp DNA sequence that includes an open reading frame that encodes a Diabrotica double parked protein. SEQ ID NO:14 presents an 122 amino acid sequence of a Diabrotica DOUBLE PARKED protein.

SEQ ID NO:15 shows an exemplary amplified fragment of double parked used for in vitro dsRNA synthesis, which was amplified using primer Pair 13 (T7 at 5′ end) and primer Pair 14 (T7 at 3′ end) (Table 2). T7 promoter sequences (SEQ ID NO:98) at the 5′ and 3′ ends are not shown.

SEQ ID NO:16 shows a DNA sequence of a double parked hairpin RNA for expression in corn cells or corn plants. A sense DNA segment (comprising bases 73 to 439 of SEQ ID NO:13) is separated from a segment comprising the antisense orientation of the sense DNA bases by an ST-LS1 intron segment (SEQ ID NO:100).

Synthetic dsRNA designed to inhibit double parked target gene sequences caused growth inhibition when administered to WCR in diet-based assays. Table 5 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 5
Results of dsRNA diet feeding assays obtained with western
corn rootworm larvae after 9 days of feeding. ANOVA analysis
found significant differences in Mean Growth Inhibition.
Means were separated using the Tukey-Kramer test.

| Sample | Dose | Repli- | Mean % | | Mean Growth | |
|---|---|---|---|---|---|---|
| Name | (ng/cm²) | cations | Mortality* | | Inhibition* | |
| double parked | 1000 | 4 | 30.64325 | (A) | 0.6285 | (B) |
| TE** | 0 | 4 | 10.66175 | (A) | 0 | (A) |
| WATER | 0 | 4 | 12.41 | (A) | 0 | (A) |
| YFP*** | 1000 | 4 | 18.38225 | (A) | 0.215 | (A) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from double parked exhibited increased efficacy in this assay over other dsRNAs screened.

### Example 7

### Identification of Diabrotica Spp. Candidate Target Gene: Discs Overgrown

A candidate target gene referred to herein as discs overgrown (SEQ ID NO:17) was identified as a gene that may lead to coleopteran pest mortality, inhibition of growth, inhibition of development, or inhibition of reproduction in WCR. The Drosophila discs overgrown (dco) gene, also known as DBT, encodes a protein with kinase activity (FLYBASE). The Drosophila discs overgrown protein has functions in cell survival and growth control (Jia et al., (2005) Developmental Cell 9:819-830; Zilian et al., (1999) Development 126:5409-5420). Loss-of-function mutations in the Drosophila dco gene cause lethality (Szabab et al., (1991) Genetics 127:525-533; Zilian et al., ibid.)

A 1400 bp clone of a Diabrotica candidate gene encoding discs overgrown (SEQ ID NO:17) was used to generate PCR amplicons for dsRNA synthesis. Bases 1010 to 1269 of SEQ ID NO:17 have been disclosed in two segments in GENBANK Accession No. EW770643.1, but SEQ ID NO:17 is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. The closest insect homolog of the Diabrotica DISCS OVERGROWN amino acid sequence (SEQ ID NO:18) is a Tribolium casetanum discs overgrown protein having GENBANK Accession No. EFA10353 (94% similar; 90% identical over the major homology region).

SEQ ID NO:17 presents a 1400 bp DNA sequence that includes an open reading frame that encodes a Diabrotica discs overgrown protein. SEQ ID NO:18 presents a 381 amino acid sequence of a DISCS OVERGROWN protein.

SEQ ID NO:19 shows an exemplary amplified fragment of discs overgrown used for in vitro dsRNA synthesis, which was amplified using primer Pair 15 (T7 at 5′ end) and primer Pair 16 (T7 at 3′ end) (Table 2). T7 promoter sequences (SEQ ID NO:98) at the 5′ and 3′ ends are not shown.

SEQ ID NO:20 shows a DNA sequence of a discs overgrown hairpin RNA for expression in corn cells or corn plants. A sense DNA segment (comprising bases 215 to 567 of SEQ ID NO:17) is separated from a segment comprising the antisense orientation of the sense DNA bases by an ST-LS1 intron segment (SEQ ID NO:100).

Synthetic dsRNA designed to inhibit discs overgrown target gene sequences caused mortality and growth inhibition when administered to WCR in diet-based assays. Table 6 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 6
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth | |
|---|---|---|---|---|---|
| Name | (ng/cm²) | cations | Mortality* | Inhibition* | |
| discs overgrown | 1000 | 4 | 47.84 (A) | 0.689 | (A) |
| TE** | 0 | 3 | 14.23 (B) | 0 | (B) |
| Water | 0 | 3 | 20.48 (B) | 0 | (B) |
| YFP*** | 1000 | 4 | 18.09 (B) | 0.04 | (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from discs overgrown exhibited increased efficacy in this assay over other dsRNAs screened.

### Example 8

### Identification of Diabrotica Spp. Candidate Target Gene: Ctf4

A candidate target gene encoding ctf4 (SEQ ID NO:21) was identified as a gene that may lead to coleopteran pest mortality, inhibition of growth, inhibition of development, or inhibition of reproduction in WCR. The Drosophila ctf4 protein has been shown to be a central member of the DNA replication fork and links the replicative MCM helicase and DNA polymerase a primase. In addition, it has been implicated as a member of a complex (the Fork Protection Complex) that promotes replication fork stability, and is thought to be important for sister chromatid cohesion (Gosnell and Christensen, (2011) BMC Molecular Biology 12:13-22). The loss of these functions causes lethality.

The first 283 bases of Diabrotica ctf4 (SEQ ID NO:21) are disclosed in U.S. Pat. No. 7,612,194. There was no significant homologous nucleotide sequence found with a search in GENBANK. The closest homolog of the Diabrotica CTF4 amino acid sequence (SEQ ID NO:22) is a Tribolium casetanum protein having GENBANK Accession No. EEZ98418.1 (90% similar; 82% identical over the homology region).

SEQ ID NO:21 presents a 1270 bp DNA sequence that includes an open reading frame that encodes a Diabrotica ctf4 protein. SEQ ID NO:22 presents a 327 amino acid sequence of a Diabrotica CTF4 protein. SEQ ID NO:278 presents a 1010 bp DNA sequence (herein referred to as ctf4 variant2 or ctf4 var2).

SEQ ID NO:23 shows an exemplary amplified fragment of ctf4 Region 1 used for in vitro dsRNA synthesis, which was amplified using primer Pair 17 (T7 at 5′ end) and primer Pair 18 (T7 at 3′ end) (Table 2). T7 promoter sequences (SEQ ID NO:98) at the 5′ and 3′ ends are not shown.

SEQ ID NO:24 shows an exemplary amplified fragment of ctf4 variant1 (herein sometimes referred to as ctf4 Var1) used for in vitro dsRNA synthesis, which was amplified using primer Pair 19 (T7 at 5′ end) and primer Pair 20 (T7 at 3′ end) (Table 2). T7 promoter sequences (SEQ ID NO:98) at the 5′ and 3′ ends are not shown.

SEQ ID NO:25 shows a DNA sequence of a ctf4 hairpin RNA for expression in corn cells or corn plants. A sense DNA segment (comprising bases 709 to 1225 of SEQ ID NO:21) is separated from a segment comprising the antisense orientation of the sense DNA bases by an ST-LS1 intron segment (SEQ ID NO:100).

Synthetic dsRNA designed to inhibit ctf4 target gene sequences caused growth inhibition when administered to WCR in diet-based assays. Table 7 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 7
Results of dsRNA diet feeding assays obtained with western
corn rootworm larvae after 9 days of feeding. ANOVA analysis
found significant differences in Mean Growth Inhibition.
Means were separated using the Tukey-Kramer test.

| Sample | Dose | Repli- | Mean % | Mean Growth |
|---|---|---|---|---|
| Name | (ng/cm²) | cations | Mortality* | Inhibition* |
| ctf4 Region | 1000 | 4 | 27.67 ± 6.05 (A) | 0.62 ± 0.06 (A) |
| 1 | | | | |
| TE** | 0 | 4 | 15.84 ± 2.72 (A) | 0.0 (B) |
| WATER | 0 | 4 | 10.18 ± 4.92 (A) | 0.00 (B) |
| YFP*** | 1000 | 4 | 16.59 ± 5.27 (A) | 0.17 ± 0.008 (B) |
| ctf4 Var1 | 1000 | 6 | 20.39 ± 5.05 (A) | 0.53 ± 0.05 (A) |
| TE buffer** | 0 | 6 | 9.31 ± 3.52 (A) | 0.00 (B) |
| WATER | 0 | 6 | 5.88 ± 1.51 (A) | 0.00 (B) |
| YFP*** | 1000 | 6 | 10.78 ± 4.66 (A) | 0.14 ± 0.06 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from ctf4 Region 1 and ctf4 Var1 exhibited increased efficacy in this assay over other dsRNAs screened.

### Example 9

### Identification of Diabrotica Spp. Candidate Target Gene: Rpl9

A candidate target gene encoding rpl9 (SEQ ID NO:26) was identified as a gene that may lead to coleopteran pest mortality, inhibition of growth, inhibition of development, or inhibition of reproduction in WCR. The Drosophila rpl9 protein is a component of the large ribosomal subunit (Schmidt et al., (1996) Molecular and General Genetics 251:381-387). The loss of rpl9 functions causes lethality.

Diabrotica rpl9 (SEQ ID NO:26) is disclosed in U.S. Patent Application No. 20070124836 and in U.S. Pat. No. 7,612,194. The Diabrotica rpl9 sequence (SEQ ID NO:26) is somewhat related to a fragment of a rpl9 gene from Hister beetle, Hister species (GENBANK Accession No. AM049014). The closest homolog of the Diabrotica RPL9 amino acid sequence (SEQ ID NO:27) is a Harpegnathos saltator L9e ribosomal protein having GENBANK Accession No. EFN86034 (97% similar; 93% identical over the homology region).

SEQ ID NO:26 presents an 815 bp DNA sequence that includes an open reading frame that encodes a Diabrotica rpl9 protein. SEQ ID NO:27 presents a 189 amino acid sequence of a Diabrotica RPL9 protein.

SEQ ID NO:28 shows an exemplary amplified fragment of rpl9 used for in vitro dsRNA synthesis, which was amplified using primer Pair 21 (T7 at 5′ end) and primer Pair 22 (T7 at 3′ end) (Table 2). T7 promoter sequences (SEQ ID NO:98) at the 5′ and 3′ ends are not shown.

SEQ ID NO:29 shows a DNA sequence of an rpl9 hairpin RNA for expression in corn cells or corn plants. A sense DNA segment (comprising bases 200 to 762 of SEQ ID NO:26) is separated from a segment comprising the antisense orientation of the sense DNA bases by an ST-LS1 intron segment (SEQ ID NO:100).

Synthetic dsRNA designed to inhibit rpl9 target gene sequences caused growth inhibition when administered to WCR in diet-based assays. Table 8 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 8
Results of dsRNA diet feeding assays obtained with western
corn rootworm larvae after 9 days of feeding. ANOVA analysis
found significant differences in Mean Growth Inhibition.
Means were separated using the Tukey-Kramer test.

| Sample | Dose | Repli- | Mean % | Mean Growth |
|---|---|---|---|---|
| Name | (ng/cm²) | cations | Mortality* | Inhibition* |
| rpl9 | 1000 | 4 | 37.67 ± 12.79 (A) | 0.77 ± 0.08 (A) |
| TE** | 0 | 4 | 7.93 ± 3.79 (A) | 0.00 (B) |
| WATER | 0 | 4 | 7.23 ± 5.46 (A) | 0.00 (B) |
| YFP*** | 1000 | 4 | 11.47 ± 7.43 (A) | 0.18 ± 0.09 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from rpl9 exhibited increased efficacy in this assay over other dsRNAs screened.

### Example 10

### Identification of Diabrotica Spp. Candidate Target Gene: Serpin Protease Inhibitor I4

A candidate target gene encoding serpin protease inhibitor I4 (SEQ ID NO:30) was identified as a gene that may lead to coleopteran pest mortality, inhibition of growth, inhibition of development, or inhibition of reproduction in WCR. Serpin protease inhibitor I4 is a serine protease inhibitor (Han et al., (2000) Febs Letters 468:194-198).

Bases 248-3165 of Diabrotica serpin protease inhibitor I4 (SEQ ID NO:30) are disclosed in U.S. Patent Application No. US20070124836-0371. There was no significant homologous nucleotide sequence found with a search in GENBANK. The closest homolog of the Diabrotica SERPIN PROTEASE INHIBITOR I4 amino acid sequence (SEQ ID NO:31) is a Tribolium casetanum protein having GENBANK Accession No. XP_001137323 (69% similar; 47% identical over the homology region).

SEQ ID NO:30 presents a 3209 bp DNA sequence that includes an open reading frame that encodes a Diabrotica serpin protease inhibitor I4 protein. SEQ ID NO:31 presents an 881 amino acid sequence of a Diabrotica SERPIN PROTEASE INHIBITOR I4 protein.

SEQ ID NO:32 shows an exemplary amplified fragment of serpin protease inhibitor I4 Region 1 used for in vitro dsRNA synthesis, which was amplified using primer Pair 23 (T7 at 5′ end) and primer Pair 24 (T7 at 3′ end) (Table 2). T7 promoter sequences (SEQ ID NO:98) at the 5′ and 3′ ends are not shown.

SEQ ID NO:33 shows an exemplary amplified fragment of serpin protease inhibitor I4 Region 2 used for in vitro dsRNA synthesis, which is amplified using primer Pair 25 (T7 at 5′ end) and primer Pair 26 (T7 at 3′ end) (Table 2). T7 promoter sequences at the 5′ and 3′ ends are not shown.

SEQ ID NO:34 shows a DNA sequence of a serpin protease inhibitor I4 hairpin RNA for expression in corn cells or corn plants. A sense DNA segment (comprising bases 1693 to 2292 of SEQ ID NO:30) is separated from a segment comprising the antisense orientation of the sense DNA bases by an ST-LS1 intron segment (SEQ ID NO:100).

Synthetic dsRNA designed to inhibit serpin protease inhibitor I4 target gene sequences caused mortality and growth inhibition when administered to WCR in diet-based assays. Table 9 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 9
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| serpin | 1000 | 4 | 41.3 ± 7.25 (A) | 0.62 ± 0.07 (A) |
| protease | | | | |
| inhibitor I4 | | | | |
| Region 1 | | | | |
| serpin | 1000 | 4 | 48.03 ± 7.25 (A) | 0.73 ± 0.07 (A) |
| protease | | | | |
| inhibitor I4 | | | | |
| Region 2 | | | | |
| TE** | 0 | 4 | 2.94 ± 7.25 (B) | 0.00 ± 0.07 (B) |
| WATER | 0 | 4 | 4.50 ± 7.25 (B) | 0.00 ± 0.07 (B) |
| YFP*** | 1000 | 4 | 3.13 ± 9.16 (B) | −0.04 ± 0.07 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from serpin protease inhibitor I4 Region 1 and serpin protease inhibitor I4 Region 2 exhibited increased efficacy in this assay over other dsRNAs screened.

### Example 11

### Identification of Diabrotica Spp. Candidate Target Gene: Myosin 3 LC

A candidate target gene encoding myosin 3 LC (SEQ ID NO:35) was identified as a gene that may lead to coleopteran pest mortality, inhibition of growth, inhibition of development, or inhibition of reproduction in WCR. The function of myosin 3 LC is unknown.

Bases 82-582 of Diabrotica myosin 3LC (SEQ ID NO:35) are disclosed in U.S. Patent Application No. US20120164205-1970. The closest homolog of the Diabrotica myosin 3 LC sequence (SEQ ID NO:35) is a Tribolium casetanum sequence having GENBANK Accession No. XM_967063.2 (85% identical over the homology region). The closest homolog of the Diabrotica MYOSIN 3 LC amino acid sequence (SEQ ID NO:36) is a Drosophila melanogaster protein having GENBANK Accession No. ACT88125.1 (100% similar; 99% identical over the homology region).

SEQ ID NO:35 presents a 722 bp DNA sequence that includes an open reading frame that encodes a Diabrotica myosin 3 LC protein. SEQ ID NO:36 presents a 150 amino acid sequence of a Diabrotica MYOSIN 3 LC protein. SEQ ID NO:279 presents a 545 bp DNA sequence (herein referred to as myosin 3 LC variant 1 or myosin 3 LC var1).

SEQ ID NO:37 shows an exemplary amplified fragment of myosin 3 LC Region 1 used for in vitro dsRNA synthesis, which was amplified using primer Pair 27 (T7 at 5′ end) and primer Pair 28 (T7 at 3′ end) (Table 2). T7 promoter sequences (SEQ ID NO:98) at the 5′ and 3′ ends are not shown.

SEQ ID NO:38 shows an exemplary amplified fragment of myosin 3 LC Region 2 used for in vitro dsRNA synthesis, which is amplified using primer Pair 29 (T7 at 5′ end) and primer Pair 30 (T7 at 3′ end) (Table 2). T7 promoter sequences at the 5′ and 3′ ends are not shown.

SEQ ID NO:39 shows a DNA sequence of a myosin 3 LC hairpin RNA for expression in corn cells or corn plants. A sense DNA segment (comprising bases 160 to 582 of SEQ ID NO:35) is separated from a segment comprising the antisense orientation of the sense DNA bases by an ST-LS1 intron segment (SEQ ID NO:100).

Synthetic dsRNA designed to inhibit myosin 3 LC target gene sequences caused growth inhibition when administered to WCR in diet-based assays. Table 10 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 10
Results of dsRNA diet feeding assays obtained with western
corn rootworm larvae after 9 days of feeding. ANOVA analysis
found significant differences in Mean Growth Inhibition.
Means were separated using the Tukey-Kramer test.

| Sample | Dose | Repli- | Mean % | Mean Growth |
|---|---|---|---|---|
| Name | (ng/cm²) | cations | Mortality* | Inhibition* |
| myosin 3 LC | 1000 | 4 | 32.58 ± 12.01 (A) | 0.68 ± 0.05 (A) |
| Region 1 | | | | |
| myosin 3 LC | 1000 | 4 | 48.16 ± 16.55 (A) | 0.63 ± 0.18 (A) |
| Region 2 | | | | |
| TE** | 0 | 4 | 12.35 ± 6.47 (A) | 0.00 (B) |
| WATER | 0 | 4 | 7.33 ± 10.9 (A) | 0.00 (B) |
| YFP*** | 1000 | 4 | 13.12 ± 14.4 (A) | 0.18 ± 0.10 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from myosin 3 LC Region 1 and myosin 3 LC Region 2 exhibited increased efficacy in this assay over other dsRNAs screened.

### Example 12

### Identification of Diabrotica Spp. Candidate Target Gene: Megator

A candidate target gene encoding megator (SEQ ID NO:40) was identified as a gene that may lead to coleopteran pest mortality, inhibition of growth, inhibition of development, or inhibition of reproduction in WCR. Megator is involved in the ribonucleoprotein complex binding.

There was no significant homologous nucleotide sequence found with a search in GENBANK. The closest homolog of the Diabrotica MEGATOR amino acid sequence (SEQ ID NO:41) is a Tribolium casetanum protein having GENBANK Accession No. gb|EFA01249 (73% similar; 58% identical over the homology region).

SEQ ID NO:40 presents a 6961 bp DNA sequence that includes an open reading frame that encodes a Diabrotica megator protein. SEQ ID NO:41 presents a 2199 amino acid sequence of a Diabrotica MEGATOR protein.

SEQ ID NO:42 shows an exemplary amplified fragment of megator Region 1 used for in vitro dsRNA synthesis, which was amplified using primer Pair 31 (T7 at 5′ end) and primer Pair 32 (T7 at 3′ end) (Table 2). T7 promoter sequences (SEQ ID NO:98) at the 5′ and 3′ ends are not shown.

SEQ ID NO:43 shows an exemplary amplified fragment of megator Region 2 used for in vitro dsRNA synthesis, which is amplified using primer Pair 33 (T7 at 5′ end) and primer Pair 34 (T7 at 3′ end) (Table 2). T7 promoter sequences at the 5′ and 3′ ends are not shown.

SEQ ID NO:44 shows a DNA sequence of a megator hairpin RNA for expression in corn cells or corn plants. A sense DNA segment (essentially bases 3813 to 4229 of SEQ ID NO:40) is separated from a segment comprising the antisense orientation of the sense DNA bases by an ST-LS1 intron segment (SEQ ID NO:100).

Synthetic dsRNA designed to inhibit megator target gene sequences caused mortality and growth inhibition when administered to WCR in diet-based assays. Table 11 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 11
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| megator Region 1 | 1000 | 4 | 40.72 ± 9.62 (A) | 0.82 ± 0.05 (A) |
| megator Region 2 | 1000 | 4 | 42.25 ± 2.325.40 (A) | 0.770 ± 0.06 (A) |
| TE** | 0 | 4 | 9.69 ± 1.945.40 (B) | 0.00 (B) |
| WATER | 0 | 6 | 12.40 ± 4.23 (B) | 0.00 (B) |
| YFP*** | 1000 | 4 | 9.16 ± 5.33 (B) | 0.27 ± 0.15 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from megator Region 1 and megator Region 2 exhibited increased efficacy in this assay over other dsRNAs screened.

### Example 13

### Identification of Diabrotica Spp. Candidate Target Gene: g Protein Beta Subunit

A candidate target gene encoding g protein beta subunit (SEQ ID NO:45) was identified as a gene that may lead to coleopteran pest mortality, inhibition of growth, inhibition of development, or inhibition of reproduction in WCR. The function of G Protein Beta Subunit is unknown.

The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. The Diabrotica g protein beta subunit (SEQ ID NO:45) is somewhat related to a fragment of a g protein beta subunit from Hydra vulgaris (GENBANK Accession No. XM_004209595). The closest homolog of the Diabrotica G PROTEIN BETA SUBUNIT amino acid sequence (SEQ ID NO:46) is a Tribolium casetanum protein having GENBANK Accession No. XP_970131 (99% similar; 99% identical over the homology region).

SEQ ID NO:45 presents a 3383 bp DNA sequence that includes an open reading frame that encodes a Diabrotica g protein beta subunit protein. SEQ ID NO:46 presents a 344 amino acid sequence of a Diabrotica G PROTEIN BETA SUBUNIT protein.

SEQ ID NO:47 shows an exemplary amplified fragment of g protein beta subunit Region 1 used for in vitro dsRNA synthesis, which was amplified using primer Pair 35 (T7 at 5′ end) and primer Pair 36 (T7 at 3′ end) (Table 2). T7 promoter sequences (SEQ ID NO:98) at the 5′ and 3′ ends are not shown.

SEQ ID NO:48 shows an exemplary amplified fragment of g protein beta subunit Region 2 used for in vitro dsRNA synthesis, which is amplified using primer Pair 37 (T7 at 5′ end) and primer Pair 38 (T7 at 3′ end) (Table 2). T7 promoter sequences at the 5′ and 3′ ends are not shown.

SEQ ID NO:49 shows a DNA sequence of a g protein beta subunit hairpin RNA for expression in corn cells or corn plants. A sense DNA segment (comprising bases 292 to 760 of SEQ ID NO:45) is separated from a segment comprising the antisense orientation of the sense DNA bases by an ST-LS1 intron segment (SEQ ID NO:100).

Synthetic dsRNA designed to inhibit g protein beta subunit target gene sequences caused mortality and growth inhibition when administered to WCR in diet-based assays. Table 12 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 12
Results of dsRNA diet feeding assays obtained with western corn
rootworm larvae after 9 days of feeding. ANOVA analysis found
significant differences in Mean % Mortality and Mean Growth
Inhibition. Means were separated using the Tukev-Kramer test.

| Sample | Dose | Repli- | Mean % | Mean Growth |
|---|---|---|---|---|
| Name | (ng/cm²) | cations | Mortality* | Inhibition* |
| g protein | 1000 | 4 | 38.83 ± 9.98 (A) | 0.76 ± 0.03 (A) |
| beta | | | | |
| subunit | | | | |
| Region 1 | | | | |
| g protein | 1000 | 4 | 50.90 ± 8.30 (A) | 0.78 ± 0.04 (A) |
| beta | | | | |
| subunit | | | | |
| Region 2 | | | | |
| TE** | 0 | 4 | 7.45 ± 2.80 (B) | 0.00 (B) |
| WATER | 0 | 4 | 4.70 ± 1.57 (B) | 0.00 (B) |
| YFP*** | 1000 | 4 | 4.61 ± 2.86 (B) | −0.07 ± 0.12 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from g protein beta subunit Region 1 and g protein beta subunit Region 2 exhibited increased efficacy in this assay over other dsRNAs screened.

### Example 14

### Identification of Diabrotica Spp. Candidate Target Gene: Flap Wing

A candidate target gene encoding flap wing (SEQ ID NO:50) was identified as a gene that may lead to coleopteran pest mortality, inhibition of growth, inhibition of development, or inhibition of reproduction in WCR. The function of flap wing is unknown.

The sequence of SEQ ID NO:50 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. The Diabrotica flap wing (SEQ ID NO:50) is somewhat related to a fragment of a sequence from Drosophila ananassae (GENBANK Accession No. XM_001963810). The closest homolog of the Diabrotica FLAP WING amino acid sequence (SEQ ID NO:51) is a Tribolium casetanum protein having GENBANK Accession No. XP_966417 (98% similar; 97% identical over the homology region).

SEQ ID NO:50 presents a 2122 bp DNA sequence that includes an open reading frame that encodes a Diabrotica flap wing protein. SEQ ID NO:51 presents a 327 amino acid sequence of a Diabrotica FLAP WING protein.

SEQ ID NO:52 shows an exemplary amplified fragment of flap wing Region 1 used for in vitro dsRNA synthesis, which was amplified using primer Pair 39 (T7 at 5′ end) and primer Pair 40 (T7 at 3′ end) (Table 2). T7 promoter sequences (SEQ ID NO:98) at the 5′ and 3′ ends are not shown.

SEQ ID NO:53 shows a DNA sequence of a flap wing hairpin RNA for expression in corn cells or corn plants. A sense DNA segment (comprising bases 580 to 1052 of SEQ ID NO:50) is separated from a segment comprising the antisense orientation of the sense DNA bases by an ST-LS1 intron segment (SEQ ID NO:100).

Synthetic dsRNA designed to inhibit flap wing target gene sequences caused mortality and growth inhibition when administered to WCR in diet-based assays. Table 13 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 13
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| flap wing | 1000 | 4 | 38.01 ± 5.51 (A) | 0.78 ± 0.05 (A) |
| Region 1 | | | | |
| TE** | 0 | 4 | 4.41 ± 2.82 (B) | 0.00 (B) |
| WATER | 0 | 4 | 4.41 ± 1.82 (B) | 0.00 (B) |
| YFP*** | 1000 | 4 | 3.13 ± 2.82 (B) | 0.15 ± 0.08 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from flap wing Region 1 exhibited increased efficacy in this assay over other dsRNAs screened.

### Example 15

### Identification of Diabrotica Spp. Candidate Target Gene: Female Sterile (2) Ketel

A candidate target gene encoding female sterile (2) ketel (SEQ ID NO:54) was identified as a gene that may lead to coleopteran pest mortality, inhibition of growth, inhibition of development, or inhibition of reproduction in WCR. Female sterile (2) ketel is involved in protein transmembrane transporter activity.

The sequence of SEQ ID NO:54 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homolog of the Diabrotica FEMALE STERILE (2) KETEL amino acid sequence (SEQ ID NO:55) is a Tribolium casetanum protein having GENBANK Accession No. XP_973263 (93% similar; 86% identical over the homology region).

SEQ ID NO:54 presents a 3472 bp DNA sequence that includes an open reading frame that encodes a Diabrotica female sterile (2) ketel protein. SEQ ID NO:55 presents a 887 amino acid sequence of a Diabrotica FEMALE STERILE (2) KETEL protein.

SEQ ID NO:56 shows an exemplary amplified fragment of female sterile (2) ketel Region 1 used for in vitro dsRNA synthesis, which was amplified using primer Pair 41 (T7 at 5′ end) and primer Pair 42 (T7 at 3′ end) (Table 2). T7 promoter sequences (SEQ ID NO:98) at the 5′ and 3′ ends are not shown.

SEQ ID NO:57 shows an exemplary amplified fragment of female sterile (2) ketel Region 2 used for in vitro dsRNA synthesis, which is amplified using primer Pair 43 (T7 at 5′ end) and primer Pair 44 (T7 at 3′ end) (Table 2). T7 promoter sequences at the 5′ and 3′ ends are not shown.

SEQ ID NO:58 shows a DNA sequence of a female sterile (2) ketel hairpin RNA for expression in corn cells or corn plants. A sense DNA segment (comprising bases 1322 to 1812 of SEQ ID NO:54) is separated from a segment comprising the antisense orientation of the sense DNA bases by an ST-LS1 intron segment (SEQ ID NO:100).

Synthetic dsRNA designed to inhibit female sterile (2) ketel target gene sequences caused mortality and growth inhibition when administered to WCR in diet-based assays. Table 14 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 14
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| female | 1000 | 4 | 45.59 ± 3.74 (A) | 0.80 ± 0.026 (A) |
| sterile | | | | |
| (2) ketel | | | | |
| Region 1 | | | | |
| female | 1000 | 4 | 31.86 ± 3.74 (A) | 0.71 ± 0.06 (A) |
| sterile | | | | |
| (2) ketel | | | | |
| Region 2 | | | | |
| Tg** | 0 | 6 | 3.92 ± 3.05 (B) | 0.00 (B) |
| WATER | 0 | 6 | 4..11 ± 3.05 (B) | 0.00 (B) |
| YFP*** | 1000 | 6 | 4.05 ± 3.05 (B) | −0.01 ± 0.09 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from female sterile (2) ketel Region 1 and female sterile (2) ketel Region 2 exhibited increased efficacy in this assay over other dsRNAs screened.

### Example 16

### Identification of Diabrotica Spp. Candidate Target Gene: Enhancer of Polycomb

A candidate target gene encoding enhancer of polycomb (SEQ ID NO:59) was identified as a gene that may lead to coleopteran pest mortality, inhibition of growth, inhibition of development, or inhibition of reproduction in WCR. The function of enhancer of polycomb is unknown.

The sequence of SEQ ID NO:59 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. The Diabrotica enhancer of polycomb sequence (SEQ ID NO:59) is somewhat related to a fragment of a sequence from the Mountain Pine Beetle, Dendroctonus ponderosae (GENBANK Accession No. APGK01059435). The closest homolog of the Diabrotica ENHANCER OF POLYCOMB amino acid sequence (SEQ ID NO:60) is a Tribolium casetanum protein having GENBANK Accession No. XP_972128 (76% similar; 66% identical over the homology region).

SEQ ID NO:59 presents a 4030 bp DNA sequence that includes an open reading frame that encodes a Diabrotica enhancer of polycomb protein. SEQ ID NO:60 presents an 852 amino acid sequence of a Diabrotica ENHANCER OF POLYCOMB protein.

SEQ ID NO:61 shows an exemplary amplified fragment of enhancer of polycomb Region 1 used for in vitro dsRNA synthesis, which was amplified using primer Pair 45 (T7 at 5′ end) and primer Pair 46 (T7 at 3′ end) (Table 2). T7 promoter sequences (SEQ ID NO:98) at the 5′ and 3′ ends are not shown.

SEQ ID NO:62 shows an exemplary amplified fragment of enhancer of polycomb Region 2 used for in vitro dsRNA synthesis, which is amplified using primer Pair 47 (T7 at 5′ end) and primer Pair 48 (T7 at 3′ end) (Table 2). T7 promoter sequences at the 5′ and 3′ ends are not shown.

SEQ ID NO:63 shows a DNA sequence of an enhancer of polycomb hairpin RNA for expression in corn cells or corn plants. A sense DNA segment (comprising bases 440 to 662 of SEQ ID NO:59) is separated from a segment comprising the antisense orientation of the sense DNA bases by an ST-LS1 intron segment (SEQ ID NO:100).

Synthetic dsRNA designed to inhibit enhancer of polycomb target gene sequences caused growth inhibition when administered to WCR in diet-based assays. Table 15 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 15
Results of dsRNA diet feeding assays obtained with western
corn rootworm larvae after 9 days of feeding. ANOVA analysis
found significant differences in Mean Growth Inhibition.
Means were separated using the Tukey-Kramer test.

| Sample | Dose | Repli- | Mean % | Mean Growth |
|---|---|---|---|---|
| Name | (ng/cm²) | cations | Mortality* | Inhibition* |
| enhancer | 1000 | 4 | 11.29 ± 6.33 (A) | 0.44 ± 0.12 (A) |
| of | | | | |
| polycomb | | | | |
| Region 1 | | | | |
| enhancer | 1000 | 4 | 9.01 ± 3.75 (A) | 0.53 ± 0.06 (A) |
| of | | | | |
| polycomb | | | | |
| Region 2 | | | | |
| TE** | 0 | 4 | 3.39 ± 1.99 (A) | 0.00 (B) |
| WATER | 0 | 4 | 6.47 ± 4.72 (A) | 0.00 (B) |
| YFP*** | 1000 | 4 | 2.94 ± 1.70 (A) | −0.01 ± 0.09 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from enhancer of polycomb Region 1 and enhancer of polycomb Region 2 exhibited increased efficacy in this assay over other dsRNAs screened.

### Example 17

### Identification of Diabrotica Spp. Candidate Target Gene: Dead Box 73D

A candidate target gene encoding dead box 73D (SEQ ID NO:64) was identified as a gene that may lead to coleopteran pest mortality, inhibition of growth, inhibition of development, or inhibition of reproduction in WCR. Dead box 73D is an ATP-dependent RNA helicase (Swiss-Prot Project, (1992) Putative ATP-dependent RNA Helicase DBP73D; Neumuller et al., (2011a) Stem Cell 8:580-593; Neumuller et al., (2011b) Supplemental Table S1).

The sequence of SEQ ID NO:64 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homologs of the Diabrotica DEAD BOX 73D amino acid sequence (SEQ ID NO:65) are a Tribolium casetanum protein having GENBANK Accession No. XM_969365.1 (73% similar; 58% identical over the homology region) and a Dendroctonus ponderosae protein having GENBANK Accession No. ENN70712 (80% similar; 65% identical over the homology region).

SEQ ID NO:64 presents a 2196 bp DNA sequence that includes an open reading frame that encodes a Diabrotica dead box 73D peptide. SEQ ID NO:65 presents a 638 amino acid sequence of a Diabrotica DEAD BOX 73D peptide.

SEQ ID NO:66 shows an exemplary amplified fragment of dead box 73D Region 1 used for in vitro dsRNA synthesis, which was amplified using primer Pair 49 (T7 at 5′ end) and primer Pair 50 (T7 at 3′ end) (Table 2). T7 promoter sequences (SEQ ID NO:98) at the 5′ and 3′ ends are not shown.

SEQ ID NO:67 shows a DNA sequence of a dead box 73D hairpin RNA for expression in corn cells or corn plants. A sense DNA segment (comprising bases 1061 to 1474 of SEQ ID NO:64) is separated from a segment comprising the antisense orientation of the sense DNA bases by an ST-LS1 intron segment (SEQ ID NO:100).

Synthetic dsRNA designed to inhibit dead box 73D target gene sequences caused growth inhibition when administered to WCR in diet-based assays. Table 16 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 16
Results of dsRNA diet feeding assays obtained with western
corn rootworm larvae after 9 days of feeding. ANOVA analysis
found significant differences in Mean Growth Inhibition.
Means were separated using the Tukey-Kramer test.

| Sample | Dose | Repli- | Mean % | Mean Growth |
|---|---|---|---|---|
| Name | (ng/cm²) | cations | Mortality* | Inhibition* |
| dead | 1000 | 4 | 22.70 ± 6.83 (A) | 0.66 ± 0.06 (A) |
| box 73D | | | | |
| Region 1 | | | | |
| TE** | 0 | 7 | 7.67 ± 3.48 (A) | 0 (B) |
| WATER | 0 | 8 | 11.45 ± 5.58 (A) | 0 (B) |
| YFP*** | 1000 | 8 | 11.79 ± 3.46 (A) | −0.03 ± 0.15 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from dead box 73D Region 1 exhibited increased efficacy in this assay over other dsRNAs screened.

### Example 18

### Identification of Diabrotica Spp. Candidate Target Gene: Cg7000

A candidate target gene encoding cg7000 (SEQ ID NO:68) was identified as a gene that may lead to coleopteran pest mortality, inhibition of growth, inhibition of development, or inhibition of reproduction in WCR. Drosophila cg7000 encodes the sensory neuron membrane protein 1 (SNMP) and may function as a scavenger receptor for signaling and lipid homeostasis (FlyBase).

The sequence of SEQ ID NO:68 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homolog of the Diabrotica cg7000 amino acid sequence (SEQ ID NO:69) is a Tribolium casetanum protein having GENBANK Accession No. XP_966331.1 (88% similar; 77% identical over the homology region).

SEQ ID NO:68 presents a 3593 bp DNA sequence that includes an open reading frame that encodes a Diabrotica cg7000 protein. SEQ ID NO:69 presents a 515 amino acid sequence of a Diabrotica CG7000 protein.

SEQ ID NO:70 shows an exemplary amplified fragment of cg7000 Region 1 used for in vitro dsRNA synthesis, which was amplified using primer Pair 51 (T7 at 5′ end) and primer Pair 52 (T7 at 3′ end) (Table 2). T7 promoter sequences (SEQ ID NO:98) at the 5′ and 3′ ends are not shown.

SEQ ID NO:71 shows a DNA sequence of a cg7000 hairpin RNA for expression in corn cells or corn plants. A sense DNA segment (comprising bases 305 to 800 of SEQ ID NO:68) is separated from a segment comprising the antisense orientation of the sense DNA bases by an ST-LS1 intron segment (SEQ ID NO:100).

Synthetic dsRNA designed to inhibit cg7000 target gene sequences caused mortality and growth inhibition when administered to WCR in diet-based assays. Table 17 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 17
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| cg7000 | 1000 | 4 | 58.68 ± 8.25 (A) | 0.68 ± .012 (A) |
| Region 1 | | | | |
| TE** | 0 | 4 | 12.50 ± 4.41 (B) | 0.00 (B) |
| WATER | 0 | 4 | 5.97 ± 2.40 (B) | 0.00 (B) |
| YFP*** | 1000 | 4 | 14.71 ± 6.12 (B) | 0.15 ± 0.14 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from cg7000 Region 1 exhibited increased efficacy in this assay over other dsRNAs screened.

### Example 19

### Identification of Diabrotica Spp. Candidate Target Gene: Heat Shock Protein 70-4 c00331

A candidate target gene encoding heat shock protein 70 c00331 (SEQ ID NO:72; herein sometimes referred to as heat shock protein 70-331 or hsp331) was identified as a gene that may lead to coleopteran pest mortality, inhibition of growth, inhibition of development, or inhibition of reproduction in WCR. The Drosophila heat shock protein 70-331 protein has been shown to be involved in protein transportation and lysosomal degradation (Gong and Golic, (2006) Genetics 172:275-286; Azad et al., (2009) PLoS ONE 4:e5371).

A segment of Diabrotica heat shock protein 70-331 sequence (SEQ ID NO:72) is disclosed in U.S. Pat. No. 7,612,194. The Diabrotica heat shock protein 70-331 (SEQ ID NO:72) is somewhat related to a fragment of a sequence from Tribolium casetanum (GENBANK Accession No. XM_965476.2). The closest homolog of the Diabrotica HEAT SHOCK PROTEIN 70-331 amino acid sequence (SEQ ID NO:73) is a Dendroctonus ponderosae protein having GENBANK Accession No. ENN75771.1 (96% similar; 93% identical over the homology region).

SEQ ID NO:72 presents a 2453 bp DNA sequence that includes an open reading frame that encodes a Diabrotica heat shock protein 70-331. SEQ ID NO:73 presents a 658 amino acid sequence of a Diabrotica HEAT SHOCK PROTEIN 70-331 protein.

SEQ ID NO:74 shows an exemplary amplified fragment of heat shock protein 70-331 Region 1 used for in vitro dsRNA synthesis, which was amplified using primer Pair 54 (T7 at 5′ end of both primers) (Table 2). T7 promoter sequences SEQ ID NO:98 at the 5′ and 3′ ends are not shown.

SEQ ID NO:75 shows a DNA sequence of a heat shock protein 70-331 hairpin RNA for expression in corn cells or corn plants. A sense DNA segment (comprising bases 123 to 600 of SEQ ID NO:72) is separated from a segment comprising the antisense orientation of the sense DNA bases by an ST-LS1 intron segment (SEQ ID NO:100).

Synthetic dsRNA designed to inhibit heat shock protein 70-331 target gene sequences caused mortality and growth inhibition when administered to WCR in diet-based assays. Table 18 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 18
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| hsp331 | 1000 | 4 | 35.14 ± 6.92 (A) | 0.65 ± 0.03 (A) |
| Region 1 | | | | |
| TE** | 0 | 4 | 11.61 ± 2.12 (B) | 0.00 ± 0.00 (C) |
| WATER | 0 | 4 | 15.59 ± 2.43 (B) | 0.00 ± 0.00 (C) |
| YFP*** | 1000 | 4 | 10.83 ± 2.60 (B) | 0.14 ± .049 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from heat shock protein 70-331 exhibited increased efficacy in this assay over other dsRNAs screened.

### Example 20

### Identification of Diabrotica Spp. Candidate Target Gene: Heat Shock Protein 70-4 c12300

A candidate target gene encoding heat shock protein 70-4 c12300 (SEQ ID NO:76; herein sometimes referred to as heat shock protein 70-12300 or hsp12300) was identified as a gene that may lead to coleopteran pest mortality, inhibition of growth, inhibition of development, or inhibition of reproduction in WCR. The Drosophila heat shock protein 70-12300 protein has been shown to be involved in protein transportation and lysosomal degradation (Gong and Golic, (2006) Genetics 172:275-286; Azad et al., (2009) PLoS ONE 4:e5371).

A segment of Diabrotica heat shock protein 70-12300 (SEQ ID NO:76) is disclosed in U.S. Patent Application No. US20070124836, U.S. Patent Application No. US201220164205, and U.S. Pat. No. 7,612,194. The Diabrotica heat shock protein 70-12300 (SEQ ID NO:76) is somewhat related to a fragment of a sequence from Tribolium casetanum (GENBANK Accession No. XM_961518.2). The closest homolog of the Diabrotica HEAT SHOCK PROTEIN 70-12300 amino acid sequence (SEQ ID NO:77) is a Tribolium casetanum protein having GENBANK Accession No. EFA12382.1 (98% similar; 94% identical over the homology region).

SEQ ID NO:76 presents a 2240 bp DNA sequence that includes an open reading frame that encodes a Diabrotica heat shock protein 70-12300. SEQ ID NO:77 presents a 648 amino acid sequence of a Diabrotica HEAT SHOCK PROTEIN 70-12300 protein.

SEQ ID NO:78 shows an exemplary amplified fragment of heat shock protein 70-12300 Region 1 used for in vitro dsRNA synthesis, which was amplified using primer Pair 53 (T7 at 5′ end of both primers)(Table 2). T7 promoter sequences (SEQ ID NO:98) at the 5′ ends are not shown.

SEQ ID NO:79 shows a DNA sequence of an heat shock protein 70-12300 hairpin RNA for expression in corn cells or corn plants. A sense DNA segment (comprising bases 84 to 500 of SEQ ID NO:76) is separated from a segment comprising the antisense orientation of the sense DNA bases by an ST-LS1 intron segment (SEQ ID NO:100).

Synthetic dsRNA designed to inhibit heat shock protein 70-12300 target gene sequences caused mortality and growth inhibition when administered to WCR in diet-based assays. Table 18 and Table 19 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 18
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| hsp12300 | 1000 | 4 | 66.67 ± 4.99 (A) | 0.83 ± 0.17 (A) |
| Region 1 | | | | |
| TE** | 0 | 4 | 11.61 ± 2.12 (B) | 0.00 ± 0.00 (C) |
| WATER | 0 | 4 | 15.59 ± 2.43 (B) | 0.00 ± 0.00 (C) |
| YFP*** | 1000 | 4 | 10.83 ± 2.60 (B) | 0.14 ± .049 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

TABLE 19
Summary of oral potency of heat shock
protein 70-12300 dsRNA on WCR larvae.

| | LC₅₀ | LC₅₀ | GI₅₀ | GI₅₀ |
|---|---|---|---|---|
| Sample Name | (ng/cm²) | Range | (ng/cm²) | Range |
| hsp12300 | 68.56 | 39.27-136.76 | 3.21 | 1.46-7.06 |

Replicated bioassays demonstrated that ingestion of dsRNA preparations derived from heat shock protein 70-12300 exhibited increased efficacy in this assay over other dsRNAs screened.

### Example 21

### Identification of Diabrotica Spp. Candidate Target Gene: Rnr1

A candidate target gene encoding rnr1 (SEQ ID NO:80) was identified as a gene that may lead to coleopteran pest mortality, inhibition of growth, inhibition of development, or inhibition of reproduction in WCR. The Drosophila rnr1 protein (ribonucleotide reductase large subunit) is the larger subunit of a ribonucleotide reductase complex that catalyzes the formation of deoxyribonucleotides from ribonucleotides. Deoxyribonucleotides in turn are used in the synthesis of DNA.

The sequence of SEQ ID NO:80 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homologs of the Diabrotica RNR1 amino acid sequence (SEQ ID NO:81) are a Tribolium casetanum protein having GENBANK Accession No. XP_968671.1 (78% similar; 62% identical over the homology region), and a Dendroctonus ponderosae protein having GENBANK Accession No. ERL85458.1 (79% similar and 65% identical over the homology region).

SEQ ID NO:80 presents a 2826 bp DNA sequence that includes an open reading frame that encodes a Diabrotica rnr1 protein. SEQ ID NO:81 presents an 807 amino acid sequence of a Diabrotica RNR1 protein.

SEQ ID NO:82 shows an exemplary amplified fragment of rnr1 Region 1 used for in vitro dsRNA synthesis, which was amplified using primer Pair 55 (T7 at 5′ end of both primers) (Table 2). T7 promoter sequences (SEQ ID NO:98) are not shown.

SEQ ID NO:83 shows an exemplary amplified fragment of rnr1 Region 2 used for in vitro dsRNA synthesis, which was amplified using primer Pair 56 (T7 at 5′ end of both primers) (Table 2). T7 promoter sequences (SEQ ID NO:98) are not shown.

SEQ ID NO:84 shows an exemplary amplified fragment of rnr1 Region 3 used for in vitro dsRNA synthesis, which was amplified using primer Pair 57 (T7 at 5′ end of both primers) (Table 2). T7 promoter sequences (SEQ ID NO:98) are not shown.

SEQ ID NO:85 shows a DNA sequence of an rnr1 hairpin RNA for expression in corn cells or corn plants. A sense DNA segment (comprising bases 219 to 509 of SEQ ID NO:80) is separated from a segment comprising the antisense orientation of the sense DNA bases by an ST-LS1 intron segment (SEQ ID NO:100).

Synthetic dsRNA designed to inhibit rnr1 target gene sequences caused mortality and growth inhibition when administered to WCR in diet-based assays. Table 20 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 20
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| rnr1 Region 1 | 500 | 4 | 64.51 ± 9.20 (A) | 0.81 ± 0.02 (A) |
| rnr1 Region 2 | 500 | 4 | 61.76 ± 7.20 (A) | 0.75 ± 0.04 (A) |
| rnr1 Region 3 | 500 | 4 | 51.87 ± 6.99 (A) | 0.70 ± 0.05 (A) |
| TE** | 0 | 4 | 17.76 ± 2.99 (B) | 0.00 ± 0.00 (B) |
| WATER | 0 | 4 | 14.70 ± 4.49 (B) | 0.00 ± 0.00 (B) |
| YFP*** | 500 | 4 | 14.70 ± 2.51 (B) | 0.14 ± .14 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from rnr1 Region 1, rnr1 Region 2, and rnr1 Region 3 exhibited increased efficacy in this assay over other dsRNAs screened.

### Example 22

### Identification of Diabrotica Spp. Candidate Target Gene: Elav

A candidate target gene encoding elav (SEQ ID NO:86) was identified as a gene that may lead to coleopteran pest mortality, inhibition of growth, inhibition of development, or inhibition of reproduction in WCR. The Drosophila elav is an mRNA polyA-binding protein (Campos et al., (1985) J. of Neurogenetics 2:197-218; Campos et al., (1987) The EMCO Journal 6:425-431).

Diabrotica elav bases 586 to 1981 and 2012-3691 of SEQ ID NO:86 have very high homology to SEQ ID NO:3566 disclosed in U.S. Patent Application No. US20120164205. The Diabrotica elav (SEQ ID NO:86) is somewhat related to a fragment of a sequence from Tribolium casetanum (GENBANK Accession No. XM_970882.2). The closest homolog of the Diabrotica ELAV amino acid sequence (SEQ ID NO:87) is a Tribolium casetanum protein having GENBANK Accession No. XP_975975.1 (91% similar; 89% identical over the homology region).

SEQ ID NO:86 presents a 3351 bp DNA sequence that includes an open reading frame that encodes a Diabrotica elav protein. SEQ ID NO:87 presents a 629 amino acid sequence of a Diabrotica ELAV protein.

SEQ ID NO:88 shows an exemplary amplified fragment of elav Region 1 used for in vitro dsRNA synthesis, which was amplified using primer Pair 58 (T7 at 5′ end of both primers) (Table 2). T7 promoter sequences (SEQ ID NO:98) are not shown.

SEQ ID NO:89 shows a DNA sequence of a elav hairpin RNA for expression in corn cells or corn plants. A sense DNA segment (comprising bases 158 to 497 of SEQ ID NO:86) is separated from a segment comprising the antisense orientation of the sense DNA bases by an ST-LS1 intron segment (SEQ ID NO:100).

Synthetic dsRNA designed to inhibit elav target gene sequences caused mortality and growth inhibition when administered to WCR in diet-based assays. Table 21 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 21
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| elav Region 1 | 1000 | 4 | 68.75 ± 3.46 (A) | 0.86 ± 0.04 (A) |
| TE** | 0 | 4 | 11.61 ± 2.12 (B) | 0.00 ± 0.00 (B) |
| WATER | 0 | 4 | 15.59 ± 2.43 (B) | 0.00 ± 0.00 (B) |
| YFP*** | 1000 | 4 | 10.83 ± 2.60 (B) | 0.14 ± .049 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from elav Region 1 exhibited increased efficacy in this assay over other dsRNAs screened.

### Example 23

### Identification of Diabrotica Spp. Candidate Target Gene: Pten

A candidate target gene encoding pten (SEQ ID NO:90) was identified as a gene that may lead to coleopteran pest mortality, inhibition of growth, inhibition of development, or inhibition of reproduction in WCR. The Drosophila pten is a phosphatase and tensin gene. The phosphatase is involved in the regulation of the cell cycle, preventing cells from growing and dividing too rapidly (Goberdhan et al., (1999) Genes and Development 13:3244-3258; Huang et al., (1999) Development 126:5365-5372; Kiger et al., (2003) J. of Biology 2:27).

The sequence of SEQ ID NO:90 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homolog of the Diabrotica PTEN amino acid sequence (SEQ ID NO:91) is a Tribolium casetanum protein having GENBANK Accession No. XP_974994.1 (76% similar; 63% identical over the homology region).

SEQ ID NO:90 presents a 1974 bp DNA sequence that includes an open reading frame that encodes a Diabrotica pten protein. SEQ ID NO:91 presents a 456 amino acid sequence of a Diabrotica PTEN protein.

SEQ ID NO:92 shows an exemplary amplified fragment of pten Region 1 used for in vitro dsRNA synthesis, which was amplified using primer Pair 59 (T7 at 5′ end of both primers) (Table 2). T7 promoter sequences (SEQ ID NO:98) are not shown.

SEQ ID NO:93 shows a DNA sequence of a pten hairpin RNA for expression in corn cells or corn plants. A sense DNA segment (comprising bases 429 to 869 of SEQ ID NO:90) is separated from a segment comprising the antisense orientation of the sense DNA bases by an ST-LS1 intron segment (SEQ ID NO:100).

Synthetic dsRNA designed to inhibit pten target gene sequences caused mortality and growth inhibition when administered to WCR in diet-based assays. Table 22 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 22
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| pten Region 1 | 1000 | 4 | 49.26 ± 5.06 (A) | 0.62 ± 0.08 (A) |
| TE** | 0 | 4 | 11.61 ± 2.12 (B) | 0.00 ± 0.00 (B) |
| WATER | 0 | 4 | 15.59 ± 2.43 (B) | 0.00 ± 0.00 (B) |
| YFP*** | 1000 | 4 | 10.83 ± 2.60 (B) | 0.14 ± .049 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from pten Region 1 exhibited increased efficacy in this assay over other dsRNAs screened.

### Example 24

### Identification of Diabrotica Spp. Candidate Target Gene: Cdc8

A candidate target gene encoding cdc8 (SEQ ID NO:94) was identified as a gene that may lead to coleopteran pest mortality, inhibition of growth, inhibition of development, or inhibition of reproduction in WCR. The Drosophila cdc8 gene encodes a thymidine kinase that functions in the synthesis of DNA and in cell division as part of the reaction chain to introduce deoxythymidine into DNA.

The sequence of SEQ ID NO:94 is novel. The sequence is not provided in public databases and is not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homologs of the Diabrotica CDC8 amino acid sequence (SEQ ID NO:95) are a Tribolium casetanum protein having GENBANK Accession No. EFA08206.1 (75% similar; 56% identical over the homology region) and a Dendroctonus ponderosae protein having GENBANK Accession No. ENN71201.1 (75% similar; 60% identical over the homology region).

SEQ ID NO:94 presents a 1019 bp DNA sequence that includes an open reading frame that encodes a Diabrotica cdc8 protein. SEQ ID NO:95 presents a 216 amino acid sequence of a Diabrotica CDC8 protein.

SEQ ID NO:96 shows an exemplary amplified fragment of cdc8 Region 1 used for in vitro dsRNA synthesis, which was amplified using primer Pair 60 (T7 at 5′ end of both primers) (Table 2). T7 promoter sequences (SEQ ID NO:98) are not shown.

SEQ ID NO:97 shows a DNA sequence of a cdc8 hairpin RNA for expression in corn cells or corn plants. A sense DNA segment (comprising bases 221 to 769 of SEQ ID NO:94) is separated from a segment comprising the antisense orientation of the sense DNA bases by an ST-LS1 intron segment (SEQ ID NO:100).

Synthetic dsRNA designed to inhibit cdc8 target gene sequences caused mortality and growth inhibition when administered to WCR in diet-based assays. Table 23 shows the results of diet-based feeding bioassays of WCR larvae following 9-day exposure to these dsRNAs, as well as the results obtained with a negative control sample of dsRNA prepared from a yellow fluorescent protein coding region (SEQ ID NO:99).

TABLE 23
Results of dsRNA diet feeding assays obtained with western corn
rootworm larvae after 9 days of feeding. ANOVA analysis found
significant differences in Mean % Mortality and Mean Growth
Inhibition. Means were separated using the Tukey-Kramer test.

| Sample | Dose | Repli- | Mean % | Mean Growth |
|---|---|---|---|---|
| Name | (ng/cm²) | cations | Mortality* | Inhibition* |
| cdc8 | 500 | 4 | 49.36 ± 6.12 (A) | 0.60 ± 0.03 (A) |
| Region 1 | | | | |
| TE** | 0 | 4 | 17.40 ± 4.07 (B) | 0.00 ± 0.00 (B) |
| WATER | 0 | 4 | 5.88 ± 7.00 (B) | 0.00 ± 0.00 (B) |
| YFP*** | 500 | 4 | 12.00 ± 2.81 (B) | −0.26 ± .049 (B) |

*Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

Replicated bioassays demonstrated that ingestion of dsRNA preparations derived from cdc8 Region 1 exhibited increased efficacy in this assay over other dsRNAs screened.

### Example 25

### Activity Testing of Annexin, Beta Spectrin, and mtRP-L4

It has previously been suggested that certain genes of Diabrotica spp. may be exploited for RNAi-mediated insect control. See U.S. Patent Publication No. 2007/0124836, which discloses 906 sequences, and U.S. Pat. No. 7,614,924, which discloses 9,112 sequences. However, it was determined that many genes suggested to have utility for RNAi-mediated insect control are not efficacious in controlling Diabrotica. It was also determined that sequences chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70 (SEQ ID NO:72 & SEQ ID NO:76), rnr1 (SEQ ID NO:80), elav (SEQ ID NO:86), pten (SEQ ID NO:90), and cdc8 (SEQ ID NO:94) each provide surprising and unexpected superior control of Diabrotica, compared to other genes suggested to have utility for RNAi-mediated insect control.

For example, annexin, beta spectrin 2, and mtRP-L4 were each suggested in U.S. Pat. No. 7,614,924 to be efficacious in RNAi-mediated insect control. SEQ ID NO:101 is the DNA sequence of annexin Region 1 (Reg1), and SEQ ID NO:102 is the DNA sequence of annexin Region 2 (Reg2). SEQ ID NO:103 is the DNA sequence of beta spectrin 2 Region 1 (Reg1), and SEQ ID NO:104 is the DNA sequence of beta spectrin 2 Region 2 (Reg2). SEQ ID NO:105 is the DNA sequence of mtRP-L4 Region 1 (Reg1), and SEQ ID NO:106 is the DNA sequence of mtRP-L4 Region 2 (Reg2). A YFP sequence (SEQ ID NO:99) was also used to produce dsRNA as a negative control.

Each of the aforementioned sequences was used to produce dsRNA by the methods of EXAMPLE 3. The strategy used to provide specific templates for dsRNA production is shown in FIG. 1. Template DNAs intended for use in dsRNA synthesis were prepared by PCR using the primer pairs in Table 2 and (as PCR template) first-strand cDNA prepared from total RNA isolated from WCR first-instar larvae. (YFP was amplified from a DNA clone.) For each selected target gene region, two separate PCR amplifications were performed. The first PCR amplification introduced a T7 promoter sequence at the 5′ end of the amplified sense strands. The second reaction incorporated the T7 promoter sequence at the 5′ ends of the antisense strands. The two PCR amplified fragments for each region of the target genes were then mixed in approximately equal amounts, and the mixture was used as transcription template for dsRNA production. See FIG. 2. Double-stranded RNA was synthesized and purified using an AMBION® MEGAscript® RNAi kit following the manufacturer's instructions (INVITROGEN). The concentrations of dsRNAs were measured using a NANODROP™ 8000 spectrophotometer (THERMO SCIENTIFIC, Wilmington, Del.). and the dsRNAs were each tested by the same diet-based bioassay methods described above. Table 24 lists the sequences of the primers used to produce the annexin Reg1, annexin Reg2, beta spectrin 2 Reg1, beta spectrin 2 Reg2, mtRP-L4 Reg1, and mtRP-L4 Reg2 dsRNA molecules. YFP primer sequences for use in the method depicted in FIG. 2 are also listed in Table 2. Table 25 presents the results of diet-based feeding bioassays of WCR larvae following 9-day exposure to these dsRNA molecules. Replicated bioassays demonstrated that ingestion of these dsRNAs resulted in no mortality or growth inhibition of western corn rootworm larvae above that seen with control samples of TE buffer, Water, or YFP protein.

TABLE 24
Primers and Primer Pairs used to amplify portions of coding regions of genes.

| | Gene | | SEQ ID | |
|---|---|---|---|---|
| | (Region) | Primer ID | NO: | Sequence |
| Pair 63 | annexin | Ann-F1_T7 | 231 | TTAATACGACTCACTATAGGGAGAG |
| | (Reg1) | | | CTCCAACAGTGGTTCCTTATC |
| | annexin | Ann-R1 | 232 | CTAATAATTCTTTTTTAATGTTCCTG |
| | (Reg1) | | | AGG |
| Pair 64 | annexin | Ann-F1 | 233 | GCTCCAACAGTGGTTCCTTATC |
| | (Reg1) | | | |
| | annexin | Ann-R1_T7 | 234 | TTAATACGACTCACTATAGGGAGAC |
| | (Reg1) | | | TAATAATTCTTTTTTAATGTTCCTGA |
| | | | | GG |
| Pair 65 | annexin | Ann-F2_T7 | 235 | TTAATACGACTCACTATAGGGAGAT |
| | (Reg2) | | | TGTTACAAGCTGGAGAACTTCTC |
| | annexin | Ann-R2 | 236 | CTTAACCAACAACGGCTAATAAGG |
| | (Reg2) | | | |
| Pair 66 | annexin | Ann-F2 | 237 | TTGTTACAAGCTGGAGAACTTCTC |
| | (Reg2) | | | |
| | annexin | Ann-R2T7 | 238 | TTAATACGACTCACTATAGGGAGAC |
| | (Reg2) | | | TTAACCAACAACGGCTAATAAGG |
| Pair 67 | beta-spect2 | Betasp2-F1_T7 | 239 | TTAATACGACTCACTATAGGGAGAA |
| | (Reg1) | | | GATGTTGGCTGCATCTAGAGAA |
| | beta-spect2 | Betasp2-R1 | 240 | GTCCATTCGTCCATCCACTGCA |
| | (Reg1) | | | |
| Pair 68 | beta-spect2 | Betasp2-F1 | 241 | AGATGTTGGCTGCATCTAGAGAA |
| | (Reg1) | | | |
| | beta-spect2 | Betasp2- | 242 | TTAATACGACTCACTATAGGGAGAG |
| | (Reg1) | R1_T7 | | TCCATTCGTCCATCCACTGCA |
| Pair 69 | beta-spect2 | Betasp2-F2_T7 | 243 | TTAATACGACTCACTATAGGGAGAG |
| | (Reg2) | | | CAGATGAACACCAGCGAGAAA |
| | beta-spect2 | Betasp2-R2 | 244 | CTGGGCAGCTTCTTGTTTCCTC |
| | (Reg2) | | | |
| Pair 70 | beta-spect2 | Betasp2-F2 | 245 | GCAGATGAACACCAGCGAGAAA |
| | (Reg2) | | | |
| | beta-spect2 | Betasp2- | 246 | TTAATACGACTCACTATAGGGAGAC |
| | (Reg2) | R2_T7 | | TGGGCAGCTTCTTGTTTCCTC |
| Pair 71 | mtRP-L4 | L4-F1_T7 | 247 | TTAATACGACTCACTATAGGGAGAA |
| | (Reg1) | | | GTGAAATGTTAGCAAATATAACATC |
| | | | | C |
| | mtRP-L4 | L4-R1 | 248 | ACCTCTCACTTCAAATCTTGACTTTG |
| | (Reg1) | | | |
| Pair 72 | mtRP-L4 | L4-F1 | 249 | AGTGAAATGTTAGCAAATATAACAT |
| | (Reg1) | | | CC |
| | mtRP-L4 | L4-R1_T7 | 250 | TTAATACGACTCACTATAGGGAGAA |
| | (Reg1) | | | CCTCTCACTTCAAATCTTGACTTTG |
| Pair 73 | mtRP-L4 | L4-F2_T7 | 251 | TTAATACGACTCACTATAGGGAGAC |
| | (Reg2) | | | AAAGTCAAGATTTGAAGTGAGAGGT |
| | mtRP-L4 | L4-R2 | 252 | CTACAAATAAAACAAGAAGGACCCC |
| | (Reg2) | | | |
| Pair 74 | mtRP-L4 | L4-F2 | 253 | CAAAGTCAAGATTTGAAGTGAGAGG |
| | (Reg2) | | | T |
| | mtRP-L4 | L4-R2_T7 | 254 | TTAATACGACTCACTATAGGGAGAC |
| | (Reg2) | | | TACAAATAAAACAAGAAGGACCCC |

TABLE 25
Results of diet feeding assays obtained with
western corn rootworm larvae after 9 days.

| | | Mean Live | | |
|---|---|---|---|---|
| | Dose | Larval Weight | Mean % | Mean Growth |
| Gene Name | (ng/cm²) | (mg) | Mortality | Inhibition |
| annexin-Reg1 | 1000 | 0.545 | 0 | −0.262 |
| annexin-Reg2 | 1000 | 0.565 | 0 | −0.301 |
| beta Spectrin2 | 1000 | 0.340 | 12 | −0.014 |
| Reg1 | | | | |
| bBeta | 1000 | 0.465 | 18 | −0.367 |
| Spectrin2 | | | | |
| Reg2 | | | | |
| mtRP-L4 Reg1 | 1000 | 0.305 | 4 | −0.168 |
| mtRP-L4 Reg2 | 1000 | 0.305 | 7 | −0.180 |
| TE buffer* | 0 | 0.430 | 13 | 0.000 |
| Water | 0 | 0.535 | 12 | 0.000 |
| YFP** | 1000 | 0.480 | 9 | −0.386 |

*TE = Tris HCl (10 mM) plus EDTA (1 mM) buffer, pH 8.
**YFP = Yellow Fluorescent Protein

### Example 26

### RNAi Constructs

Construction of Plant Transformation Vectors

Entry vectors that comprise a target gene hairpin-RNA construct which comprises a segment of a target gene sequence selected from the list comprising chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70-331 (SEQ ID NO:72), heat shock protein 70-12300 (SEQ ID NO:76), rnr1 (SEQ ID NO:80), elav (SEQ ID NO:86), pten (SEQ ID NO:90), and cdc8 (SEQ ID NO:94) are assembled using a combination of chemically synthesized fragments (DNA2.0, Menlo Park, Calif.) and standard molecular cloning methods. Intramolecular hairpin formation by RNA primary transcripts is facilitated by arranging (within a single transcription unit) two copies of a target gene segment in opposite orientation to one another, the two segments being separated by an ST-LS1 intron sequence (SEQ ID NO:100; Vancanneyt et al. (1990) Mol. Gen. Genet. 220(2):245-50). Thus, the primary mRNA transcript contains the two target gene segment sequences as large inverted repeats of one another, separated by the intron sequence. A copy of a maize ubiquitin 1 promoter (U.S. Pat. No. 5,510,474) is used to drive production of the primary mRNA hairpin transcript, and a fragment comprising a 3′ untranslated region from a maize peroxidase 5 gene (ZmPer5 3′UTR v2; U.S. Pat. No. 6,699,984) is used to terminate transcription of the hairpin-RNA-expressing gene.

Entry vectors described above are used in standard GATEWAY® recombination reactions with a typical binary destination vector to produce target gene hairpin RNA expression transformation vectors for Agrobacterium-mediated maize embryo transformations.

A negative control binary vector, which comprises a gene that expresses a YFP hairpin dsRNA, is constructed by means of standard GATEWAY® recombination reactions with a typical binary destination vector, for example, pDAB109805, and entry vector, for example, pDAB101670. An entry vector comprises a YFP hairpin sequence (SEQ ID NO:277) under the expression control of a maize ubiquitin 1 promoter (as above) and a fragment comprising a 3′ untranslated region from a maize peroxidase 5 gene (as above) is constructed.

An exemplary binary destination vector, comprises a herbicide tolerance gene (aryloxyalknoate dioxygenase; AAD-1 v3) (U.S. Pat. No. 7,838,733(B2), and Wright et al. (2010) Proc. Natl. Acad. Sci. U.S.A. 107:20240-5) under the regulation of a sugarcane bacilliform badnavirus (SCBV) promoter (Schenk et al. (1999) Plant Molec. Biol. 39:1221-30). A synthetic 5′UTR sequence, comprising sequences from a Maize Streak Virus (MSV) coat protein gene 5′UTR and intron 6 from a maize Alcohol Dehydrogenase 1 (ADH1) gene, is positioned between the 3′ end of the SCBV promoter segment and the start codon of the AAD-1 coding region. A fragment comprising a 3′ untranslated region from a maize lipase gene (ZmLip 3′UTR; U.S. Pat. No. 7,179,902) is used to terminate transcription of the AAD-1 mRNA.

Hairpin-RNA-forming sequences of target genes are disclosed: chitin synthase (SEQ ID NO:7), outer membrane translocase (SEQ ID NO:12), double parked (SEQ ID NO:16), discs overgrown (SEQ ID NO:20), ctf4 (SEQ ID NO:25), rpl9 (SEQ ID NO:29), serpin protease inhibitor I4 (SEQ ID NO:34), myosin 3 LC (SEQ ID NO:39), megator (SEQ ID NO:44), g-protein beta subunit (SEQ ID NO:49), flap wing (SEQ ID NO:53), female sterile 2 ketel (SEQ ID NO:58), enhancer of polycomb (SEQ ID NO:63), dead box 73D (SEQ ID NO:67), cg7000 (SEQ ID NO:71), heat shock protein 70-331 (SEQ ID NO:75) heat shock protein 70-12300 (SEQ ID NO:79), rnr1 (SEQ ID NO:85), elav (SEQ ID NO:89), pten (SEQ ID NO:93), and cdc8 (SEQ ID NO:97).

### Example 27

### Production of Transgenic Maize Tissues Comprising Insecticidal Hairpin dsRNAs

Agrobacterium-Mediated Transformation

Agrobacterium-mediated transformation is used to produce transgenic maize cells, tissues, and plants that produce one or more insecticidal dsRNA molecules through expression of a chimeric gene stably-integrated into the plant genome (for example, at least one dsRNA molecule is produced, including a dsRNA molecule targeting a gene comprising any of the following: chitin synthase (SEQ ID NO:1), outer membrane translocase (SEQ ID NO:8), double parked (SEQ ID NO:13), discs overgrown (SEQ ID NO:17), ctf4 (SEQ ID NO:21), rpl9 (SEQ ID NO:26), serpin protease inhibitor I4 (SEQ ID NO:30), myosin 3 LC (SEQ ID NO:35), megator (SEQ ID NO:40), g-protein beta subunit (SEQ ID NO:45), flap wing (SEQ ID NO:50), female sterile 2 ketel (SEQ ID NO:54), enhancer of polycomb (SEQ ID NO:59), dead box 73D (SEQ ID NO:64), cg7000 (SEQ ID NO:68), heat shock protein 70 (SEQ ID NO:72), heat shock protein 70-12300 SEQ ID NO:76), rnr1 (SEQ ID NO:80), elav (SEQ ID NO:86), pten (SEQ ID NO:90), and cdc8 (SEQ ID NO:94)). Maize transformation methods employing superbinary or binary transformation vectors are known in the art, as described, for example, in U.S. Pat. No. 8,304,604, which is herein incorporated by reference in its entirety. Transformed tissues are selected by their ability to grow on Haloxyfop-containing medium and are screened for dsRNA production, as appropriate. Portions of such transformed tissue cultures are presented to neonate corn rootworm larvae for bioassay, essentially as described in EXAMPLE 29.

Agrobacterium Culture Initiation

Glycerol stocks of Agrobacterium strain DAt13192 cells (WO 2012/016222A2) harboring a binary transformation vector prepared as described above (EXAMPLE 26) are streaked on AB minimal medium plates (Watson, et al., (1975) J. Bacteriol. 123:255-264) containing appropriate antibiotics and are grown at 20° C. for 3 days. The cultures are then streaked onto YEP plates (gm/L: yeast extract, 10; Peptone, 10; NaCl 5) containing the same antibiotics and are incubated at 20° C. for 1 day.

Agrobacterium Culture

On the day of an experiment, a stock solution of Inoculation Medium and acetosyringone is prepared in a volume appropriate to the number of constructs in the experiment and pipetted into a sterile, disposable, 250 mL flask. Inoculation Medium (Frame et al. (2011) Genetic Transformation Using Maize Immature Zygotic Embryos. IN Plant Embryo Culture Methods and Protocols: Methods in Molecular Biology. T. A. Thorpe and E. C. Yeung, (Eds), Springer Science and Business Media, LLC. pp 327-341) contains: 2.2 gm/L MS salts; 1×ISU Modified MS Vitamins (Frame et al., ibid.) 68.4 gm/L sucrose; 36 gm/L glucose; 115 mg/L L-proline; and 100 mg/L myo-inositol; at pH 5.4.) Acetosyringone is added to the flask containing Inoculation Medium to a final concentration of 200 μM from a 1 M stock solution in 100% dimethyl sulfoxide and the solution is thoroughly mixed.

For each construct, 1 or 2 inoculating loops-full of Agrobacterium from the YEP plate are suspended in 15 mL of the Inoculation Medium/acetosyringone stock solution in a sterile, disposable, 50 mL centrifuge tube, and the optical density of the solution at 550 nm (OD₅₅₀) is measured in a spectrophotometer. The suspension is then diluted to OD₅₅₀ of 0.3 to 0.4 using additional Inoculation Medium/acetosyringone mixture. The tube of Agrobacterium suspension is then placed horizontally on a platform shaker set at about 75 rpm at room temperature and shaken for 1 to 4 hours while embryo dissection is performed.

Ear Sterilization and Embryo Isolation

Maize immature embryos are obtained from plants of Zea mays inbred line B104 (Hallauer et al. (1997) Crop Science 37:1405-1406) grown in the greenhouse and self- or sib-pollinated to produce ears. The ears are harvested approximately 10 to 12 days post-pollination. On the experimental day, de-husked ears are surface-sterilized by immersion in a 20% solution of commercial bleach (ULTRA CLOROX® GERMICIDAL BLEACH, 6.15% sodium hypochlorite; with two drops of TWEEN 20) and shaken for 20 to 30 min, followed by three rinses in sterile deionized water in a laminar flow hood. Immature zygotic embryos (1.8 to 2.2 mm long) are aseptically dissected from each ear and randomly distributed into microcentrifuge tubes containing 2.0 mL of a suspension of appropriate Agrobacterium cells in liquid Inoculation Medium with 200 μM acetosyringone, into which 2 μL of 10% BREAK-THRU® S233 surfactant (EVONIK INDUSTRIES; Essen, Germany) is added. For a given set of experiments, embryos from pooled ears are used for each transformation.

Agrobacterium Co-Cultivation

Following isolation, the embryos are placed on a rocker platform for 5 minutes. The contents of the tube are then poured onto a plate of Co-cultivation Medium, which contains 4.33 gm/L MS salts; 1×ISU Modified MS Vitamins; 30 gm/L sucrose; 700 mg/L L-proline; 3.3 mg/L Dicamba in KOH (3,6-dichloro-o-anisic acid or 3,6-dichloro-2-methoxybenzoic acid); 100 mg/L myo-inositol; 100 mg/L Casein Enzymatic Hydrolysate; 15 mg/L AgNO₃; 200 μM acetosyringone in DMSO; and 3 gm/L GELZAN™, at pH 5.8. The liquid Agrobacterium suspension is removed with a sterile, disposable, transfer pipette. The embryos are then oriented with the scutellum facing up using sterile forceps with the aid of a microscope. The plate is closed, sealed with 3M™ MICROPORE™ medical tape, and placed in an incubator at 25° C. with continuous light at approximately 60 μmol m⁻² s⁻¹ of Photosynthetically Active Radiation (PAR).

Callus Selection and Regeneration of Transgenic Events

Following the Co-Cultivation period, embryos are transferred to Resting Medium, which is composed of 4.33 gm/L MS salts; 1×ISU Modified MS Vitamins; 30 gm/L sucrose; 700 mg/L L-proline; 3.3 mg/L Dicamba in KOH; 100 mg/L myo-inositol; 100 mg/L Casein Enzymatic Hydrolysate; 15 mg/L AgNO₃; 0.5 gm/L MES (2-(N-morpholino)ethanesulfonic acid monohydrate; PHYTOTECHNOLOGIES LABR.; Lenexa, Kans.); 250 mg/L Carbenicillin; and 2.3 gm/L GELZANG™; at pH 5.8. No more than 36 embryos are moved to each plate. The plates are placed in a clear plastic box and incubated at 27° C. with continuous light at approximately 50 μmol m⁻² s⁻¹ PAR for 7 to 10 days. Callused embryos are then transferred (<18/plate) onto Selection Medium I, which is comprised of Resting Medium (above) with 100 nM R-Haloxyfop acid (0.0362 mg/L; for selection of calli harboring the AAD-1 gene). The plates are returned to clear boxes and incubated at 27° C. with continuous light at approximately 50 μmol m⁻² s⁻¹ PAR for 7 days. Callused embryos are then transferred (<12/plate) to Selection Medium II, which is comprised of Resting Medium (above) with 500 nM R-Haloxyfop acid (0.181 mg/L). The plates are returned to clear boxes and incubated at 27° C. with continuous light at approximately 50 μmol m⁻² s⁻¹ PAR for 14 days. This selection step allows transgenic callus to further proliferate and differentiate.

Proliferating embryogenic calli are transferred (<9/plate) to Pre-Regeneration medium. Pre-Regeneration Medium contains 4.33 gm/L MS salts; 1×ISU Modified MS Vitamins; 45 gm/L sucrose; 350 mg/L L-proline; 100 mg/L myo-inositol; 50 mg/L Casein Enzymatic Hydrolysate; 1.0 mg/L AgNO₃; 0.25 gm/L MES; 0.5 mg/L naphthaleneacetic acid in NaOH; 2.5 mg/L abscisic acid in ethanol; 1 mg/L 6-benzylaminopurine; 250 mg/L Carbenicillin; 2.5 gm/L GELZAN™; and 0.181 mg/L Haloxyfop acid; at pH 5.8. The plates are stored in clear boxes and incubated at 27° C. with continuous light at approximately 50 μmol m⁻² s⁻¹ PAR for 7 days. Regenerating calli are then transferred (<6/plate) to Regeneration Medium in PHYTATRAYS™ (SIGMA-ALDRICH) and incubated at 28° C. with 16 hours light/8 hours dark per day (at approximately 160 μmol m⁻² s⁻¹ PAR) for 14 days or until shoots and roots develop. Regeneration Medium contains 4.33 gm/L MS salts; 1×ISU Modified MS Vitamins; 60 gm/L sucrose; 100 mg/L myo-inositol; 125 mg/L Carbenicillin; 3 gm/L GELLAN™ gum; and 0.181 mg/L R-Haloxyfop acid; at pH 5.8. Small shoots with primary roots are then isolated and transferred to Elongation Medium without selection. Elongation Medium contains 4.33 gm/L MS salts; 1×ISU Modified MS Vitamins; 30 gm/L sucrose; and 3.5 gm/L GELRITE™: at pH 5.8.

Transformed plant shoots selected by their ability to grow on medium containing Haloxyfop are transplanted from PHYTATRAYS™ to small pots filled with growing medium (PROMIX BX; PREMIER TECH HORTICULTURE), covered with cups or HUMI-DOMES (ARCO PLASTICS), and then hardened-off in a CONVIRON™ growth chamber (27° C. day/24° C. night, 16-hour photoperiod, 50-70% RH, 200 μmol m⁻² s⁻¹ PAR). In some instances, putative transgenic plantlets are analyzed for transgene relative copy number by quantitative real-time PCR assays using primers designed to detect the AAD1 herbicide tolerance gene integrated into the maize genome. Further, RNA qPCR assays are used to detect the presence of the ST-LS1 intron sequence in expressed dsRNAs of putative transformants. Selected transformed plantlets are then moved into a greenhouse for further growth and testing.

Transfer and establishment of T₀ plants in the greenhouse for bioassay and seed production

When plants reach the V3-V4 stage, they are transplanted into IE CUSTOM BLEND (PROFILE/METRO MIX 160) soil mixture and grown to flowering in the greenhouse (Light Exposure Type: Photo or Assimilation; High Light Limit: 1200 PAR; 16-hour day length; 27° C. day/24° C. night).

Plants to be used for insect bioassays are transplanted from small pots to TINUS™ 350-4 ROOTRAINERS® (SPENCER-LEMAIRE INDUSTRIES, Acheson, Alberta, Canada;) (one plant per event per ROOTRAINER®). Approximately four days after transplanting to ROOTRAINERS®, plants are infested for bioassay.

Plants of the T₁ generation are obtained by pollinating the silks of T₀ transgenic plants with pollen collected from plants of non-transgenic elite inbred line B104 or other appropriate pollen donors, and planting the resultant seeds. Reciprocal crosses are performed when possible.

### Example 28

### Molecular Analyses of Transgenic Maize Tissues

Molecular analyses (e.g. RNA qPCR) of maize tissues are performed on samples from leaves and roots that are collected from greenhouse grown plants on the same days that root feeding damage is assessed.

Results of RNA qPCR assays for the Per5 3′UTR are used to validate expression of hairpin transgenes. (A low level of Per5 3′UTR detection is expected in nontransformed maize plants, since there is usually expression of the endogenous Per5 gene in maize tissues.) Results of RNA qPCR assays for the ST-LS1 intron sequence (which is integral to the formation of dsRNA hairpin molecules) in expressed RNAs are used to validate the presence of hairpin transcripts. Transgene RNA expression levels are measured relative to the RNA levels of an endogenous maize gene.

DNA qPCR analyses to detect a portion of the AAD1 coding region in genomic DNA are used to estimate transgene insertion copy number. Samples for these analyses are collected from plants grown in environmental chambers. Results are compared to DNA qPCR results of assays designed to detect a portion of a single-copy native gene, and simple events (having one or two copies of the transgenes) are advanced for further studies in the greenhouse.

Additionally, qPCR assays designed to detect a portion of the spectinomycin-resistance gene (SpecR; harbored on the binary vector plasmids outside of the T-DNA) are used to determine if the transgenic plants contain extraneous integrated plasmid backbone sequences.

Hairpin RNA Transcript Expression Level: Per 5 3′UTR qPCR

Callus cell events or transgenic plants are analyzed by real time quantitative PCR (qPCR) of the Per 5 3′UTR sequence to determine the relative expression level of the full length hairpin transcript, as compared to the transcript level of an internal maize gene (SEQ ID NO:275; GENBANK Accession No. BT069734), which encodes a TIP41-like protein (i.e. a maize homolog of GENBANK Accession No. AT4G34270; having a tBLASTX score of 74% identity). RNA is isolated using an RNaeasy™ 96 kit (QIAGEN, Valencia, Calif.). Following elution, the total RNA is subjected to a DNase1 treatment according to the kit's suggested protocol. The RNA is then quantified on a NANODROP 8000 spectrophotometer (THERMO SCIENTIFIC) and concentration is normalized to 25 ng/μL. First strand cDNA is prepared using a High Capacity cDNA synthesis kit (INVITROGEN) in a 10 μL reaction volume with 5 μL denatured RNA, substantially according to the manufacturer's recommended protocol. The protocol is modified slightly to include the addition of 10 μL of 100 μM T20VN oligonucleotide (IDT) (SEQ ID NO:255; TTTTTTTTTTTTTTTTTTTTVN, where V is A, C, or G, and N is A, C, G, or T/U) into the 1 mL tube of random primer stock mix, in order to prepare a working stock of combined random primers and oligo dT.

Following cDNA synthesis, samples are diluted 1:3 with nuclease-free water, and stored at −20° C. until assayed.

Separate real-time PCR assays for the Per5 3′ UTR and TIP41-like transcript are performed on a LIGHTCYCLER™ 480 (ROCHE DIAGNOSTICS, Indianapolis, Ind.) in 10 μL reaction volumes. For the Per5 3′UTR assay, reactions are run with Primers P5U76S (F) (SEQ ID NO:256) and P5U76A (R) (SEQ ID NO:257), and a ROCHE UNIVERSAL PROBE™ (UPL76; Catalog No. 4889960001; labeled with FAM). For the TIP41-like reference gene assay, primers TIPmxF (SEQ ID NO:258) and TIPmxR (SEQ ID NO:259), and Probe HXTIP (SEQ ID NO:260) labeled with HEX (hexachlorofluorescein) are used.

All assays include negative controls of no-template (mix only). For the standard curves, a blank (water in source well) is also included in the source plate to check for sample cross-contamination. Primer and probe sequences are set forth in Table 26. Reaction components recipes for detection of the various transcripts are disclosed in Table 27, and PCR reactions conditions are summarized in Table 28. The FAM (6-Carboxy Fluorescein Amidite) fluorescent moiety is excited at 465 nm and fluorescence is measured at 510 nm; the corresponding values for the HEX (hexachlorofluorescein) fluorescent moiety are 533 nm and 580 nm.

TABLE 26
Oligonucleotide sequences used for molecular
analyses of transcript levels intransgenic maize.

| | | SEQ | |
|---|---|---|---|
| | | ID | |
| Target | Oligonucleotide | NO. | Sequence |
| Per5 | P5U76S (F) | 256 | TTGTGATGTTGGTGGC |
| 3′UTR | | | GTAT |
| Per5 | P5U76A (R) | 257 | TGTTAAATAAAACCCC |
| 3′UTR | | | AAAGATCG |
| Per5 | Roche UPL76 | NAv** | Roche Diagnostics |
| 3′UTR | (FAM-Probe) | | Catalog Number |
| | | | 488996001 |
| TIP41 | TIPmxF | 258 | TGAGGGTAATGCCAAC |
| | | | TGGTT |
| TIP41 | TIPmxR | 259 | GCAATGTAACCGAGTG |
| | | | TCTCTCAA |
| TIP41 | HXTIP | 260 | TTTTTGGCTTAGAGTT |
| | (HEX-Probe) | | GATGGTGTACTGATGA |

*TIP41-like protein.
**NAv Sequence Not Available from the supplier.

TABLE 27
PCR reaction recipes for transcript detection.

| | |
|---|---|
| Per5 3′UTR | TIP-like Gene |

| | |
|---|---|
| Component | Final Concentration |

| | | |
|---|---|---|
| Roche Buffer | 1 X | 1 X |
| P5U76S (F) | 0.4 μM | 0 |
| P5U76A (R) | 0.4 μM | 0 |
| Roche UPL76 (FAM) | 0.2 μM | 0 |
| HEXtipZM F | 0 | 0.4 μM |
| HEXtipZM R | 0 | 0.4 μM |
| HEXtipZMP (HEX) | 0 | 0.2 μM |
| cDNA (2.0 μL) | NA | NA |
| Water | To 10 μL | To 10 μL |

TABLE 28
Thermocycler conditions for RNA qPCR.
Per5 3′UTR and TIP41-like Gene Detection

| Process | Temp. | Time | | No. Cycles |
|---|---|---|---|---|
| Target Activation | 95° C. | 10 | min | 1 |
| Denature | 95° C. | 10 | sec | 40 |
| Extend | 60° C. | 40 | sec | |
| Acquire FAM or HEX | 72° C. | 1 | sec | |
| Cool | 40° C. | 10 | sec | 1 |

Data are analyzed using LIGHTCYCLER™ Software v1.5 by relative quantification using a second derivative max algorithm for calculation of Cq values according to the supplier's recommendations. For expression analyses, expression values are calculated using the ΔΔCt method (i.e., 2−(Cq TARGET−Cq REF)), which relies on the comparison of differences of Cq values between two targets, with the base value of 2 being selected under the assumption that, for optimized PCR reactions, the product doubles every cycle.

Hairpin transcript size and integrity: Northern Blot Assay

In some instances, additional molecular characterization of the transgenic plants is obtained by the use of Northern Blot (RNA blot) analysis to determine the molecular size of the hairpin RNA in transgenic plants expressing a hairpin dsRNA.

All materials and equipment are treated with RNAzap (AMBION/INVITROGEN) before use. Tissue samples (100 mg to 500 mg) are collected in 2 mL SAFELOCK EPPENDORF tubes, disrupted with a KLECKO™ tissue pulverizer (GARCIA MANUFACTURING, Visalia, Calif.) with three tungsten beads in 1 mL of TRIzol (INVITROGEN) for 5 min, then incubated at room temperature (RT) for 10 min. Optionally, the samples are centrifuged for 10 min at 4° C. at 11,000 rpm and the supernatant is transferred into a fresh 2 mL SAFELOCK EPPENDORF tube. After 200 μL of chloroform are added to the homogenate, the tube is mixed by inversion for 2 to 5 min, incubated at RT for 10 minutes, and centrifuged at 12,000×g for 15 min at 4° C. The top phase is transferred into a sterile 1.5 mL EPPENDORF tube, 600 μL of 100% isopropanol are added, followed by incubation at RT for 10 min to 2 hr, then centrifuged at 12,000×g for 10 min at 4° to 25° C. The supernatant is discarded and the RNA pellet is washed twice with 1 mL of 70% ethanol, with centrifugation at 7,500×g for 10 min at 4° to 25° C. between washes. The ethanol is discarded and the pellet is briefly air dried for 3 to 5 min before resuspending in 50 μL of nuclease-free water.

Total RNA is quantified using the NANODROP8000® (THERMO-FISHER) and samples are normalized to 5 μg/10 μL. 10 μL of glyoxal (AMBION/INVITROGEN) are then added to each sample. Five to 14 ng of DIG RNA standard marker mix (ROCHE APPLIED SCIENCE, Indianapolis, Ind.) are dispensed and added to an equal volume of glyoxal. Samples and marker RNAs are denatured at 50° C. for 45 min and stored on ice until loading on a 1.25% SEAKEM GOLD agarose (lONZA, Allendale, N.J.) gel in NORTHERNMAX 10× glyoxal running buffer (AMBION/INVITROGEN). RNAs are separated by electrophoresis at 65 volts/30 mA for 2 hr and 15 min.

Following electrophoresis, the gel is rinsed in 2×SSC for 5 min and imaged on a GEL DOC station (BIORAD, Hercules, Calif.), then the RNA is passively transferred to a nylon membrane (MILLIPORE) overnight at RT, using 10×SSC as the transfer buffer (20×SSC consists of 3 M sodium chloride and 300 mM trisodium citrate, pH 7.0). Following the transfer, the membrane is rinsed in 2×SSC for 5 minutes, the RNA is UV-crosslinked to the membrane (AGILENT/STRATAGENE), and the membrane is allowed to dry at RT for up to 2 days.

The membrane is prehybridized in ULTRAHYB buffer (AMBION/INVITROGEN) for 1 to 2 hr. The probe consists of a PCR amplified product containing the sequence of interest, (for example, the antisense sequence portion hairpins of chitin synthase (SEQ ID NO:7), outer membrane translocase (SEQ ID NO:12), double parked (SEQ ID NO:16), discs overgrown (SEQ ID NO:20), ctf4 (SEQ ID NO:25), rpl9 (SEQ ID NO:29), serpin protease inhibitor I4 (SEQ ID NO:34), myosin 3 LC (SEQ ID NO:39), megator (SEQ ID NO:44), g-protein beta subunit (SEQ ID NO:49), flap wing (SEQ ID NO:53), female sterile 2 ketel (SEQ ID NO:58), enhancer of polycomb (SEQ ID NO:63), dead box 73D (SEQ ID NO:67), cg7000 (SEQ ID NO:71), heat shock protein 70-331 (SEQ ID NO:75) heat shock protein 70-12300 (SEQ ID NO:79), rnr1 (SEQ ID NO:85), elav (SEQ ID NO:89), pten (SEQ ID NO:93), or cdc8 (SEQ ID NO:97), as appropriate) labeled with digoxygenin by means of a ROCHE APPLIED SCIENCE DIG procedure. Hybridization in recommended buffer is overnight at a temperature of 60° C. in hybridization tubes. Following hybridization, the blot is subjected to DIG washes, wrapped, exposed to film for 1 to 30 minutes, then the film is developed, all by methods recommended by the supplier of the DIG kit.

Transgene Copy Number Determination

Maize leaf pieces approximately equivalent to 2 leaf punches are collected in 96-well collection plates (QIAGEN). Tissue disruption is performed with a KLECKO™ tissue pulverizer in BIOSPRINT96 AP1 lysis buffer (supplied with a BIOSPRINT96 PLANT KIT;) with one stainless steel bead. Following tissue maceration, genomic DNA (gDNA) is isolated in high throughput format using a BIOSPRINT96 PLANT KIT and a BIOSPRINT96 extraction robot. Genomic DNA is diluted 2:3 DNA:water prior to setting up the qPCR reaction.

qPCR analysis Transgene detection by hydrolysis probe assay is performed by real-time PCR using a LIGHTCYCLER®480 system. Oligonucleotides to be used in hydrolysis probe assays to detect the ST-LS1 intron sequence (SEQ ID NO:100), or to detect a portion of the SpecR gene (i.e. the spectinomycin resistance gene borne on the binary vector plasmids; SEQ ID NO:272; SPC1 oligonucleotides in Table 29), are designed using LIGHTCYCLER® PROBE DESIGN Software 2.0. Further, oligonucleotides to be used in hydrolysis probe assays to detect a segment of the AAD-1 herbicide tolerance gene (SEQ ID NO:276; GAAD1 oligonucleotides in Table 29) are designed using PRIMER EXPRESS software (APPLIED BIOSYSTEMS). Table 29 shows the sequences of the primers and probes. Assays are multiplexed with reagents for an endogenous maize chromosomal gene (Invertase (SEQ ID NO:275); GENBANK Accession No: U16123; referred to herein as IVR1), which serves as an internal reference sequence to ensure gDNA is present in each assay. For amplification, LIGHTCYCLER®480 PROBE MASTER MIX (ROCHE APPLIED SCIENCE) is prepared at 1× final concentration in a 10 μL volume multiplex reaction containing 0.4 μM of each primer and 0.2 μM of each probe (Table 30). A two step amplification reaction is performed as outlined in Table 31. Fluorophore activation and emission for the FAM- and HEX-labeled probes are as described above; CY5 conjugates are excited maximally at 650 nm and fluoresce maximally at 670 nm.

Cp scores (the point at which the fluorescence signal crosses the background threshold) are determined from the real time PCR data using the fit points algorithm (LightCycler® software release 1.5) and the Relative Quant module (based on the ΔΔCt method). Data are handled as described previously (above, RNA qPCR).

TABLE 29
Sequences of primers and probes
(with fluorescent conjugate)
used for gene copy number determinations and
binary vector plasmid backbone detection.

| | SEQ | |
|---|---|---|
| | ID | |
| Name | NO: | Sequence |
| ST-LS1-F | 261 | GTATGTTTCTGCTTCTACCTTTGAT |
| ST-LS1-R | 262 | CCATGTTTTGGTCATATATTAGAAAAGTT |
| ST-LS1-P (FAM) | 263 | AGTAATATAGTATTTCAAGTATTTTTTTC |
| | | AAAAT |
| GAAD1-F | 264 | TGTTCGGTTCCCTCTACCAA |
| GAAD1-R | 265 | CAACATCCATCACCTTGACTGA |
| GAAD1-P (FAM) | 266 | CACAGAACCGTCGCTTCAGCAACA |
| IVR1-F | 267 | TGGCGGACGACGACTTGT |
| IVR1-R | 268 | AAAGTTTGGAGGCTGCCGT |
| IVR1-P (HEX) | 269 | CGAGCAGACCGCCGTGTACTTCTACC |
| SPC1A | 270 | CTTAGCTGGATAACGCCAC |
| SPC1S | 271 | GACCGTAAGGCTTGATGAA |
| TQSPEC (CY5*) | 272 | CGAGATTCTCCGCGCTGTAGA |

*CY5 = Cyanine-5

TABLE 30
Reaction components for gene copy number analyses
and binary vector plasmid backbone detection.

| Component | Amt. (μL) | Stock | Final Conc'n |
|---|---|---|---|
| 2x Buffer | 5.0 | 2x | 1x |
| Appropriate Forward Primer | 0.4 | 10 μM | 0.4 |
| Appropriate Reverse Primer | 0.4 | 10 μM | 0.4 |
| Appropriate Probe | 0.4 | 5 μM | 0.2 |
| IVR1-Forward Primer | 0.4 | 10 μM | 0.4 |
| IVR1-Reverse Primer | 0.4 | 10 μM | 0.4 |
| IVRl-Probe | 0.4 | 5 μM | 0.2 |
| H₂O | 0.6 | NA* | NA |
| gDNA | 2.0 | ND** | ND |
| Total | 10.0 | | |

*NA = Not Applicable
**ND = Not Determined

TABLE 31
Thermocycler conditions for DNA qPCR.
Genomic copy number analyses

| Process | Temp. | Time | | No. Cycles |
|---|---|---|---|---|
| Target Activation | 95° C. | 10 | min | 1 |
| Denature | 95° C. | 10 | sec | 40 |
| Extend & Acquire | 60° C. | 40 | sec | |
| FAM, HEX, or CY5 | | | | |
| Cool | 40° C. | 10 | sec | 1 |

### Example 29

### Plant Bioassay of Transgenic Maize

In Vitro Insect Bioassays

Bioactivity of dsRNAs of the subject invention produced in plant cells are demonstrated by bioassay methods. See, e.g., Baum et al. (2007) Nat. Biotechnol. 25(11):1322-1326. One is able to demonstrate efficacy, for example, by feeding various plant tissues or tissue pieces derived from a plant producing an insecticidal dsRNA to target insects in a controlled feeding environment. Alternatively, extracts are prepared from various plant tissues derived from a plant producing the insecticidal dsRNA and the extracted nucleic acids are dispensed on top of artificial diets for bioassays as previously described herein. The results of such feeding assays are compared to similarly conducted bioassays that employ appropriate control tissues from host plants that do not produce an insecticidal dsRNA, or to other control samples.

In Vivo Insect Bioassays with Transgenic Maize Events

Two western corn rootworm larvae (1 to 3 days old) hatched from washed eggs are selected and placed into each well of the bioassay tray. The wells are then covered with a “pull n' peel” tab cover (BIO-CV-16, BIO-SERV) and placed in a 28° C. incubator with an 18 hr/6 hr light/dark cycle. Nine days after the initial infestation, the larvae are assessed for mortality, which is calculated as the percentage of dead insects out of the total number of insects in each treatment. The insect samples are frozen at −20° C. for two days, then the insect larvae from each treatment are pooled and weighed. The percent of growth inhibition is calculated as the mean weight of the experimental treatments divided by the mean of the average weight of two control well treatments. The data are expressed as a Percent Growth Inhibition (of the Negative Controls). Mean weights that exceed the control mean weight are normalized to zero.

Insect Bioassays in the Greenhouse

Western corn rootworm (WCR, Diabrotica virgifera virgifera LeConte) eggs are received in soil from CROP CHARACTERISTICS (Farmington, Minn.). WCR eggs are incubated at 28° C. for 10 to 11 days. Eggs are washed from the soil, placed into a 0.15% agar solution, and the concentration is adjusted to approximately 75 to 100 eggs per 0.25 mL aliquot. A hatch plate is set up in a Petri dish with an aliquot of egg suspension to monitor hatch rates.

The soil around the maize plants growing in ROOTRAINERS® is infested with 150 to 200 WCR eggs. The insects are allowed to feed for 2 weeks, after which time a “Root Rating” is given to each plant. A Node-Injury Scale is utilized for grading essentially according to Oleson et al. (2005) J. Econ. Entomol. 98(1):1-8. Plants which pass this bioassay are transplanted to 5-gallon pots for seed production. Transplants are treated with insecticide to prevent further rootworm damage and insect release in the greenhouses. Plants are hand pollinated for seed production. Seeds produced by these plants are saved for evaluation at the Ti and subsequent generations of plants.

Greenhouse bioassays include two kinds of negative control plants. Transgenic negative control plants are generated by transformation with vectors harboring genes designed to produce a yellow fluorescent protein (YFP) or a YFP hairpin dsRNA (See EXAMPLE 26). Nontransformed negative control plants are grown from seeds of line B104. Bioassays are conducted with negative controls included in each set of plant materials.

### Example 30

### Transgenic Zea mays Comprising Coleopteran Pest Sequences

Ten to 20 transgenic T₀ Zea mays plants are generated as described in EXAMPLE 27. A further 10-20 T₁ Zea mays independent lines expressing hairpin dsRNA for an RNAi construct are obtained for corn rootworm challenge. Hairpin dsRNA may be derived as set forth in SEQ ID NO:7, SEQ ID NO:12, SEQ ID NO:16, SEQ ID NO:20, SEQ ID NO:25, SEQ ID NO:29, SEQ ID NO:34, SEQ ID NO:39, SEQ ID NO:44, SEQ ID NO:49, SEQ ID NO:53, SEQ ID NO:58, SEQ ID NO:63, SEQ ID NO:67, SEQ ID NO:71, SEQ ID NO:75, SEQ ID NO:79, SEQ ID NO:85, SEQ ID NO:89, SEQ ID NO:93, and SEQ ID NO:97, or otherwise further comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45), SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94. Additional hairpin dsRNAs may be derived, for example, from coleopteran pest sequences such as, for example, Caf1-180 (U.S. Patent Application Publication No. 2012/0174258), VatpaseC (U.S. Patent Application Publication No. 2012/0174259), Rho1 (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), or RPS6 (U.S. Patent Application Publication No. 2013/0097730). These are confirmed through RT-PCR or other molecular analysis methods. Total RNA preparations from selected independent T₁ lines are optionally used for RT-PCR with primers designed to bind in the ST-LS1 intron of the hairpin expression cassette in each of the RNAi constructs. In addition, specific primers for each target gene in an RNAi construct are optionally used to amplify and confirm the production of the pre-processed mRNA required for siRNA production in planta. The amplification of the desired bands for each target gene confirms the expression of the hairpin RNA in each transgenic Zea mays plant. Processing of the dsRNA hairpin of the target genes into siRNA is subsequently optionally confirmed in independent transgenic lines using RNA blot hybridizations.

Moreover, RNAi molecules having mismatch sequences with more than 80% sequence identity to target genes affect corn rootworms in a way similar to that seen with RNAi molecules having 100% sequence identity to the target genes. The pairing of mismatch sequence with native sequences to form a hairpin dsRNA in the same RNAi construct delivers plant-processed siRNAs capable of affecting the growth, development and viability of feeding coleopteran pests.

In planta delivery of dsRNA, siRNA or miRNA corresponding to target genes and the subsequent uptake by coleopteran pests through feeding results in down-regulation of the target genes in the coleopteran pest through RNA-mediated gene silencing. When the function of a target gene is important at one or more stages of development, the growth, development, and reproduction of the coleopteran pest is affected, and in the case of at least one of WCR, NCR, SCR, MCR, D. balteata LeConte, D. u. tenella, D. speciosa Germar, and D. u. undecimpunctata Mannerheim, leads to failure to successfully infest, feed, develop, and/or reproduce, or leads to death of the coleopteran pest. The choice of target genes and the successful application of RNAi is then used to control coleopteran pests.

Phenotypic Comparison of Transgenic RNAi Lines and Nontransformed Zea mays

Target coleopteran pest genes or sequences selected for creating hairpin dsRNA have no similarity to any known plant gene sequence. Hence it is not expected that the production or the activation of (systemic) RNAi by constructs targeting these coleopteran pest genes or sequences will have any deleterious effect on transgenic plants. However, development and morphological characteristics of transgenic lines are compared with nontransformed plants, as well as those of transgenic lines transformed with an “empty” vector having no hairpin-expressing gene. Plant root, shoot, foliage and reproduction characteristics are compared. There is no observable difference in root length and growth patterns of transgenic and nontransformed plants. Plant shoot characteristics such as height, leaf numbers and sizes, time of flowering, floral size and appearance are similar. In general, there are no observable morphological differences between transgenic lines and those without expression of target iRNA molecules when cultured in vitro and in soil in the greenhouse.

### Example 31

### Transgenic Zea mays Comprising a Coleopteran Pest Sequence and Additional RNAi Constructs

A transgenic Zea mays plant comprising a heterologous coding sequence in its genome that is transcribed into an iRNA molecule that targets an organism other than a coleopteran pest is secondarily transformed via Agrobacterium or WHISKERS™ methodologies (see Petolino and Arnold (2009) Methods Mol. Biol. 526:59-67) to produce one or more insecticidal dsRNA molecules (for example, at least one dsRNA molecule including a dsRNA molecule targeting a gene comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45), SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94). Plant transformation plasmid vectors prepared essentially as described in EXAMPLE 26 are delivered via Agrobacterium or WHISKERS™-mediated transformation methods into maize suspension cells or immature maize embryos obtained from a transgenic Hi II or B104 Zea mays plant comprising a heterologous coding sequence in its genome that is transcribed into an iRNA molecule that targets an organism other than a coleopteran pest.

### Example 32

### Transgenic Zea mays Comprising an RNAi Construct and Additional Coleopteran Pest Control Sequences

A transgenic Zea mays plant comprising a heterologous coding sequence in its genome that is transcribed into an iRNA molecule that targets a coleopteran pest organism (for example, at least one dsRNA molecule including a dsRNA molecule targeting a gene comprising SEQ ID NO:1, SEQ ID NO:8, SEQ ID NO:13, SEQ ID NO:17, SEQ ID NO:21, SEQ ID NO:26, SEQ ID NO:30, SEQ ID NO:35, SEQ ID NO:40, SEQ ID NO:45), SEQ ID NO:50, SEQ ID NO:54, SEQ ID NO:59, SEQ ID NO:64, SEQ ID NO:68, SEQ ID NO:72, SEQ ID NO:76, SEQ ID NO:80, SEQ ID NO:86, SEQ ID NO:90, or SEQ ID NO:94) is secondarily transformed via Agrobacterium or WHISKERS™ methodologies to produce one or more insecticidal protein molecules, for example, Cry 3, Cry 6, Cry34 and Cry 35 insecticidal proteins. Plant transformation plasmid vectors prepared essentially as described in EXAMPLE 26 are delivered via Agrobacterium or WHISKERS™-mediated transformation methods into maize suspension cells or immature maize embryos obtained from a transgenic B104 Zea mays plant comprising a heterologous coding sequence in its genome that is transcribed into an iRNA molecule that targets a coleopteran pest organism. Doubly-transformed plants are obtained that produce iRNA molecules and insecticidal proteins for control of coleopteran pests.

What may be claimed is:

1. An isolated nucleic acid comprising at least one polynucleotide operably linked to a heterologous promoter, wherein the polynucleotide is selected from the group consisting of:
SEQ ID NO:1; the complement of SEQ ID NO:1; a fragment of at least 15 contiguous nucleotides of SEQ ID NO:1; the complement of a fragment of at least 15 contiguous nucleotides of SEQ ID NO:1; a native coding sequence of a Coleopteran organism comprising SEQ ID NO:1; the complement of a native coding sequence of a Coleopteran organism comprising SEQ ID NO:1; a fragment of at least 15 contiguous nucleotides of a native coding sequence of a Coleopteran organism comprising SEQ ID NO:1; the complement of a fragment of at least 15 contiguous nucleotides of a native coding sequence of a Coleopteran organism comprising SEQ ID NO:1; native coding sequence of a Coleopteran organism comprising SEQ ID NOs:3-7; the complement of a native coding sequence of a Coleopteran organism comprising of SEQ ID NOs:3-7; a fragment of at least 15 contiguous nucleotides of a native coding sequence of a Coleopteran organism comprising SEQ ID NOs:3-7; the complement of a fragment of at least 15 contiguous nucleotides of a native coding sequence of a Coleopteran organism comprising SEQ ID NOs:3-7;
SEQ ID NO:8; the complement of SEQ ID NO:8; a fragment of at least 15 contiguous nucleotides of SEQ ID NO:8; the complement of a fragment of at least 15 contiguous nucleotides of SEQ ID NO:8; a native coding sequence of a Coleopteran organism comprising SEQ ID NO:8; the complement of a native coding sequence of a Coleopteran organism comprising SEQ ID NO:8; a fragment of at least 15 contiguous nucleotides of a native coding sequence of a Coleopteran organism comprising SEQ ID NO:8; the complement of a fragment of at least 15 contiguous nucleotides of a native coding sequence of a Coleopteran organism comprising SEQ ID NO:8; native coding sequence of a Coleopteran organism comprising SEQ ID NOs:10-12; the complement of a native coding sequence of a Coleopteran organism comprising of SEQ ID NOs:10-12; a fragment of at least 15 contiguous nucleotides of a native coding sequence of a Coleopteran organism comprising SEQ ID NOs:10-12; the complement of a fragment of at least 15 contiguous nucleotides of a native coding sequence of a Coleopteran organism comprising SEQ ID NOs:10-12;
SEQ ID NO:13; the complement of SEQ ID NO:13; a fragment of at least 15 contiguous nucleotides of SEQ ID NO:13; the complement of a fragment of at least 15 contiguous nucleotides of SEQ ID NO:13; a native coding sequence of a Coleopteran organism comprising SEQ ID NO:13; the complement of a native coding sequence of a Coleopteran organism comprising SEQ ID NO:13; a fragment of at least 15 contiguous nucleotides of a native coding sequence of a Coleopteran organism comprising SEQ ID NO:13; the complement of a fragment of at least 15 contiguous nucleotides of a native coding sequence of a Coleopteran organism comprising SEQ ID NO:13; native coding sequence of a Coleopteran organism comprising SEQ ID NOs:15-16; the complement of a native coding sequence of a Coleopteran organism comprising of SEQ ID NOs:15-16; a fragment of at least 15 contiguous nucleotides of a native coding sequence of a Coleopteran organism comprising SEQ ID NOs:15-16; the complement of a fragment of at least 15 contiguous nucleotides of a native coding sequence of a Coleopteran organism comprising SEQ ID NOs:15-16;
SEQ ID NO:17; the complement of SEQ ID NO:17; a fragment of at least 15 contiguous nucleotides of SEQ ID NO:17; the complement of a fragment of at least 15 contiguous nucleotides of SEQ ID NO:17; a native coding sequence of a coleopteran organism comprising SEQ ID NO:17; the complement of a native coding sequence of a coleopteran organism comprising SEQ ID NO:17; a fragment of at least 15 contiguous nucleotides of a native coding sequence of a coleopteran organism comprising SEQ ID NO:17; the complement of a fragment of at least 15 contiguous nucleotides of a native coding sequence of a Coleopteran organism comprising SEQ ID NO:17; native coding sequence of a Coleopteran organism comprising SEQ ID NOs:19-20; the complement of a native coding sequence of a Coleopteran organism comprising of SEQ ID NOs:19-20; a fragment of at least 15 contiguous nucleotides of a native coding sequence of a Coleopteran organism comprising SEQ ID NOs:19-20; the complement of a fragment of at least 15 contiguous nucleotides of a native coding sequence of a Coleopteran organism comprising SEQ ID NOs:19-20;
SEQ ID NO:21; the complement of SEQ ID NO:21; a fragment of at least 15 contiguous nucleotides of SEQ ID NO:21; the complement of a fragment of at least 15 contiguous nucleotides of SEQ ID NO:21; a native coding sequence of a coleopteran organism comprising SEQ ID NO:21; the complement of a native coding sequence of a coleopteran organism comprising SEQ ID NO:21; a fragment of at least 15 contiguous nucleotides of a native coding sequence of a coleopteran organism comprising SEQ ID NO:21; the complement of a fragment of at least 15 contiguous nucleotides of a native coding sequence of a Coleopteran organism comprising SEQ ID NO:21; native coding sequence of a Coleopteran organism comprising SEQ ID NOs:23-25; the complement of a native coding sequence of a Coleopteran organism comprising of SEQ ID NOs:23-25; a fragment of at least 15 contiguous nucleotides of a native coding sequence of a Coleopteran organism comprising SEQ ID NOs:23-25; the complement of a fragment of at least 15 contiguous nucleotides of a native coding sequence of a Coleopteran organism comprising SEQ ID NOs:23-25;
SEQ ID NO:26; the complement of SEQ ID NO:26; a fragment of at least 15 contiguous nucleotides of SEQ ID NO:26; the complement of a fragment of at least 15 contiguous nucleotides of SEQ ID NO:26; a native coding sequence of a coleopteran organism comprising SEQ ID NO:26; the complement of a native coding sequence of a coleopteran organism comprising SEQ ID NO:26; a fragment of at least 15 contiguous nucleotides of a native coding sequence of a coleopteran organism comprising SEQ ID NO:26; the complement of a fragment of at least 15 contiguous nucleotides of a native coding sequence of a Coleopteran organism comprising SEQ ID NO:26; native coding sequence of a Coleopteran organism comprising SEQ ID NOs:28-29; the complement of a native coding sequence of a Coleopteran organism comprising of SEQ ID NOs:28-29; a fragment of at least 15 contiguous nucleotides of a native coding sequence of a Coleopteran organism comprising SEQ ID NOs:28-29; the complement of a fragment of at least 15 contiguous nucleotides of a native coding sequence of a Coleopteran organism comprising SEQ ID NOs:28-29;
SEQ ID NO:30; the complement of SEQ ID NO:30; a fragment of at least 15 contiguous nucleotides of SEQ ID NO:30; the complement of a fragment of at least 15 contiguous nucleotides of SEQ ID NO:30; a native coding sequence of a coleopteran organism comprising SEQ ID NO:30; the complement of a native coding sequence of a coleopteran organism comprising SEQ ID NO:30; a fragment of at least 15 contiguous nucleotides of a native coding sequence of a coleopteran organism comprising SEQ ID NO:30; the complement of a fragment of at least 15 contiguous nucleotides of a native coding sequence of a Coleopteran organism comprising SEQ ID NO:30; native coding sequence of a Coleopteran organism comprising SEQ ID NOs:32-34; the complement of a native coding sequence of a Coleopteran organism comprising of SEQ ID NOs:32-34; a fragment of at least 15 contiguous nucleotides of a native coding sequence of a Coleopteran organism comprising SEQ ID NOs:32-34; the complement of a fragment of at least 15 contiguous nucleotides of a native coding sequence of a Coleopteran organism comprising SEQ ID NOs:32-34;
SEQ ID NO:35; the complement of SEQ ID NO:35; a fragment of at least 15 contiguous nucleotides of SEQ ID NO:35; the complement of a fragment of at least 15 contiguous nucleotides of SEQ ID NO:35; a native coding sequence of a coleopteran organism comprising SEQ ID NO:35; the complement of a native coding sequence of a coleopteran organism comprising SEQ ID NO:35; a fragment of at least 15 contiguous nucleotides of a native coding sequence of a coleopteran organism comprising SEQ ID NO:35; the complement of a fragment of at least 15 contiguous nucleotides of a native coding sequence of a Coleopteran organism comprising SEQ ID NO:35; native coding sequence of a Coleopteran organism comprising SEQ ID NOs:37-39; the complement of a native coding sequence of a Coleopteran organism comprising of SEQ ID NOs:37-39; a fragment of at least 15 contiguous nucleotides of a native coding sequence of a Coleopteran organism comprising SEQ ID NOs:37-39; the complement of a fragment of at least 15 contiguous nucleotides of a native coding sequence of a Coleopteran organism comprising SEQ ID NOs:37-39;
SEQ ID NO:40; the complement of SEQ ID NO:40; a fragment of at least 15 contiguous nucleotides of SEQ ID NO:40; the complement of a fragment of at least 15 contiguous nucleotides of SEQ ID NO:40; a native coding sequence of a coleopteran organism comprising SEQ ID NO:40; the complement of a native coding sequence of a coleopteran organism comprising SEQ ID NO:40; a fragment of at least 15 contiguous nucleotides of a native coding sequence of a coleopteran organism comprising SEQ ID NO:40; the complement of a fragment of at least 15 contiguous nucleotides of a native coding sequence of a Coleopteran organism comprising SEQ ID NO:40; native coding sequence of a Coleopteran organism comprising SEQ ID NOs:42-44; the complement of a native coding sequence of a Coleopteran organism comprising of SEQ ID NOs:42-44; a fragment of at least 15 contiguous nucleotides of a native coding sequence of a Coleopteran organism comprising SEQ ID NOs:42-44; the complement of a fragment of at least 15 contiguous nucleotides of a native coding sequence of a Coleopteran organism comprising SEQ ID NOs:42-44;
SEQ ID NO:45; the complement of SEQ ID NO:45; a fragment of at least 15 contiguous nucleotides of SEQ ID NO:45; the complement of a fragment of at least 15 contiguous nucleotides of SEQ ID NO:1; a native coding sequence of a coleopteran organism comprising SEQ ID NO:45; the complement of a native coding sequence of a coleopteran organism comprising SEQ ID NO:45; a fragment of at least 15 contiguous nucleotides of a native coding sequence of a coleopteran organism comprising SEQ ID NO:45; the complement of a fragment of at least 15 contiguous nucleotides of a native coding sequence of a Coleopteran organism comprising SEQ ID NO:45; native coding sequence of a Coleopteran organism comprising SEQ ID NOs:47-49; the complement of a native coding sequence of a Coleopteran organism comprising of SEQ ID NOs:47-49; a fragment of at least 15 contiguous nucleotides of a native coding sequence of a Coleopteran organism comprising SEQ ID NOs:47-49; the complement of a fragment of at least 15 contiguous nucleotides of a native coding sequence of a Coleopteran organism comprising SEQ ID NOs:47-49;
SEQ ID NO:50; the complement of SEQ ID NO:50; a fragment of at least 15 contiguous nucleotides of SEQ ID NO:50; the complement of a fragment of at least 15 contiguous nucleotides of SEQ ID NO:50; a native coding sequence of a coleopteran organism comprising SEQ ID NO:50; the complement of a native coding sequence of a coleopteran organism comprising SEQ ID NO:50; a fragment of at least 15 contiguous nucleotides of a native coding sequence of a coleopteran organism comprising SEQ ID NO:50; the complement of a fragment of at least 15 contiguous nucleotides of a native coding sequence of a Coleopteran organism comprising SEQ ID NO:50; native coding sequence of a Coleopteran organism comprising SEQ ID NOs:52-53; the complement of a native coding sequence of a Coleopteran organism comprising of SEQ ID NOs:52-53; a fragment of at least 15 contiguous nucleotides of a native coding sequence of a Coleopteran organism comprising SEQ ID NOs:52-53; the complement of a fragment of at least 15 contiguous nucleotides of a native coding sequence of a Coleopteran organism comprising SEQ ID NOs:52-53;
SEQ ID NO:54; the complement of SEQ ID NO:54; a fragment of at least 15 contiguous nucleotides of SEQ ID NO:54; the complement of a fragment of at least 15 contiguous nucleotides of SEQ ID NO:54; a native coding sequence of a coleopteran organism comprising SEQ ID NO:54; the complement of a native coding sequence of a coleopteran organism comprising SEQ ID NO:54; a fragment of at least 15 contiguous nucleotides of a native coding sequence of a coleopteran organism comprising SEQ ID NO:54; the complement of a fragment of at least 15 contiguous nucleotides of a native coding sequence of a Coleopteran organism comprising SEQ ID NO:54; native coding sequence of a Coleopteran organism comprising SEQ ID NOs:56-58; the complement of a native coding sequence of a Coleopteran organism comprising of SEQ ID NOs:56-58; a fragment of at least 15 contiguous nucleotides of a native coding sequence of a Coleopteran organism comprising SEQ ID NOs:56-58; the complement of a fragment of at least 15 contiguous nucleotides of a native coding sequence of a Coleopteran organism comprising SEQ ID NOs:56-85;
SEQ ID NO:59; the complement of SEQ ID NO:59; a fragment of at least 15 contiguous nucleotides of SEQ ID NO:59; the complement of a fragment of at least 15 contiguous nucleotides of SEQ ID NO:59; a native coding sequence of a coleopteran organism comprising SEQ ID NO:59; the complement of a native coding sequence of a coleopteran organism comprising SEQ ID NO:59; a fragment of at least 15 contiguous nucleotides of a native coding sequence of a coleopteran organism comprising SEQ ID NO:59; the complement of a fragment of at least 15 contiguous nucleotides of a native coding sequence of a Coleopteran organism comprising SEQ ID NO:59; native coding sequence of a Coleopteran organism comprising SEQ ID NOs:61-63; the complement of a native coding sequence of a Coleopteran organism comprising of SEQ ID NOs:61-63; a fragment of at least 15 contiguous nucleotides of a native coding sequence of a Coleopteran organism comprising SEQ ID NOs:61-63; the complement of a fragment of at least 15 contiguous nucleotides of a native coding sequence of a Coleopteran organism comprising SEQ ID NOs:61-63;
SEQ ID NO:64; the complement of SEQ ID NO:64; a fragment of at least 15 contiguous nucleotides of SEQ ID NO:64; the complement of a fragment of at least 15 contiguous nucleotides of SEQ ID NO:64; a native coding sequence of a coleopteran organism comprising SEQ ID NO:64; the complement of a native coding sequence of a coleopteran organism comprising SEQ ID NO:64; a fragment of at least 15 contiguous nucleotides of a native coding sequence of a coleopteran organism comprising SEQ ID NO:64; the complement of a fragment of at least 15 contiguous nucleotides of a native coding sequence of a Coleopteran organism comprising SEQ ID NO:64; native coding sequence of a Coleopteran organism comprising SEQ ID NOs:66-67; the complement of a native coding sequence of a Coleopteran organism comprising of SEQ ID NOs:66-67; a fragment of at least 15 contiguous nucleotides of a native coding sequence of a Coleopteran organism comprising SEQ ID NOs:66-67; the complement of a fragment of at least 15 contiguous nucleotides of a native coding sequence of a Coleopteran organism comprising SEQ ID NOs:66-67;
SEQ ID NO:68; the complement of SEQ ID NO:68; a fragment of at least 15 contiguous nucleotides of SEQ ID NO:68; the complement of a fragment of at least 15 contiguous nucleotides of SEQ ID NO:68; a native coding sequence of a coleopteran organism comprising SEQ ID NO:68; the complement of a native coding sequence of a coleopteran organism comprising SEQ ID NO:68; a fragment of at least 15 contiguous nucleotides of a native coding sequence of a coleopteran organism comprising SEQ ID NO:68; the complement of a fragment of at least 15 contiguous nucleotides of a native coding sequence of a Coleopteran organism comprising SEQ ID NO:68; native coding sequence of a Coleopteran organism comprising SEQ ID NOs:70-71; the complement of a native coding sequence of a Coleopteran organism comprising of SEQ ID NOs:70-71; a fragment of at least 15 contiguous nucleotides of a native coding sequence of a Coleopteran organism comprising SEQ ID NOs:70-71; the complement of a fragment of at least 15 contiguous nucleotides of a native coding sequence of a Coleopteran organism comprising SEQ ID NOs:70-71;
SEQ ID NO:72; the complement of SEQ ID NO:72; a fragment of at least 15 contiguous nucleotides of SEQ ID NO:72; the complement of a fragment of at least 15 contiguous nucleotides of SEQ ID NO:72; a native coding sequence of a coleopteran organism comprising SEQ ID NO:72; the complement of a native coding sequence of a coleopteran organism comprising SEQ ID NO:72; a fragment of at least 15 contiguous nucleotides of a native coding sequence of a coleopteran organism comprising SEQ ID NO:72; the complement of a fragment of at least 15 contiguous nucleotides of a native coding sequence of a Coleopteran organism comprising SEQ ID NO:72; native coding sequence of a Coleopteran organism comprising SEQ ID NOs:74-75; the complement of a native coding sequence of a Coleopteran organism comprising of SEQ ID NOs:74-75; a fragment of at least 15 contiguous nucleotides of a native coding sequence of a Coleopteran organism comprising SEQ ID NOs:74-75; the complement of a fragment of at least 15 contiguous nucleotides of a native coding sequence of a Coleopteran organism comprising SEQ ID NOs:74-75;
SEQ ID NO:76; the complement of SEQ ID NO:76; a fragment of at least 15 contiguous nucleotides of SEQ ID NO:76; the complement of a fragment of at least 15 contiguous nucleotides of SEQ ID NO:76; a native coding sequence of a coleopteran organism comprising SEQ ID NO:76; the complement of a native coding sequence of a coleopteran organism comprising SEQ ID NO:76; a fragment of at least 15 contiguous nucleotides of a native coding sequence of a coleopteran organism comprising SEQ ID NO:76; the complement of a fragment of at least 15 contiguous nucleotides of a native coding sequence of a Coleopteran organism comprising SEQ ID NO:76; native coding sequence of a Coleopteran organism comprising SEQ ID NOs:78-79; the complement of a native coding sequence of a Coleopteran organism comprising of SEQ ID NOs:78-79; a fragment of at least 15 contiguous nucleotides of a native coding sequence of a Coleopteran organism comprising SEQ ID NOs:78-79; the complement of a fragment of at least 15 contiguous nucleotides of a native coding sequence of a Coleopteran organism comprising SEQ ID NOs:78-79;
SEQ ID NO:80; the complement of SEQ ID NO:80; a fragment of at least 15 contiguous nucleotides of SEQ ID NO:80; the complement of a fragment of at least 15 contiguous nucleotides of SEQ ID NO:80; a native coding sequence of a coleopteran organism comprising SEQ ID NO:80; the complement of a native coding sequence of a coleopteran organism comprising SEQ ID NO:80; a fragment of at least 15 contiguous nucleotides of a native coding sequence of a coleopteran organism comprising SEQ ID NO:80; the complement of a fragment of at least 15 contiguous nucleotides of a native coding sequence of a Coleopteran organism comprising SEQ ID NO:80; native coding sequence of a Coleopteran organism comprising SEQ ID NOs:82-85; the complement of a native coding sequence of a Coleopteran organism comprising of SEQ ID NOs:82-85; a fragment of at least 15 contiguous nucleotides of a native coding sequence of a Coleopteran organism comprising SEQ ID NOs:82-85; the complement of a fragment of at least 15 contiguous nucleotides of a native coding sequence of a Coleopteran organism comprising SEQ ID NOs:82-85;
SEQ ID NO:86; the complement of SEQ ID NO:86; a fragment of at least 15 contiguous nucleotides of SEQ ID NO:86; the complement of a fragment of at least 15 contiguous nucleotides of SEQ ID NO:86; a native coding sequence of a coleopteran organism comprising SEQ ID NO:86; the complement of a native coding sequence of a coleopteran organism comprising SEQ ID NO:86; a fragment of at least 15 contiguous nucleotides of a native coding sequence of a coleopteran organism comprising SEQ ID NO:86; the complement of a fragment of at least 15 contiguous nucleotides of a native coding sequence of a Coleopteran organism comprising SEQ ID NO:86; native coding sequence of a Coleopteran organism comprising SEQ ID NOs:88-89; the complement of a native coding sequence of a Coleopteran organism comprising of SEQ ID NOs:88-89; a fragment of at least 15 contiguous nucleotides of a native coding sequence of a Coleopteran organism comprising SEQ ID NOs:88-89; the complement of a fragment of at least 15 contiguous nucleotides of a native coding sequence of a Coleopteran organism comprising SEQ ID NOs:88-89;
SEQ ID NO:90; the complement of SEQ ID NO:90; a fragment of at least 15 contiguous nucleotides of SEQ ID NO:90; the complement of a fragment of at least 15 contiguous nucleotides of SEQ ID NO:90; a native coding sequence of a coleopteran organism comprising SEQ ID NO:90; the complement of a native coding sequence of a coleopteran organism comprising SEQ ID NO:90; a fragment of at least 15 contiguous nucleotides of a native coding sequence of a coleopteran organism comprising SEQ ID NO:90; the complement of a fragment of at least 15 contiguous nucleotides of a native coding sequence of a Coleopteran organism comprising SEQ ID NO:90; native coding sequence of a Coleopteran organism comprising SEQ ID NOs:92-93; the complement of a native coding sequence of a Coleopteran organism comprising of SEQ ID NOs:92-93; a fragment of at least 15 contiguous nucleotides of a native coding sequence of a Coleopteran organism comprising SEQ ID NOs:92-93; the complement of a fragment of at least 15 contiguous nucleotides of a native coding sequence of a Coleopteran organism comprising SEQ ID NOs:92-93;
SEQ ID NO:94; the complement of SEQ ID NO:94; a fragment of at least 15 contiguous nucleotides of SEQ ID NO:94; the complement of a fragment of at least 15 contiguous nucleotides of SEQ ID NO:94; a native coding sequence of a coleopteran organism comprising SEQ ID NO:94; the complement of a native coding sequence of a coleopteran organism comprising SEQ ID NO:94; a fragment of at least 15 contiguous nucleotides of a native coding sequence of a coleopteran organism comprising SEQ ID NO:94; the complement of a fragment of at least 15 contiguous nucleotides of a native coding sequence of a Coleopteran organism comprising SEQ ID NO:94; native coding sequence of a Coleopteran organism comprising SEQ ID NOs:96-97; the complement of a native coding sequence of a Coleopteran organism comprising of SEQ ID NOs:96-97; a fragment of at least 15 contiguous nucleotides of a native coding sequence of a Coleopteran organism comprising SEQ ID NOs:96-97; and the complement of a fragment of at least 15 contiguous nucleotides of a native coding sequence of a Coleopteran organism comprising SEQ ID NOs:96-97.

2. The polynucleotide of claim 1, wherein the polynucleotide is selected from the group consisting of SEQ ID NO:1, SEQ ID NO:3, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:13, SEQ ID NO:15, SEQ ID NO:16, SEQ ID NO:17, SEQ ID NO:19, SEQ ID NO:20, SEQ ID NO:21, SEQ ID NO:23, SEQ ID NO:24, SEQ ID NO:25, SEQ ID NO:26, SEQ ID NO:28, SEQ ID NO:29, SEQ ID NO:30, SEQ ID NO:32, SEQ ID NO:33, SEQ ID NO:34, SEQ ID NO:35, SEQ ID NO:37, SEQ ID NO:38, SEQ ID NO:39, SEQ ID NO:40, SEQ ID NO:42, SEQ ID NO:43, SEQ ID NO:44, SEQ ID NO:45, SEQ ID NO:47, SEQ ID NO:48, SEQ ID NO:49, SEQ ID NO:50, SEQ ID NO:52, SEQ ID NO:53, SEQ ID NO:54, SEQ ID NO:56, SEQ ID NO:57, SEQ ID NO:58, SEQ ID NO:59, SEQ ID NO:61, SEQ ID NO:62, SEQ ID NO:63, SEQ ID NO:64, SEQ ID NO:66, SEQ ID NO:67, SEQ ID NO:68, SEQ ID NO:70, SEQ ID NO:71, SEQ ID NO:72, SEQ ID NO:74, SEQ ID NO:75, SEQ ID NO:76, SEQ ID NO:78, SEQ ID NO:79, SEQ ID NO:80, SEQ ID NO:82, SEQ ID NO:83, SEQ ID NO:84, SEQ ID NO:85, SEQ ID NO:86, SEQ ID NO:88, SEQ ID NO:89, SEQ ID NO:90, SEQ ID NO:92, SEQ ID NO:93, SEQ ID NO:94, SEQ ID NO:96, SEQ ID NO:97, and the complements of any of the foregoing.

3. A plant transformation vector comprising the polynucleotide of claim 1.

4. The polynucleotide of claim 1, wherein the organism is selected from the group consisting of D. v. virgifera LeConte (western corn rootworm, “WCR”); D. barberi Smith and Lawrence (northern corn rootworm, “NCR”); D. u. howardi Barber (southern corn rootworm, “SCR”); D. v. zeae Krysan and Smith (Mexican corn rootworm, “MCR”); D. balteata LeConte; D. u. tenella; D. speciosa Germar; and D. u. undecimpunctata Mannerheim.

5. A ribonucleic acid (RNA) molecule transcribed from the polynucleotide of claim 1.

6. A double-stranded ribonucleic acid molecule produced from the expression of the polynucleotide of claim 1.

7. The double-stranded ribonucleic acid molecule of claim 6, wherein contacting the polynucleotide sequence with coleopteran pest inhibits the expression of an endogenous nucleotide sequence specifically complementary to the polynucleotide.

8. The double-stranded ribonucleic acid molecule of claim 7, wherein contacting said ribonucleotide molecule with a coleopteran pest kills or inhibits the growth, and/or feeding of the pest.

9. The double stranded RNA of claim 6, comprising a first, a second and a third RNA segment, wherein the first RNA segment comprises the polynucleotide, wherein the third RNA segment is linked to the first RNA segment by the second polynucleotide sequence, and wherein the third RNA segment is substantially the reverse complement of the first RNA segment, such that the first and the third RNA segments hybridize when transcribed into a ribonucleic acid to form the double-stranded RNA.

10. The RNA of claim 5, selected from the group consisting of a double-stranded ribonucleic acid molecule and a single-stranded ribonucleic acid molecule of between about 15 and about 30 nucleotides in length.

11. A plant transformation vector comprising the polynucleotide of claim 1, wherein the heterologous promoter is functional in a plant cell.

12. A cell transformed with the polynucleotide of claim 1.

13. The cell of claim 12, wherein the cell is a prokaryotic cell.

14. The cell of claim 12, wherein the cell is a eukaryotic cell.

15. The cell of claim 14, wherein the cell is a plant cell.

16. A plant transformed with the polynucleotide of claim 1.

17. A seed of the plant of claim 16, wherein the seed comprises the polynucleotide.

18. A commodity product produced from the plant of claim 16, wherein the commodity product comprises a detectable amount of the polynucleotide.

19. The plant of claim 16, wherein the at least one polynucleotide is expressed in the plant as a double-stranded ribonucleic acid molecule.

20. The cell of claim 15, wherein the cell is a maize cell.

21. The plant of claim 16, wherein the plant is maize.

22. The plant of claim 16, wherein the at least one polynucleotide is expressed in the plant as a ribonucleic acid molecule, and the ribonucleic acid molecule inhibits the expression of an endogenous polynucleotide that is specifically complementary to the at least one polynucleotide when a coleopteran pest ingests a part of the plant.

23. The polynucleotide of claim 1, further comprising at least one additional polynucleotide that encodes an RNA molecule that inhibits the expression of an endogenous pest gene.

24. A plant transformation vector comprising the polynucleotide of claim 23, wherein the additional polynucleotide(s) are each operably linked to a heterologous promoter functional in a plant cell.

25. A method for controlling a coleopteran pest population, the method comprising:
providing in a host plant of a coleopteran pest a transformed plant cell comprising the polynucleotide of claim 1, wherein the polynucleotide is expressed to produce a ribonucleic acid molecule that functions upon contact with a coleopteran pest belonging to the population to inhibit the expression of a target sequence within the r coleopteran pest and results in decreased growth and/or survival of the coleopteran pest or pest population, relative to the same pest species on a plant of the same host plant species that does not comprise the polynucleotide.

26. The method according to claim 25, wherein the ribonucleic acid molecule is a double-stranded ribonucleic acid molecule.

27. The method according to claim 25, wherein the coleopteran pest population is reduced relative to a population of the same pest species infesting a host plant of the same host plant species lacking the transformed plant cell.

28. The method according to claim 25, wherein the ribonucleic acid molecule is a double-stranded ribonucleic acid molecule.

29. The method according to claim 26, wherein the coleopteran pest population is reduced relative to a coleopteran pest population infesting a host plant of the same species lacking the transformed plant cell.

30. A method for improving the yield of a corn crop, the method comprising:
introducing the nucleic acid of claim 1 into a corn plant to produce a transgenic corn plant; and
cultivating the corn plant to allow the expression of the at least one polynucleotide; wherein expression of the at least one polynucleotide inhibits the development or growth of a coleopteran pest and loss of yield due to infection by the coleopteran pest.

31. The method according to claim 30, wherein expression of the at least one polynucleotide produces an RNA molecule that suppresses at least a first target gene in a coleopteran pest that has contacted a portion of the corn plant.

32. A method for producing a transgenic plant cell, the method comprising:
transforming a plant cell with a vector comprising the nucleic acid of claim 1;
culturing the transformed plant cell under conditions sufficient to allow for development of a plant cell culture comprising a plurality of transformed plant cells;
selecting for transformed plant cells that have integrated the at least one polynucleotide into their genomes;
screening the transformed plant cells for expression of a ribonucleic acid (RNA) molecule encoded by the at least one polynucleotide; and
selecting a plant cell that expresses the RNA.

33. The method according to claim 32, wherein the RNA molecule is a double-stranded RNA molecule.

34. A method for producing a coleopteran pest-resistant transgenic plant, the method comprising:
providing the transgenic plant cell produced by the method of claim 32; and
regenerating a transgenic plant from the transgenic plant cell, wherein expression of the ribonucleic acid molecule encoded by the at least one polynucleotide is sufficient to modulate the expression of a target gene in a coleopteran pest that contacts the transformed plant

35. A method for producing a transgenic plant cell, the method comprising:
transforming a plant cell with a vector comprising a means for providing coleopteran pest resistance to a plant;
culturing the transformed plant cell under conditions sufficient to allow for development of a plant cell culture comprising a plurality of transformed plant cells;
selecting for transformed plant cells that have integrated the means for providing coleopteran pest resistance to a plant into their genomes;
screening the transformed plant cells for expression of a means for inhibiting expression of an essential gene in a coleopteran pest; and
selecting a plant cell that expresses the means for inhibiting expression of an essential gene in a coleopteran pest.

36. A method for producing a coleopteran pest-resistant transgenic plant, the method comprising:
providing the transgenic plant cell produced by the method of claim 44; and
regenerating a transgenic plant from the transgenic plant cell, wherein expression of the means for inhibiting expression of an essential gene in a coleopteran pest is sufficient to modulate the expression of a target gene in a coleopteran pest that contacts the transformed plant.

37. The nucleic acid of claim 1, further comprising a polynucleotide encoding a polypeptide from Bacillus thuringiensis.

38. The nucleic acid of claim 37, wherein the polypeptide from B. thuringiensis is selected from a group comprising Cry1, Cry3, Cry6, Cry7, Cry8, Cry9, Cry14, Cry18, Cry22, Cry23, Cry34, Cry35, Cry36, Cry37, Cry43, Cry55, Cyt1, and Cyt2.

39. The cell of claim 15, wherein the cell comprises a polynucleotide encoding a polypeptide from Bacillus thuringiensis.

40. The cell of claim 39, wherein the polypeptide from B. thuringiensis is selected from a group comprising Cry1, Cry3, Cry6, Cry7, Cry8, Cry9, Cry14, Cry18, Cry22, Cry23, Cry34, Cry35, Cry36, Cry37, Cry43, Cry55, Cyt1, and Cyt2.

41. The plant of claim 16, wherein the plant comprises a polynucleotide encoding a polypeptide from Bacillus thuringiensis.

42. The plant of claim 41, wherein the polypeptide from B. thuringiensis is selected from a group comprising Cry1, Cry3, Cry6, Cry7, Cry8, Cry9, Cry14, Cry18, Cry22, Cry23, Cry34, Cry35, Cry36, Cry37, Cry43, Cry55, Cyt1, and Cyt2.

43. The method according to claim 32, wherein the transformed plant cell comprises a nucleotide sequence encoding a polypeptide from Bacillus thuringiensis

44. The method according to claim 41, wherein the polypeptide from B. thuringiensis is selected from a group comprising Cry1, Cry3, Cry6, Cry7, Cry8, Cry9, Cry14, Cry18, Cry22, Cry23, Cry34, Cry35, Cry36, Cry37, Cry43, Cry55, Cyt1, and Cyt2.

45. A method for improving the yield of a plant crop, the method comprising:
introducing a nucleic acid of into a corn plant to produce a transgenic plant, wherein the nucleic acid comprises more than one of
a polynucleotide encoding at least one siRNA targeting a gene selected from the group consisting of: chitin synthase, outer membrane translocase, double parked, discs overgrown, ctf4, rpl9, serpin protease inhibitor I4, myosin 3 LC, megator, g-protein beta subunit, flap wing, female sterile 2 ketel, enhancer of polycomb, dead box 73D, cg7000, heat shock protein 70-331, heat shock protein 70-12300, rnr1, elav, pten, and cdc8;
a polynucleotide encoding an insecticidal polypeptide from Bacillus thuringiensis, and
cultivating the plant to allow the expression of the at least one polynucleotide; wherein expression of the at least one polynucleotide inhibits coleopteran pest development or growth and loss of yield due to coleopteran pest infection.

46. The method according to claim 43, wherein the plant is maize, soybean or cotton.

47. The double stranded RNA of claim 6, comprising a first, a second and a third RNA segment, wherein the first RNA segment comprises the polynucleotide, wherein the third RNA segment is linked to the first RNA segment by the second polynucleotide sequence, and wherein the third RNA segment is substantially the reverse complement of the first RNA segment, such that the first and the third RNA segments hybridize when transcribed into a ribonucleic acid to form the double-stranded RNA.
